(19) United States
(12) Patent Application Publication
(10) Pub. No.: US 2018/0235917 A1
(43) Pub. Date: Aug. 23, 2018

(54) COMPOSITIONS, METHODS AND KITS FOR TREATMENT OF DIABETES AND/OR HYPERLIPIDEMIA

(71) Applicant: NuSirt Sciences, Inc., Nashville, TN (US)

(72) Inventors: Michael ZEMEL, Knoxville, TN (US); Antje BRUCKBAUER, Knoxville, TN (US)

(73) Assignee: NuSirt Sciences, Inc., Nashville, TN (US)

(21) Appl. No.: 15/513,115

(22) PCT Filed: Sep. 23, 2015
(86) PCT No.: PCT/US2015/051793

§ 371 Date: Mar. 21, 2017

Related U.S. Application Data
(60) Provisional application No. 62/054,921, filed on Sep. 24, 2014.

(51) Int. Cl. A61K 31/198; A61K 31/455; A61K 31/155; A61K 31/706; A61K 45/06; A61P 3/10; A61P 3/06

(52) U.S. Cl. CPC A61K 31/198; A61K 31/455; A61K 31/155; A61K 2300/00; A61K 45/06; A61P 3/10; A61P 3/06; A61K 31/706

(57) ABSTRACT

Compositions, methods and kits for treatment of diabetes and/or hyperlipidemia are provided herein. Such compositions can contain synergizing amounts of leucine and/or one or more leucine metabolites in combination with nicotinic acid, nicotinamide riboside and/or nicotinic acid metabolites, and with at least one or more anti-diabetic agents. Such compositions can contain sub-therapeutic amounts of nicotinic acid, nicotinamide riboside and/or nicotinic acid metabolites, and/or sub-therapeutic amounts of one or more anti-diabetic agents that can achieve the same therapeutic efficacy as therapeutic amounts of said compositions in diabetes and/or hyperlipidemia medicaments. The composition can also reduce the side effects associated with treatment using anti-diabetic agents and/or nicotinic acid.

### CROSS-REFERENCE TO RELATED APPLICATIONS

This present application claims priority to U.S. Provisional Application Ser. No. 62/054,921, filed Sep. 24, 2014, which is hereby incorporated by reference in its entirety.

### BACKGROUND

Metabolic disorders, such as hyperlipidemia, diabetes, high cholesterol, arteriosclerosis, hypertension and obesity, and the related diseases present a significant burden to public health. For instance, obesity, clinically defined as a body mass index of over 30 kg/m2, is estimated to affect 35.7% of the U.S. adult population. In the U.S., obesity is estimated to cause roughly 110,000-365,000 deaths per year. Obesity can result in hyperlipidemia, characterized by an excess of lipids, including cholesterol, cholesterol esters, phospholipids, and triglycerides, in the bloodstream. Additionally, obesity can result in diabetes and other related diseases. Diabetes is a metabolic disorder characterized by high blood glucose levels or low glucose tolerance, and is estimated to affect 8% of the U.S. population. Obesity is also associated with vascular disease, cancer, renal disease, infectious diseases, external causes, intentional self-harm, nervous system disorders, and chronic pulmonary disease (N Engl J Med 2011; 364:829-841). Metabolic syndrome, in which subjects present with central obesity and at least two other metabolic disorders (such as high cholesterol, high blood pressure, or diabetes), is estimated to affect 25% of the U.S. population.

Nicotinic acid a form of vitamin B3 (niacin) has been used to treat hyperlipidemia which is one of the symptoms of obesity and other conditions. When taken in high doses (1-4 g/day typically; maximum clinical dose is 6 g/day), nicotinic acid can treat hyperlipidemia, as it can lower total lipid, LDL, cholesterol, triglycerides, and lipoprotein, or raise HDL lipoprotein in the bloodstream. It can also reduce atherosclerotic plaque progression and coronary heart disease morbidity and mortality.

Diabetes, also sometimes associate with obesity, can be treated with anti-diabetic agents such as metformin. Metformin, along with phenformin and buformin, is a form of biguanide, which is a guanide. When ingested (1000-2550 mg/day), metformin can treat diabetes by increasing insulin sensitivity, increasing glucose uptake in the gut, increasing glucose utilization, and lowering blood glucose level. Metformin does not increase the amount of insulin produced by the body; thus generally does not cause hypoglycemia, as many other diabetes medications can do.

Many efforts have been attempted to develop treatments for metabolic disorders such as hyperlipidemia and diabetes. However, both nicotinic acid and metformin can have significant side-effect and hence can be generally poorly tolerated. For instance, one significant side-effect of nicotinic acid involves severe cutaneous vasodilation and flushing responses. Such well-documented side-effects have limited the prescription of nicotinic acid. (Carlson L A. Nicotinic acid: the broad-spectrum lipid drug. A 50th anniversary review. J Int Med 2005; 258:94-114). Current anti-diabetic agents such as metformin is associated with other adverse effects. Amongst them are common adverse gastrointestinal effects that cause discomfort and limit effective dosing, as well as the rare but serious adverse event of lactic acidosis. While side effects are somewhat attenuated in sustained (SR) and extended (ER) release preparations, the side effects persist sufficiently to limit the usage of these otherwise effective drugs.

### SUMMARY OF THE INVENTION

There remains a great need for treatments that can address glycemic control and hyperlipidemia in patients with minimal side effects. The present invention addresses these needs and provides related advantages as well.

The subject application provides compositions, methods, and kits useful for inducing an increase in insulin sensitivity, glucose utilization, fatty acid oxidation, and/or a reduction in lipid accumulation in a subject; and thus is useful for preventing and treatment of diabetes and/or hyperlipidemia.

In one aspect of the invention, the compositions, methods, and kits can contain amounts of (a) leucine and/or at least one or more leucine metabolites in combination, (b) one or more agents selected from the group consisting of nicotinic acid, nicotinamide riboside, and nicotinic acid metabolite, and (c) at least one or more anti-diabetic agents.

In another aspect of the invention, the compositions, methods, and kits can contain amounts of (a) leucine and/or at least one or more leucine metabolites in combination and (b) at least one or more anti-diabetic agents that is a guanide. The guanide may have a dimethyl structure, for example, metformin, dimethylguanidine, and/or galegine.

Such compositions can contain sub-therapeutic amounts of nicotinic acid, nicotinamide riboside, nicotinic acid metabolites and/or anti-diabetic agents that have the same effectiveness in treating diabetes and/or hyperlipidemia as therapeutic amounts of such components. The present invention also addresses the side effects of treating subjects with anti-diabetic agents (e.g. lactic acidosis and hypoglycemia) for diabetes, and the side effects of high doses of nicotinic acid (e.g. cutaneous vasodilation and flushing responses) for hyperlipidemia, that are prevalent in certain diabetes or hyperlipidemia medications.

The invention provides for a composition comprising (a) leucine and/or at least one or more leucine metabolites; and (b) one or more agents selected from the group consisting of nicotinic acid, nicotinamide riboside, and nicotinic acid metabolite; and (c) at least one or more anti-diabetic agents. The weight percentage of component (a) can be between about 80%-98% of the total composition. The weight percentage of component (b) can be between about 1%-5% of the total composition. The weight percentage of component (c) can be between about 1%-15% of the total composition.

The amount of leucine in the composition can be at least about 250 mg. The amount of one or more leucine metabolites can be at least about 25 mg. The amount of leucine and/or one or more leucine metabolites can be less than about 1 or 3 g.

The amount of one or more agents selected from the group consisting of nicotinic acid, nicotinamide riboside, and nicotinic acid metabolite can be at least about 1 mg. The amount of one or more agents selected from the group consisting of nicotinic acid, nicotinamide riboside, and nicotinic acid metabolite can be less than about 250 mg. The amount of one or more agents selected from the group consisting of nicotinic acid, nicotinamide riboside, and nicotinic acid metabolite can be between about 1-100 mg. The amount of one or more agents selected from the group consisting of nicotinic acid, nicotinamide riboside, and nicotinic acid metabolite can be less than about 1 g.

The amount of the one or more anti-diabetic agent and/or any analog thereof can be between 0.1-2550 mg. The amount of the one or more anti-diabetic agent and/or any analog thereof can be between 0.1-500 mg. The amount of the one or more anti-diabetic agent and/or any analog thereof can be between 1-200 mg. The amount of the one or more anti-diabetic agents and any analog thereof can be at least about 0.1 mg. The amount of the one or more anti-diabetic agents and/or any analog thereof can be less than about 2.5 g.

The one or more leucine metabolites can be selected from the group consisting of keto-isocaproic acid (KIC), alpha-hydroxy-isocaproic acid, and HMB.

The one or more anti-diabetic agent can be selected from the group consisting of biguanide, metformin, phenformin, buformin, galegine, dimethylguanidine, guanide, thiazolidinedione, rosiglitazone, meglitinides, alpha glucosidase inhibitors, sulfonylureas, incretins, ergot alkaloids, DPP inhibitors, and any combination thereof.

The one or more anti-diabetic agent and/or any analog thereof can be a guanide.

The invention provides for a composition comprising (a) leucine and/or at least one or more leucine metabolites; and (b) one or more agents selected from the group consisting of nicotinic acid, nicotinamide riboside, and nicotinic acid metabolite; and (c) at least one or more anti-diabetic agents. The component (a) in the composition can be leucine. The component (b) in the composition can be nicotinic acid. The component (c) in the composition can be metformin. The component (c) in the composition can be an analog of metformin, or a precursor of metformin.

The invention provides for a composition comprising (a) leucine and/or at least one or more leucine metabolites; and (b) one or more agents selected from the group consisting of nicotinic acid, nicotinamide riboside, and nicotinic acid metabolite; and (c) at least one or more anti-diabetic agents. The molar ratio of component (a) to component (b) in said composition can be greater than about 20 or 200 or 2000. The molar ratio of component (a) to component (c) in said composition can be greater than about 20 or 200 or 2000.

The composition can be substantially free of nicotinamide. The composition does not contain nicotinamide. The composition can be substantially free of nicotinic acid metabolites. The composition can be substantially free of each of nicotinyl CoA, nicotinuric acid, nicotinate mononucleotide, nicotinate adenine dinucleotide, and nicotinamide adenine dinucleotide.

The composition can be substantially free of non-branched amino acids. The composition can be substantially free of each amino acid selected from the group consisting of alanine, arginine, asparagine, aspartic acid, cysteine, glutamic acid, glutamine, glycine, histidine, lysine, methionine, phenylalanine, proline, serine, threonine, tryptophan, valine, isoleucine and tyrosine. The composition contains less than about 0.1% of each free amino acid selected from the group consisting of alanine, arginine, asparagine, aspartic acid, cysteine, glutamic acid, glutamine, glycine, histidine, lysine, methionine, phenylalanine, proline, serine, threonine, tryptophan, valine, isoleucine and tyrosine. The composition can be substantially free of each of alanine, glycine, glutamic acid, and proline. The composition can contain less than about 10% of non-leucine amino acids.

The composition can be substantially free of resveratrol. The composition may not contain resveratrol.

The amount of one or more agents selected from the group consisting of nicotinic acid, nicotinamide riboside, and nicotinic acid metabolite can, when administered to a subject, yield a serum level of the agent(s) that can be between about 1-1000 nM. The amount of one or more agents selected from the group consisting of nicotinic acid, nicotinamide riboside, and nicotinic acid metabolite can, when administered to a subject, yield a serum level of the agent(s) that can be between about 10-500 nM. The amount of one or more agents selected from the group consisting of nicotinic acid, nicotinamide riboside, and nicotinic acid metabolite can, when administered to a subject, yield a serum level of the agent(s) that can be between about 1-100 nM.

The amount of leucine and/or one or more leucine metabolites can, when administered to a subject, yield a serum level of the leucine and/or one or more leucine metabolites that can be between 0.3-0.5 mM. The amount of leucine and/or one or more leucine metabolites can, when administered to a subject, yield a serum level of the leucine and/or one or more leucine metabolites that can be about 0.5 mM.

The amount of the one or more anti-diabetic agent and/or any analog thereof can, when administered to a subject, yield a serum level of the one or more anti-diabetic agent or any analog thereof that can be between about 1-100 μM.

The invention provides for a composition comprising (a) leucine and/or at least one or more leucine metabolites; and (b) one or more agents selected from the group consisting of nicotinic acid, nicotinamide riboside, and nicotinic acid metabolite; and (c) at least one or more anti-diabetic agents. The amount of nicotinic acid and/or nicotinic acid metabolites can be insufficient to reduce lipid content in the absence of component (a) and/or component (c). The amount of component (a) and (b) and (c) synergistically lowers lipid accumulation, increases fat oxidation, increases insulin sensitivity, increases glucose utilization, or increases activation of one or more components in the sirtuin pathway in said subject when administered to the subject as compared to administering a subject component (a) or component (b) or component (c) alone. The one or more components in the sirtuin pathway can be SIRT1, and/or SIRT3, and/or AMPK, and/or PCG1α.

The invention provides for a composition comprising (a) leucine and/or at least one or more leucine metabolites; and (b) one or more agents selected from the group consisting of nicotinic acid, nicotinamide riboside, and nicotinic acid metabolite; and (c) at least one or more anti-diabetic agents. The amount of component (a) and (b) and (c) can synergistically increase fat oxidation in said subject when administered to the subject as compared to administering a subject component (a) and component (b), or component (a) and component (c)

The composition can be contained in a foodstuff. A portion of the leucine and/or one or more leucine metabolites can be in a free form or salt form. The composition can be formulated for oral administration. The composition can be a tablet, a capsule, a pill, a granule, an emulsion, a gel, a plurality of beads encapsulated in a capsule, a powder, a suspension, a liquid, a semi-liquid, a semi-solid, a syrup, a slurry or a chewable form. The composition can be formulated in a unit dosage form.

The invention provides for a composition comprising (a) leucine and/or at least one or more leucine metabolites; and (b) one or more agents selected from the group consisting of nicotinic acid, nicotinamide riboside, and nicotinic acid metabolite; and (c) at least one or more anti-diabetic agents. Component (a) and component (b) and component (c) can be separately packaged. Component (a) and component (b) and component (c) can be mixed.

The composition can further comprise one or more therapeutic agents that can be capable of lowering lipid accumulation, and/or increasing fat oxidation, and/or increasing insulin sensitivity, and/or increasing glucose utilization. The one or more therapeutic agents can be selected from the group consisting of HMG-CoA inhibitor, fibrate, bile acid sequestrant, ezetimibe, lomitapide, phytosterols, CETP antagonist, orlistat, and any combination thereof.

In another aspect, the invention provides for a method of reducing atherosclerotic plaque size in a subject in need thereof, comprising administering to said subject a dose of a composition described herein comprising an amount of leucine and/or one or more leucine metabolites and an amount of nicotinic acid and/or nicotinic acid metabolites to effect a reduction of the total atherosclerotic plaque size in the subject.

The invention provides for a method of lowering lipid accumulation in a subject in need thereof, comprising administering to said subject a composition described herein to effect a lowering of the lipid accumulation in the subject. The invention provides for a method of increasing insulin sensitivity in a subject in need thereof, comprising administering to said subject a composition described herein to effect an increasing the insulin sensitivity in the subject. The invention provides for a method of increasing glucose utilization in a subject in need thereof, comprising administering to said subject a composition described herein to effect increasing the glucose utilization in the subject. The invention provides for a method of increasing fat oxidation in a subject in need thereof, comprising administering to said subject a composition described herein to effect increasing the fat oxidation in the subject.

The invention provides for a method of treating diabetes and/or hyperlipidemia comprising administering to the subject a composition described herein over a time period, over a time period, during which the subject exhibits one or more of (1) an increase in insulin sensitivity, glucose utilization, or fat oxidation or (2) a reduction in lipid accumulation. The invention provides for a method of reducing atherosclerotic plaque size in a subject in need thereof, comprising administering to said subject a dose of a composition described herein to effect a reduction in the total atherosclerotic plaque size in the subject.

The invention provides for a kit comprising a multi-day supply of unit dosages of a composition described herein and instructions directing the administration of said multi-day supply over a period of multiple days.

Another aspect of the invention provides for a composition comprising: (a) leucine and/or at least one or more leucine metabolites; and (b) at least one or more anti-diabetic agents comprising a guanide. Component (b) can further contain a dimethyl structure. The guanide can be galegine. The guanide can be dimethylguanidine. The weight percentage of component (a) can be between about 80%-98% of the total composition. The weight percentage of component (b) can be between about 2%-20% of the total composition.

The amount of leucine can be at least about 250 mg. The amount of one or more leucine metabolites can be at least about 25 mg. The amount of leucine and/or one or more leucine metabolites can be less than about 1 or 3 g.

The amount of the one or more anti-diabetic agent and/or any analog thereof can be between 100-2550 mg. The amount of the one or more anti-diabetic agents and any analog thereof can be at least about 0.1 mg. The amount of the one or more anti-diabetic agents and/or any analog thereof can be less than about 2.5 g.

The one or more leucine metabolites can be selected from the group consisting of keto-isocaproic acid (KIC), alpha-hydroxy-isocaproic acid, and HMB.

Another aspect of the invention provides for a composition comprising: (a) leucine and/or at least one or more leucine metabolites; and (b) at least one or more anti-diabetic agents comprising a guanide. The component (a) in the composition can be leucine. The molar ratio of component (a) to component (b) in said composition can be greater than about 20 or 200 or 2000.

The composition can be substantially free of non-branched amino acids. The composition can be substantially free of each amino acid selected from the group consisting of alanine, arginine, asparagine, aspartic acid, cysteine, glutamic acid, glutamine, glycine, histidine, lysine, methionine, phenylalanine, proline, serine, threonine, tryptophan, valine, isoleucine and tyrosine. The composition contains less than about 0.1% of each free amino acid selected from the group consisting of alanine, arginine, asparagine, aspartic acid, cysteine, glutamic acid, glutamine, glycine, histidine, lysine, methionine, phenylalanine, proline, serine, threonine, tryptophan, valine, isoleucine and tyrosine. The composition contains less than about 10% of non-leucine amino acids. The composition can be substantially free of each of alanine, glycine, glutamic acid, and proline.

The composition can include resveratrol or be substantially free of resveratrol. The composition may or may not contain resveratrol.

The amount of leucine and/or one or more leucine metabolites can, when administered to a subject, yield a serum level of the leucine and/or one or more leucine metabolites that can be between 0.3-0.5 mM. The amount of leucine and/or one or more leucine metabolites can, when administered to a subject, yield a serum level of the leucine and/or one or more leucine metabolites that can be about 0.5 mM.

The amount of the one or more anti-diabetic agent and/or any analog thereof can, when administered to a subject, yield a serum level of the one or more anti-diabetic agent or any analog thereof that can be between about 10-100 μM.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent application file contains at least one drawing executed in color. Copies of this patent or patent application with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a diagram showing a sirtuin pathway.

FIG. 2 depicts two FDG-PET images showing the synergistic effects of resveratrol and HMB on glucose uptake using FDG-PET scanning analysis.

FIG. 3 depicts a graph showing interactive effects of chlorogenic acid (500 nM) with HMB (5 μM) and resveratrol (200 nM) on fatty acid oxidation in C2C12 myotubes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 4 depicts a graph showing interactive effects of chlorogenic acid (500 nM) and HMB (5 μM) on fatty acid oxidation (data expressed as % change from control value. *p=0.05)

FIG. 5 depicts a graph showing interactive effects of chlorogenic acid (500 nM) with HMB (5 μM) and leucine (0.5 mM) on Sirt1 activity in 3T3-L1 adipocytes (data expressed as % change from control value; *p=0.005; **p=0.0001).

FIG. 6 depicts a graph showing interactive effects of chlorogenic acid (500 nM) with HMB (5 μM) and leucine (0.5 mM) on glucose utilization (*p=0.045; **p=0.007). Glucose utilization was measured as extracellular acidification response to glucose injection. Response to insulin (5 nM) is included for reference.

FIG. 7 depicts a graph showing interactive effects of caffeic acid (1 μM) with leucine (0.5 mM) and resveratrol (200 nM) on fatty acid oxidation in C2C12 myotubes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 8 depicts a graph showing interactive effects of caffeic acid (1 μM) with HMB (5 μM) and resveratrol (200 nM) on fatty acid oxidation in C2C12 myotubes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 9 depicts a graph showing interactive effects of caffeic acid (1 μM), HMB (5 μM), leucine (0.5 mM) and resveratrol (200 nM) on fatty acid oxidation in C2C12 myotubes and 3T3-L1 adipocytes (data expressed as % change from control value;*p=0.05; **p=0.016).

FIG. 10 depicts a graph showing interactive effects of quinic acid (500 nM) with HMB (5 μM) and resveratrol (200 nM) on fatty acid oxidation in 3T3-L1 adipocytes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 11 depicts a graph showing interactive effects of quinic acid (500 nM) with leucine (0.5 mM) and resveratrol (200 nM) on fatty acid oxidation in 3T3-L1 adipocytes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 12 depicts a graph showing interactive effects of quinic acid (500 nM), HMB (5 μM), leucine (0.5 mM) and resveratrol (200 nM) on fatty acid oxidation in C2C12 myotubes and 3T3-L1 adipocytes (data expressed as % change from control value; *p=0.05; **p=0.012).

FIG. 13 depicts a graph showing interactive effects of quinic acid (500 nM), HMB (5 μM), leucine (0.5 mM) and resveratrol (200 nM) on AMPK activity (data expressed as % change from control value; *p=0.0001).

FIG. 14 depicts a graph showing interactive effects of quinic acid (500 nM), HMB (5 μM), leucine (0.5 mM) and resveratrol (200 nM) on glucose utilization. Glucose utilization was measured as extracellular acidification response to glucose injection (*p=0.05; **p=0.0003).

FIG. 15 depicts a graph showing interactive effects of cinnamic acid (500 nM) with HMB (5 μM) and resveratrol (200 nM) on fatty acid oxidation in C2C12 myotubes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 16 depicts a graph showing interactive effects of cinnamic acid (500 nM) with leucine (0.5 mM) and resveratrol (200 nM) on fatty acid oxidation in C2C12 myotubes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 17 depicts a graph showing interactive effects of cinnamic acid (500 nM) with HMB (5 μM), leucine (0.5 mM) and resveratrol (200 nM) on fatty acid oxidation in 3T3-L1 adipocytes (data expressed as % change from control value; *p=0.004; **p=0.006).

FIG. 18 depicts a graph showing interactive effects of cinnamic acid (500 nM) with HMB (5 μM), leucine (0.5 mM) and resveratrol (200 nM) on fatty acid oxidation in C2C12 myotubes (data expressed as % change from control value; *p=0.02; **p=0.05).

FIG. 19 depicts a graph showing interactive effects of cinnamic acid (500 nM) with HMB (5 μM), leucine (0.5 mM) and resveratrol (200 nM) on AMPK activity (data expressed as % change from control value; *p=0.0001).

FIG. 20 depicts a graph showing interactive effects of ferulic acid (500 nM) with HMB (5 μM) and resveratrol (200 nM) on fatty acid oxidation in 3T3-L1 adipocytes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 21 depicts a graph showing interactive effects of ferulic acid (500 nM) with HMB (5 μM), leucine (0.5 mM) and resveratrol (200 nM) on fatty acid oxidation in 3T3-L1 adipocytes (data expressed as % change from control value; *p=0.018)

FIG. 22 depicts a graph showing interactive effects of ferulic acid (500 nM) with leucine (0.5 mM) and resveratrol (200 nM) on fatty acid oxidation in C2C12 myotubes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 23 depicts a graph showing interactive effects of ferulic acid (500 nM) with HMB (5 μM), leucine (0.5 mM) and resveratrol (200 nM) on fatty acid oxidation in 3T3-L1 adipocytes (data expressed as % change from control value; *p=0.034).

FIG. 24 depicts a graph showing interactive effects of ferulic acid (500 nM) with HMB (5 μM), leucine (0.5 mM) and resveratrol (200 nM) on AMPK activity (data expressed as % change from control value; *p=0.05).

FIG. 25 depicts a graph showing interactive effects of piceatannol (1 nM) with leucine (0.5 mM) and resveratrol (200 nM) on fatty acid oxidation in 3T3-L1 adipocytes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 26 depicts a graph showing interactive effects of piceatannol (1 nM) with HMB (5 μM) and resveratrol (200 nM) on fatty acid oxidation in 3T3-L1 adipocytes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 27 depicts a graph showing interactive effects of piceatannol (1 nM) with HMB (5 μM), leucine (0.5 mM) and resveratrol (200 nM) on fatty acid oxidation in C2C12 myotubes (data expressed as % change from control value; *p=0.039).

FIG. 28 depicts a graph showing interactive effects of epigallocatechin gallate (EGCG) (1 μM), HMB (5 μM), leucine (0.5 mM) and resveratrol (200 nM) on glucose utilization in C2C12 myotubes. Glucose utilization was measured as extracellular acidification response to glucose injection (*p=0.015; **p=0.017).

FIG. 29 depicts a graph showing effects of fucoxanthin (100 nM) with HMB (5 μM) and resveratrol (200 nM) on fatty acid oxidation in 3T3-L1 adipocytes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 30 depicts a graph showing interactive effects of fucoxanthin (100 nM) leucine (0.5 mM) and resveratrol (200 nM) on fatty acid oxidation in 3T3-L1 adipocytes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 31 depicts a graph showing interactive effects of fucoxanthin (100 nM) with HMB (5 μM), leucine (0.5 mM) and resveratrol (200 nM) on fatty acid oxidation in 3T3-L1 adipocytes (data expressed as % change from control value; *p=0.033; **p=0.05).

FIG. 32 depicts a graph showing interactive effects of fucoxanthin (100 nM), HMB (5 μM) and leucine (0.5 mM) on glucose utilization in C2C12 myotubes. Glucose utilization was measured as extracellular acidification response to glucose injection (*p<0.04).

FIG. 33 depicts a graph showing interactive effects of fucoxanthin (100 nM), HMB (5 μM), leucine (0.5 mM) and resveratrol (200 nM) on glucose utilization in 3T3-L1 adipocytes. Glucose utilization was measured as extracellular acidification response to glucose injection (*p=0.02; **p=0.003).

FIG. 34 depicts a graph showing interactive effects of grape seed extract (1 μg/mL) with HMB (5 μM) and resveratrol (200 nM) on fatty acid oxidation in 3T3-L1 adipocytes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 35 depicts a graph showing interactive effects of grape seed extract (1 μg/mL) with HMB (5 μM) and resveratrol (200 nM) on fatty acid oxidation in 3T3-L1 adipocytes (data expressed as % change from control value; *p=0.04).

FIG. 36 depicts a graph showing interactive effects of grape seed extract (1 μg/mL) with HMB (5 μM), leucine (0.5 mM) and resveratrol (200 nM) on AMPK activity in 3T3-L1 adipocytes and C2C12 myotubes (data expressed as % change from control value; *p=0.01).

FIG. 37 depicts a graph showing interactive effects of metformin (0.1 mM) with HMB (5 μM), leucine (0.5 mM) and resveratrol (200 nM) on fatty acid oxidation in C2C12 myotubes (data expressed as % change from control value; *p=0.03; **p=0.0001; ***p=0.001).

FIG. 38 depicts a graph showing interactive effects of metformin (0.1 mM) with HMB (5 μM) and leucine (0.5 mM) on glucose utilization in C2C12 myotubes. Glucose utilization was measured as extracellular acidification response to glucose injection (*p=0.03).

FIG. 39 depicts a graph showing interactive effects of metformin (0.1 mM) with HMB (5 μM), leucine (0.5 mM) and resveratrol (200 nM) on AMPK activity in C2C12 myotubes (data expressed as % change from control value; *p=0.031; **p=0.026; ***p=0.017).

FIG. 40 depicts a graph showing interactive effects of metformin (0.1 mM) with HMB (5 μM) and leucine (0.5 mM) on mitochondrial biogenesis (*p=0.001; **p=0.013).

FIG. 41 depicts a graph showing interactive effects of rosiglitazone (1 nM) with HMB (5 μM), leucine (0.5 mM) and resveratrol (200 nM) on fatty acid oxidation in C2C12 myotubes (data expressed as % change from control value; *p=0.009).

FIG. 42 depicts a graph showing interactive effects of rosiglitazone (1 nM) with HMB (5 μM), leucine (0.5 mM) and resveratrol (200 nM) on fatty acid oxidation in 3T3-L1 adipocytes (data expressed as % change from control value; *p=0.004; **p=0.023; ***p=0.003).

FIG. 43 depicts a graph showing interactive effects of rosiglitazone (1 nM) with HMB (5 μM), leucine (0.5 mM) and resveratrol (200 nM) on glucose utilization in C2C12 myotubes. Glucose utilization was measured as extracellular acidification response to glucose injection (*p=0.05; **p=0.001).

FIG. 44 depicts a graph showing interactive effects of caffeine (10 nM) with HMB (5 μM), leucine (0.5 mM), resveratrol (200 nM) and metformin (0.1 mM) on fatty acid oxidation in C2C12 myotubes (data expressed as % change from control value; *p=0.03; **p=0.05; ***p=0.013).

FIG. 45 depicts a graph showing interactive effects of caffeine (10 nM) with HMB (5 μM), leucine (0.5 mM), resveratrol (200 nM) and metformin (0.1 mM) on fatty acid oxidation in 3T3-L1 adipocytes (data expressed as % change from control value. *p=0.008).

FIG. 46 depicts a graph showing interactive effects of caffeine (10 nM) with HMB (5 μM) and resveratrol (200 nM) on fatty acid oxidation in 3T3-L1 adipocytes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 47 depicts a graph showing interactive effects of theophylline (1 μM) with HMB (5 μM), leucine (0.5 mM) and resveratrol (200 nM) on fatty acid oxidation in C2C12 myotubes (data expressed as % change from control value; *p=0.03; **p=0.05).

FIG. 48 depicts a graph showing interactive effects of theophylline (1 μM) with HMB (5 μM), leucine (0.5 mM) and resveratrol (200 nM) on fatty acid oxidation in 3T3-L1 adipocytes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 49 depicts a graph showing interactive effects of theophylline (1 μM) with HMB (5 μM), leucine (0.5 mM) and resveratrol (200 nM) on fatty acid oxidation in 3T3-L1 adipocytes (data expressed as % change from control value; *p=0.006).

FIG. 50 depicts a graph showing interactive effects of cocoa extract/theobromine (0.1 μg/mL) with HMB (5 μM), leucine (0.5 mM) and resveratrol (200 nM) on fatty acid oxidation in 3T3-L1 adipocytes. Fatty acid oxidation was measured as O₂ consumption response to palmitate injection and is expressed as % change from pre-injection baseline (vertical line shows the time of palmitate injection; data points to the left of this line are baseline measurements and those to the right of the line show the O₂ consumption response).

FIG. 51 depicts a graph showing interactive effects of cocoa extract/theobromine (0.1 μg/mL) with HMB (5 μM), leucine (0.5 mM) and resveratrol (200 nM) on fatty acid oxidation in 3T3-L1 adipocytes (data expressed as % change from control value; *p=0.021; **p=0.00035).

FIG. 52 depicts a graph showing effects of a standard dose of metformin (here 1.5 g metformin/kg diet), a low dose of metformin (here 0.75 g metformin/kg diet) and a very lose dose of metformin (here 0.25 g metformin/kg diet) compared with the low dose of metformin+12.5 mg resveratrol and 2 g CaHMB/kg diet and with the very lose dose of metformin+12.5 mg resveratrol and 2 g CaHMB/kg diet on plasma insulin in db/db mice (*p<0.02 vs. control).

FIG. 53 depicts a graph showing effects of a standard dose of metformin (here 1.5 g metformin/kg diet), a low dose of metformin (here 0.75 g metformin/kg diet) and a very lose dose of metformin (here 0.25 g metformin/kg diet) compared with the low dose of metformin+12.5 mg resveratrol and 2 g CaHMB/kg diet and with the very lose dose of metformin+12.5 mg resveratrol and 2 g CaHMB/kg diet on HOMA_{IR} (homeostatic assessment of insulin resistance) in db/db mice (*p<0.025 vs. control).

FIG. 54 depicts a graph showing effects of a standard dose of metformin (here 1.5 g metformin/kg diet), a low dose of metformin (here 0.75 g metformin/kg diet) and a very lose dose of metformin (here 0.25 g metformin/kg diet) compared with the low dose of metformin+12.5 mg resveratrol and 2 g CaHMB/kg diet and with the very lose dose of metformin+12.5 mg resveratrol and 2 g CaHMB/kg diet on 30-minute plasma glucose response to insulin (0.75 U/kg body weight) in db/db mice (*p<0.02 vs. control).

FIG. 55 depicts a graph showing effects of a standard dose of metformin (here 1.5 g metformin/kg diet), a low dose of metformin (here 0.75 g metformin/kg diet) and a very lose dose of metformin (here 0.25 g metformin/kg diet) compared with the low dose of metformin+12.5 mg resveratrol and 2 g CaHMB/kg diet and with the very lose dose of metformin+12.5 mg resveratrol and 2 g CaHMB/kg diet on visceral fat mass in db/db mice (*p<0.03 vs. control).

FIG. 56 depicts a graph showing effects of a standard dose of metformin (here 1.5 g metformin/kg diet), a low dose of metformin (here 0.75 g metformin/kg diet) and a very lose dose of metformin (here 0.25 g metformin/kg diet) compared with the low dose of metformin+12.5 mg resveratrol and 2 g CaHMB/kg diet and with the very lose dose of metformin+12.5 mg resveratrol and 2 g CaHMB/kg diet on visceral fat mass in db/db mice (*p<0.05 vs. control).

FIG. 57 illustrates the chemical structures of nicotinic acid and nicotinamide riboside.

FIG. 58 illustrates the effects of nicotinic acid and leucine, and/or resveratrol on Sirt1 activation in C2C12 myotubes. NA refers to nicotinic acid; Leu refers to leucine; R refers to resveratrol. *p<0.05; **p=0.0001. Data expressed as % change from control value.

FIG. 59 illustrates the effects of nicotinic acid and leucine, and/or resveratrol on P-AMPK/AMPK ratio in 3T3-L1 adipocytes. NA refers to nicotinic acid; Leu refers to leucine; R refers to resveratrol. *p<0.01. Data expressed as % change from control value.

FIG. 60 FIG. 4 illustrates the effects of leucine (0.5 mM)/nicotinic acid (10 nM) on lipid levels in C. elegans (*p=0.012). NA refers to nicotinic acid; Leu refers to leucine.

FIG. 61 illustrates the effects of four weeks treatment with Leucine (Leu, 24 g/kg diet), Leu (24 g/kg diet)+nicotinic acid (NA, 50 mg/kg diet), Leu (24 g/kg diet)+NA (250 mg/kg diet) and NA (1,000 mg/kg diet) added to a Western Diet (WD) on plasma total cholesterol in LDL receptor knockout mice.

FIG. 62 illustrates the effects of four weeks treatment with Leucine (Leu, 24 g/kg diet), Leu (24 g/kg diet)+nicotinic acid (NA, 50 mg/kg diet), Leu (24 g/kg diet)+NA (250 mg/kg diet) and NA (1,000 mg/kg diet) added to a Western Diet (WD) on plasma cholesterol esters in LDL receptor knockout mice.

FIG. 63 illustrates the effects of four weeks treatment with Leucine (Leu, 24 g/kg diet), Leu (24 g/kg diet)+nicotinic acid (NA, 50 mg/kg diet), Leu (24 g/kg diet)+NA (250 mg/kg diet) and NA (1,000 mg/kg diet) added to a Western Diet (WD) on plasma triglycerides in LDL receptor knockout mice.

FIG. 64 illustrates the effects of eight weeks treatment with Leucine (Leu, 24 g/kg diet), Leu (24 g/kg diet)+nicotinic acid (NA, 50 mg/kg diet), Leu (24 g/kg diet)+NA (250 mg/kg diet) and NA (1,000 mg/kg diet) added to a Western Diet (WD) on plasma total cholesterol in LDL receptor knockout mice.

FIG. 65 illustrates the effects of eight weeks treatment with Leucine (Leu, 24 g/kg diet), Leu (24 g/kg diet)+nicotinic acid (NA, 50 mg/kg diet), Leu (24 g/kg diet)+NA (250 mg/kg diet) and NA (1,000 mg/kg diet) added to a Western Diet (WD) on plasma cholesterol esters in LDL receptor knockout mice.

FIG. 66 illustrates the effects of eight weeks treatment with nicotine acid (1,000 mg/kg diet) on atherosclerotic lesion size in LDL receptor knockout mice. Shown are Oil Red O stained aortic histology slides.

FIG. 67 illustrates the effects of eight weeks treatment with Leucine (Leu, 24 g/kg diet), Leu (24 g/kg diet)+nicotinic acid (NA, 50 mg/kg diet), and NA (1,000 mg/kg diet) added to a Western Diet (WD) on total lesion area in LDL receptor knockout mice.

FIG. 68 illustrates the effects of eight weeks treatment with Leucine (Leu, 24 g/kg diet), Leu (24 g/kg diet)+nicotinic acid (NA, 50 mg/kg diet), and NA (1,000 mg/kg diet) added to a Western Diet (WD) on Lipid Deposition Area, as observed by the Oil Red O positive area in LDL receptor knockout mice.

FIG. 69 illustrates the effects of eight weeks treatment with Leucine (Leu, 24 g/kg diet), Leu+nicotinic acid (NA, 50 mg/kg diet), and NA (1,000 mg/kg diet) added to a Western Diet (WD) on aortic macrophage infiltration in LDL receptor knockout mice.

FIG. 70 illustrates the quantitative effects of eight weeks treatment with Leucine (Leu, 24 g/kg diet), Leu+nicotinic acid (NA, 50 mg/kg diet), and NA (1,000 mg/kg diet) added to a Western Diet (WD) on aortic macrophage infiltration (measured as percent CD 68 positive area) in LDL receptor knockout mice.

FIG. 71 illustrates the effects of nicotinic acid and leucine on the lifespan of C. elegans. NA refers to nicotinic acid; Leu refers to leucine. *p<0.0001. Data expressed as % survival over time.

FIG. 72 shows interactive effects of leucine (0.5 mM), metformin and nicotinic acid (1 μM) on glucose utilization response to 5 nM insulin in C2C12 myotubes. The vertical line shows glucose injection. Values to the left of this line are baseline values and values to the right show response to injection.

FIG. 73 illustrates area under the curve quantitation of the interactive effects of leucine, metformin and nicotinic acid on glucose utilization response to 5 nM insulin in C2C12 myotubes.

FIG. 74 illustrates area under the curve quantitation of the interactive effects of leucine (0.5 mM), metformin and nicotinic acid (1 nM) on fat oxidation, as measured by palmitate-induced increases in oxygen consumption, in C2C12 myotubes.

FIG. 75 shows interactive effects of leucine, metformin and nicotinic acid (1 nM) on lipid accumulation, as measured by Oil Red O, in HepG2 hepatocytes.

FIG. 76 shows Glucose Tolerance Test (GTT) after 6 weeks of HFD feeding. Before randomization to treatment groups, GTT was performed. Data were presented as means±SEM (n=10). * indicates significant different from all other groups (p<0.0001).

FIG. 77 shows body weight after 6 weeks of HFD feeding. Body weight before start of indicated treatments. Data were presented as means±SEM (n=10). * indicates significant different from all other groups (p<0.0001).

FIG. 78A shows Glucose Tolerance Test (GTT) with resveratrol. Glucose levels were measured at 15, 30, 60, 90 and 120 min after glucose injection. Data were presented as means±SEM (n=10). * indicates significant different from HFD (p<0.001), ** indicates significant different from all groups (p<0.001).

FIG. 78B shows the area under the curve calculated from the GTT data presented in FIG. 78A. Data were presented as means±SEM (n=10). * indicates significant different from HFD (p<0.001), ** indicates significant different from all groups (p<0.001).

FIG. 79A shows Insulin Tolerance Test (ITT) with resveratrol. Glucose levels were measured at 15, 30, 60, 90 and 120 min after insulin injection. Data were presented as means±SEM (n=10). * indicates significant different from HFD (p<0.001), ** indicates significant different from all groups (p<0.001).

FIG. 79B shows the % change in glucose response from baseline at 30 min after insulin injection calculated from the ITT data presented in FIG. 79A. Data were presented as means±SEM (n=10). * indicates significant different from HFD (p<0.001), ** indicates significant different from all groups (p<0.001).

FIG. 80A shows Glucose Tolerance Test (GTT) from study 2 without resveratrol. Glucose levels were measured at 15, 30, 60, 90 and 120 min after glucose injection. Data were presented as means±SEM (n=10). * indicates significant different from HFD (p<0.003), ** indicates significant different from HFD and Met 1.5 (p<0.001).

FIG. 80B shows the area under the curve calculated from the GTT data present in FIG. 80A. Data were presented as means±SEM (n=10). * indicates significant different from HFD (p<0.003), ** indicates significant different from HFD and Met 1.5 (p<0.001).

FIG. 81A shows Insulin Tolerance Test (ITT) without resveratrol. Glucose levels were measured at 15, 30, 60, 90 and 120 min after insulin injection. Data were presented as means±SEM (n=10). * indicates significant different from HFD (p<0.003), ** indicates significant different from HFD and Met 1.5 (p<0.001).

FIG. 81B shows the % change in glucose response from baseline at 30 min after insulin injection calculated from the ITT data presented in FIG. 81A. Data were presented as means±SEM (n=10). * indicates significant different from HFD (p<0.003), ** indicates significant different from HFD and Met 1.5 (p<0.001).

FIG. 82 shows fasting glucose from mice treated with leucine/metformin without resveratrol. After 5 weeks of treatment fasting glucose was measured. Data were presented as means±SEM (n=10). * indicates significant different from HFD (p<0.0001), ** indicates significant different from HFD but not different from LFD (p<0.0001).

FIG. 83 shows fasting insulin from mice treated with leucine/metformin without resveratrol. After 5 weeks of treatment fasting insulin was measured. Data were presented as means±SEM (n=10). * indicates significant different from HFD (p<0.0001), ** indicates significant different from HFD but not different from LFD (p<0.0001).

FIG. 84 shows Homeostatic Assessment of Insulin Resistance (HOMA_{IR}) from mice treated with leucine/metformin without resveratrol. After 5 weeks of treatment Homeostatic Assessment of Insulin Resistance (HOMA_{IR}) was measured. Data were presented as means±SEM (n=10). * indicates significant different from HFD (p<0.0001), ** indicates significant different from HFD but not different from LFD (p<0.0001).

FIG. 85 shows Sirt activity after leucine/metformin treatment. Differentiated adipocytes were treated with metformin (0.1 mM), Leucine (0.5 mM) or the combination for 24 to 48 hours. Sirt1 activity was measured in cell extract. Data were represented as mean±SEM (n=4). * indicates significant difference to control (p=0.05), ** indicates significant difference to all other groups (p<0.02).

FIG. 86 shows fatty acid oxidation after leucine/metformin treatment. Differentiated adipocytes were treated with metformin (0.1 mM), Leucine (0.5 mM) or the combination for 24 to 48 hours. Oxygen consumption rate was measured after 200 μM palmitate injection (points A & C). Data are represented as mean±SD (n=5).

FIG. 87A shows P-AMPK and AMPK activity after leucine/metformin treatment. Western blot was performed with antibodies against P-AMPK and AMPK. Differentiated adipocytes were treated with metformin (0.1 mM), Leucine (0.5 mM) or the combination for 24 to 48 hours.

FIG. 87B shows quantification of the blots from FIG. 87A in fold change. Data was normalized to total volume intensity of blot, then the ratio was calculated and represented as mean±SEM of fold-change to control. * indicates significant difference to control and metformin (p<0.04). Differentiated adipocytes were treated with metformin (0.1 mM), Leucine (0.5 mM) or the combination for 24 to 48 hours.

FIG. 88A shows P-AMPK and AMPK activity in muscle of diet-induced obesity mice treated with leucine/metformin. Western blot data are collected from muscle of diet-induced obesity mice. Showing are representative Western blot data of P-AMPK and AMPK. The quantification of their ratios from gastrocnemius muscle of DIO-mice fed a HFD with indicated treatments for 6 weeks were shown. Quantification of each blot was normalized to β-actin. * indicates significant difference to LFD and HFD (p≤0.01).

FIG. 88B shows P-ACC and ACC activity in muscle of diet-induced obesity mice treated with leucine/metformin. Western blot data are collected from muscle of diet-induced obesity mice. Showing are representative Western blot data of P-ACC and ACC and the quantification of their ratios from gastrocnemius muscle of DIO-mice fed a HFD with indicated treatments for 6 weeks were shown in FIG. 88A. Quantification of each blot was normalized to β-actin. * indicates significant difference to LFD and HFD (p≤0.01).

FIG. 89A shows post-prandial glucose levels measured at 60 min after glucose injection in treatment of HMB/metformin for 1 week. Data were presented as means±SEM (n=10). * indicates significant different from HFD (p<0.003), ** indicates significant different from HFD and Met 1.5 (p<0.001).

FIG. 89B shows Glucose Tolerance Test (GTT) area under the curve after glucose injection in treatment of HMB/metformin for 4 weeks. Data were presented as means±SEM (n=10). * indicates significant different from HFD (p<0.003), ** indicates significant different from HFD and Met 1.5 (p<0.001).

FIG. 89C shows post-prandial glucose levels measured at 60 min after glucose injection in treatment of leucine/metformin for 1 week. Data were presented as means±SEM (n=10). * indicates significant different from HFD (p<0.003), ** indicates significant different from HFD and Met 1.5 (p<0.001).

FIG. 89D shows post-prandial glucose levels measured at 60 min after glucose injection in treatment of leucine/metformin for 2 weeks. Data were presented as means±SEM (n=10). * indicates significant different from HFD (p<0.003), ** indicates significant different from HFD and Met 1.5 (p<0.001).

FIG. 90 illustrates interactive effects of leucine (0.5 mM) and galegine (5 μM) on glucose utilization response to 5 nM insulin in C2C12 myotubes. The vertical line shows glucose injection. Values to the left of this line are baseline values and values to the right show response to injection.

FIG. 91 shows area under the curve quantitation of the interactive effects of leucine (0.5 mM) and galegine (5 μM) on glucose utilization response to 5 nM insulin in C2C12 myotubes.

FIG. 92 shows the interactive effects of leucine (0.5 mM) and dimethylguanidine (DMG) (10 μM) on glucose utilization response to 5 nM insulin in 3T3-L1 adipocytes. The vertical line shows glucose injection. Values to the left of this line are baseline values and values to the right show response to injection.

FIG. 93 shows area under the curve quantitation of the interactive effects of leucine (0.5 mM) and galegine (5 μM) on fat oxidation, as measured by palmitate-induced increases in oxygen consumption, in C2C12 myotubes.

FIG. 94 shows quantitation of the interactive effects of nicotinic acid in full dose (1000 mg/kg) or combination of nicotinic acid in reduced dose (50 mg/kg) with leucine (24 g/kg) and metformin (0.5 g/kg) on fasting blood glucose following 8 weeks of treatment.

FIG. 95 shows quantitation of the interactive effects of nicotinic acid in full dose (1000 mg/kg) or combination of nicotinic acid in reduced dose (50 mg/kg) with leucine (24 g/kg) and metformin (0.5 g/kg) on the 8-week change from baseline to end of study on fasting plasma insulin.

FIG. 96 shows quantitation of the interactive effects of nicotinic acid in full dose (1000 mg/kg) or combination of nicotinic acid in reduced dose (50 mg/kg) with leucine (24 g/kg) and metformin (0.5 g/kg) on calculated homeostatic assessment of insulin resistance (HOMAir) following 8 weeks of treatment.

FIG. 97 shows quantitation of the interactive effects of nicotinic acid in full dose (1000 mg/kg) or combination of nicotinic acid in reduced dose (50 mg/kg) with leucine (24 g/kg) and metformin (0.5 g/kg) on LDL level change in blood serum.

FIG. 98 shows quantitation of the interactive effects of nicotinic acid in full dose (1000 mg/kg) or combination of nicotinic acid in reduced dose (50 mg/kg) with leucine (24 g/kg) and metformin (0.5 g/kg) on cholesterol level change in blood serum.

FIG. 99 shows quantitation of the interactive effects of nicotinic acid in full dose (1000 mg/kg) or combination of nicotinic acid in reduced dose (50 mg/kg) with leucine (24 g/kg) and metformin (0.5 g/kg) on triglycerides level change in blood serum.

FIG. 100 shows histological images of heart in mice. Atherosclerosis is visualized by Oil Red O staining. Showing are the effects of nicotinic acid in full dose (1000 mg/kg) or combination of nicotinic acid in reduced dose (50 mg/kg) with leucine (24 g/kg) and metformin (0.5 g/kg) on atherosclerosis.

FIG. 101 shows quantification of atherosclerosis in mice heart and aorta. Atherosclerosis is quantified by calculating the area positively stained with Oil Red O. Showing are the effects of nicotinic acid in full dose (1000 mg/kg) or combination of nicotinic acid in reduced dose (50 mg/kg) with leucine (24 g/kg) and metformin (0.5 g/kg) on atherosclerosis. Lines show standard error; (**) indicates p<0.01 in 1 way ANOVA.

### DETAILED DESCRIPTION OF THE INVENTION

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “subject” or “individual” includes mammals. Non-limiting examples of mammals include humans and mice, including transgenic and non-transgenic mice. The methods described herein can be useful in both human therapeutics, pre-clinical, and veterinary applications. In some embodiments, the subject is a mammal, and in some embodiments, the subject is human. Other mammals include, and are not limited to, apes, chimpanzees, orangutans, monkeys; domesticated animals (pets) such as dogs, cats, guinea pigs, hamsters, mice, rats, rabbits, and ferrets; domesticated farm animals such as cows, buffalo, bison, horses, donkey, swine, sheep, and goats; or exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, pandas, giant pandas, hyena, seals, sea lions, and elephant seals.

The terms “administer”, “administered”, “administers” and “administering” are defined as the providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments of the subject application, oral routes of administering a composition can be preferred.

As used herein, “agent” or “biologically active agent” refers to a biological, pharmaceutical, or chemical compound or other moiety. Non-limiting examples include simple or complex organic or inorganic molecule, a peptide, a protein, a peptide nucleic acid (PNA), an oligonucleotide (including e.g., aptomer and polynucleotides), an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The term “effective amount” or “therapeutically effective amount” refers to that amount of a compound described herein that is sufficient to affect the intended application including but not limited to disease or condition treatment, as defined below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or down regulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

The term “energy metabolism,” as used herein, refers to the transformation of energy that accompanies biochemical reactions in the body, including cellular metabolism and mitochondrial biogenesis. Energy metabolism can be quantified using the various measurements described herein, for example and without limitations, weight-loss, fat-loss, insulin sensitivity, fatty acid oxidation, glucose utilization, triglyceride content, Sirt 1 expression level, AMPK expression level, oxidative stress, and mitochondrial biomass.

The term “isolated”, as applied to the subject components, for example a sirtuin pathway activator, including but not limited to one or more agents selected from the group consisting of nicotinic acid, nicotinamide riboside, and nicotinic acid metabolite, leucine and leucine metabolites (such as HMB), and resveratrol, refers to a preparation of the substance devoid of at least some of the other components that can also be present where the substance or a similar substance naturally occurs or is initially obtained from. Thus, for example, an isolated substance can be prepared by using a purification technique to enrich it from a source mixture. Enrichment can be measured on an absolute basis, such as weight per volume of solution, or it can be measured in relation to a second, potentially interfering substance present in the source mixture. Increasing enrichment of the embodiments of this invention are increasingly more preferred. Thus, for example, a 2-fold enrichment is preferred, 10-fold enrichment is more preferred, 100-fold enrichment is more preferred, 1000-fold enrichment is even more preferred. A substance can also be provided in an isolated state by a process of artificial assembly, such as by chemical synthesis.

A “sub-therapeutic amount” of an agent, an activator or a therapy is an amount less than the effective amount of that agent, activator or therapy for an intended application, but when combined with an effective or sub-therapeutic amount of another agent or therapy can produce a desired result, due to, for example, synergy in the resulting efficacious effects, and/or reduced side effects.

A “synergistic” or “synergizing” effect can be such that the one or more effects of the combination compositions are greater than the one or more effects of each component alone, or they can be greater than the sum of the one or more effects of each component alone. The synergistic effect can be about, or greater than about 10, 20, 30, 50, 75, 100, 110, 120, 150, 200, 250, 350, or 500% or even more than the effect on a subject with one of the components alone, or the additive effects of each of the components when administered individually. The effect can be any of the measurable effects described herein.

The term “substantially free”, as used herein, refers to compositions that have less than about 10%, less than about 5%, less than about 1%, less than about 0.5%, less than 0.1% or even less of a specified component. For example a composition that is substantially free of non-branched chain amino acids can have less than about 1% of the non-branched chain amino acid lysine. The percentage can be determined as a percent of the total composition or a percent of a subset of the composition. For example, a composition that is substantially free of non-branched chain amino acids can have less than 1% of the non-branched chain amino acids as a percent of the total composition, or as a percent of the amino acids in the composition. The percentages can be mass, molar, or volume percentages.

The terms “clinical significance” or “clinically significant” indicate behaviors and symptoms that are considered to be outside the range of normal, and are marked by distress and impairment of daily functioning. For example, a clinically significant cutaneous vasodilation would be a level sufficient to elicit patient complaint regarding discomfort secondary to acute vasodilatation, including flushing, itching and/or tingling. Levels of cutaneous vasodilation can also be measured by any methods known in the medical art, such as the methods including laser-Doppler flowmeter that are disclosed in Saumet J. L. et al., “Non-invasive measurement of skin blood flow: comparison between plethysmography, laser-Doppler flowmeter and heat thermal clearance method” Int. J. Microcirc. Clin. Exp. 1986; 5:73-83. A clinically significant level of cutaneous vasodilation can also be a level that is statistically significant. A clinically significant level of cutaneous vasodilation can also be a level that is not statistically significant.

The terms “lipid content” or “lipid level” refer to the content or level of lipid or lipoprotein molecules measured inside of a subject. It can be the concentration of the lipid molecules in a circulating bloodstream, or a total quantity of body fat. The lipid or lipoprotein molecules can include triglyceride, cholesterol, LDL, or HDL.

### Compositions

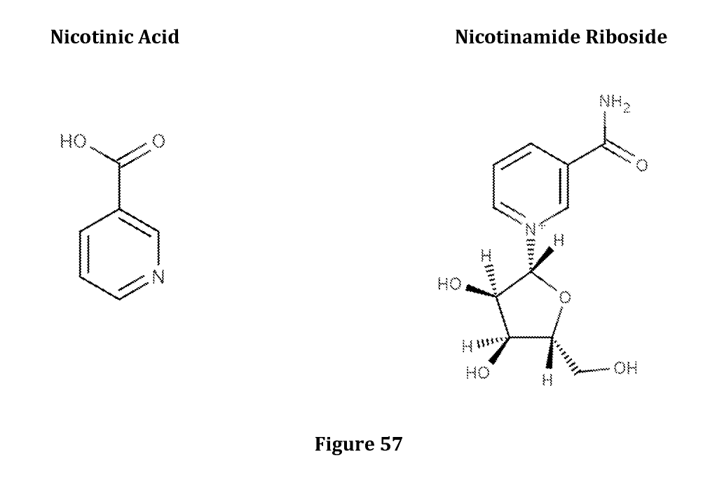

In one aspect, the invention provides for compositions comprising a combination of (a) leucine and/or one or more leucine metabolites, (b) one or more agents selected from the group consisting of nicotinic acid, nicotinamide riboside, and nicotinic acid metabolite, and (c) an anti-diabetic agent. The chemical structures for nicotinic acid and nicotinamide riboside are shown in FIG. 57

In another aspect, the invention provides for compositions comprising a combination of (a) leucine and/or at least one or more leucine metabolites in combination and (b) at least one or more anti-diabetic agents that is a guanide. The guanide may have a dimethyl structure, for example, metformin, dimethylguanidine, and/or galegine. The amounts of nicotinic acid and/or the anti-diabetic agent can be sub-therapeutic.

The compositions can further comprise resveratrol or one or more therapeutic agents that is capable of lowering lipid level or treating diabetes. The combination when administered to a subject can be used to modulate metabolic pathways and for treatment of diabetes and/or hyperlipidemia. In some embodiments, the anti-diabetic agent is a sirtuin pathway activator. In a related embodiment, the anti-diabetic agent is a biguanide such as metformin or any analog thereof. In still a related embodiment, the anti-diabetic agent is a guanide such as galegine, dimethylguanidine and/or any analog thereof. In some embodiments, the invention provides a method of potentiating the therapeutic efficacy of a composition comprising administering simultaneously or sequentially to a subject component (a) and component (b) and component (c) of the invention, wherein the administration of (a) and (b) and (c) is in an amount that increases insulin sensitivity, increases glucose utilization, increases fat oxidation and reduces lipid levels, and further wherein component (c) is a biguanide (e.g. metformin or analogs thereof). In some embodiments, the increase in insulin sensitivity is at least about a 1-fold increase (e.g. at least about 1, 2, 3, 4, 5, 6, 8, 10, 15, 20, or 50 fold). In some embodiments, the combination of these components can be useful for lowering lipid content, lowering total cholesterol level, lowering LDL level, lowering triglyceride level, lowering lipid accumulation, increasing fat oxidation, or increasing HDL level. In some embodiments, the components are formulated to provide a synergistic effect, including but not limited to further reduction of the lipid content or reduction in dosing amounts leading to reduced side effects to the subject, and/or increase of insulin sensitivity. The combination can be particularly effective in lowering the lipid content while causing a reduced degree of cutaneous vasodilation and reduced risk of hyperglycemia and glucose intolerance in a subject as compared to a dose of nicotinic acid alone that has the same effectiveness as the composition in lowering lipid content. The reduced risk of hyperglycemia and glucose intolerance is particularly advantageous in that it allows for nicotinic acid to be used for patients affected by both hyperlipidemia and diabetes. The amount of one or more agents selected from the group consisting of nicotinic acid, nicotinamide riboside, and nicotinic acid metabolite in the composition can be a sub-therapeutic amount in the absence of leucine and/or one or more leucine metabolites. The combination can also be particularly effective in increasing insulin sensitivity and increasing glucose utilization while causing a reduced degree of lactic acidosis and/or hypoglycemia in a subject as compared to a dose of anti-diabetic agent such as metformin alone that has the same effectiveness as the composition in increasing insulin sensitivity and lowering blood glucose level. The amount of anti-diabetic agents such as metformin or any analog thereof in the composition can be a sub-therapeutic amount in the absence of leucine and/or one or more leucine metabolites, with one or more agents selected from the group consisting of nicotinic acid, nicotinamide riboside, and nicotinic acid metabolite.

In some embodiments, the amount of (a) leucine and/or one or more leucine metabolites, (b) one or more agents selected from the group consisting of nicotinic acid, nicotinamide riboside, and nicotinic acid metabolite, and/or (c) an anti-diabetic agent in the disclosed compositions are formulated to provide a synergistic effect that increases insulin sensitivity, increases glucose utilization, lowers plasma glucose level, lowers lipid accumulation, increases fat oxidation, and/or activates one or more components in the sirtuin pathway in a subject when administered to the subject as compared to administering a subject component (a) and component (b), or component (a) and component (c), or component (b) and component (c). Compositions including nicotinic acid are described in U.S. patent application Ser. No. 14/472,081, PCT Application No. PCT/US14/026816, filed Mar. 13, 2014, and U.S. Provisional Application No. 61/800,363, filed Mar. 15, 2013, each of which are hereby incorporated by reference in their entirety.

The invention also provides for compositions for treatment of diabetes and/or hyperlipidemia. The composition provides for a combination of medicaments of potentiating the therapeutic efficacy of one or more anti-diabetic agents selected from the group consisting of biguanide, guanide, meglitinide, sulfonylurea, thiazolidinedione, alpha glucosidase inhibitor, and ergot alkaloid, comprising administering simultaneously or sequentially to a subject (a) a sub-therapeutic amount of said anti-diabetic agent, and (b) one or more branched amino acids, wherein the administration of (a) and (b) is effective in ameliorating a diabetic symptom of said subject. Examples of diabetic symptoms include, but are not limited to, polyuria, polydipsia, weight loss, polyphagia, blurred vision, hypertension, abnormalities of lipoprotein metabolism, and periodontal disease. The biguanide can be metformin. The one or more anti-diabetic agent can comprise glipizide and/or metformin. The one or more anti-diabetic agent can be thiazolidinedione.

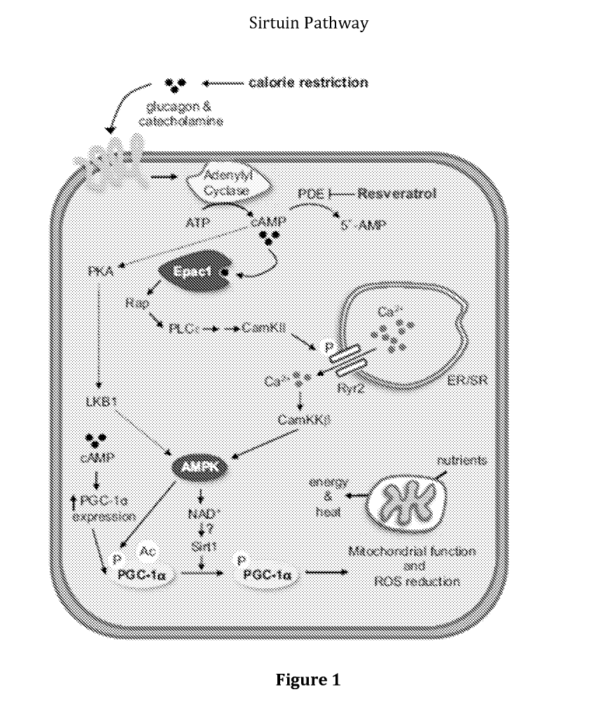

The invention further provides for compositions that can increase or modulate the output of a sirtuin pathway. The sirtuin pathway includes, without limitation, signaling molecules such as, Sirt1, Sirt3, and AMPK. The output of the pathway can be determined by the expression level and/or the activity of the pathway and/or a physiological effect. In some embodiments, activation of the Sirt1 pathway includes stimulation of PGC1-α and/or subsequent stimulation of mitochondrial biogenesis and fatty acid oxidation. In general, a sirtuin pathway activator is a compound that activates or increases one or more components of a sirtuin pathway. An increase or activation of a sirtuin pathway can be observed by an increase in the activity of a pathway component protein. For example, the protein can be Sirt1, PGC1-α, AMPK, Epac1, Adenylyl cyclase, Sirt3, or any other proteins and their respective associated proteins along the signaling pathway depicted in FIG. 1 (Park et. al., “Resveratrol Ameliorates Aging-Related Metabolic Phenotypes by Inhibiting cAMP Phosphodiesterases,” Cell 148, 421-433 Feb. 3, 2012). Non-limiting examples of physiological effects that can serve as measures of sirtuin pathway output include mitochondrial biogenesis, fatty acid oxidation, glucose uptake, palmitate uptake, oxygen consumption, carbon dioxide production, weight loss, heat production, visceral adipose tissue loss, respiratory exchanger ratio, insulin sensitivity, inflammation marker level, vasodilation, browning of fat cells, and irisin production. Examples of indicia of browning of fat cells include, without limitation, increased fatty acid oxidation, and expression of one or more brown-fat-selective genes (e.g. Ucp1, Cidea, Prdm16, and Ndufs1). Any SIRT pathway activator, including chlorogenic acid, quinic acid, sorbitol, myo-inositol, maltitol, cinnamic acid, ferulic acid, piceatannol, ellagic acid, epigallocatechin gallate, fucoxanthin, grape seed extract, metformin, rosiglitazone, PDE inhibitors, caffeine, theophylline, theobromine, and isobutylmethylxanthine, can be used in combination with other components described herein, including compositions including (a) leucine, anti-diabetic agents and one or more agents selected from the group consisting of nicotinic acid, nicotinamide riboside and nicotinic acid metabolites, (b) leucine and guanides, and (c) leucine and one or more agents selected from the group consisting of nicotinic acid, nicotinamide riboside and nicotinic acid metabolites.

In some embodiments, the sirtuin-pathway activator or AMPK pathway activator can be a polyphenol. For example, the polyphenol can be chlorogenic acid, resveratrol, caffeic acid, piceatannol, ellagic acid, epigallocatechin gallate (EGCG), grape seed extract, or any analog thereof. In some embodiments, the activator can be resveratrol, and an analog thereof, or metabolites thereof. For example, the activator can be pterostilbene or a small molecule analog of resveratrol. Examples of small molecule analogs of resveratrol are described in U.S. Patent Application Nos. 20070014833, 20090163476, and 20090105246, which are incorporated herein by reference in its entirety.

In other embodiments, the sirtuin-pathway activator or AMPK pathway activator can be irisin, quinic acid, cinnamic acid, ferulic acid, fucoxanthin, a biguanide (such as metformin), rosiglitazone, or any analog thereof. Alternatively the sirtuin-pathway activator or AMPK pathway activator can be isoflavones, pyroloquinoline (PQQ), quercetin, L-carnitine, lipoic acid, coenzyme Q10, pyruvate, 5-aminoimidazole-4-carboxamide ribotide (ALCAR), bezfibrate, oltipraz, and/or genistein.

In some embodiments, the composition can comprise combinations of metformin, resveratrol, nicotine and a branched chain amino acid or metabolites thereof. For example, a composition can comprise metformin, resveratrol, nicotine and HMB or the composition can comprise metformin, resveratrol, nicotine and leucine. Combinations of metformin, resveratrol, nicotine and a branched chain amino acid can cause an increase in fatty acid oxidation of over 700, 800, 900, 1000, 1200, 1400, 1600, or 1800%, and/or an increase of insulin sensitivity of at least about a 1-fold increase (e.g. at least about 1, 2, 3, 4, 5, 6, 8, 10, 15, 20, or 50 fold). In some embodiments, the combinations can contain no resveratrol.

In some embodiments, the sirtuin-pathway activator can be an agent that stimulates the expression of Fndc5, PGC1-α, or UCP1. The expression can be measured in terms of the gene or protein expression level. Alternatively, the sirtuin pathway activator can be irisin. Methods for increasing the level of irisin are described in Boström et al., “A PGC1-α-dependent myokine that drives brown-fat-like development of white fat and thermogenesis,” Nature, Jan. 11, 2012.

In some embodiments, the composition can comprise synergistic combinations of sirtuin pathway activators. For example, a composition can comprise synergistic amounts of metformin and a PDE inhibitor. In some embodiments, the composition comprises metformin and caffeine.

In some embodiments, the activator is a flavones or chalcone. In one embodiment, exemplary sirtuin activators are those described in Howitz et al. (2003) Nature 425: 191 and include, for example, resveratrol (3,5,4′-Trihydroxy-trans-stilbene), butein (3,4,2′,4′-Tetrahydroxychalcone), piceatannol (3,5,3′,4′-Tetrahydroxy-trans-stilbene), isoliquiritigenin (4,2′,4′-Trihydroxychalcone), fisetin (3,7,3′,4′-Tetrahyddroxyflavone), quercetin (3,5,7,3′,4′-Pentahydroxyflavone), Deoxyrhapontin (3,5-Dihydroxy-4′-methoxystilbene 3-O-β-D-glucoside); trans-Stilbene; Rhapontin (3,3′,5-Trihydroxy-4′-methoxystilbene 3-O-β-D-glucoside); cis-Stilbene; Butein (3,4,2′,4′-Tetrahydroxychalcone); 3,4,2′4′6′-Pentahydroxychalcone; Chalcone; 7,8,3′,4′-Tetrahydroxyflavone; 3,6,2′,3′-Tetrahydroxyflavone; 4′-Hydroxyflavone; 5,4′-Dihydroxyflavone 5,7-Dihydroxyflavone; Morin (3,5,7,2′,4′-Pentahydroxyflavone); Flavone; 5-Hydroxyflavone; (−)-Epicatechin (Hydroxy Sites: 3,5,7,3′,4′); (−)-Catechin (Hydroxy Sites: 3,5,7,3′,4′); (−)-Gallocatechin (Hydroxy Sites: 3,5,7,3′,4′,5′) (+)-Catechin (Hydroxy Sites: 3,5,7,3′,4′); 5,7,3′,4′,5′-pentahydroxyflavone; Luteolin (5,7,3′,4′-Tetrahydroxyflavone); 3,6,3′,4′-Tetrahydroxyflavone; 7,3′,4′,5′-Tetrahydroxyflavone; Kaempferol (3,5,7,4′-Tetrahydroxyflavone); 6-Hydroxyapigenin (5,6,7,4′-Tetrahydoxyflavone); Scutellarein); Apigenin (5,7,4′-Trihydroxyflavone); 3,6,2′,4′-Tetrahydroxyflavone; 7,4′-Dihydroxyflavone; Daidzein (7,4′-Dihydroxyisoflavone); Genistein (5,7,4′-Trihydroxyflavanone); Naringenin (5,7,4′-Trihydroxyflavanone); 3,5,7,3′,4′-Pentahydroxyflavanone; Flavanone; Pelargonidin chloride (3,5,7,4′-Tetrahydroxyflavylium chloride); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole-4-ethanaminium inner salt); Caffeic Acid Phenyl Ester; MCI-186 (3-Methyl-1-phenyl-2-pyrazolin-5-one); HBED (N,N′-Di-(2-hydroxybenzyl) ethylenediamine-N,N′-diacetic acid-H2O); Ambroxol (trans-4-(2-Amino-3,5-dibromobenzylamino) cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol.2HCl). Analogs and derivatives thereof can also be used.

The subject application provides compositions useful for inducing an increase in fatty acid oxidation and mitochondrial biogenesis in a subject. Such compositions contain: HMB in combination with resveratrol; leucine in combination with resveratrol; both leucine and HMB in combination with resveratrol; KIC in combination with resveratrol; both KIC and HMB in combination with resveratrol; both KIC and leucine in combination with resveratrol; or KIC, HMB and leucine in combination with resveratrol.

In another embodiment, the subject composition comprises leucine and/or one or more leucine metabolites; and one or more agents selected from the group consisting of nicotinic acid, nicotinamide riboside, and nicotinic acid metabolite; and an anti-diabetic agent such as biguanide (e.g. metformin or any analog thereof), wherein the composition comprises at least about 250 mg of leucine and/or at least about 10, 20, 25, 30, 35, 40, 45, 50, 55, 60 mg of the one or more leucine metabolites, and further wherein the composition is substantially free of each of the amino acids including but are not limited to: alanine, glycine, glutamic acid and proline.

In yet another embodiment, the subject composition comprises leucine and/or one or more leucine metabolites; and an amount of one or more agents selected from the group consisting of nicotinic acid, nicotinamide riboside, and nicotinic acid metabolite; and an anti-diabetic agent such as biguanide (e.g. metformin or any analog thereof) or guanide, wherein the composition comprises at least about 250 mg of leucine and/or at least about 25 mg of the one or more leucine metabolites, and further wherein the amount of one or more agents selected from the group consisting of nicotinic acid, nicotinamide riboside, and nicotinic acid metabolite is insufficient to demonstrate a therapeutic effect such as reducing lipid content in the absence of the leucine and/or one or more leucine metabolites with an anti-diabetic agent such as metformin or any analog thereof. For example, the composition comprises at least about 1 mg of one or more agents selected from the group consisting of nicotinic acid, nicotinamide riboside, and nicotinic acid metabolite. In some embodiments, the amount of the one or more agents selected from the group consisting of nicotinic acid, nicotinamide riboside, and nicotinic acid metabolite is sub-therapeutic when administered without leucine and/or one or more leucine metabolites.

In yet another embodiment, the subject composition comprises leucine and/or one or more leucine metabolites; and an amount of one or more agents selected from the group consisting of nicotinic acid, nicotinamide riboside, and nicotinic acid metabolite; and an anti-diabetic agent such as biguanide (e.g. metformin or any analog thereof) or guanide, wherein the composition comprises at least about 250 mg of leucine and/or at least about 25 mg of the one or more leucine metabolites, and further wherein the amount of anti-diabetic agent may be a sub-therapeutic amount, and/or an amount that is synergistic with one or more other compounds in the composition or one or more other compounds administered simultaneously or in close temporal proximity with the composition.

In yet another embodiment, the subject composition comprises leucine and/or one or more leucine metabolites; and an anti-diabetic agent such as guanide (e.g. dimethylguanidine, metformin or galegine, or any analog thereof), wherein the composition comprises at least about 250 mg of leucine and/or at least about 25 mg of the one or more leucine metabolites, and further wherein the amount of anti-diabetic agent may be a sub-therapeutic amount, and/or an amount that is synergistic with one or more other compounds in the composition or one or more other compounds administered simultaneously or in close temporal proximity with the composition.

In some embodiments, the anti-diabetic agent is administered in a very low dose, a low dose, a medium dose, or a high dose, which describes the relationship between two doses, and generally do not define any particular dose range. For example, a daily very low dose of metformin may comprise about, less than about, or more than about 5 mg/kg, 10 mg/kg, 25 mg/kg, 50 mg/kg, 75 mg/kg, 100 mg/kg, or more; a daily low dose of metformin may comprise about, less than about, or more than about 75 mg/kg, 100 mg/kg, 150 mg/kg, 175 mg/kg, 200 mg/kg, or more; a daily medium dose of metformin may comprise about, less than about, or more than about 150 mg/kg, 175 mg/kg, 200 mg/kg, 250 mg/kg, 300; and a daily high dose of metformin may comprise about, less than about, or more than about 200 mg/kg, 250 mg/kg, 300 mg/kg, 350 mg/kg, 400 mg/kg, 500 mg/kg, 700 mg/kg, or more. In some embodiments, the amount of biguanide such as metformin is sub-therapeutic and when administered with combinations of leucine and nicotinic acids, or metabolites of leucine and/or of nicotinic acids, is sufficient to significantly increase fat oxidation when compared to administering combinations of leucine and nicotinic acids, or metabolites of leucine and/or of nicotinic acids.

In some embodiments a unit dosage can comprise metformin or any analog thereof in about, less than about, or more than about the indicated amounts (e.g. 25, 50, 100, 150, 200, 250, 300, 400, 500, or more mg) in combination with one or more other components in about, less than about, or more than about the indicated amounts (such as 10, 20, 30, 40, 50, 75, 100, or more mg of resveratrol; 50, 100, 200, 300, 400, 500 or more mg of HMB; and/or 400, 500, 600, 700, 800, 900, 1000, 1100, 1250, or more mg of leucine and/or leucine metabolites; and/or 1, 5, 10, 50, 100, 150, 200, 250 mg of one or more agents selected from the group consisting of nicotinic acid, nicotinamide riboside, and nicotinic acid metabolite). In some embodiments, a unit dosage can comprise about, less than about or more than about 50 mg metformin, 500 mg beta hydroxy, beta methyl butyrate and with or without 50 mg resveratrol. A unit dosage can also comprise about, less than about or more than about 50 mg metformin, 1.125 g leucine and with or without 50 mg resveratrol. In some embodiments, a unit dosage can comprise about, less than about or more than about 50 mg metformin, 1.125 g leucine, 50 mg nicotinic acid, and with or without 50 mg resveratrol. A unit dosage can comprise about, less than about or more than about 50 mg metformin, 1.125 g leucine, 250 mg nicotinic acid, and with or without 50 mg resveratrol. A unit dosage can also comprise about, less than about or more than about 50 mg metformin, 1.125 g leucine, 50 mg nicotinic acid and with or without 50 mg resveratrol. A unit dosage can comprise about, less than about or more than about 50 mg metformin, 1.125 g leucine, 250 mg nicotinic acid, and with or without 50 mg resveratrol. In some embodiments, a unit dosage can comprise about, less than about or more than about 100 mg metformin, 500 mg beta hydroxy, beta methyl butyrate and 50 mg resveratrol. A unit dosage can also comprise about, less than about or more than about 100 mg metformin, 1.125 g leucine and with or without 50 mg resveratrol. In some embodiments, a unit dosage can comprise about, less than about or more than about 100 mg metformin, 1.125 g leucine, 50 mg nicotinic acid, and with or without 50 mg resveratrol. A unit dosage can comprise about, less than about or more than about 100 mg metformin, 1.125 g leucine, 250 mg nicotinic acid, and with or without 50 mg resveratrol. A unit dosage can also comprise about, less than about or more than about 100 mg metformin, 1.125 g leucine, 50 mg nicotinic acid and with or without 50 mg resveratrol. A unit dosage can comprise about, less than about or more than about 100 mg metformin, 1.125 g leucine, 250 mg nicotinic acid, and with or without 50 mg resveratrol.

In still yet another embodiment, the subject composition comprises leucine and/or one or more leucine metabolites; and, optionally, one or more agents selected from the group consisting of nicotinic acid, nicotinamide riboside, and nicotinic acid metabolite; and an anti-diabetic agent such as biguanide (e.g. metformin or any analog thereof) or guanide (e.g., galegine, or dimethylguanidine), wherein the composition is effective in treating diabetes and/or hyperlipidemia. The composition can be particularly effective in lowering lipid content and lowering lipid accumulation in a subject in need thereof while causing a reduced degree of cutaneous vasodilation in the subject as compared to a dose of nicotinic acid alone that has the same effectiveness as the composition in lowering lipid content. In some embodiments, the composition is effective in lowering lipid content in a subject in need thereof without causing a clinically significant cutaneous vasodilation.

In yet another embodiment, the subject composition comprises leucine and/or one or more leucine metabolites; an anti-diabetic agent such as biguanide (e.g. metformin or any analog thereof) or guanide or any derivatives thereof and, optionally, an amount of one or more agents selected from the group consisting of nicotinic acid, nicotinamide riboside, and nicotinic acid metabolite, wherein the composition is effective in treating diabetes and/or hyperlipidemia in a subject in need thereof. The composition is effective in increasing insulin sensitivity and increasing glucose utilization in a subject in need thereof while reducing the degree of lactic acidosis and/or hypoglycemia in the subject as compared to a dose of nicotinic acid alone that has the same effectiveness as the composition in lowering lipid content. In some embodiments, the composition is effective in increasing insulin sensitivity and increasing glucose utilization in a subject in need thereof without causing a clinically significant lactic acidosis and/or hypoglycemia.

In yet another embodiment, the subject composition comprises leucine and/or one or more leucine metabolites; and, optionally, an amount of one or more agents selected from the group consisting of nicotinic acid, nicotinamide riboside, and nicotinic acid metabolite; and an anti-diabetic agent such as biguanide (e.g. metformin or any analog thereof) or guanide or any derivatives thereof, wherein the composition is effective in treating diabetes and/or hyperlipidemia. The composition is effective in increasing insulin sensitivity and increasing glucose utilization in a subject in need thereof, wherein the increase in insulin sensitivity is at least about a 1-fold increase (e.g. at least about 1, 2, 3, 4, 5, 6, 8, 10, 15, 20, or 50 fold).

In another embodiment, the subject composition comprises (a) leucine and/or one or more leucine metabolites; and (b) one or more agents selected from the group consisting of nicotinic acid, nicotinamide riboside, and nicotinic acid metabolite; and (c) an anti-diabetic agent such as biguanide (e.g. metformin or any analog thereof) or guanide or a derivative thereof, wherein the mass ratio of (a) to (b), (a) to (c), and/or (b) to (c) is at least about 10, 15, 20, 25, 30, 35, 40, 45, 50, 55, 60, 65, 70, 75, or 100, and wherein the composition comprises at least about 1 mg of the one or more agents selected from the group consisting of nicotinic acid, nicotinamide riboside, and nicotinic acid metabolite. As described herein, a dosing of at least about 1 mg of one or more agents selected from the group consisting of nicotinic acid, nicotinamide riboside, and nicotinic acid metabolite can provide a sub-therapeutic dosing that can be effective when combined with a sufficient mass ratio of leucine or leucine metabolites.

In some embodiments, the subject composition comprises (a) leucine and/or one or more leucine; and (b) one or more agents selected from the group consisting of nicotinic acid, nicotinamide riboside, and nicotinic acid metabolite; and (c) an anti-diabetic agent such as biguanide (e.g. metformin and any analog thereof) or guanide or a derivative thereof, wherein 1) component (a) and component (b), and 2) component (a) and component (c) have synergistic effects, and further wherein component (b) and component (c) may or may not have synergistic effects. The synergistic effects can be synergistically enhances a decrease in weight gain of the subject, a decrease in lipid content, a decrease in lipid accumulation, a decrease in LDL level, a decrease in cholesterol level, a decrease in triglyceride level, an increase in HDL level, an increase in fat oxidation, an increase in insulin sensitivity, an increase in glucose utilization, or an increase in activation of Sirt1 in the subject.

In one aspect of the invention, the subject composition comprises (a) leucine and/or one or more leucine; and (b) optionally, one or more agents selected from the group consisting of nicotinic acid, nicotinamide riboside, and nicotinic acid metabolite; and (c) an anti-diabetic agent such as a guanide or biguanide (e.g. metformin and any analog thereof). The composition can further comprise at least about 0.01, 0.05, 0.1, 0.5, or 1 μg of resveratrol. In some embodiments, the composition is substantially free of resveratrol.

In yet another embodiment, the subject composition comprises (a) leucine and/or one or more leucine metabolites; and (b) one or more agents selected from the group consisting of nicotinic acid, nicotinamide riboside, and nicotinic acid metabolite; and (c) an anti-diabetic agent such as a biguanide (e.g. metformin and any analog thereof), wherein (b) are present in an amount, when administered to a subject, yields a circulating level of about 1-100 nM of the agent(s) in the subject. In some embodiments, the circulating level of the agent(s) is less than about or more than about or is 10 nM. These targeted circulating levels correspond to treatment concentrations described herein (see Examples), which were shown to provide beneficial effects on hyperlipidemic conditions in a subject.

### Branched Chain Amino Acids

The invention provides for compositions that include branched chain amino acids. Branched chain amino acids can have aliphatic side chains with a branch carbon atom that is bound to two or more other atoms. The other atoms may be carbon atoms. Examples of branched chain amino acids include leucine, isoleucine, and valine. Branched chain amino acids may also include other compounds, such as 4-hydroxyisoleucine. In some embodiments, the compositions may be substantially free of one or more, or all of non-branched chain amino acids. For example, the compositions can be free of alanine, arginine, asparagine, aspartic acid, cysteine, glutamic acid, glutamine, glycine, histidine, lysine, methionine, phenylalanine, proline, serine, threonine, tryptophan, and/or tyrosine. In some embodiments, the compositions may be substantially free of isoleucine and/or valine.

In some embodiments, any of the compositions described herein can include salts, derivatives, metabolites, catabolites, anabolites, precursors, and analogs of any of the branched chain amino acids. For example, the metabolites of branched chain amino acids can include hydroxymethylbutyrate (HMB), α-hydroxyisocaproic acid, and keto-isocaproic acid (KIC), keto isovalerate, and keto isocaproate. Non-limiting exemplary anabolites of branched chain amino acids can include glutamate, glutamine, threonine, α-ketobytyrate, α-aceto-α-hydroxy butyrate, α,β-dihydroxy-β-methylvalerate, α-keto-β-methylvalerate, α,β-dihydroxy isovalerate, and α-keto isovalerate.

In certain embodiments of the invention, any of the compositions disclosed herein can be formulated such that they do not contain (or exclude) one or more amino acids selected from the group consisting of lysine, glutamate, proline, arginine, valine, isoleucine, aspartic acid, asparagine, glycine, threonine, serine, phenylalanine, tyrosine, histidine, alanine, tryptophan, methionine, glutamine, taurine, carnitine, cystine and cysteine. The compositions can be substantially free of any non-branched chain amino acids. The mass or molar amount of a non-branched chain amino acid can be less than 0.01, 0.1, 0.5, 1, 2, or 5% of the total composition.

### Leucine and Leucine Metabolites

The invention provides for compositions that include leucine and/or leucine metabolites. The leucine and/or leucine metabolites can be used in free form. The term “free,” as used herein in reference to a component, indicates that the component is not incorporated into a larger molecular complex. For example a composition can include free leucine that is not incorporated in a protein or free hydroxymethylbutyrate. The leucine can be L-leucine. The leucine and/or leucine metabolites can be in a salt form.

Without being limited to theory, ingestion of branched chain amino acids, such as leucine, can stimulate sirtuin signaling, including Sirt1 and Sirt3, as well as AMPK signaling, one or more of which can favorably modulate inflammatory cytokine patterns. In some embodiments, ingestion of leucine can increase insulin sensitivity in blood stream, increase glucose utilization, and stimulate fat oxidation. In some embodiments, any of the compositions described herein can include salts, derivatives, metabolites, catabolites, anabolites, precursors, and analogs of leucine. For example, the metabolites can include hydroxymethylbutyrate (HMB), keto-isocaproic acid (KIC), and keto isocaproate. The HMB can be in a variety of forms, including calcium 3-hydroxy-3-methylbutyrate hydrate. Compositions and methods relating to leucine are described in U.S. Pat. Nos. 8,617,886 and 8,623,924, each of which is hereby incorporated in their entirety by reference.

In certain embodiments of the invention, any of the compositions disclosed herein can be formulated such that they do not contain (or exclude) one or more amino acids selected from the group consisting of lysine, glutamate, proline, arginine, valine, isoleucine, aspartic acid, asparagine, glycine, threonine, serine, phenylalanine, tyrosine, histidine, alanine, tryptophan, methionine, glutamine, taurine, carnitine, glutamic acid, phenylalanine, cystine and cysteine.

In some embodiments, the compositions can be substantially free of one or more, or all of non-branched chain or non-leucine amino acids. For example, the compositions can be free of alanine, arginine, asparagine, aspartic acid, cysteine, glutamic acid, glutamine, glycine, histidine, lysine, methionine, phenylalanine, proline, serine, threonine, tryptophan, and/or tyrosine. In some embodiments, the compositions can be substantially free of isoleucine and/or valine. The subject compositions can be substantially free of the individual amino acids alanine, glycine, glutamic acid, and proline. The subject compositions can be substantially free of one or more of the individual amino acids alanine, glycine, glutamic acid, and proline. The subject compositions can be substantially free of alanine. The subject compositions can be substantially free of glycine. The subject compositions can be substantially free of valine. The compositions can be substantially free of any non-branched chain amino acids. The mass or molar amount of a non-branched chain amino acid can be less than about 0.01, 0.1, 0.5, 1, 2, 5, or 10% of the total composition or of the total amino acids in the composition. The mass or molar amount of a non-leucine amino acid can be less than about 0.01, 0.1, 0.5, 1, 2, 5, or 10% of the total composition or of the total amino acids in the composition.

For clarity, the amino acids described herein can be intact amino acids existing in free form or salt form thereof. For example, the subject compositions can be substantially free of free amino acids, such as alanine, glycine, glutamic acid, and proline. The mass or molar amount of a non-branched chain amino acid, any amino acid, or any non-leucine amino acid can be less than about 0.01, 0.1, 0.5, 1, 2, 5, or 10% of the total composition, of the total amino acids in the composition, or of the total free amino acids in the composition.

### Nicotinic Acid, Nicotinamide Riboside and Nicotinic Acid Metabolites

The invention provides for compositions that include one or more agents selected from the group consisting of nicotinic acid, nicotinamide riboside, and nicotinic acid metabolite. The one or more agents selected from the group consisting of nicotinic acid, nicotinamide riboside, and nicotinic acid metabolite can be used in free form. The term “free”, as used herein in reference to a component, indicates that the component is not incorporated into a larger molecular complex. In some embodiments, the nicotinic acid can be comprised in niacin. Alternatively, the one or more agents selected from the group consisting of nicotinic acid, nicotinamide riboside, and nicotinic acid metabolite can be in a salt form.

In some embodiments, the compositions can be substantially free of nicotinamide and/or nicotinamide metabolites. The nicotinamide and/or nicotinamide metabolites can counteract the effects of nicotinic acid or nicotinamide riboside. Nicotinamide can be harmful to the liver in high doses (as disclosed in http://www.livestrong.com/article/448906-therapeutic-levels-of-niacin-to-lower-cholesterol-levels/#ixzz2NO3KhDZu). The mass or molar amount of nicotinamide and/or nicotinamide metabolites can be less than about 0.01, 0.1, 0.5, 1, 2, 5, or 10% of the total composition. The mass or molar amount of nicotinamide and/or nicotinamide metabolites can be less than about 0.01, 0.1, 0.5, 1, 2, 5, or 10% of the total composition.

Without being limited to theory, ingestion of one or more agents selected from the group consisting of nicotinic acid, nicotinamide riboside, and nicotinic acid metabolite can lower lipid content, lower triglyceride level, lower LDL level, lower total cholesterol level, lower lipid accumulation, or increase HDL level. The ingestion of one or more agents selected from the group consisting of nicotinic acid, nicotinamide riboside, and nicotinic acid metabolite can also increase fat oxidation or stimulate sirtuin signaling, including increase activation of Sirt1 and Sirt3. In some embodiments, any of the compositions described herein can include salts, derivatives, metabolites, catabolites, anabolites, precursors, and analogs of nicotinic acid. For example, the metabolites can include nicotinyl CoA, nicotinuric acid, nicotinate mononucleotide, nicotinate adenine dinucleotide, or nicotinamide adenine dinucleotide. In some embodiments, the compositions cannot comprise nicotinamide. In some embodiments, the compositions comprise nicotinamide. In some embodiments, the compositions can be substantially free of nicotinic acid metabolites.

### Anti-Diabetic Agents

Anti-diabetic agents, also known as drugs used to treat diabetes mellitus, or diabetes as normally referred to, by lowering glucose levels in the blood. Examples of anti-diabetic agents include biguanides (such as metformin), guanidine and/or derivatives thereof (such as galegine), thiazolidinediones and meglitinides (such as repaglinide, pioglitazone, and rosiglitazone), alpha glucosidase inhibitors (such as acarbose), sulfonylureas (such as tolbutamide, acetohexamide, tolazamide, chlorpropamide, glipizide, glyburide, glimepiride, gliclazide), incretins, ergot alkaloids (such as bromocriptine), and DPP inhibitors (such as sitagliptin, vildagliptin, saxagliptin, lingliptin, dutogliptin, gemigliptin, alogliptin, and berberine). In some embodiments, the guanide may comprise a dimethyl structure. The guanide with a dimethyl structure can include dimethylguanidine, galegine, and metformin. In some embodiments, the guanide can include a dimethyl structure that activates the Sirt1/AMPK pathway. The anti-diabetic agent can be an oral anti-diabetic agent and thus are called oral hypoglycemic agents or oral antihyperglycemic agents. The anti-diabetic agent can also be injectable anti-diabetic drugs, including insulin, amylin analogues (such as pramlintide), and inretin mimetics (such as exenatide and liraglutide).

In some embodiments, the anti-diabetic agent is a biguanide including, metformin, phenformin, and buformin. Without being limited to theory, ingestion of a biguanide, such as metformin, has pharmaceutical efficacy in preventing the production of glucose in the liver, increasing sensitivity to insulin and helping the body respond better to its own insulin, and reducing sugar absorption by the intestines. In some embodiments, a biguanide can be a sirtuin pathway activator or an AMPK pathway activator, which increases sensitivity to insulin, hence improving the efficiency of glucose uptake from the blood. In a preferred embodiment of the present invention, a biguanide is metformin.

Metformin can be synthesized from equimolar amounts of 2-cyanoguanidine and dimethylamine in the presence of hydrochloric acid to yield the biguanide, metformin hydrochloride. A common characteristic of metformin, biguanides and guanides is the presence of dimethyl structure. Examples of guanides containing dimethyl structure include, but are not limited to, galegine and dimethylguanidine.

Metformin is known as a medicament for influencing the body's sensitivity to insulin and lowering blood glucose level, and is a FDA approved anti-diabetic agent to treat diabetes and related diseases such as heart disease, blindness and kidney diseases. Commercially available metformin includes Glucophage, Glucopage XR, Glumetza, Fortamet and Riomet. Currently, there are two forms of manufactured metformin, the immediate release Metformin IR and the slow release Metformin SR. Common side effects of metformin include gastrointestinal discomfort, bloating, flatulence, nausea/vomiting and diarrhea. Less common reactions include hypoglycemia, myalgia, lightheadedness, dyspnea, rash, increased sweating, taste disorder, and flu-like syndrome. Lactic acidosis is a rare side effect of anti-diabetic medicaments containing metformin. In general, metformin does not increase insulin concentration in the blood or cause hypoglycemia when used alone. Treatment of metformin has good effect on LDL cholesterol while has no effect on blood pressure. In addition, treatment of metformin can decrease triglycerides. In some cases, metformin can be administrated as a monotherapy or in combination with other medicaments, for example, metformin with sitagliptin (commercially known as Janumet), metformin with pioglitazone (commercially known as Competact), and metformin with vildagliptin (commercially known as Eucreas). In a preferred embodiment of the present invention, a sub-therapeutic amount of metformin and any analog thereof is administrated with leucine and/or leucine metabolites in combination with nicotine acid and/or nicotinamide riboside and/or nicotinic acid metabolites to lower lipid levels, to lower lipid accumulation, to increase fat oxidation, to increase glucose utilization and to increase insulin sensitivity for diabetes control in a subject with a reduction in side effects such as lactic acidosis and/or hypoglycemia. In some embodiments, the combination of sub-therapeutic amount of metformin with leucine and nicotinic acid described herein is sufficient to lower lipid levels, to lower lipid accumulation, to increase fat oxidation, to increase glucose utilization and to increase insulin sensitivity for diabetes control without causing a clinically significant lactic acidosis and/or hypoglycemia.

In some embodiments, the compositions can comprise guanidine and guanidine derivatives, including the alkaloid galegine, which can be isolated from the French lilac, also known as Goats rue (Galega officinalis) as described in Witters L. A., “The blooming of the French lilac,” J. Clin Invest 108, 1105-1107 Oct. 15, 2001. In some cases, the toxicity of guanidine can preclude its development as a therapeutic, and the galegine effects can be less potent than those of metformin. In some embodiments, galegine and metformin can act via the same pathway, by dose-responsive activation of AMPK, as described in Mooney et al., “Mechanisms underlying the metabolic actions of galegine that contribute to weight loss in mice,” Br. J Pharmacol 153, 1669-1677 Feb. 25, 2008. In some embodiments, guanides containing the dimethyl structure, such as galegine and dimethylguanidine, synergize with leucine, thereby increasing the efficacy of the guanide, which allows for use of such guanides for as a therapeutic treatment for diabetes at non-toxic doses of such guanides.

### Therapeutic Agents

The subject compositions can further include one or more pharmaceutically active agent or therapeutic agents other than one or more agents selected from the group consisting of nicotinic acid, nicotinamide riboside, and nicotinic acid metabolite, or biguanide and/or any analog thereof. The therapeutic agents or pharmaceutically active agents can be any agent that is known in the art. For example, the combination compositions can further comprise a pharmaceutically active anti-hyperlipidemic agent, and/or a pharmaceutically active anti-diabetic agent, or a dietary supplement that also have beneficial effects on lipid content, and/or insulin sensitivity. The anti-hyperlipidemic agents and/or anti-diabetic agent can be in a sub-therapeutic amount in lowering levels of total lipid content or triglyceride, LDL or cholesterol levels, or increasing the HDL level, or increasing glucose utilization, or increasing insulin sensitivity, or increasing fat oxidation. The anti-hyperlipidemic agent can be an oral agent or injectable agent. The types of the anti-hyperlipidemic agents known in the art can include, but are not limited to, HMG-CoA inhibitors (or statins), fibrates, nicotinic acid, bile acid sequestrants (resins), cholesterol absorption inhibitors (ezetimibe), lomitapide, phytosterols, orlistat or others. The statin type anti-hyperlipidemic agents can include but are not limited to: atorvastatin, fluvastatin, pravastatin, lovastatin, simvastatin, pitavastatin, cerivastatin, rosuvastatin, or lovastatin/niacin ER. The cholesterol absorption inhibitors can include but are not limited to ezetimibe, and combination of ezetimibe with simvastatin. The fibrate type of anti-hyperlipidemic agents can include but are not limited to: gemfibrozil, fenofibrate, fenofibric acid, clofibrate, or micronized fenofibrate. The bile acid sequestrants can include but are not limited to: colestipol, cholestyramine, or colesevelam. Other types of anti-hyperlipidemic agent can include dextrothyroxine sodium or icosapent. The types of anti-diabetic agents known in the art can include, but are not limited to, biguanides (such as metformin), thiazolidinediones and meglitinides (such as repaglinide, pioglitazone, and rosiglitazone), alpha glucosidase inhibitors (such as acarbose), sulfonylureas (such as tolbutamide, acetohexamide, tolazamide, chlorpropamide, glipizide, glyburide, glimepiride, gliclazide), incretins, ergot alkaloids (such as bromocriptine), and DPP inhibitors (such as sitagliptin, vildagliptin, saxagliptin, lingliptin, dutogliptin, gemigliptin, alogliptin, and berberine). The anti-diabetic agent can be an oral anti-diabetic agent and thus are called oral hypoglycemic agents or oral antihyperglycemic agents. The anti-diabetic agent can also be injectable anti-diabetic drugs, including insulin, amylin analogues (such as pramlintide), and inretin mimetics (such as exenatide and liraglutide). These examples are provided for discussion purposes only, and are intended to demonstrate the broad scope of applicability of the invention to a wide variety of drugs. It is not meant to limit the scope of the invention in any way.

In some embodiments, one or more components described herein, such as resveratrol, leucine, HMB, and KIC can be combined with two or more pharmaceutically active agents. For example, a sirtuin pathway activator can be combined with glipizide and metformin, glyburide and metformin, pioglitazone and glimepiride, pioglitazone and metformin, repaglinide and metformin, rosiglitazone and glimepiride, rosiglitazone and metformin, or sitagliptin and metformin. In some embodiments, leucine can be combined with metformin and nicotinic acids with or without resveratrol.

The subject composition can further comprise one or more therapeutic agents that are herbs and/or supplements. The herbs and/or supplements can have therapeutic effects that are unproven scientifically. The examples of the herbs and/or the supplements can be, but are not limited to: Acai, Alfalfa, Aloe, Aloe Vera, Aristolochic Acids, Asian Ginseng, Astragalus, Bacillus coagulans, Belladonna, Beta-carotene, Bifidobacteria, Bilberry, Bilberry, Biotin, Bitter Orange, Black Cohosh, Black Cohosh, Black psyllium, Black tea, Bladderwrack, Blessed thistle, Blond psyllium, Blueberry, Blue-green algae, Boron, Bromelain, Butterbur, Calcium, Calendula, Cancell/Cantron/Protocel, Cartilage (Bovine and Shark), Cassia cinnamon, Cat's Claw, Chamomile, Chasteberry, Chondroitin sulfate, Chromium, Cinnamon, Clove, Coenzyme Q-10, Colloidal Silver Products, Cranberry, Creatine, Dandelion, Dandelion, Devil's claw, DHEA, Dong quai, Echinacea, Ephedra, Essiac/Flor-Essence, Eucalyptus, European Elder (Elderberry), European Mistletoe, Evening Primrose Oil, Fenugreek, Feverfew, Fish oil, Flaxseed, Flaxseed oil, Folate, Folic acid, Garlic, Ginger, Gingko, Ginseng, Glucosamine hydrochloride, Glucosamine sulfate, Goldenseal, Grape Seed Extract, Green Tea, Hawthorn, Hoodia, Horse Chestnut, Horsetail, Hydrazine Sulfate, Iodine, Iron, Kava, Lactobacillus, Laetrile/Amygdalin, L-arginine, Lavender, Licorice, Lycium, Lycopene, Magnesium, Manganese, Melatonin, Milk Thistle, Mistletoe Extracts, Noni, Oral Probiotics, Pantothenic acid (Vitamin B5), Passionflower, PC-SPES, Pennyroyal, Peppermint, Phosphate salts, Pomegranate, Propolis, Pycnogenol, Pyridoxine (Vitamin B6), Red Clover, Red yeast, Riboflavin (Vitamin B2), Roman chamomile, Saccharomyces boulardii, S-Adenosyl-L-Methionine (SAMe), Sage, Saw Palmetto, Selected Vegetables/Sun's Soup, Selenium, Senna, Soy, St. John's Wort, sweet orange essence, Tea Tree Oil, Thiamine (Vitamin B1), Thunder God Vine, Turmeric, Valerian, Vitamin A, Vitamin B12, Vitamin C, Vitamin D, Vitamin E, Vitamin K, Wild yam, Yohimbe, Zinc or 5-HTP.

The amount of pharmaceutical agent, or any other component used in a combination composition described herein, can be used in an amount that is sub-therapeutic. In some embodiments, using sub-therapeutic amounts of an agent or component can reduce the side-effects of the agent. Use of sub-therapeutic amounts can still be effective, particularly when used in synergy with other agents or components.

A sub-therapeutic amount of the agent or component such as metformin or nicotinic acid, can be such that it is an amount below which would be considered therapeutic. For example, FDA guidelines can suggest a specified level of dosing to treat a particular condition, and a sub-therapeutic amount would be any level that is below the FDA suggested dosing level. The sub-therapeutic amount can be about 1, 5, 10, 15, 20, 25, 30, 35, 50, 75, 90, or 95% less than the amount that is considered to be a therapeutic amount. The therapeutic amount can be assessed for individual subjects, or for groups of subjects. The group of subjects can be all potential subjects, or subjects having a particular characteristic such as age, weight, race, gender, genetic variations, or physical activity level.

In the case of metformin hydrochloride, the physician suggested starting dose is 1000 mg daily, with subject specific dosing having a range of 500 mg to a maximum of 2550 mg daily (metformin hydrochloride extended-release tablets label www.accessdata.fda.gov/drugsatfda_docs/label/2008/021574s010lbl.pdf). The particular dosing for a subject can be determined by a clinician by titrating the dose and measuring the therapeutic response. The therapeutic dosing level can be determined by measuring fasting plasma glucose levels and measuring glycosylated hemoglobin. A sub-therapeutic amount can be any level that would be below the recommended dosing of metformin. For example, if a subject's therapeutic dosing level is determined to be 700 mg daily, a dose of 600 mg would be a sub-therapeutic amount. Alternatively, a sub-therapeutic amount can be determined relative to a group of subjects rather than an individual subject. For example, if the average therapeutic amount of metformin for subjects with weights over 300 lbs is 2000 mg, then a sub-therapeutic amount can be any amount below 2000 mg. In some embodiments, the dosing can be recommended by a healthcare provider including, but not limited to a patient's physician, nurse, nutritionist, pharmacist, or other health care professionals. A health care professional may include a person or entity that is associated with the health care system. Examples of health care professionals may include surgeons, dentists, audiologists, speech pathologists, physicians (including general practitioners and specialists), physician assistants, nurses, midwives, pharmaconomists/pharmacists, dietitians, therapists, psychologists, physical therapists, phlebotomists, occupational therapists, optometrists, chiropractors, clinical officers, emergency medical technicians, paramedics, medical laboratory technicians, radiographers, medical prosthetic technicians, social workers, and a wide variety of other human resources trained to provide some type of health care service.

In the case of nicotinic acid administered alone to lower lipid content, the physician suggested starting dose is 1000-3000 mg daily, with subject specific dosing having a range of 1 mg to a maximum of 1000 mg daily when administered with leucine and/or leucine metabolites. The particular dosing for a subject can be determined by a clinician by titrating the dose and measuring the therapeutic response. The therapeutic dosing level can be determined by measuring fasting plasma cholesterol and LDL levels without causing clinically significant cutaneous vasodilation. A sub-therapeutic amount can be any level that would be below the recommended dosing of nicotinic acid. For example, if a subject's therapeutic dosing level is determined to be 700 mg daily, a dose of 600 mg would be a sub-therapeutic amount. Alternatively, a sub-therapeutic amount can be determined relative to a group of subjects rather than an individual subject. For example, if the average therapeutic amount of nicotinic acid, nicotinamide riboside or nicotinic acid metabolites for subjects with weights over 300 lbs is 2000 mg, then a sub-therapeutic amount can be any amount below 2000 mg. In some embodiments, the dosing can be recommended by a healthcare provider including, but not limited to a patient's physician, nurse, nutritionist, pharmacist, or other health care professional. A health care professional can include a person or entity that is associated with the health care system. Examples of health care professionals can include surgeons, dentists, audiologists, speech pathologists, physicians (including general practitioners and specialists), physician assistants, nurses, midwives, pharmaconomists/pharmacists, dietitians, therapists, psychologists, physical therapists, phlebotomists, occupational therapists, optometrists, chiropractors, clinical officers, emergency medical technicians, paramedics, medical laboratory technicians, radiographers, medical prosthetic technicians, social workers, and a wide variety of other human resources trained to provide some type of health care service.

In the case of nicotinic acid, nicotinamide riboside, or nicotinic acid metabolites, the therapeutically effective level of the nicotinic acid, nicotinamide riboside, nicotinic acid metabolites can be a circulating level between about 1-100 nM. A sub-therapeutic level of the nicotinic acid, nicotinamide riboside, or nicotinic acid metabolites, by itself or in any combination, can be any circulating level at least about, less than about, or more than about 1, 2.5, 5, 10, 15, 20, 25, 30, 35, 40, 45, 50, 60, 70, 80, 90 or 100 nM. The sub-therapeutic level of the one or more agents selected from the group consisting of nicotinic acid, nicotinamide riboside, and nicotinic acid metabolite, in a subject composition formulated for administration can be less than about 1, 5, 10, 20, 30, 50, 60, 70, 80, 90, 100, 125, 150, 175, 200, 250, 300, 350, 400, 450, 500, 600, 700, 750, 800, 900 or 1000 mg of the one or more agents selected from the group consisting of nicotinic acid, nicotinamide riboside, and nicotinic acid metabolite.

Any of the components described herein, including leucine, HMB, KIC, nicotinic acid, nicotinamide riboside, and resveratrol can be used in a subject composition in free form, isolated form, purified from a natural source, and/or purified or prepared from a synthetic source. The natural source can be an animal source or plant source. The components can be pure to at least about 95, 97, 99, 99.5, 99.9, 99.99, or 99.999%.

### Dosing Amounts

The invention provides for compositions comprising a combination of (a) leucine and/or one or more leucine metabolites, (b) one or more agents selected from the group consisting of nicotinic acid, nicotinamide riboside, and nicotinic acid metabolite, and (c) an anti-diabetic agent. In some embodiments, the weight percentage of component (a) is between about 60-100%, 70-95%, 80-95%, 80-98% or 85-90% of the total composition. In some embodiments, the weight percentage of component (b) is between about 0-50%, 1-5%, 1-30%, 2-25%, or 5-20% of the total composition. In some embodiments, the weight percentages of component (c) is between about 0-30%, 0.5-20%, 1-20%, 5-20%, 1-10%, 1-15% or 1-5% of the total composition.

The compositions, methods, and kits can contain amounts of (a) leucine and/or at least one or more leucine metabolites in combination and (b) at least one or more anti-diabetic agents that is a guanide. In some embodiments, the weight percentage of component (a) is between about 60-100%, 70-95%, 80-95%, 80-98% or 85-90% of the total composition. In some embodiments, the weight percentages of component (b) is between about 0-30%, 0.5-20%, 1-20%, 5-20%, 1-10%, 1-15% or 1-5% of the total composition.

The invention provides for compositions that are combinations of isolated components, such as leucine, metabolites of leucine, such as HMB, guanides, biguanide, such as metformin, nicotinic acid, nicotinamide riboside, and/or resveratrol, that have been isolated from one or more sources. The invention provides for compositions that are enriched in leucine, metabolites of leucine, such as HMB; biguanide, such as metformin; nicotinic acid and/or nicotinamide riboside; and with our without resveratrol. The components can be isolated from natural sources or created from synthetic sources and then enriched to increase the purity of the components. Additionally, leucine can be isolated from a natural source and then be enriched by one or more separations. The isolated and enriched components, such as leucine, can then be combined and formulated for administration to a subject.

In some embodiments, a composition comprises an amount of one or more agents selected from the group consisting of nicotinic acid, nicotinamide riboside, and nicotinic acid metabolite. The amount of one or more agents selected from the group consisting of nicotinic acid, nicotinamide riboside, and nicotinic acid metabolite can be a sub-therapeutic amount, and/or an amount that is synergistic with one or more other compounds in the composition or one or more of the compounds administered simultaneously or in close temporal proximity with the composition. In some embodiments, the one or more agents selected from the group consisting of nicotinic acid, nicotinamide riboside, and nicotinic acid metabolite is administered in a low dose, a medium dose, or a high dose, which describes the relationship between two doses, and generally do not define any particular dose range. The compositions can be administered to a subject such that the subject is administered a selected total daily dose of the composition. The total daily dose can be determined by the sum of doses administered over a 24 hour period.

In some embodiments, a composition comprises an amount of a sirtuin pathway activator, such as a polyphenol (e.g. resveratrol) or a biguanide (e.g. metformin and any analogs thereof). The amount of sirtuin pathway activator may be a sub-therapeutic amount, and/or an amount that is synergistic with one or more other compounds in the composition or one or more other compounds administered simultaneously or in close temporal proximity with the composition. In some embodiments, the sirtuin pathway activator is administered in a low dose, a medium dose, or a high dose, which describes the relationship between two doses, and generally do not define any particular dose range. For example, a daily low dose of resveratrol may comprise about, less than about, or more than about 0.5 mg/kg, 1 mg/kg, 2.5 mg/kg, 5 mg/kg, 7.5 mg/kg, 10 mg/kg, or more; a daily medium dose of resveratrol may comprise about, less than about, or more than about 12.5 mg/kg, 15 mg/kg, or more; and a daily high dose of resveratrol may comprise about, less than about, or more than about 20 mg/kg, 25 mg/kg, 50 mg/kg, 75 mg/kg, 100 mg/kg, 125 mg/kg, 150 mg/kg, 175 mg/kg, 200 mg/kg, 225 mg/kg, 250 mg/kg, 300 mg/kg, 350 mg/kg, 400 mg/kg, or more. In yet another example, a dosage administered to a subject can be suggested by a physician or a health care provider as described herein.

A dose, which can be a unit dose, can comprise about, more than about, or less than about 200, 250, 400, 500, 550, 600, 700, 800, 900, 1000, 1100, 1250, 1300 or more mg of leucine. The leucine can be free leucine. In some embodiments, a unit dose can comprise at least about 1000 mg of free leucine. The composition can comprise between about 10-1250, 200-1250, or 500-1250 mg of leucine. A dose, which can be a unit dose, can comprise about, more than about, or less than about 10, 15, 20, 25, 30, 35, 40, 45, 50, 100, 200, 250, 400, 500, 550, 600, 700, 800, 900, 1000, 1250, 1300 or more mg of a leucine metabolite, such as HMB or KIC. The leucine metabolite can be a free leucine metabolite. The composition can comprise between about 10-900, 50-750, or 400-650 mg of the leucine metabolite, such as HMB or KIC. In some embodiments, a unit dose can comprise at least about 400 mg of free HMB. The amount of leucine and leucine metabolites as described herein can be administered daily or simultaneously. The amount as described herein can be administered in one dose or separately in multiple doses daily.

In some embodiments, a daily dose of leucine can be about, less than about, or more than about 0.5, 0.75, 1, 1.25, 1.5, 1.75, 2, 2.5, 3, or more g/day. A daily dose of leucine can be between about 0.25-3 or 0.5-3.0 g/day. A daily dose of HMB can be about, less than about, or more than about 0.2, 0.4, 0.5, 0.75, 1, 1.5, 2, 2.5, 3, or more g/day. A daily dose of HMB can be between about 0.20-3.0 g/day. A daily dose of KIC can be about, less than about, or more than about 0.2, 0.4, 0.5, 0.75, 1, 1.25, 1.5, 1.75, 2, 2.5, 3, or more g/day. A daily dose of KIC can be between about 0.2-3.0 g/day.

The dose of leucine or metabolites thereof, can be a therapeutic dose. The dose of leucine or metabolite thereof can be a sub-therapeutic dose. A sub-therapeutic dose of leucine can be about, less than about, or more than 0.25, 0.5, 0.75, 1, 1.25, 1.5, 1.75, 2, 2.5, 3, or more g. A sub-therapeutic dose of leucine can be between about 0.25-3.0 g. A sub-therapeutic dose of leucine can be about, less than about, or more than about 0.25, 0.5, 0.75, 1, 1.25, 1.5, 1.75, 2, 2.5, 3, or more g/day. A sub-therapeutic dose of leucine can be between about 0.25-3.0 g/day. In some embodiments, the compositions comprises less than 3.0 g daily dosage of leucine. A sub-therapeutic dose of HMB can be about, less than about, or more than about 0.05, 0.1, 0.2, 0.4, 0.5, 0.75, 1, 1.5, 2, 2.5, 3, or more g. A sub-therapeutic dose of HMB can be between about 0.05-3.0 g. A sub-therapeutic dose of HMB can be about 0.05, 0.1, 0.2, 0.4, 0.5, 0.75, 1, 1.5, 2, 2.5, 3, or more g/day. A sub-therapeutic dose of HMB can be between about 0.05-3.0 g/day. A sub-therapeutic dose of KIC can be about 0.1, 0.2, 0.4, 0.5, 0.75, 1, 1.25, 1.5, 1.75, 2, 2.5, 3, or more g. A sub-therapeutic dose of KIC can be between about 0.1-3.0 g. A sub-therapeutic dose of KIC can be about 0.1, 0.2, 0.4, 0.5, 0.75, 1, 1.25, 1.5, 1.75, 2, 2.5, 3, or more g/day. A sub-therapeutic dose of KIC can be between about 0.1-3.0 g/day.

A dose, which can be a unit dose, can comprise nicotinic acid, nicotinamide riboside or nicotinic acid metabolites, that can be about, more than about, or less than about 0.01, 0.05, 0.1, 0.5, 1, 2, 5, 10, 20, 40, 60, 80, 100, 200, 250, 400, 500, 800, 1000, or 1500 mg of the nicotinic acid, nicotinamide riboside, or nicotinic acid metabolites. The composition can comprise between about 1-100, or 5-50, or 10-20 mg of the one or more agents selected from the group consisting of nicotinic acid, nicotinamide riboside, and nicotinic acid metabolite. In some embodiments, a unit dose can comprise at least about 1 mg of one or more agents selected from the group consisting of nicotinic acid, nicotinamide riboside, and nicotinic acid metabolite. In some embodiments, a unit dose can comprise less than 250 mg of one or more agents selected from the group consisting of nicotinic acid, nicotinamide riboside, and nicotinic acid metabolite. The dosage can be adjusted for the intended subject administered. For example, a dose that is suitable for a canine can be less than the dose that is suitable for a human. The amount of nicotinic acid, nicotinamide riboside and/or nicotinic acid metabolites as described herein can be administered daily or simultaneously. The amount as described herein can be administered in one dose or separately in multiple doses daily.

A dose of the anti-diabetic agent, which can be a unit dose, can comprise a thiazolidinedione, guanide or a biguanide such as dimethylguanidine, galegine, rosiglitazone, metformin or any analogs thereof, that can be about, more than about, or less than about 0.01, 0.05, 0.1, 0.5, 1, 5, 10, 25, 50, 100, 150, 200, 250, 300, 350, 400, 450, 500, 1000, 1500, 2000, or 2550 mg per day. A dose of the anti-diabetic agent, which can be a unit dose, can comprise a guanide such as galegine or dimethylguanidine, or a biguanide such as metformin or any analogs thereof that can be between about 1-2550, 5-500, 5-50, 10-25, 20-50, 30-75, 10-100, 0.01-10, 0.05-20, 0.05-50, 0.1-10, 1-10, 1-20 or 25-2550 mg per day. The particular dosing of the anti-diabetic agent for a subject can be determined by a physician or a health care provider as described herein.

In some embodiments, the composition comprises both nicotinic acid and nicotinamide riboside, and the total amount of nicotinic acid and nicotinamide riboside can be about, more than about, or less than about 0.01, 0.05, 0.1, 0.5, 1, 2, 5, 10, 20, 40, 60, 80, 100, 200, 250, 400, 500, 600, 800, 900, 1000, or 1500 mg.

In other embodiments, a daily dose of one or more agents selected from the group consisting of nicotinic acid, nicotinamide riboside, and nicotinic acid metabolite can be about, more than about, or less than about 0.0001 mg/kg (mg of one or more agents selected from the group consisting of nicotinic acid, nicotinamide riboside, and nicotinic acid metabolite/kg of the subject receiving the dose), 0.005 mg/kg, 0.01 mg/kg, 0.5 mg/kg, 1 mg/kg, 2.5 mg/kg, 5 mg/kg, 7.5 mg/kg, 10 mg/kg, 12.5 mg/kg, 15 mg/kg, 20 mg/kg, 25 mg/kg, 50 mg/kg, 75 mg/kg, 100 mg/kg, or more.

A dose, which can be a unit dose, can comprise about, less than about, or more than about 1, 5, 10, 25, 35, 50, 51, 75, 100, 150, 200, 250, 300, 350, 400, 450, 500, or more mg of resveratrol. The composition can comprise between about 5-500, 30-250, or 35-100 mg of resveratrol. In some embodiments, a unit dose can comprise at least about 35 mg of resveratrol. The amount of resveratrol as described herein can be administered daily or simultaneously. The amount as described herein can be administered in one dose or separately in multiple doses daily.

A daily low dose of resveratrol can comprise about, less than about, or more than about 0.5 mg/kg (mg of resveratrol/kg of the subject receiving the dose), 1 mg/kg, 2.5 mg/kg, 5 mg/kg, 7.5 mg/kg, 10 mg/kg, 12.5 mg/kg, 15 mg/kg, 20 mg/kg, 25 mg/kg, 50 mg/kg, or more; a daily medium dose of resveratrol can comprise about, less than about, or more than about 20 mg/kg, 25 mg/kg, 50 mg/kg, 75 mg/kg, 100 mg/kg, 125 mg/kg, 150 mg/kg, 175 mg/kg, 200 mg/kg, 250 mg/kg, or more; and a daily high dose of resveratrol can comprise about, less than about, or more than about 150 mg/kg, 175 mg/kg, 200 mg/kg, 225 mg/kg, 250 mg/kg, 300 mg/kg, 350 mg/kg, 400 mg/kg, or more. The dosing range as defined to low, medium or high can be dependent on the subject receiving the dose and vary from subject to subject.

In some embodiments, a composition, which can be formulated as a unit dose, can comprise (a) at least about 250 mg of leucine and/or at least about 25 mg of the one or more leucine metabolites. The composition can further comprise at least about 35 mg of resveratrol.

In some embodiments of the invention, the combination compositions can have a specified ratio of leucine and/or metabolites thereof to nicotinic acid and/or nicotinamide metabolites and/or nicotinic acid metabolites, and to an anti-diabetic agent such as metformin or any analogs thereof. The specified ratio can provide for effective and/or synergistic treatment of hyperlipidemic conditions, which, for example, can be measured as a reduction in total lipid content, reduction in cholesterol level, reduction in triglyceride level, reduction in LDL level, reduction in body weight, reduction in lipid accumulation, increase in HDL level, increase in fat oxidation, increase in insulin sensitivity, and/or increase in glucose utilization. The ratio of leucine amino acids and/or metabolites thereof to a nicotinic acid and/or nicotinamide riboside and/or nicotinic acid metabolite, and to an anti-diabetic agent such as metformin or any analogs thereof can be a mass ratio, a molar ratio, or a volume ratio.

In some embodiments, a composition can comprise (a) leucine and/or metabolites thereof (including HMB) and (b) one or more agents selected from the group consisting of nicotinic acid, nicotinamide riboside, and nicotinic acid metabolite, and/or (c) an anti-diabetic agent such as metformin or any analogs thereof, where the mass ratio of (a) to (b), (a) to (c), and/or (b) to (c) can be about, less than about, or greater than about 0.1, 0.5, 1, 2, 5, 10, 15, 20, 25, 50, 75, 100, 200, 300, 350, 400, 450, 500, 550, 600, 650, 700, 750, or 800. In some embodiments, the mass ratio of (a) to (b), (a) to (c), and/or (b) to (c) is at least about 20. In some embodiments, the mass ratio of (a) to (b), (a) to (c), and/or (b) to (c) is at least about 25. In some embodiments, the mass ratio of (a) to (b), (a) to (c), and/or (b) to (c) is at least about 50. The composition can also comprise a minimal amount of one or more agents selected from the group consisting of nicotinic acid, nicotinamide riboside, and nicotinic acid metabolite, such as 5, 10 or 50 mg of the one or more agents selected from the group consisting of nicotinic acid, nicotinamide riboside, and nicotinic acid metabolite or a range of one or more agents selected from the group consisting of nicotinic acid, nicotinamide riboside, and nicotinic acid metabolite amount, such as between about 5-250 mg of one or more agents selected from the group consisting of nicotinic acid, nicotinamide riboside, and nicotinic acid metabolite.

In some embodiments, a composition can comprise (a) leucine and/or metabolites thereof (including HMB) and (b) an anti-diabetic agent such as a guanide or metformin or any analogs thereof, where the mass ratio of (a) to (b), can be about, less than about, or greater than about 0.1, 0.5, 1, 2, 5, 10, 15, 20, 25, 50, 75, 100, 200, 300, 350, 400, 450, 500, 550, 600, 650, 700, 750, or 800. In some embodiments, the mass ratio of (a) to (b) is at least about 20. In some embodiments, the mass ratio of (a) to (b) is at least about 25. In some embodiments, the mass ratio of (a) to (b) is at least about 50.

In other embodiments, a composition can comprise (a) one or more agents selected from the group consisting of nicotinic acid, nicotinamide riboside, and nicotinic acid metabolite and (b) resveratrol, where the mass ratio of (a) to (b) can be about, less than about, or greater than about 0.01, 0.05, 0.1, 0.5, 1, 2, 5, 10, 20, 50, 100, 200, 300, 350, 400, 450, 500, 550, 600, or 650.

In other embodiments, a composition can comprise (a) one or more agents selected from the group consisting of nicotinic acid, nicotinamide riboside, and nicotinic acid metabolite and (b) an anti-diabetic agent such as a guanide or metformin or any analogs thereof, where the mass ratio of (a) to (b) can be about, less than about, or greater than about 0.01, 0.05, 0.1, 0.5, 1, 2, 5, 10, 20, 50, 100, 200, 300, 350, 400, 450, 500, 550, 600, or 650.

In some embodiments, the dosing of leucine, any metabolites of leucine, nicotinic acid, nicotinamide riboside, any nicotinic acid metabolites, and resveratrol can be designed to achieve a specified physiological concentration or circulating level of leucine, metabolites of leucine, nicotinic acid, nicotinamide riboside, metabolites of nicotinic acid and/or resveratrol. The physiological concentration can be a circulating level as measured in the serum or blood stream of a subject. The subject can be a human or an animal. A selected dosing can be altered based on the characteristics of the subject, such as weight, rate of energy metabolism, genetics, ethnicity, height, or any other characteristic.

In some embodiments, a selected dose of a composition can be administered to a subject such that the subject achieves a desired circulating level of a particular component. The desired circulating level of a component can be either a therapeutically effective level or a sub-therapeutic level.

The amount of leucine in a unit dose can be such that the circulating level of leucine in a subject is about or greater than about 0.25 mM, 0.5 mM, 0.75 mM, or 1 mM. A dosing of about 1,125 mg leucine (e.g., free leucine), can achieve a circulating level of leucine in a subject that is about 0.5 mM. A dosing of about 300 mg leucine (e.g., free leucine), can achieve a circulating level of leucine in a subject that is about 0.25 mM.

The desired circulating level of the leucine can be at least about 0.25, 0.3, 0.5, 0.75, 1 mM or more of leucine. The desired circulating level of the leucine can be between about 0.25-1, 0.25-0.5, 0.3-0.5, 0.5-1 or 0.5-0.75 mM. The desired circulating level of the leucine metabolites can be at least about, less than about, or more than about 0.1, 0.25, 0.3, 0.5, 0.75, 1, 10, 20, 40, 60 μM or more of a leucine metabolite (such as HMB). The desired circulating level of the leucine metabolites can be between about 0.1-60, 0.1-30, 0.25-30, 0.25-60, 0.5-40 or 0.5-60 μM of a leucine metabolite (such as HMB). The desired circulating level of the KIC can be at least about 0.25, 0.5, 0.75, 1 mM or more of KIC. The desired circulating level of the KIC can be between about 0.25-1, 0.25-0.5, 0.3-0.5, 0.5-1 or 0.5-0.75 mM KIC.

The desired circulating or serum level of the one or more agents selected from the group consisting of nicotinic acid, nicotinamide riboside, and nicotinic acid metabolite can be at least about, less than about, or more than about 0.1, 0.25, 0.5, 0.75, 1, 10, 20, 40, 60, 80, 100, 120, 200, 400, 500, 1000, 1500, 2000, 2550, or 3000 nM or more of the one or more agents selected from the group consisting of nicotinic acid, nicotinamide riboside, and nicotinic acid metabolite. The desired circulating or serum level of the one or more agents selected from the group consisting of nicotinic acid, nicotinamide riboside, and nicotinic acid metabolite can be between about 0.1-3000, 0.1-10, 10-100, 10-500, 100-1000, 1-10, 1-100, 1000-3000 or 1-1000 nM of the one or more agents selected from the group consisting of nicotinic acid, nicotinamide riboside, and nicotinic acid metabolite. The therapeutically effective level of one or more agents selected from the group consisting of nicotinic acid, nicotinamide riboside, and nicotinic acid metabolite can be between 44-111 μM, which corresponds to about 10-20 μg/mL.

The desired circulating level of the of the anti-diabetic agent can be at least about, less than about, or more than about 0.01, 0.03, 0.05, 0.08, 0.1, 0.12, 0.15, 0.2, 0.25, 0.3, 1, 5, 10, 20, 30, 40, 50, 75, 100, 150, 200, 500 μM or more of the anti-diabetic agent such as galegine, dimethylguanidine, metformin or any analogs thereof. The desired circulating level of the of the anti-diabetic agent can be between about 0.01-500, 0.01-1, 1-10, 10-100, 100-500, 1-100 or 1-200 μM of the anti-diabetic agent such as galegine, dimethylguanidine, metformin or any analogs thereof. The selected dose can be chosen based on the characteristics of the subject, such as weight, height, ethnicity, or genetics.

The desire circulating level of the resveratrol can be at least about, less than about, or more than about 40, 60, 80, 100, 120, 150, 200, 300, 400, 800, 1600, 3000, or 5000 nM or more of the resveratrol. The desire circulating level of the resveratrol can be between about 40-5000 nM of the resveratrol. The selected dose can be chosen based on the characteristics of the subject, such as weight, height, ethnicity, or genetics.

In some embodiments, a composition comprises leucine and nicotinic acid in amounts that are effective to achieve a circulating level of about 0.3-1 mM leucine and about 1-100 nM nicotinic acid in a subject.

An oral dosing of about 1,125 mg leucine can achieve a circulating level of leucine in a subject that is about 0.5 mM leucine. An oral dosing of about 300 mg leucine can achieve a circulating level of leucine in a subject that is about 0.25 mM.

An oral dosing of about 500 mg of HMB can achieve a circulating level of HMB in a subject that is about 5 μM HMB. An oral dosing of about 100 mg of HMB can achieve a circulating level of HMB in a subject that is about 0.8 μM HMB.

An oral dosing of about 50 mg metformin or any analogs thereof can achieve a circulating level of metformin in a subject that is about 1-3 μM metformin or any analogs thereof. An oral dosing of about 1000 mg metformin or any analogs thereof can achieve a circulating level of metformin or any analogs thereof in a subject that is about 10 μM.

An oral dosing of about 3,000 mg nicotinic acid or nicotinamide riboside can achieve a circulating level of nicotinic acid or nicotinamide riboside in a subject that is about 10 μM nicotinic acid or nicotinamide riboside. An oral dosing of about 50 mg nicotinic acid or nicotinamide riboside can achieve a circulating level of nicotinic acid or nicotinamide riboside in a subject that is about 10-100 nM nicotinic acid or nicotinamide riboside.

An oral dosing of about 1100 mg of resveratrol can achieve a circulating level of resveratrol in a subject that is about 0.5 mM resveratrol. An oral dosing of about 50 mg of resveratrol can achieve a circulating level of resveratrol in a subject that is about 200 nM resveratrol.

In some embodiments, the compositions can be formulated to achieve a desired circulating molar or mass ratios achieved after administering one or more compositions to a subject. The compositions can be a combination composition described herein. The molar ratio can be adjusted to account for the bioavailability, the uptake, and the metabolic processing of the one or more components of a combination composition. For example, if the bioavailability of a component is low, then the molar amount of a that component can be increased relative to other components in the combination composition. In some embodiments, the circulating molar or mass ratio is achieved within about 0.1, 0.5, 0.75, 1, 3, 5, or 10, 12, 24, or 48 hours after administration. The circulating molar or mass ratio can be maintained for a time period of about or greater than about 0.1, 1, 2, 5, 10, 12, 18, 24, 36, 48, 72, or 96 hours.

In some embodiments, the circulating molar ratio of leucine to nicotinic acid or nicotinamide riboside is about, less than about, or greater than about 1, 5, 10, 20, 50, 100, 500, 1000, 5000, or 10000. In some embodiments, the circulating molar ratio of HMB to nicotinic acid or nicotinamide riboside is about or greater than about, or less than about 0.01, 0.05, 0.1, 0.5, 1, 5, 10, 20, 50, or 100. In some embodiments, the circulating molar ratio of a nicotinic acid or nicotinamide riboside to resveratrol is about, less than about, or greater than about 0.01, 0.05, 0.1, 0.5, 1, 5, 10, 20, 50, or 100.

In some embodiments of the invention, the following amounts of leucine, HMB, KIC, and/or resveratrol are to be administered to a subject: about, less than about, or more than about 0.5, 0.75, 1, 1.25, 1.5, 1.75, 2, 2.5, 3, or more g/day of leucine, and/or between about 0.5-3.0 g/day of leucine; about, less than about, or more than about 0.2, 0.4, 0.5, 0.75, 1, 1.5, 2, 2.5, 3, or more g/day of HMB, and/or between about 0.20-3.0 g/day of HMB; about, less than about, or more than about 0.2, 0.4, 0.5, 0.75, 1, 1.25, 1.5, 1.75, 2, 2.5, 3, or more g/day of KIC, and/or between about 0.2-3.0 g/day of KIC; and/or about, less than about, or more than about 10, 25, 50, 51, 75, 100, 150, 200, 250, 300, 350, 400, 450, 500, or more mg/day of resveratrol, and/or between about 10-500 mg/day of resveratrol. Thus, one embodiment provides a composition comprising leucine in an amount of between about 0.75-3.0 g and resveratrol in an amount between about 50 to 500 mg. Another embodiment provides a composition comprising HMB in an amount between about 0.40-3.0 g and resveratrol in an amount between about 50-500 mg Another embodiment provides for a composition comprising leucine in an amount between about 0.75-3.0 g, HMB in an amount between about 0.40-3.0 g and resveratrol in an amount between about 50-500 mg (or 50 to 500 mg). Another aspect of the invention provides compositions comprising synergizing amounts of resveratrol and leucine; resveratrol and HMB; resveratrol, leucine and HMB; resveratrol and KIC; resveratrol, KIC and leucine; resveratrol, KIC, and HMB; or resveratrol, KIC, leucine and HMB. In some embodiments, a synergizing amount of resveratrol is an amount range about, less than about, or more than about 35, 50, 75, 100, 150, 200, 250, 300, 350, 400, 450, or 500 mg resveratrol, and/or between about 35-500 mg resveratrol in combination with leucine and/or HMB. Synergizing amounts of leucine and/or KIC in a composition containing leucine and/or KIC and resveratrol can range from about, less than about, or more than about 0.5, 0.75, 1, 1.5, 2, 2.5, 3 or more grams, and/or between about 0.50 to 3.0 g; or in an amount range about, less than about, or more than about 0.75, 1, 1.25, 1.5, 1.75, 2, 2.5, 3 or more grams, and/or between about 0.75-3.0 g. Synergizing amounts of HMB provided in a composition containing HMB and resveratrol contains HMB in an amount of about, less than about, or more than about 0.2, 0.4, 0.5, 0.75, 1, 1.5, 2, 2.5, 3, or more grams, and/or between about 0.2-3.0 g. In some embodiments, where combinations of leucine and KIC are used in a composition, the total amount of leucine and KIC is about, less than about, or more than about 0.7, 0.75, 1, 1.5, 2, 2.5, or 3 grams, and/or between about 0.7-3.0 grams.

Another embodiment provides for a composition containing synergizing amounts of HMB, leucine and resveratrol. In such compositions, the total amount of leucine and HMB within the composition can be about, less than about, or more than about 0.7, 0.75, 1, 1.5, 2, 2.5, 3 grams, and/or between about 0.7-3.0 grams. Compositions containing both leucine and HMB can contain amounts of leucine and HMB that total about, less than about, or more than about 0.7, 1, 1.25, 1.5, 1.75, 2, 2.5, 3, or more grams, and/or between about 0.75-3.0 grams; or between about 0.75-3.0 grams, or between about 1.0-3.0 grams within the composition and resveratrol in synergizing amounts (about, less than about or more than about 35, 50, 75, 100, 150, 200, 250, 300, 350, 400, 450, or 500 mg resveratrol, and/or between about 35-500 mg resveratrol) or an amount of resveratrol between about 50-500. Yet another embodiment provides for a composition containing synergizing amounts of HMB, leucine, KIC and resveratrol. In such compositions, the total amount of leucine, KIC and HMB within the composition can be about, less than about, or more than about 0.7, 0.75, 1, 1.5, 2, 2.5, 3 grams, and/or between about 0.7-3.0 grams. Thus, compositions containing leucine, KIC and HMB can contain amounts of leucine, KIC and HMB that total about, less than about, or more than about 0.7, 0.75, 1, 1.5, 2, 2.5, 3 grams, and/or between about 0.75-3.0 grams, or between about 1.0-3.0 grams within the composition and resveratrol in synergizing amounts (about, less than about, or more than about 35, 50, 75, 100, 150, 200, 250, 300, 350, 400, 450, or 500 mg resveratrol, and/or between about 35-500 mg resveratrol) or an amount of resveratrol between about 50-500 mg.

Still other embodiments provide compositions comprising: a) about, less than about, or more than about 50, 60, 70, 80, 90, 100, or more mg, and/or between about 50 to 100 mg resveratrol, and about, less than about, or more than about 400, 425, 450, 475, 500, or more mg, and/or between about 400 mg to 500 mg HMB; b) about, less than about, or more than about 50, 60, 70, 80, 90, 100, or more mg, and/or between about 50 to 100 mg resveratrol, and about, less than about, or more than about 750, 850, 950, 1050, 1150, 1250 or more mg, and/or between about 750 mg to 1250 mg leucine; c) about, less than about, or more than about 50, 60, 70, 80, 90, 100, or more mg, and/or between about 50 to 100 mg resveratrol, and about, less than about, or more than about 750, 850, 950, 1050, 1150, 1250 or more mg, and/or between about 750 mg to 1250 mg KIC; or d) about, less than about, or more than about 50, 60, 70, 80, 90, 100, or more mg, and/or 50 mg to about 100 mg resveratrol and: i) a combination of HMB and KIC in an amount of about, less than about, or more than about 400, 500, 600, 700, 800, 900, 1000, 1100, 1250, or more mg, and/or between about 400 mg and about 1250 mg; ii) a combination of HMB and leucine in an amount of about, less than about, or more than about 400, 500, 600, 700, 800, 900, 1000, 1100, 1250, or more mg, and/or between about 400 mg and about 1250 mg; iii) a combination of KIC and leucine in an amount of about, less than about, or more than about 400, 500, 600, 700, 800, 900, 1000, 1100, 1250, or more mg, and/or between about 400 mg and about 1250 mg; or iv) a combination of HMB, KIC and leucine in an amount of about, less than about, or more than about 400, 500, 600, 700, 800, 900, 1000, 1100, 1250, or more mg, and/or between about 400 mg and about 1250 mg.

In some embodiments a unit dosage can comprise resveratrol in combination with one or more other components. In some embodiments, a unit dosage comprises one or more of: about, less than about, or more than about 50, 100, 200, 300, 400, 500 or more mg of HMB, and/or between about 50-500 mg of HMB; about, less than about, or more than about 10, 20, 30, 40, 50, 75, 100, or more mg resveratrol, and/or between about 10-100 mg resveratrol; and about, less than about, or more than about 400, 500, 600, 700, 800, 900, 1000, 1100, 1250, 1500, or more mg of leucine, and/or between about 400-1500 mg of leucine. A unit dosage can comprise about, less than about, or more than about 500 mg beta hydroxyl, beta methyl butyrate and about, less than about, or more than about 50 mg resveratrol. A unit dosage can comprise about, less than about, or more than about 500 mg beta hydroxy, beta methyl butyrate; and about, less than about, or more than about 50 mg resveratrol; and about, less than about, or more than about 15 mg vitamin B6. A unit dosage can comprise about, less than about, or more than about 1.125 g leucine and about, less than about, or more than about 50 mg resveratrol.

In some embodiments a unit dosage can comprise chlorogenic acid (e.g. about, less than about, or more than about 25, 50, 75, 100, 150, 200, or mg) in combination with one or more other components in about, less than about, or more than about the indicated amounts. A unit dosage can comprise 500 mg beta hydroxy, beta methyl butyrate (e.g. 50, 100, 200, 300, 400, 500 or more mg) and 100 mg chlorogenic acid. In some embodiments a unit dosage can comprise quinic acid in about, less than about, or more than about the indicated amounts (e.g. 10, 15, 20, 25, 30, 40, 50, or more mg), in combination with one or more other components in about, less than about, or more than about the indicated amounts. A unit dosage can comprise 500 mg beta hydroxy, beta methyl butyrate (e.g. 50, 100, 200, 300, 400, 500 or more mg) and 25 mg quinic acid.

In some embodiments a unit dosage can comprise fucoxanthin in about, less than about, or more than about the indicated amounts (e.g. 0.5, 0.75, 1, 1.25, 1.5, 1.75, 2, 2.25, 2.5, 3, 5, or more mg) in combination with one or more other components in about, less than about, or more than about the indicated amounts. A unit dosage can comprise 500 mg beta hydroxy, beta methyl butyrate (e.g. 50, 100, 200, 300, 400, 500 or more mg) and 2.5 mg fucoxanthin.

In some embodiments, a composition comprises an amount of an antidiabetic agent, such as a biguanide (e.g. metformin). The amount of antidiabetic agent may be a sub-therapeutic amount, and/or an amount that is synergistic with one or more other compounds in the composition or one or more of the compounds administered simultaneously or in close temporal proximity with the composition. In some embodiments, the antidiabetic agent is administered in a very low dose, a low dose, a medium dose, or a high dose, which describes the relationship between two doses, and generally do not define any particular dose range. For example, a daily very low dose of metformin may comprise about, less than about, or more than about 5 mg/kg, 10 mg/kg, 25 mg/kg, 50 mg/kg, 75 mg/kg, 100 mg/kg, or more; a daily low dose of metformin may comprise about, less than about, or more than about 75 mg/kg, 100 mg/kg, 150 mg/kg, 175 mg/kg, 200 mg/kg, or more; a daily medium dose of metformin may comprise about, less than about, or more than about 150 mg/kg, 175 mg/kg, 200 mg/kg, 250 mg/kg, 300; and a daily high dose of metformin may comprise about, less than about, or more than about 200 mg/kg, 250 mg/kg, 300 mg/kg, 350 mg/kg, 400 mg/kg, 500 mg/kg, 700 mg/kg, or more.

A unit dosage can comprise an amount of anti-diabetic agent, such as guanide (e.g. galegine and dimethylguanidine) and/or a biguanide (e.g. metformin) or a thiazolidinedione, that is between about 0.01-10, 0.01-50, 0.1-10, 0.5-20, 0.5-50, 1-50, 1-100, 5-100, 5-500 or 100-2550 mg. In some embodiments a unit dosage can comprise an anti-diabetic agent, such as guanide (e.g. galegine and dimethylguanidine) and/or a biguanide (e.g. metformin) or a thiazolidinedione in about, less than about, or more than about the indicated amounts (e.g. 0.01, 0.05, 0.1, 0.5, 1, 2, 5, 10, 15, 20, 25, 50, 100, 150, 200, 250, 300, 400, 500, 1000, 2000, 2550, 3000 or more mg, in combination with one or more other components in about, less than about, or more than about the indicated amounts (such as 0.01, 0.05, 0.1, 0.5, 1, 5, 10, 20, 30, 40, 50, 75, 100, or more mg of nicotinic acid, and/or 0.01-100 mg of nicotinic acid; 50, 100, 200, 300, 400, 500 or more mg HMB, and/or 50-500 mg HMB; and/or 400, 500, 600, 700, 800, 900, 1000, 1100, 1250, or more mg of leucine, and/or 400-1250 mg of leucine). A unit dosage can comprise about, less than about or more than about 50 mg metformin, 500 mg beta hydroxy, beta methyl butyrate and 50 mg resveratrol. A unit dosage can comprise about, less than about or more than about 50 mg metformin, 1.125 g leucine and 50 mg resveratrol. A unit dosage can comprise about, less than about or more than about 100 mg metformin, 500 mg beta hydroxy, beta methyl butyrate and 50 mg resveratrol. A unit dosage can comprise about, less than about or more than about 100 mg metformin, 1.125 g leucine and 50 mg resveratrol. A unit dosage can comprise about, less than about or more than about 250 mg metformin, 500 mg beta hydroxy, beta methyl butyrate and 50 mg resveratrol. A unit dosage can comprise about, less than about or more than about 250 mg metformin, 1.125 g leucine and 50 mg resveratrol.

In some embodiments of the invention, the combination compositions can have a specified ratio of branched chain amino acids and/or metabolites thereof to a sirtuin pathway activator. The specified ratio can provide for effective and/or synergistic regulation of energy metabolism. For example, the specified ratio can cause a decrease in weight gain of a subject, a decrease in visceral adipose volume of a subject, an increase in fat oxidation of a subject, an increase in insulin sensitivity of a subject, an increase of glucose uptake in muscle of a subject, a decrease in inflammation markers, and/or an increase in body temperature. Such beneficial effects can result from, in part, an increase in mitochondrial biogenesis, or a variety of other changes in the energy metabolism pathway. The ratio of branched chain amino acids and/or metabolites thereof to a sirtuin pathway activator can be a mass ratio, a molar ratio, or a volume ratio.

In some embodiments, the molar ratio of (a) branched chain amino acids and/or metabolites thereof to (b) a sirtuin pathway activator is about or greater than about 20, 25, 30, 35, 40, 45, 50, 55, 60, 65, 70, 75, 80, 85, 90, 100, 120, or 150. In other embodiments, the molar ratio of one or more branched chain amino acids and/or metabolites thereof to sirtuin pathway activator contained in the subject compositions is about or greater than about 20, 30, 40, 50, 60, 70, 80, 90, 95, 90, 95, 100, 105, 110, 120, 130, 140, 150, 160, 170, 180, 190, 200, 220, 250, 300, 350, 400, or 500. In some embodiments, the molar ratio of component (a) to (b) in said composition is greater than about 20, 40, 60, 80, 100, 120, or 150. In some embodiments, the molar ratio of component (a) to (b) in said composition is greater than about 80, 100, 120, or 150. In some embodiments, the molar ratio of component (a) to (b) in said composition is greater than about 80, 100, 120, or 150. In some embodiments, the molar ratio of component (a) to (b) in said composition is greater than about 200, 250, or 300. In some embodiments, the molar ratio of component (a) to (b) in said composition is greater than about 40, 150, 250, or 500.

In some embodiments, the molar or mass ratios are circulating molar or mass ratios achieved after administration one or more compositions to a subject. The compositions can be a combination composition described herein. The molar ratio of a combination composition in a dosing form can be adjusted to achieve a desired circulating molar ratio. The molar ratio can be adjusted to account for the bioavailiability, the uptake, and the metabolic processing of the one or more components of a combination composition. For example, if the bioavailiability of a component is low, then the molar amount of a that component can be increased relative to other components in the combination composition. In some embodiments, the circulating molar or mass ratio is achieved within about 0.1, 0.5, 0.75, 1, 3, 5, or 10, 12, 24, or 48 hours after administration. The circulating molar or mass ratio can be maintained for a time period of about or greater than about 0.1, 1, 2, 5, 10, 12, 18, 24, 36, 48, 72, or 96 hours.

In some embodiments, the circulating molar ratio of leucine to resveratrol (or sirtuin pathway activator) is about or greater than about 1000, 1500, 2000, 2550, 3000, 3500, 4000, 10000, 50000, or more. In some embodiments, the mass ratio of leucine to resveratrol is about or greater than about 750, 1000, 1200, 1500, 1700, 2000, or 2550.

The circulating molar ratio of HMB to resveratrol (or sirtuin pathway activator) can be about or greater than about 3, 5, 10, 15, 20, 25, 30, 35, 40, 50, 60, 75, 100, 250, 500, or more. In some embodiments, the mass ratio of HMB to resveratrol is about or greater than about 1, 3, 6, 9, 12, 15, 20, or 25.

In some embodiments, the circulating mass ratio of HMB to resveratrol (or sirtuin pathway activator) is about or greater than about 100, 120, 140, 160, 180, 200, 220, or 250. In some embodiments, the mass ratio of HMB to resveratrol is about or greater than about 400, 600, 800, 1000, 1200, or 1400.

In some embodiments, the circulating molar ratio of HMB to chlorogenic acid is about or greater than about 5, 10, 20, or 40. In some embodiments, the molar ratio of leucine to chlorogenic acid is about or greater than about 500, 1000, 2000, or 4000.

Any of the components described herein, including leucine, HMB, KIC, nicotinic acid, nicotinamide riboside, biguanide (e.g. metformin or any analog thereof) and resveratrol can be used in a subject composition in free form, isolated form, purified from a natural source, and/or purified or prepared from a synthetic source. The natural source can be an animal source or plant source. The components can be pure to at least about 95, 97, 99, 99.5, 99.9, 99.99, or 99.999%.

### Dosing Forms

The compositions described herein can be compounded into a variety of different dosage forms. It can be used orally as a tablet, a capsule, a pill, a granule, an emulsion, a gel, a plurality of beads encapsulated in a capsule, a powder, a suspension, a liquid, a semi-liquid, a semi-solid, a syrup, a slurry, a chewable form, caplets, soft gelatin capsules, lozenges or solution. Alternatively, the compositions can be formulated for inhalation or for intravenous delivery. The compositions can also be formulated as a nasal spray or for injection when in solution form. In some embodiments, the composition can be a liquid composition suitable for oral consumption.

Compositions formulated for inhalation can be packaged in an inhaler using techniques known in the art. An inhaler can be designed to dispense 0.25, 0.5, or 1 unit dose per inhalation. An inhaler can have a canister that holds the subject composition formulated for inhalation, a metering valve that allows for a metered quantity of the formulation to be dispensed with each actuation, and an actuator or mouthpiece that allows for the device to be operated and direct the subject composition into the subject's lungs. The formulated composition can include a liquefied gas propellant and possibly stabilizing excipients. The actuator can have a mating discharge nozzle that connects to the canister and a dust cap to prevent contamination of the actuator. Upon actuation, the subject composition can be volatized, which results in the formation of droplets of the subject composition. The droplets can rapidly evaporate resulting in micrometer-sized particles that are then inhaled by the subject. Inhalers and methods for formulating compositions for inhalation are described in are described in U.S. Pat. Nos. 5,069,204, 7,870,856 and U.S. Patent Application No. 2010/0324002, which are incorporated herein by reference in its entirety.

Compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, including liquid dosage forms (e.g., a suspension or slurry), and oral solid dosage forms (e.g., a tablet or bulk powder). Oral dosage forms can be formulated as tablets, pills, dragees, capsules, emulsions, lipophilic and hydrophilic suspensions, liquids, gels, syrups, slurries, suspensions and the like, for oral ingestion by an individual or a patient to be treated. Such dosage forms can be prepared by any of the methods of formulation. For example, the active ingredients can be brought into association with a carrier, which constitutes one or more necessary ingredients. Capsules suitable for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. Optionally, the inventive composition for oral use can be obtained by mixing a composition a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The liquid forms, in which the formulations disclosed herein can be incorporated for administration orally or by injection, include aqueous solution, suitably flavored syrups, aqueous or oil suspensions, and flavored emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin.

A subject can be treated by combination of an injectable composition and an orally ingested composition.

Liquid preparations for oral administration can take the form of, for example, solutions, syrups or suspensions, or they can be presented as a dry product for reconstitution with water or other suitable vehicles before use. Such liquid preparations can be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

The preparation of pharmaceutical compositions of this invention, including oral and inhaled formulations, can be conducted in accordance with generally accepted procedures for the preparation of pharmaceutical preparations. See, for example, Remington's Pharmaceutical Sciences 18th Edition (1990), E. W. Martin ed., Mack Publishing Co., PA. Depending on the intended use and mode of administration, it can be desirable to process the magnesium-counter ion compound further in the preparation of pharmaceutical compositions. Appropriate processing can include mixing with appropriate non-toxic and non-interfering components, sterilizing, dividing into dose units, and enclosing in a delivery device.

This invention further encompasses anhydrous compositions and dosage forms comprising an active ingredient, since water can facilitate the degradation of some compounds. For example, water can be added (e.g., 5%) in the arts as a means of simulating long-term storage in order to determine characteristics such as shelf-life or the stability of formulations over time. Anhydrous compositions and dosage forms of the invention can be prepared using anhydrous or low moisture containing ingredients and low moisture or low humidity conditions. Compositions and dosage forms of the invention which contain lactose can be made anhydrous if substantial contact with moisture and/or humidity during manufacturing, packaging, and/or storage is expected. An anhydrous composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastic or the like, unit dose containers, blister packs, and strip packs.

An ingredient described herein can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Binders suitable for use in dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Lubricants which can be used to form compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the composition.

Lubricants can be also be used in conjunction with tissue barriers which include, but are not limited to, polysaccharides, polyglycans, seprafilm, interceed and hyaluronic acid.

Disintegrants can be used in the compositions of the invention to provide tablets that disintegrate when exposed to an aqueous environment. Too much of a disintegrant can produce tablets which can disintegrate in the bottle. Too little can be insufficient for disintegration to occur and can thus alter the rate and extent of release of the active ingredient(s) from the dosage form. Thus, a sufficient amount of disintegrant that is neither too little nor too much to detrimentally alter the release of the active ingredient(s) can be used to form the dosage forms of the compounds disclosed herein. The amount of disintegrant used can vary based upon the type of formulation and mode of administration, and can be readily discernible to those of ordinary skill in the art. About 0.5 to about 15 weight percent of disintegrant, or about 1 to about 5 weight percent of disintegrant, can be used in the pharmaceutical composition. Disintegrants that can be used to form compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Examples of suitable fillers for use in the compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

When aqueous suspensions and/or elixirs are desired for oral administration, the active ingredient therein can be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

In one embodiment, the composition can include a solubilizer to ensure good solubilization and/or dissolution of the compound of the present invention and to minimize precipitation of the compound of the present invention. This can be especially important for compositions for non-oral use, e.g., compositions for injection. A solubilizer can also be added to increase the solubility of the hydrophilic drug and/or other components, such as surfactants, or to maintain the composition as a stable or homogeneous solution or dispersion.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof. A non-exhaustive list of examples of excipients includes monoglycerides, magnesium stearate, modified food starch, gelatin, microcrystalline cellulose, glycerin, stearic acid, silica, yellow beeswax, lecithin, hydroxypropylcellulose, croscarmellose sodium, and crospovidone.

The compositions described herein can also be formulated as extended-release, sustained-release or time-release such that one or more components are released over time. Delayed release can be achieved by formulating the one or more components in a matrix of a variety of materials or by microencapsulation. The compositions can be formulated to release one or more components over a time period of 1, 4, 6, 8, 12, 16, 20, 24, 36, or 48 hours. The release of the one or more components can be at a constant or changing rate.

In some embodiments, a subject composition described herein can be formulated in as matrix pellets in which particles of the subject composition are embedded in a matrix of water-insoluble plastic and which are enclosed by a membrane of water-insoluble plastic containing embedded particles of lactose, produces and maintains plasma levels of the subject composition within the targeted therapeutic range. In other embodiments, a subject composition can be formulated as a sustained release tablet obtained by coating core granules composed mainly of the subject composition with a layer of a coating film composed of a hydrophobic material and a plastic excipient and optionally containing an enteric polymer material to form coated granules and then by compressing the coated granules together with a disintegrating excipient. Sustained release formulations are described in U.S. Pat. Nos. 4,803,080, and 6,426,091, which are herein incorporated by reference in its entirety.

Using the controlled release dosage forms provided herein, the one or more cofactors can be released in its dosage form at a slower rate than observed for an immediate release formulation of the same quantity of components. In some embodiments, the rate of change in the biological sample measured as the change in concentration over a defined time period from administration to maximum concentration for an controlled release formulation is less than about 80%, 70%, 60%, 50%, 40%, 30%, 20%, or 10% of the rate of the immediate release formulation. Furthermore, in some embodiments, the rate of change in concentration over time is less than about 80%, 70%, 60%, 50%, 40%, 30%, 20%, or 10% of the rate for the immediate release formulation.

In some embodiments, the rate of change of concentration over time is reduced by increasing the time to maximum concentration in a relatively proportional manner. For example, a two-fold increase in the time to maximum concentration can reduce the rate of change in concentration by approximately a factor of 2. As a result, the one or more cofactors can be provided so that it reaches its maximum concentration at a rate that is significantly reduced over an immediate release dosage form. The compositions of the present invention can be formulated to provide a shift in maximum concentration by 24 hours, 16 hours, 8 hours, 4 hours, 2 hours, or at least 1 hour. The associated reduction in rate of change in concentration can be by a factor of about 0.05, 0.10, 0.25, 0.5 or at least 0.8. In certain embodiments, this is accomplished by releasing less than about 30%, 50%, 75%, 90%, or 95% of the one or more cofactors into the circulation within one hour of such administration. Optionally, the controlled release formulations exhibit plasma concentration curves having initial (e.g., from 2 hours after administration to 4 hours after administration) slopes less than 75%, 50%, 40%, 30%, 20% or 10% of those for an immediate release formulation of the same dosage of the same cofactor.

In some embodiments, the rate of release of the cofactor as measured in dissolution studies is less than about 80%, 70%, 60% 50%, 40%, 30%, 20%, or 10% of the rate for an immediate release formulation of the same cofactor over the first 1, 2, 4, 6, 8, 10, or 12 hours.

The controlled release formulations provided herein can adopt a variety of formats. In some embodiments, the formulation is in an oral dosage form, including liquid dosage forms (e.g., a suspension or slurry), and oral solid dosage forms (e.g., a tablet or bulk powder), such as, but not limited to those, those described herein.

The controlled release tablet of a formulation disclosed herein can be of a matrix, reservoir or osmotic system. Although any of the three systems is suitable, the latter two systems can have more optimal capacity for encapsulating a relatively large mass, such as for the inclusion of a large amount of a single cofactor, or for inclusion of a plurality of cofactors, depending on the genetic makeup of the individual. In some embodiments, the slow-release tablet is based on a reservoir system, wherein the core containing the one or more cofactors is encapsulated by a porous membrane coating which, upon hydration, permits the one or more cofactors to diffuse through. Because the combined mass of the effective ingredients is generally in gram quantity, an efficient delivery system can provide optimal results.

Thus, tablets or pills can also be coated or otherwise compounded to provide a dosage form affording the advantage of prolonged action. For example, the tablet or pill can comprise an inner dosage an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings such materials including a number of polymeric acids and mixtures of polymeric acids with such materials as shellac, acetyl alcohol and cellulose acetate. In some embodiments, a formulation comprising a plurality of cofactors can have different cofactors released at different rates or at different times. For example, there can be additional layers of cofactors interspersed with enteric layers.

Methods of making sustained release tablets are known in the art, e.g., see U.S. Patent Publications 2006/051416 and 2007/0065512, or other references disclosed herein. Methods such as described in U.S. Pat. Nos. 4,606,909, 4,769,027, 4,897,268, and 5,395,626 can be used to prepare sustained release formulations of the one or more cofactors determined by the genetic makeup of an individual. In some embodiments, the formulation is prepared using OROS® technology, such as described in U.S. Pat. Nos. 6,919,373, 6,923,800, 6,929,803, and 6,939,556. Other methods, such as described in U.S. Pat. Nos. 6,797,283, 6,764,697, and 6,635,268, can also be used to prepare the formulations disclosed herein.

In some embodiments, the compositions can be formulated in a food composition. For example, the compositions can be a beverage or other liquids, solid food, semi-solid food, with or without a food carrier. For example, the compositions can include a black tea supplemented with any of the compositions described herein. The composition can be a dairy product supplemented any of the compositions described herein. In some embodiments, the compositions can be formulated in a food composition. For example, the compositions can comprise a beverage, solid food, semi-solid food, or a food carrier.

In some embodiments, liquid food carriers, such as in the form of beverages, such as supplemented juices, coffees, teas, sodas, flavored waters, and the like can be used. For example, the beverage can comprise the formulation as well as a liquid component, such as various deodorant or natural carbohydrates present in conventional beverages. Examples of natural carbohydrates include, but are not limited to, monosaccharides such as, glucose and fructose; disaccharides such as maltose and sucrose; conventional sugars, such as dextrin and cyclodextrin; and sugar alcohols, such as xylitol and erythritol. Natural deodorant such as taumatin, stevia extract, levaudioside A, glycyrrhizin, and synthetic deodorant such as saccharin and aspartame can also be used. Agents such as flavoring agents, coloring agents, and others can also be used. For example, pectic acid and the salt thereof, alginic acid and the salt thereof, organic acid, protective colloidal adhesive, pH controlling agent, stabilizer, a preservative, glycerin, alcohol, or carbonizing agents can also be used. Fruit and vegetables can also be used in preparing foods or beverages comprising the formulations discussed herein.

Alternatively, the compositions can be a snack bar supplemented with any of the compositions described herein. For example, the snack bar can be a chocolate bar, a granola bar, or a trail mix bar. In yet another embodiment, the present dietary supplement or food compositions are formulated to have suitable and desirable taste, texture, and viscosity for consumption. Any suitable food carrier can be used in the present food compositions. Food carriers of the present invention include practically any food product. Examples of such food carriers include, but are not limited to food bars (granola bars, protein bars, candy bars, etc.), cereal products (oatmeal, breakfast cereals, granola, etc.), bakery products (bread, donuts, crackers, bagels, pastries, cakes, etc.), beverages (milk-based beverage, sports drinks, fruit juices, alcoholic beverages, bottled waters), pastas, grains (rice, corn, oats, rye, wheat, flour, etc.), egg products, snacks (candy, chips, gum, chocolate, etc.), meats, fruits, and vegetables. In an embodiment, food carriers employed herein can mask the undesirable taste (e.g., bitterness). Where desired, the food composition presented herein exhibit more desirable textures and aromas than that of any of the components described herein. For example, liquid food carriers can be used according to the invention to obtain the present food compositions in the form of beverages, such as supplemented juices, coffees, teas, and the like. In other embodiments, solid food carriers can be used according to the invention to obtain the present food compositions in the form of meal replacements, such as supplemented snack bars, pasta, breads, and the like. In yet other embodiments, semi-solid food carriers can be used according to the invention to obtain the present food compositions in the form of gums, chewy candies or snacks, and the like.

The dosing of the combination compositions can be administered about, less than about, or more than about 1, 2, 3, 4, 5, 6, 7, 8, 9, 10 or more times a daily. A subject can receive dosing for a period of about, less than about, or greater than about 1, 2, 3, 4, 5, 6, 7, 8, 9, 10, 11, 12, 13, 14 or more days, weeks or months. A unit dose can be a fraction of the daily dose, such as the daily dose divided by the number of unit doses to be administered per day. A unit dose can be a fraction of the daily dose that is the daily dose divided by the number of unit doses to be administered per day and further divided by the number of unit doses (e.g. tablets) per administration. The number of unit doses per administration can be about, less than about, or more than about 1, 2, 3, 4, 5, 6, 7, 8, 9, 10, or more. The number of doses per day can be about, less than about, or more than about 1, 2, 3, 4, 5, 6, 7, 8, 9, 10, or more. The number of unit doses per day can be determined by dividing the daily dose by the unit dose, and can be about, less than about, or more than about 1, 2, 3, 4, 5, 6, 7, 8, 9, 10, 11, 12, 13, 14, 15, 6, 17, 18, 19, 20, or more unit doses per day. For example, a unit dose can be about ½, ⅓, ¼, ⅕, ⅙, 1/7, ⅛, 1/9, 1/10. A unit dose can be about one-third of the daily amount and administered to the subject three times daily. A unit dose can be about one-half of the daily amount and administered to the subject twice daily. A unit dose can be about one-fourth of the daily amount with two unit doses administered to the subject twice daily. In some embodiments, a unit dose comprises about, less than about, or more than about 50 mg resveratrol. In some embodiments, a unit dose comprises about, less than about, or more than about 550 mg leucine. In some embodiments, a unit dose comprises about, less than about, or more than about 200 mg of one or more leucine metabolites.

In some embodiments, a unit dose (e.g. a unit dose comprising one or more leucine metabolites, such as HMB) is administered as one unit dose two times per day. A unit dose can comprise more than one capsule, tablet, vial, or entirely.

Compositions disclosed herein can further comprise a flavorant and can be a solid, liquid, gel or emulsion.

When the subject composition administered further comprises one or more therapeutic agents, and the therapeutic agents have a shorter half-life than the leucine and/or leucine metabolites, or the one or more agents selected from the group consisting of nicotinic acid, nicotinamide riboside, and nicotinic acid metabolite, the unit dose forms of the therapeutic agent and the leucine and/or leucine metabolites, or one or more agents selected from the group consisting of nicotinic acid, nicotinamide riboside, and nicotinic acid metabolite can be adjusted accordingly.

### Methods

The composition is particularly useful for diabetes control and ameliorating a hyperlipidemic condition. In one embodiment, the invention provides for methods for increasing insulin sensitivity, increasing glucose uptake, increasing glucose utilization, lowering blood glucose level, increasing fat oxidation, lowering lipid accumulation, reducing total lipid content or lowering level of total cholesterol, LDL, or triglyceride, increasing HDL level, or reducing atherosclerotic plaque size comprising administering to a subject in need thereof any of the subject compositions. The level or content described herein can be a circulating concentration in serum or blood stream, or a total amount in the subject's body. In some embodiments, the subject composition is useful in increasing weight loss of the subject, and increasing Sirt1 activation of the subject. In various embodiments of the invention, a composition is administered to the subject in an amount that delivers synergizing amounts of leucine and/or a metabolite thereof, nicotinic acid and/or nicotinamide riboside and/or a nicotinic acid metabolite, and an anti-diabetic such as a biguanide (e.g. metformin or any analog thereof), with or without resveratrol sufficient to ameliorate a hyperlipidemic condition and for diabetes control of the subject. In some embodiments, nicotinic acid, nicotinamide riboside or nicotinic acid metabolites can induce a side effect (e.g., cutaneous vasodilation) if it is administered to a subject at its therapeutic dose without leucine or leucine metabolites or an anti-diabetic agent such as metformin or any analogs thereof. In some embodiments, an anti-diabetic agent such as metformin or any analogs thereof can cause a side effect (e.g., gastrointestinal distress) if it is administered to a subject at its therapeutic dose without leucine or leucine metabolites or nicotinic acid. Methods described herein can also be useful for ameliorating the side-effect without losing the therapeutic effectiveness of nicotinic acid, nicotinamide riboside or nicotinic acid metabolites. A description of various aspects, features, embodiments, and examples, is provided herein.

The subject methods comprising the use of leucine and/or leucine metabolite and an anti-diabetic such as an anti-diabetic such as a biguanide (e.g. metformin and any analog thereof) with one or more agents selected from the group consisting of nicotinic acid, nicotinamide riboside, and nicotinic acid metabolite can be applicable for administering to a subject that is suffering from diabetes mellitus and/or hyperlipidemia, at risk of suffering from diabetes mellitus and/or hyperlipidemia, and/or suffering from a condition that is associated with diabetes mellitus and/or hyperlipidemia such as cardiovascular conditions. In some cases, an effective amount of an additional therapeutic or a pharmaceutically active agent that is known in the medical art (e.g., an anti-hyperlipidemic agent) can be administered to a subject in conjunction with any of the subject compositions.

Hyperlipidemia can be characterized by a high level of total lipid content or level in a subject. Hyperlipidemia can also be accompanied by a high level of body weight or BMI of a subject. The types of lipid can include cholesterol, cholesterol esters, phospholipids and triglycerides. The content or level of the lipids can be a circulating level that is measured in the bloodstream, plasma or serum of the subject. The content of the lipids can also be correlated by the body weight of the subject. These lipids can be transported in the blood as large lipoproteins including chylomicrons, very low-density lipoproteins (VLDL), intermediate-density lipoprotein (IDL), low-density lipoproteins (LDL) and high-density lipoproteins (HDL) based on their density. Most triglycerides can be transported in chylomicrons or VLDL and most cholesterol can be carried in LDL and HDL. High levels of lipid in the circulation can cause lipid accumulation on the walls of arteries, and further result in atherosclerotic plaque formation and therefore narrow the arteries. The subject that is suffering from hyperlipidemia can be at high risk of acquiring a cardiovascular condition. Hyperlipidemia can also be characterized by a high level of some lipoproteins or a low level of HDL. The condition that the subject is suffering from or at risk of suffering from can be a condition that is associated with an abnormal level of lipoproteins or lipids in the subject. The subject composition can be used to change the level of the one or more lipids or lipoproteins in the subject. In some embodiments, the type of lipids or lipoproteins that its level can be affected by the subject compositions and methods can be one or more lipoproteins and/or lipids including but not limited to: total cholesterol, triglyceride, HDL, IDL, VLDL or LDL.

A number of methods can be used to assess the levels of lipoproteins and/or lipids in a subject. These methods can differ from one another in the type of sample and the analytical technique used. The type of sample that can be used to measure such levels include but are not limited to: serum, plasma, whole blood, red blood cells or tissue samples. Where desired, the level of lipoproteins and/or lipids can be measured under a fasting condition, e.g., without taking food for at least about 8 hours, 10 hours, 12 hours, 15 hours, 24 hours, or even longer.

The size of atherosclerotic plaque or lesion can be measured by any methods that are known in the art. For examples, methods described in Phan B A et al., “Effects of niacin on glucose levels, coronary stenosis progression, and clinical events in subjects with normal baseline glucose levels (100 mg/dl): a combined analysis of the Familial Atherosclerosis Treatment Study (FATS), HDL-Atherosclerosis Treatment Study (HATS), Armed Forces Regression Study (AFREGS), and Carotid Plaque Composition by MRI during lipid-lowering (CPC) study”, Am J Cardiol. 2013 Feb. 1; 111(3):352-5, and Lehman S J et al., “Assessment of Coronary Plaque Progression in Coronary CT Angiography Using a Semi-Quantitative Score”, JACC Cardiovasc Imaging. 2009 November; 2(11): 1262-1270. Non-limiting example of the method to measure the size of atherosclerotic plaque or lesion can be quantitative coronary angiography.

In some embodiments, the amounts of the nicotinic acid, nicotinamide riboside and/or nicotinic acid metabolites in the composition, if administered to a subject alone and without leucine, a leucine metabolite, and an anti-diabetic such as a biguanide (e.g. metformin or any analog thereof), or resveratrol, can cause no therapeutic effect in the subject. Additionally, the amounts of leucine, a leucine metabolite, or resveratrol, if administered to the subject without the nicotinic acid, nicotinamide riboside or nicotinic acid metabolites and an anti-diabetic such as a biguanide (e.g. metformin or any analog thereof), can have no therapeutic effect on the subject. Further, the amounts of an anti-diabetic such as a biguanide (e.g. metformin or any analog thereof) in the composition, if administered to a subject alone and without leucine or resveratrol, a leucine metabolite, nicotinic acid, nicotinamide riboside and/or nicotinic acid metabolites, can cause no therapeutic effect in the subject. However, when the nicotinic acid, nicotinamide riboside and/or nicotinic acid metabolites is administered in conjunction with either leucine, a leucine metabolite, or resveratrol, and an anti-diabetic such as a biguanide (e.g. metformin or any analog thereof), a therapeutic effect can be observed. The “therapeutic effect” described herein is a lowered total lipid content, decreased total cholesterol level, decreased triglyceride level, increased HDL level, decreased LDL level or reduced atherosclerotic plaque in the subject administered. Accordingly, the invention provides a method for administering a composition comprising (a) leucine and/or one or more metabolites thereof, (b) one or more agents selected from the group consisting of nicotinic acid, nicotinamide riboside, and nicotinic acid metabolite, and (c) an anti-diabetic such as a biguanide (e.g. metformin or any analog thereof) present in a sub-therapeutic amount, wherein the composition is effective in increasing treating hyperlipidemic conditions as compared to that of component (b) when it is used alone, and/or in increasing treating diabetes mellitus as compared to that of component (c) when it is used alone. The amount of leucine and/or one or more metabolites in the composition can also be a sub-therapeutic amount.

Quantification of the therapeutic effect can show that the effect of a composition that comprises (a) leucine or a leucine metabolite and (b) a sub-therapeutic amount of nicotinic acid, nicotinamide riboside or a nicotinic acid metabolite, and (c) an anti-diabetic such as a biguanide such as metformin and any analog thereof is greater than the predicted effect of administering (a) or (b) or (c) alone, assuming simple additive effects of (a) and (b) and (c), and thus the effect is synergistic. The synergistic effect can be quantified as the measured effect above the predicted simple additive effect of the components of the composition. For example, if administration of component (a) alone yields an effect of 10% relative to control, administration of component (b) alone yields an effect of 15% relative to control, administration of component (c) alone yields an effect of 15% relative to control, and administration of a composition comprising both (a) and (b) and (c) yields an effect of 60% relative to control, the synergistic effect would be 60%−(15%+10%+15%), or 25%.

In some embodiments, a therapeutic amount of nicotinic acid, nicotinamide riboside and/or nicotinic acid metabolites can cause a side effect that can be characterized by an increased in cutaneous vasodilation. The increase in the cutaneous vasodilation can be clinically significant. A sub-therapeutic amount of nicotinic acid, nicotinamide riboside and/or nicotinic acid metabolites cannot cause a clinically significant cutaneous vasodilation, or can reduce the degree of cutaneous vasodilation in the subject administered as compared to a therapeutic amount of nicotinic acid, nicotinamide riboside and/or nicotinic acid metabolites. The subject compositions and methods described herein comprise a sub-therapeutic amount of nicotinic acid, nicotinamide riboside and/or nicotinic acid metabolites, to be used with leucine and/or leucine metabolites to result a therapeutic degree of effect of the sub-therapeutic amount of the nicotinic acid, nicotinamide riboside and/or nicotinic acid metabolites without causing the degree of side effect that can normally be caused by a therapeutic amount of nicotinic acid, nicotinamide riboside and/or nicotinic acid metabolites when used without leucine and/or leucine metabolites. Levels of cutaneous vasodilation can be measured by any methods known in the medical art, such as the methods including laser-Doppler flowmeter. With the same level of therapeutic effect (e.g. lowering cholesterol level by at least 5%), the level of cutaneous vasodilation caused by the subject compositions as compared to nicotinic acid, nicotinamide riboside and/or nicotinic acid metabolites without leucine and/or leucine metabolites can be lower. For example, less than about 1%, 5%, 10%, 15%, 20%, 25%, 30%, 35%, 40%, 45%, 50%, 60%, 70%, 80% or 90% of the level that is caused by a therapeutic amount of nicotinic acid, nicotinamide riboside and/or nicotinic acid metabolites.

The amount of leucine and/or leucine metabolites can be at least about 25, 50, 75, 100, 150, 200, 250, 300, 350, 400, 450, 500 mg. The sub-therapeutic amount of nicotinic acid, nicotinamide riboside, and/or nicotinic acid metabolites can be less than 1 g, 500, 250, 100, 50 or 10 mg. The amount of nicotinic acid, nicotinamide riboside, and/or nicotinic acid metabolites can be between about 1-100 mg. The amount of nicotinic acid, nicotinamide riboside, and/or nicotinic acid metabolites can be capable of achieving a circulating level of nicotinic acid, nicotinamide riboside, and/or nicotinic acid metabolites that is about 1-100 nM, higher than about 100 nM or at least about 10 nM.

In some embodiments a unit dosage can comprise metformin in about, less than about, or more than about the indicated amounts (e.g. 25, 50, 100, 150, 200, 250, 300, 400, 500, or more mg) in combination with one or more other components in about, less than about, or more than about the indicated amounts (such as 10, 20, 30, 40, 50, 75, 100, or more mg of resveratrol; 50, 100, 200, 300, 400, 500 or more mg HMB; and/or 400, 500, 600, 700, 800, 900, 1000, 1100, 1250, or more mg of leucine). A unit dosage can comprise about, less than about or more than about 50 mg metformin, 500 mg beta hydroxy, beta methyl butyrate and 50 mg resveratrol. A unit dosage can comprise about, less than about or more than about 50 mg metformin, 1.125 g leucine and 50 mg resveratrol. A unit dosage can comprise about, less than about or more than about 100 mg metformin, 500 mg beta hydroxy, beta methyl butyrate and 50 mg resveratrol. A unit dosage can comprise about, less than about or more than about 100 mg metformin, 1.125 g leucine and 50 mg resveratrol. A unit dosage can comprise about, less than about or more than about 250 mg metformin, 500 mg beta hydroxy, beta methyl butyrate and 50 mg resveratrol. A unit dosage can comprise about, less than about or more than about 250 mg metformin, 1.125 g leucine and 50 mg resveratrol. In some embodiments, a composition further comprises a PDE inhibitor in a synergizing amount. In some embodiments, a metformin composition further comprises a sirtuin pathway activator in a synergizing amount. In some embodiments, resveratrol in an example composition is replaced with a PDE inhibitor or a sirtuin pathway activator in a synergizing amount. In compositions comprising a PDE inhibitor or methods comprising administration of a PDE inhibitor (separately from or concurrently with one or more other components), the PDE inhibitor may be provided in an amount that produces a peak plasma concentration of about, less than about, or more than about 0.1, 1, 5, 10, 25, 50, 100, 500, 1000, 2550, 5000, 10000, or more nM.

Accordingly, the multi-component compositions described herein (such as nicotinic acid/leucine/a biguanide/metformin, nicotinic acid/leucine/resveratrol/a biguanide or metformin, nicotinamide riboside/leucine/a biguanide/metformin, and nicotinamide riboside/leucine/resveratrol/a biguanide/metformin) can have a beneficial or synergistic effect on increasing insulin sensitivity, increasing glucose uptake, increasing glucose utilization, lowering blood glucose level, lowering total lipid content, lowering lipid accumulation, decreasing total cholesterol level, decreasing triglyceride level, increasing HDL level, increasing fat oxidation, and/or decreasing LDL level. In some embodiments, the compositions and methods described herein can be effective to change the level of lipoproteins and/or lipids in the subject by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% or even higher as compared to an initial level of lipoproteins and/or lipids prior to administration of it to a subject. The level can be lowered by about 19%-24%, 14%-29%, 12%-35%, 10-40%, 8%-45%, 5%-50%, 2%-60%, or 1%-70%. The level can be a circulating level.

Accordingly, the multi-component compositions described herein (such as nicotinic acid/leucine/a biguanide/metformin, nicotinic acid/leucine/resveratrol/a biguanide or metformin, nicotinamide riboside/leucine/a biguanide/metformin, and nicotinamide riboside/leucine/resveratrol/a biguanide/metformin) can have a beneficial or synergistic effect on increasing insulin sensitivity, increasing glucose uptake, increasing glucose utilization, lowering blood glucose level, lowering total lipid content, lowering lipid accumulation, decreasing total cholesterol level, decreasing triglyceride level, increasing HDL level, increasing fat oxidation, and/or decreasing LDL level. In some embodiments, the compositions and methods described herein can be effective to change the insulin sensitivity and/or glucose utilization in the subject by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% or even higher as compared to an initial level of lipoproteins and/or lipids prior to administration of it to a subject. The level can be lowered by about 19%-24%, 14%-29%, 12%-35%, 10-40%, 8%-45%, 5%-50%, 2%-60%, or 1%-70%. The level can be a circulating level.

In some embodiments, the compositions and methods described herein can be effective to reduce the atherosclerotic plaque size in a subject by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% or even higher as compared to an initial size of atherosclerotic plaque prior to administration of it to a subject. The level can be reduced by about 19%-24%, 14%-29%, 12%-35%, 10-40%, 8%-45%, 5%-50%, 2%-60%, or 1%-70%.

The invention provides a method for administering a food composition comprising: (a) leucine and/or a metabolite thereof; (b) nicotinic acid and/or nicotinamide riboside and/or a nicotinic acid metabolite; and (c) an anti-diabetic such as a biguanide (e.g. metformin or any analog thereof), wherein (a) and (b) and (c) are present in an amount that synergistically effect a decrease in weight gain of a subject, a decrease in lipid content, a decrease in LDL level, an increase in HDL level, a decrease in cholesterol level, a decrease in triglyceride level, an increase in activation of Sirt1 in the subject, an increase in fat oxidation of a subject, an increase in insulin sensitivity of a subject, an increase of glucose uptake in muscle of a subject, and (d) a food carrier. In some embodiments, the weight percentage of component (a) is between about 60-100%, 70-95%, 80%-95%, or 85-90% of the total composition. In some embodiments, the weight percentage of component (b) is between about 0-50%, 1-30%, 2-25%, or 5%-20% of the total composition. In some embodiments, the weight percentages of component (c) is between about 0-30%, 0.5-20%, 1-20%, 5-20%, 1-10%, or 1%-5% of the total composition.

The invention provides for a method of treating diabetes, comprising administering to the subject any of the compositions described herein over a time period, wherein the insulin sensitivity in the subject is increased over the time period. Insulin sensitivity can be increased by about or greater than about 1, 2, 3, 5, 10, 20, 50, 100, or 200%. In some embodiments, a branched chain amino acid (or a metabolite thereof) and/or a sirtuin pathway activator are administered in an amount that reduces the therapeutically effective dose of metformin for a subject. In some embodiments, the therapeutically effective dose of metformin is reduced by about or more than about 50%, 60%, 70%, 80%, 90%, 95%, 97.5%, 99.9%, 99.99%, or more. In some embodiments, administration of compositions of the invention reduces body fat (e.g. visceral fat) by about or more than about 5%, 10%, 15%, 20%, 25%, 50%, or more.

Insulin sensitivity can be measured using a variety of techniques, including HOMA_{IR}. HOMA_{IR}, which is the homeostasis model assessment of insulin resistance can be used as a screening index of changes in insulin sensitivity. HOMA_{IR} can be calculated via standard formula from fasting plasma insulin and glucose as follows: HOMA_{IR}=[Insulin (uU/mL)×glucose (mM)]/22.5.

In some embodiments, insulin signaling can also be measured. Insulin signaling can be measured by measuring total and phosphorylated Akt, GSK-3β, IGF-1R, IR, IRS-1, p70S6K and PRAS40 in tissue lysates via the Luminex Kits “Akt Pathway Total 7-Plex Panel” (Cat# LHO0002) and “Akt Pathway Phospho 7-Plex Panel” (Cat# LHO0001) from Invitrogen Life Science.

Administration of compositions disclosed herein that increase SIRT1 and SIRT3 activity can be useful in any subject in need of metabolic activation of hepatocytes, adipocytes or one or more of their muscles, e.g., skeletal muscle, smooth muscle or cardiac muscle or muscle cells thereof. A subject can be a subject having cachexia or muscle wasting. Increasing SIRT3 activity can also be used to increase or maintain body temperature, e.g., in hypothermic subjects and increasing SIRT1 activity is beneficial for treating hyperlipidemia, diabetes mellitus and impaired glucose tolerance and reducing inflammatory responses in a subject. Increase in metabolic activation of hepatocytes, adipocytes or one or more of their muscles can be useful in lowering the lipid content and increasing weight loss of the subject. The content or levels of the lipids and lipoproteins can be lowered.

Increasing SIRT3 activity can also be used for treating or preventing hyperlipidemia, cardiovascular diseases, reducing blood pressure by vasodilation, increasing cardiovascular health, and increasing the contractile function of vascular tissues, e.g., blood vessels and arteries (e.g., by affecting smooth muscles). Generally, activation of SIRT3 can be used to stimulate the metabolism of hepatocytes, adipocytes or any type of muscle, e.g., muscles of the gut or digestive system, or the urinary tract, and thereby can be used to control gut motility, e.g., constipation, and incontinence. SIRT3 activation can also be useful in erectile dysfunction. It can also be used to stimulate sperm motility, e.g., and be used as a fertility drug. Other embodiments in which it would be useful to increase SIRT3 include repair of muscle, such as after a surgery or an accident, increase of muscle mass; and increase of athletic performance.

Thus the invention provides methods in which beneficial effects are produced by of nicotinic acid and/or nicotinamide riboside and/or any metabolites thereof, and an anti-diabetic such as biguanide (e.g. metformin or any analog thereof), along with leucine and/or leucine metabolites that increase the protein or activity level of SIRT1 or SIRT3. The activity of SIRT1 and SIRT3 can be increased in muscle cells and/or hepatocytes in the subject. These methods effectively facilitate, increase or stimulate one or more of the following: mimic the benefits of calorie restriction or exercise in the hepatocyte or muscle cells, increase mitochondrial biogenesis or metabolism, increase mitochondrial activity and/or endurance in the hepatocytes or muscle cells, sensitize the muscle cells to insulin, increase fatty acid oxidation in the muscle cell, decrease reactive oxygen species (ROS) in the muscle cell, increase PGC-1α and/or UCP3 and/or GLUT4 expression in the hepatocytes or muscle cells, and activate AMP activated protein kinase (AMPK) in the hepatocytes or muscle cells. Various types of muscle cells can be contacted in accordance with the invention. In some embodiments, the muscle cell is a skeletal muscle cell. In certain embodiments, the muscle cell is a cell of a slow-twitch muscle, such as a soleus muscle cell.

Glucose uptake can be measured using in vivo or in vitro techniques. For example, glucose uptake can be measure in vivo using a PET scan in conjunction with labeled glucose or glucose analog. Measurements of glucose uptake can be quantified from the PET scan or by any other technique known in the art. In some embodiments, the glucose uptake can be measured by quantitation of exogenously administered 18-F-deoxyglucose uptake via PET.

ROS/Oxidative Stress can be measured by drawing blood into EDTA-treated tubes, centrifuging to separate plasma, and aliquoting samples for individual assays. Plasma can be maintained at −80° C. under nitrogen to prevent oxidative changes prior to measurements. Plasma malonaldehyde (MDA) can be measured using a fluorometric assay, and plasma 8-isoprostane F_{2α} was measured by ELISA (Assay Designs, Ann Arbor, Mich.).

Another embodiment provides for the administration of a composition comprising synergizing amounts of leucine and resveratrol to the subject in an amount sufficient to increase fatty acid oxidation within the cells of the subject. Yet other embodiments provide for the administration of a composition comprising synergizing amounts of leucine, HMB and resveratrol to a subject in an amount sufficient to increase fatty acid oxidation in the subject.

The compositions can be administered to a subject orally or by any other methods. Methods of oral administration include administering the composition as a liquid, a solid, or a semi-solid that can be taken in the form of a dietary supplement or a food stuff.

The compositions can be administered periodically. For example, the compositions can be administered one, two, three, four times a day, or even more frequent. The subject can be administered every 1, 2, 3, 4, 5, 6 or 7 days. In some embodiments, the compositions are administered three times daily. The administration can be concurrent with meal time of a subject. The period of treatment or diet supplementation can be for about 1, 2, 3, 4, 5, 6, 7, 8, or 9 days, 2 weeks, 1-11 months, or 1 year, 2 years, 3, years, 4 years, 5 years or even longer. In some embodiments of the invention, the dosages that are administered to a subject can change or remain constant over the period of treatment. For example, the daily dosing amounts can increase or decrease over the period of administration.

The length of the period of administration and/or the dosing amounts can be determined by a physician or any other type of clinician. The physician or clinician can observe the subject's response to the administered compositions and adjust the dosing based on the subject's performance. For example, dosing for subjects that show reduced effects in energy regulation can be increased to achieve desired results.

In some embodiments, the components in the compositions can be administered together at the same time in the same route, or administered separately. The components in the compositions can also be administered subsequently. In some embodiments, leucine and/or leucine metabolites in the compositions can be administered to a subject in conjunction with nicotinic acid, nicotinamide riboside and/or nicotinic acid metabolites. In some embodiments, the components in the compositions can be administered at the same or different administration route. For example, leucine and/or leucine metabolites, and/or biguanide (e.g. metformin or any analog thereof) can be administered orally while nicotinic acid, nicotinamide riboside and/or nicotinic acid metabolites can be administered via intravenous injection. Each of the metabolites can be administered via the same or different administration routes.

In some embodiments, the composition administered to a subject can be optimized for a given subject. For example, the ratio of leucine and/or leucine metabolites to nicotinic acid, nicotinamide riboside and/or nicotinic acid metabolites, or to an anti-diabetic such as a biguanide (e.g. metformin or any analog thereof) or the particular components in a combination composition can be adjusted. The ratio and/or particular components can be selected after evaluation of the subject after being administered one or more compositions with varying ratios of and/or leucine metabolites to nicotinic acid, nicotinamide riboside and/or nicotinic acid metabolites or varying combination composition components.

Another aspect of the invention provides for achieving desired effects in one or more subjects after administration of a combination composition described herein for a specified time period. For example, the beneficial effects of the compositions described herein can be observed after administration of the compositions to the subject for 1, 2, 3, 4, 6, 8, 10, 12, 24, or 52 weeks.

The invention provides for a method of treating subjects, comprising identifying a pool of subjects amenable to treatment. The identifying step can include one or more screening tests or assays. For example, subjects that are identified as diabetic or hyperlipidemic, or that have above average or significantly greater than average body mass indices (BMI) and/or weight can be selected for treatment. The subject can be overweight or obese, which can be indicated by an above ideal body weight of the subject or a BMI that is higher than 25, 30, 40, or 50. The subject can weight more than about 50, 75, 100, 125, 150, 160, 170, 180, 190, 200, 210, 220, 230, 240, 250, 260, 270, 280, 290, 300, 310, 320, 330, 340, 350, 360, 370, 380, 390, or 400 lbs. The subjects that have been on a high fat diet can be selected for treatment as well. The identified subjects can then be treated with one or more compositions described herein. For example, they can be treated with a combination composition comprising nicotinic acid, a branched-chain amino acid and an anti-diabetic such as a biguanide such as metformin or any analog thereof.

The invention also provides for methods of manufacturing the compositions described herein. In some embodiments, the manufacture of a composition described herein comprises mixing or combining two or more components. These components can include an anti-diabetic (such as a biguanide like metformin or any analog thereof), nicotinic acid, nicotinamide riboside and/or nicotinic acid metabolites, and leucine or metabolites thereof (such as HMB, or KIC), and/or a sirtuin or AMPK pathway activator (a polyphenol or polyphenol precursor like resveratrol). The amount or ratio of components can be that as described herein. For example, the mass ratio of leucine compared with resveratrol can be greater than about 80.

In some embodiments, the compositions can be combined or mixed with a pharmaceutically active or therapeutic agent, a carrier, and/or an excipient. Examples of such components are described herein. The combined compositions can be formed into a unit dosage as tablets, capsules, gel capsules, slow-release tablets, or the like.

In some embodiments, the composition is prepared such that a solid composition containing a substantially homogeneous mixture of the one or more components is achieved, such that the one or more components are dispersed evenly throughout the composition so that the composition can be readily subdivided into equally effective unit dosage forms such as tablets, pills and capsules.

### Kits

The invention also provides kits. The kits include one or more compositions described herein, in suitable packaging, and can further comprise written material that can include instructions for use, discussion of clinical studies, listing of side effects, and the like. Such kits can also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information can be based on the results of various studies, for example, studies using experimental animals involving in vivo models and studies based on human clinical trials. A kit can comprise one or more unit doses described herein. In some embodiments, a kit comprises about, less than about, or more than about 1, 2, 3, 4, 5, 6, 7, 8, 9, 10, 11, 12, 13, 14, 15, 16, 17, 18, 19, 20, 30, 31, 60, 90, 120, 150, 180, 210, or more unit doses. Instructions for use can comprise dosing instructions, such as instructions to take 1, 2, 3, 4, 5, 6, 7, 8, 9, 10, or more unit doses 1, 2, 3, 4, 5, 6, 7, 8, 9, 10, or more times per day. For example, a kit can comprise a unit dose supplied as a tablet, with each tablet package separately, multiples of tablets packaged separately according to the number of unit doses per administration (e.g. pairs of tablets), or all tablets packaged together (e.g. in a bottle). As a further example, a kit can comprise a unit dose supplied as a bottled drink, the kit comprising 1, 2, 3, 4, 5, 6, 7, 8, 9, 10, 11, 12, 13, 14, 24, 28, 36, 48, 72, or more bottles.

The kit can further contain another agent. In some embodiments, the compound of the present invention and the agent are provided or packaged as separate compositions in separate containers within the kit. In some embodiments, the compound of the present invention and the agent are provided or packaged as a single composition within a container in the kit. Suitable packaging and additional articles for use (e.g., measuring cup for liquid preparations, foil wrapping to minimize exposure to air, and the like) are known in the art and can be included in the kit. Kits described herein can be provided, marketed and/or promoted to health providers, including physicians, nurses, pharmacists, formulary officials, and the like. Kits can also, in some embodiments, be marketed directly to the consumer.

In some embodiments, a kit can comprise a multi-day supply of unit dosages. The unit dosages can be any unit dosage described herein. The kit can comprise instructions directing the administration of the multi-day supply of unit dosages over a period of multiple days. The multi-day supply can be a one-month supply, a 30-day supply, or a multi-week supply. The multi-day supply can be a 90-day, 180-day, 3-month or 6-month supply. The kit can include packaged daily unit dosages, such as packages of 1, 2, 3, 4, or 5 unit dosages. The kit can be packaged with other dietary supplements, vitamins, and meal replacement bars, mixes, and beverages.

### EXAMPLES

### Example 1: Weight Gain, Fat Oxidation, and Insulin Sensitivity in Animals Treated with Resveratrol and Leucine or HMB

Six week old male c57/BL6 mice were fed a high-fat diet with fat increased to 45% of energy (Research Diets D12451) for 6 weeks to induce obesity. At the end of this obesity induction period, animals were randomly divided into the following seven different diet treatment groups with 10 animals per group (overall 70 animals) and maintained on these diets for 6 weeks:
Group 1 (labeled “control group”): high-fat diet only (same as in obesity induction period (Research Diets D12451)).
This diet was modified for groups 2 to 7 in the following way:
Group 2 (labeled “low dose resveratrol”): high-fat diet mixed with 12.5 mg resveratrol/kg diet.
Group 3 (labeled “high dose resveratrol”): high-fat diet mixed with 225 mg resveratrol/kg diet.
Group 4 (labeled “low dose HMB”): high-fat diet mixed with 2 g of the calcium salt of hydroxymethylbutyrate, a naturally occurring metabolite of leucine (CaHMB).
Group 5 (labeled “low dose resveratrol plus low dose CaHMB”): high fat-diet mixed with 12.5 mg of resveratrol/kg diet and 2 g CaHMB/kg diet.
Group 6 (labeled “low dose resveratrol plus high dose HMB”): high fat-diet mixed with 12.5 mg of resveratrol/kg diet and 10 g CaHMB/kg diet.
Group 7 (labeled “low dose resveratrol plus leucine”): high fat-diet mixed with 12.5 mg of resveratrol/kg diet and leucine increased to 200% of its normal level (from 1.21 to 2.42% by weight) of the control diet.

The animals were housed in polypropylene cages at a room temperature of 22±2° C. and regime of 12 h light/dark cycle. The animals had free access to water and their experimental food throughout the experiment. At the of the treatment period (6 weeks) all animals were humanely euthanized, and blood and tissues collected for further experiments.

Oxygen Consumption/Substrate Utilization:

at the end of the obesity induction period (day 0 of treatment group) and at 2 weeks and 6 weeks of treatment, oxygen consumption and substrate utilization was measured via metabolic chambers using the Comprehensive Lab Animal Monitoring Systems (CLAMS, Columbus Instruments, Columbus, Ohio) in subgroups of each treatment group. Each animal was placed in individual cages without bedding that allow automated, non-invasive data collection. Each cage is an indirect open circuit calorimeter that provides measurement of oxygen consumption, carbon dioxide production, and concurrent measurement of food intake. All mice were acclimatized to the chambers for 24 hours prior to the experiment and maintained under the regular 12:12 light:dark cycle with free access to water and food. All experiments were started in the morning and data were collected for 24 hours. Each chamber was passed with 0.61 of air/min and was sampled for 2 min at 32-minute intervals. Exhaust O₂ and CO₂ content from each chamber was compared with ambient O₂ and CO₂ content. Food consumption was measured by electronic scales.

microPET/CT (Glucose and Palmitate Uptake):

at the end of the treatment period (6 weeks of treatment) subgroups of each treatment diet group (5 animals/group, 35 animals total) were used to measure whole body glucose and palmitate uptake via PET/CT Imaging. To visualize these compounds using microPET imaging, the glucose or palmitate was labeled with fluorine-18 (108 mins half-life) or carbon-11 (20 mins half-life), respectively. Each mouse was fasted for 4 hours, then anesthetized using 1-3% isoflurane delivered by nose cone or in a mouse-sized induction chamber purpose-built for small animal imaging protocols. While under anesthesia the mice were injected iv with <2 mCi of each tracer, then be left for a period of time (minutes to up ˜1 hour) to allow the uptake of the tracer. During the scan, mice were kept warm using a thermostatically controlled heated bed and were treated with ophthalmic ointment prior to scanning Following the live scan the mice were returned to their cage and revived. Mice were monitored constantly during this time. Following live data acquisition the mice were sacrificed by isoflurane overdose and organs harvested for further experiments.

RNA Extraction:

The Ambion ToTALLY RNA isolation kit (Ambion, Inc., Austin, Tex., USA) was used to extract total RNA from tissue according to the manufacturer's instruction. The concentration, purity and quality of the isolated RNA will be assessed by measuring the 260/280 ratio (1.8-2.0) and 260/230 ratio (close to 2.0) by using the ND-1000 Spectrophotometer (NanoDrop Technologies Inc., Del. USA). Biomarkers of the sirtuin-pathway, cytokines, and inflammatory markers (including but not limited to C-reactive protein, IL-6, MCP-1, and adiponectin molecules) can be assessed at the RNA level.

Gene Expression:

Expression of 18S, Sirt1, Sirt3, PGC1-α, cytochrome c oxidase subunit VIIc1 (COX 7), mitochondrial NADH dehydrogenase, nuclear respiratory factor 1 (NRF1), uncoupling protein (UCP2 (adipocyte)/UCP3 (myocyte), p53, AMPK, Akt/PKB, and GLUT4 is measured via quantitative real-time PCR using an ABI 7300 Real-Time PCR system (Applied Biosystems, Branchburg, N.J.) with a TaqMan® core reagent kit. All primers and probe sets can be obtained from Applied Biosystems TaqMan® Assays-on-Demand and utilized accordingly to manufacturer's instructions. Pooled RNA from each cell type are serial-diluted in the range of 0.0156-50 ng and were used to establish a standard curve; total RNA for each unknown sample is also diluted in this range. RT-PCR reactions are performed according to the instructions of the ABI Real-Time PCR system and TaqMan Real Time PCR Core Kit. Expression of each gene of interest is then normalized using the corresponding 18S quantitation.

SIRT1 Activity:

SIRT1 activity was measured by using the SIRT1 Fluorimetric Drug Discovery Kit (BML-AK555, ENZO Life Sciences International, Inc. PA, USA). In this assay, SIRT1 activity is assessed by the degree of deacetylation of a standardized substrate containing an acetylated lysine side chain. The substrate utilized is a peptide containing amino acids 379-382 of human p53 (Arg-His-Lys-Lys[Ac]), an established target of SIRT1 activity; SIRT1 activity is directly proportional to the degree of deacetylation of Lys-382. Samples were incubated with peptide substrate (25 μM), and NAD⁺ (500 μM) in a phosphate-buffered saline solution at 37° C. on a horizontal shaker for 45 minutes. The reaction was stopped with the addition of 2 mM nicotinamide and a developing solution that binds to the deacetylated lysine to form a fluorophore. Following 10 minutes incubation at 37° C., fluorescence was read in a plate-reading fluorometer at an excitation wavelength of 360 nm and an emission wavelength of 450 nm. Resveratrol (100 mM) served as a SIRT1 activator and suramin sodium (25 mM) as a SIRT1 inhibitor; wells including each were utilized as positive and negative controls in each set of reactions. A standard curve was constructed using deacetylated substrate (0-10 μM). Data was normalized to cellular protein concentration measured via BCA-assay.

Western Blot Analysis:

Tissue samples (adipose and muscle) is homogenized in ice-cold RIPA lysis buffer containing 150 mM sodium chloride, 1.0% Triton X-100, 0.5% sodium deoxycholate, 0.1% SDS and 50 mM Tris (pH 8.0), aprotinin (1 μg/ml), Leupeptin (10 μg/ml), Pepstatin A (1 μg/ml), 1 mM PMSF, 5 mM EDTA, 1 mM EGTA, 10 mM NaF, 1 mM Na Orthovanadate with an electric homogenizer, then maintained on constant agitation for 2 hours at 4° C. and centrifuged at 4,000 g for 30 min at 4° C. Aliquots of supernatants (containing 15-25 μg of total protein) is treated with 2× Laemmli sample buffer containing 100 mM dithiothreitol and run on 10% (for or 15% SDS-PAGE (for Sirt3). The resolved proteins is transferred to PVDF membrane and blocked in 5% nonfat dry milk in Tris-buffered saline containing 0.1% Tween 10, pH 7.5. After membranes are blocked, the membranes are rinsed in TBST, incubated overnight with appropriate antibody, rinsed in TBST, and incubated for 120 min with horseradish peroxidase-conjugated anti-rabbit IgG. Antibody-bound protein is visualized with enhanced chemiluminescence (ECL, Amersh).

The following antibodies are used: Anti-Sirt3 antibody (Cell Signaling Technology, Beverly, Mass.), Anti-Idh2 (Isocitrate dehydrogenase 2) (Santa Cruz, Calif.), Anti-COX antibody (Santa Cruz).

Low doses of resveratrol and HMB exerted no significant independent effect on body weight, weight gain, visceral adipose tissue mass, fat oxidation, respiratory exchange ratio (RER), or heat production, while the high dose of resveratrol significantly increased both heat production and skeletal muscle fat oxidation and decreased RER, indicating a whole-body shift towards fat oxidation Table 1); however, high dose resveratrol exerted no significant effect on body weight, weight gain, or visceral adipose tissue mass. In contrast with the lack of independent effects of a low dose of resveratrol or HMB, combining a low dose of resveratrol with either HMB or leucine resulted in significant reductions in body weight, weight gain, visceral adipose tissue mass, fat oxidation and heat production, and an associated decrease in RER (Table 1).

TABLE 1
Effects of resveratrol, leucine and HMB on body weight, weight gain, adiposity and fat oxidation in diet-induced obese mice.¹

| | | Low | High | Low | Low Resv/ | Low Resv/ | Low Resv/ | |
|---|---|---|---|---|---|---|---|---|
| | Control | Resveratrol² | Resveratrol³ | HMB⁴ | Low HMB | High HMB⁵ | Leucine⁶ | P value |
| Weight (g) | 40.5 ± 0.5^{a} | 40.8 ± 2.5^{a} | 38.7 ± 1.2^{a} | 40.3 ± 2.1^{a} | 36.2 ± 3.2^{b} | 34.4 ± 1.1^{b} | 38.3 ± 2.3^{b} | P < 0.05 |
| Weight | 22.4 ± 1.1^{a} | 20.9 ± 1.5^{a} | 22.3 ± 2.4^{a} | 22.5 ± 1.2 | 18.2 ± 1.2^{b} | 19.2 ± 1.0^{b} | 19.2 ± 1.6^{b} | p < 0.01 |
| gain (g) | | | | | | | | |
| Visceral | 6556 ± 143 | 6551 ± 575^{a} | 6031 ± 323^{a} | 6184 ± 460^{a} | 5302 ± 324^{b} | 4879 ± 243^{b} | 4259 ± 321^{b} | p < 0.01 |
| Adipose | | | | | | | | |
| Volume | | | | | | | | |
| (mm³) | | | | | | | | |
| Fat oxidation | 1.34 ± 0.15^{a} | 1.51 ± 0.44^{a} | 2.29 ± 0.11^{b} | 1.90 ± 0.29^{a} | 2.09 ± 0.30^{b} | 1.97 ± 0.28^{b} | 1.76 ± 0.09^{a,b} | P < 0.05 |
| (PET | | | | | | | | |
| palmitate | | | | | | | | |
| uptake; | | | | | | | | |
| Muscle | | | | | | | | |
| SUV) | | | | | | | | |
| Respiratory | 0.850 ± 0.008^{a} | 0.847 ± 0.008^{a} | 0.825 ± 0.007^{b} | 0.844 ± 0.012^{a} | 0.815 ± 007^{b} | 0.8818 ± 0.09^{b} | 0.811 ± 0.010^{b} | P < 0.01 |
| Exchange | | | | | | | | |
| Ratio (24 | | | | | | | | |
| hr RER) | | | | | | | | |
| Heat | 0.521 ± 0.015^{a} | 0.517 ± 0.014^{a} | 0.552 ± 0.015^{b} | 0.526 ± 0.011^{a} | 0.544 ± 0.010^{b} | 0.547 ± 0.009^{b} | 0.550 ± 0.012^{b} | P < 0.05 |
| Production | | | | | | | | |

¹non-matching letter superscripts in each row denote significant differences at the indicated p value
²Low resveratrol: 12.5 mg resveratrol/kg diet
³High resveratrol: 225 mg resveratrol/kg diet
⁴Low HMB: 2 g hydroxymethylbutyrate (calcium salt)
⁵Leucine: Leucine increased two-fold, from 1.21% in other diets to 2.42%

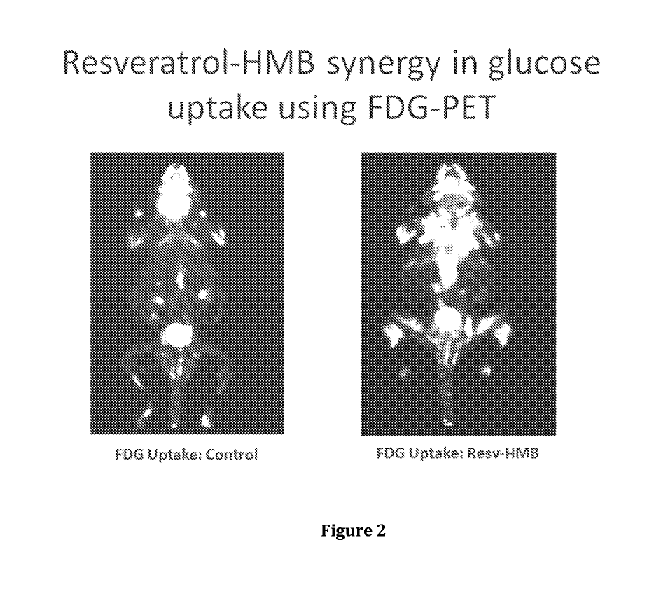

Table 2 shows the effects of the dietary treatments on indices of insulin sensitivity. None of the treatments exerted any effect on plasma glucose. Neither resveratrol at either dose nor HMB exerted any significant effect on plasma insulin or on muscle glucose uptake. However, the combination of a low dose of resveratrol with either HMB or leucine resulted in significant, marked decreases in plasma insulin. This reduction in insulin with no change in plasma glucose reflects significant improvements in muscle and whole-body insulin sensitivity, as demonstrated by significant and substantial decreases in HOMA_{IR} (homeostatic assessment of insulin resistance) and corresponding increases in skeletal muscle ¹⁸F-deoxyglucose uptake (Table 2 and FIG. 2).

TABLE 2
Effects of resveratrol, leucine and HMB on indices of insulin sensitivity in diet-induced obese mice.¹

| | | Low | High | Low | Low Resv/ | Low Resv/ | Low Resv/ | |
|---|---|---|---|---|---|---|---|---|
| | Control | Resveratrol² | Resveratrol³ | HMB⁴ | Low HMB | High HMB⁵ | Leucine⁶ | P value |
| Glucose (mM) | 4.97 ± 0.60^{a} | 5.14 ± 0.85^{a} | 5.14 ± 0.75^{a} | 4.28 ± 0.49^{a} | 4.67 ± 0.49^{a} | 4.33 ± 0.41^{a} | 5.05 ± 0.92^{a} | NS |
| Insulin | 12.5 ± 3.4^{a} | 10.4 ± 1.6^{a} | 10.1 ± 2.7^{a} | 8.3 ± 1.1^{a} | 5.8 ± 0.7^{b} | 3.9 ± 1.2^{b} | 5.5 ± 1.4^{b} | P < 0.005 |
| (μU/mL) | | | | | | | | |
| HOMA_{IR} | 2.61 ± 0.82^{a} | 2.41 ± 0.66^{a} | 0.59 ± 0.26^{b} | 1.93 ± 0.32^{a} | 1.18 ± 0.25^{c} | 0.87 ± 0.31^{b} | 1.14 ± 0.37^{c} | P < 0.01 |
| Muscle Glucose | 3.64 ± 0.88^{a} | 3.63 ± 1.29^{a} | 3.87 ± 0.32^{a} | 2.99 ± 0.42^{a} | 5.90 ± 0.41^{b} | 5.93 ± 1.63^{b} | 5.68 ± 0.75^{b} | P < 0.02 |
| Uptake (¹⁸F- | | | | | | | | |
| deoxyglucose | | | | | | | | |
| SUV) | | | | | | | | |

¹non-matching letter superscripts in each row denote significant differences at the indicated p value
²Low resveratrol: 12.5 mg resveratrol/kg diet
³High resveratrol: 225 mg resveratrol/kg diet
⁴Low HMB: 2 g hydroxymethylbutyrate (calcium salt)
⁵Leucine: Leucine increased two-fold, from 1.21% in other diets to 2.42%

### Example 2—Synergistic Effects of Polyphenol and Related Compounds with Anti-Diabetic Agents on Sirtuin Activation and Downstream Pathways

Compounds were tested for potential to independently or synergistically modulate sirtuin signaling either by direct stimulation or indirect via upstream signaling via AMPK. One or more of these compounds, including chlorogenic acid, quinic acid, sorbitol, myo-inositol, maltitol, cinnamic acid, ferulic acid, piceatannol, ellagic acid, epigallocatechin gallate, fucoxanthin, grape seed extract, metformin, rosiglitazone, PDE inhibitors, caffeine, theophylline, theobromine, and isobutylmethylxanthine, can be used in combination with other components described herein, including compositions including (a) leucine, anti-diabetic agents and one or more agents selected from the group consisting of nicotinic acid, nicotinamide riboside and nicotinic acid metabolites, (b) leucine and guanides, and (c) leucine and one or more agents selected from the group consisting of nicotinic acid, nicotinamide riboside and nicotinic acid metabolites, to synergistically or independently modulate the SIRT pathway and/or augment the effects of such other components. A key outcome of Sirt1 signaling is stimulation of PGC1-α and subsequent stimulation of mitochondrial biogenesis and fatty acid oxidation. Accordingly, fatty acid oxidation, measured as palmitate-induced oxygen consumption as described below, was utilized as a sensitive first level of screening for aerobic mitochondrial metabolism. A dose-response curve for fatty acid oxidation was established for each compound studied, and a “sub-therapeutic dose” was defined as the highest dose that exerted no effect in this system. This dose, typically found to be in the 200-1000 nM range for most compounds studied, was then used to evaluate synergistic effects with leucine, HMB, or sub-therapeutic doses of other compounds. These experiments were conducted in fully differentiated adipocytes (3T3-L1) and myotubes (C2C12). To evaluate the impact of these combinations on cross-talk between adipose and muscle tissues, adipocytes were treated for 48 hours, the medium collected (conditioned media, CM) and then exposed to myotubes; similar experiments were conducted with myotubes treated, CM collected, and exposed to adipocytes. Following assessment of fatty acid oxidation, Sirt1 activity, AMPK activity, mitochondrial biogenesis and glucose utilization (measured as glucose-induced extracellular acidification in the absence of fatty acids in the media) were assessed for lead combinations and appropriate controls.

Cell Culture:

C2C12 and 3T3-L1 preadipocytes (American Type Culture Collection) were plated at a density of 8000 cells/cm² (10 cm² dish) and grown in Dulbecco's modified eagle's medium (DMEM) containing 10% fetal bovine serum (FBS), and antibiotics (growth medium) at 37° C. in 5% CO₂. Confluent 3T3-L1 preadipocytes were induced to differentiate with a standard differentiation medium consisting of DMEM medium supplemented with 10% FBS, 250 nM dexamethasone, 0.5 mM 3-Isobutyl-1-methylxanthine (IBMX) and 1% penicillin-streptomycin. Preadipocytes were maintained in this differentiation medium for 3 days and subsequently cultured in growth medium. Cultures were re-fed every 2-3 days to allow >90% cells to reach fully differentiation before conducting chemical treatment. For differentiation of C2C12 cells, cells were grown to 100% confluence, transferred to differentiation medium (DMEM with 2% horse serum and 1% penicillin-streptomycin), and fed with fresh differentiation medium every day until myotubes were fully formed (3 days).

### Measurements:

Fatty Acid Oxidation:

Cellular oxygen consumption was measured using a Seahorse Bioscience XF24 analyzer (Seahorse Bioscience, Billerica, Mass.) in 24-well plates at 37° C., as described by Feige et al. (Feige J, Lagouge M, Canto C, Strehle A, Houten S M, Milne J C, Lambert P D, Mataki C, Elliott P J, Auwerx J. Specific SIRT1 activation mimics low energy levels and protects against diet-induced metabolic disorders by enhancing fat oxidation. Cell Metabolism 2008; 8:347-358) with slight modifications. Cells were seeded at 40,000 cells per well, differentiated as described above, treated for 24 hours with the indicated treatments, washed twice with non-buffered carbonate-free pH 7.4 low glucose (2.5 mM) DMEM containing carnitine (0.5 mM), equilibrated with 550 μL of the same media in a non-CO₂ incubator for 45 minutes, and then inserted into the instrument for 15 minutes of further equilibration, followed by O₂ consumption measurement. Three successive baseline measures at five-minute intervals were taken prior to injection of palmitate (200 μM final concentration). Four successive 5-minute measurements of O₂ consumption were then conducted, followed by 10 minute re-equilibration and another 3-4 5-minute measurements. This measurement pattern was then repeated over a 4-6 hour period. Data for each sample were normalized to the pre-palmitate injection baseline for that sample and expressed as % change from that baseline. Pre-palmitate injection values were 371±14 pmol O₂/minute for myotubes and 193±11 pmol O₂/minute for adipocytes. The area under of the curve of O₂ consumption change from baseline for each sample was then calculated and used for subsequent analysis.

Glucose Utilization:

In the absence of a fatty acid source and oxidative metabolism, glycolysis and subsequent lactate production results in extracellular acidification, which was also measured using a Seahorse Bioscience XF24 analyzer. Cells were prepared and equilibrated similar to the methods described above for fatty acid oxidation, with the exclusion of carnitine from the medium. Following instrument equilibration and three baseline measurements, glucose was injected to a final concentration of 10 mM in each well. Measurements were taken as described above utilizing the sensors for extracellular acidification rather than O₂ consumption. Insulin (final concentration of 5 nM) was added to some wells as a positive control. Data for each sample were normalized to the pre-glucose injection baseline for that sample and expressed as % change from that baseline. The area under of the curve of extracellular acidification change from baseline for each sample was then calculated and used for subsequent analysis.

Mitochondrial Biogenesis:

Mitochondrial biogenesis was assessed as change in mitochondrial mass, as described by Sun et al. (Sun X and Zemel M B (2009) Leucine modulation of mitochondrial mass and oxygen consumption in skeletal muscle cells and adipocytes. Nutrition and Metabolism 6:26 (doi:10.1.1186/1743-707S-6-26)). The mitochondrial probe NAO (Invitrogen, Carlsbad, Calif.) was used to analyze mitochondrial mass by fluorescence (excitation 485 nm and emission 520 nm), and quantitative data were obtained with a fluorescence microplate reader (Synergy HT, BioTek Instruments, Winooski, Vt.). The intensity of fluorescence was expressed as arbitrary units per μg protein and normalized to control values within each assay.

AMPK Activity:

AMP-activated protein kinase (AMPK) was measured using a commercial kit (CycLex AMPK Kinase Assay Kit, CycLex Co, Ltd, Nagano, Japan). The assay is based upon AMPK phosphorylation of IRS-1 S789. Phosphorylated IRS-1 S789 is then detected by an anti-phospho-mouse IRS-1 S789 monoclonal antibody, which is then bound to horseradish peroxidase conjugated anti-mouse IgG which catalyzes a chromogenic reaction with tetra-methylbenzidine. Color formation is proportional to AMPK activity and was measured in 96-well ELISA plates at dual wavelengths (450/540 nm) using a microplate reader (Synergy HT, BioTek Instruments, Winooski, Vt.). These values are expressed as fluorescent units/mg protein and normalized to control values within each assay.

Sirt1 Activity:

SIRT1 activity was measured by using the SIRT1 Fluorimetric Drug Discovery Kit (BML-AK555, ENZO Life Sciences International, Inc. PA, USA). The assay measures SIRT1 activity by the degree of deacetylation of a standardized substrate containing an acetylated lysine side chain. The substrate utilized is a peptide containing amino acids 379-382 of human p53 (Arg-His-Lys-Lys[Ac]), an established target of SIRT1 activity; SIRT1 activity is directly proportional to the degree of deacetylation of Lys-382. Samples were incubated with peptide substrate (25 μM), and NAD⁺ (500 μM) in a phosphate-buffered saline solution at 37° C. on a horizontal shaker for 45 minutes. The reaction was stopped with the addition of 2 mM nicotinamide and a developing solution that binds to the deacetylated lysine to form a fluorophore. Following 10 minutes incubation at 37° C., fluorescence was read in a plate-reading fluorometer (Synergy HT, BioTek Instruments, Winooski, Vt.) at an excitation wavelength of 360 nm and an emission wavelength of 450 nm. Resveratrol (100 mM) served as a SIRT1 activator (positive control) and suramin sodium (25 mM) as a SIRT1 inhibitor (negative control). A standard curve was constructed using deacetylated substrate (0-10 μM).

Statistics:

Data were analyzed via one-way analysis of variance and least significant difference test was used to separate significantly different group means.

### Results:

Resveratrol-Leucine and Resveratrol-HMB:

Leucine (0.5 mM) and HMB (5 μM) stimulated Sirt1 activity and fatty acid oxidation by 30-50%, similar to the effects of 10 μM resveratrol, while lower levels of resveratrol (here 200 nM) exerted no effect; leucine, HMB and a low dose of resveratrol exerted no independent effects on Sirt3. However, the combination of either leucine or HMB with 200 nM resveratrol resulted in a ˜90% stimulation of Sirt1, a ˜60% stimulation of both Sirt3 and 91%-118% increases in fatty acid oxidation (p<0.005).

The concentrations of leucine and HMB in all experiments described below are 0.5 mM (leucine) and 5 μM (HMB). Each of the compounds studied in combination with leucine or HMB were studied at concentrations that exerted no independent effect on the variables under study in order to assess potential synergies. These concentrations are defined for each compound below.

Chlorogenic Acid:

Chlorogenic acid is a naturally occurring polyphenol described as a hydroxycinnamic acid; it is an ester of caffeic acid and L-quinic acid (evaluated below). Chlorogenic acid dose-response curves indicate concentrations of 500 nM or below exert no effect; accordingly, this was the concentration used in synergy experiments.

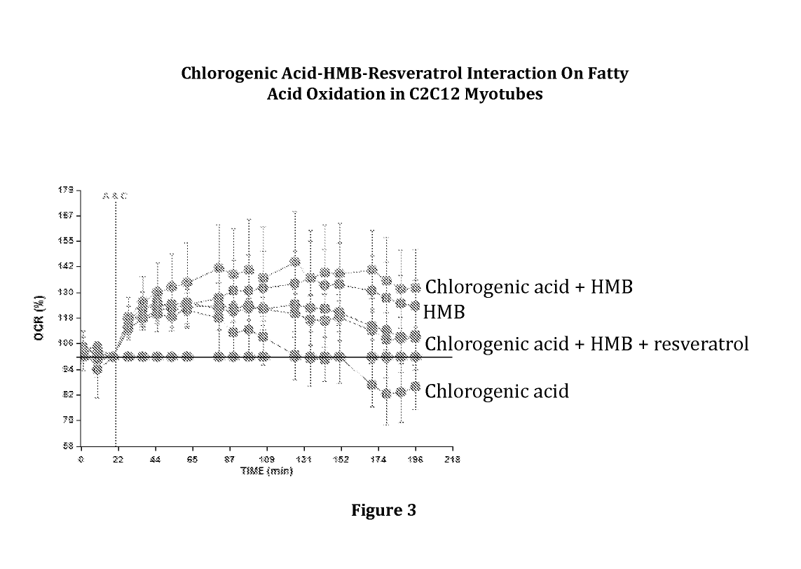

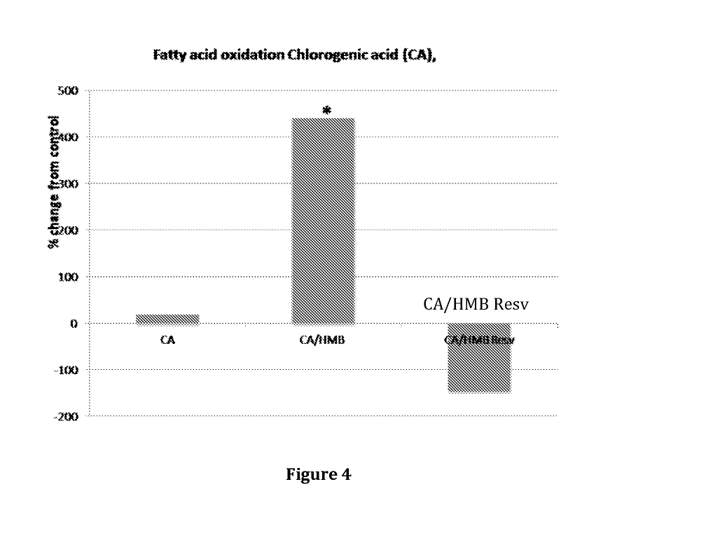

FIG. 3 shows the effects of the chlorogenic acid combinations in myotubes. Chlorogenic acid (500 nM)/HMB elicited a 42% increase in fatty acid oxidation with 6 hour treatment (p=0.003) and 441% over 24 hours (p=0.05) in skeletal muscle cells (myotubes), while no significant effect was observed in adipocytes. Notably, adding resveratrol (200 nM) attenuated or eliminated these effects, suggesting potential competition for a common site of action (FIG. 4).

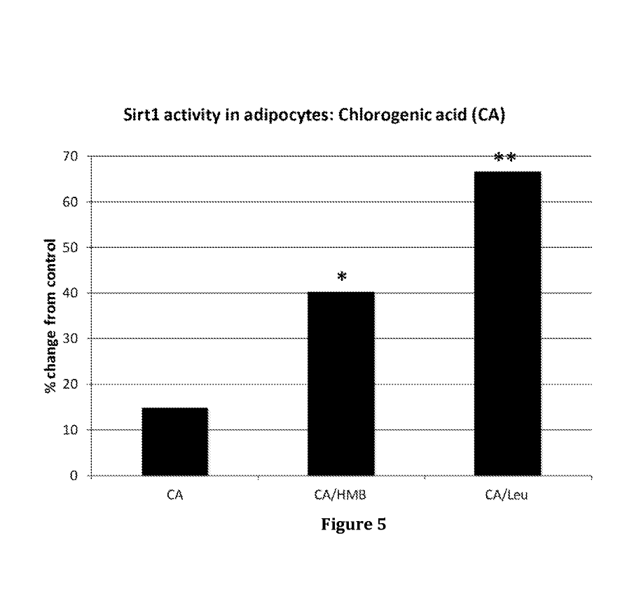

The chlorogenic acid/HMB combination stimulated adipocyte Sirt1 activity 40% (p=0.005) while the chlorogenic acid/leucine combination stimulated Sirt1 by 67% (p=0.0001) (FIG. 5) and more modestly stimulated AMPK activity (30-35%, NS: p=0.078). In contrast to myotubes, the chlorogenic acid/HMB and chlorogenic acid/leucine combinations exerted no direct effect on adipocyte fatty acid oxidation; however, adipocyte conditioned media experiments demonstrated that treatment of adipocytes with these combinations for 48 hours resulted in conditioned media that stimulated myotube fatty acid oxidation by 76% (p=0.013).

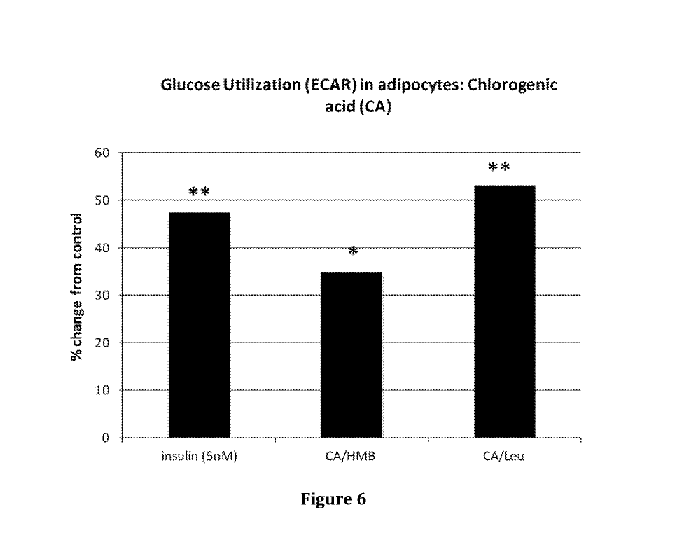

Both chlorogenic acid-leucine and chlorogenic acid-HMB exerted significant effects on glucose utilization as measured by extracellular acidification responses to glucose addition (chlorogenic acid-leucine: 53%, p=0.007; chlorogenic acid-HMB: 35%, p=0.045; FIG. 6).

Caffeic Acid:

Caffeic acid is another naturally occurring phenolic compound described as another hydroxycinnamic acid. Caffeic acid dose-response curves indicate concentrations of 1 μM or below exert no effect; accordingly, this was the concentration used in synergy experiments.

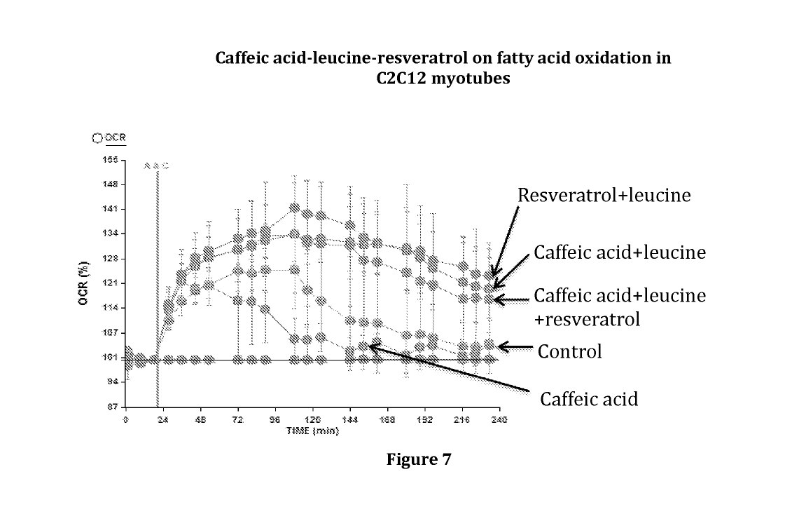

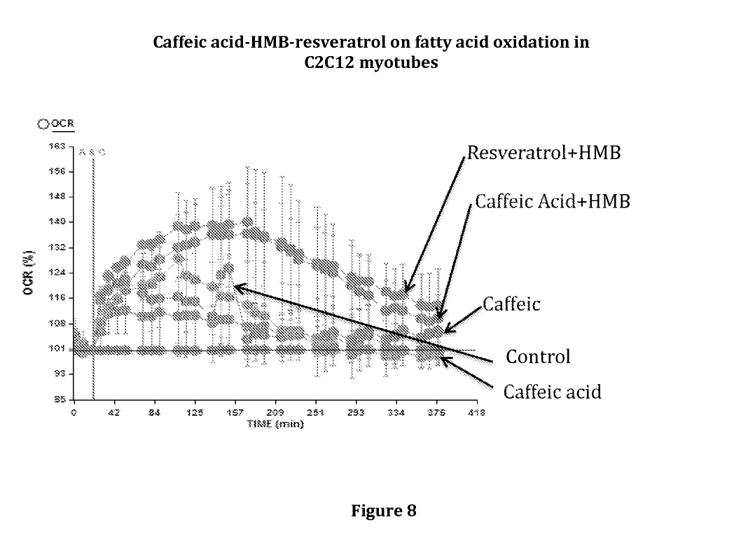

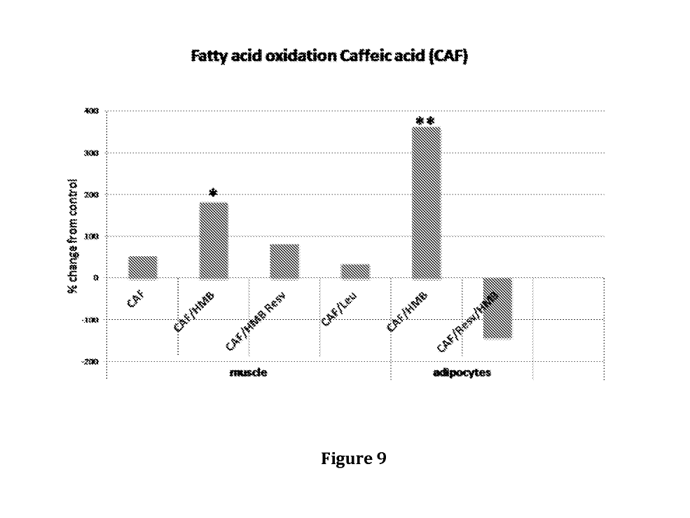

FIGS. 7 and 8 show the effects of the caffeic acid combinations in myotubes, and the quantitative data is summarized in FIG. 9. The caffeic acid-leucine combination exerted a modest, non-statistically significant increase in myotube fatty acid oxidation (35%), while the caffeic acid-HMB combination exerted significant effects on fatty acid oxidation in both adipocytes (361%, p=0.05) and myotubes (182%, p=0.016). These effects were inhibited by the addition of 200 nM resveratrol, suggesting competition, similar to that seen with chlorogenic acid (FIG. 8).

Quinic Acid:

Quinic acid is a naturally occurring polyol found in coffee beans and some other plant products. Although not a polyphenol, it is evaluated here because it is a component of chlorogenic acid and may be produced via hydrolysis of chlorogenic acid. Quinic acid dose-response curves indicate concentrations of 500 nM or below exert no effect; accordingly, this was the concentration used in synergy experiments.

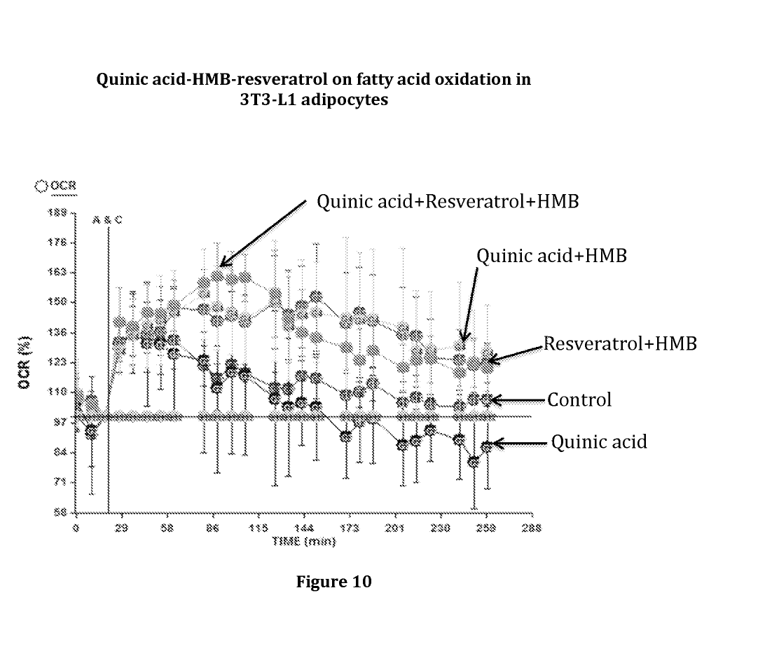

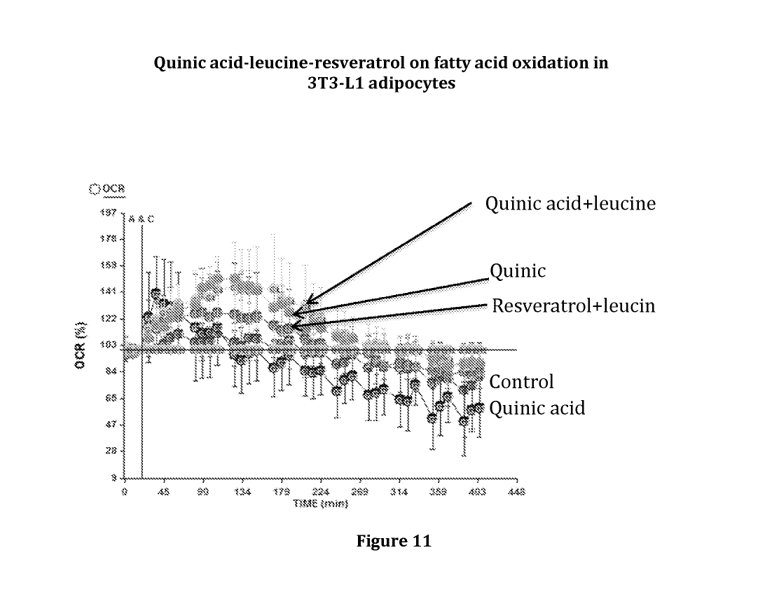

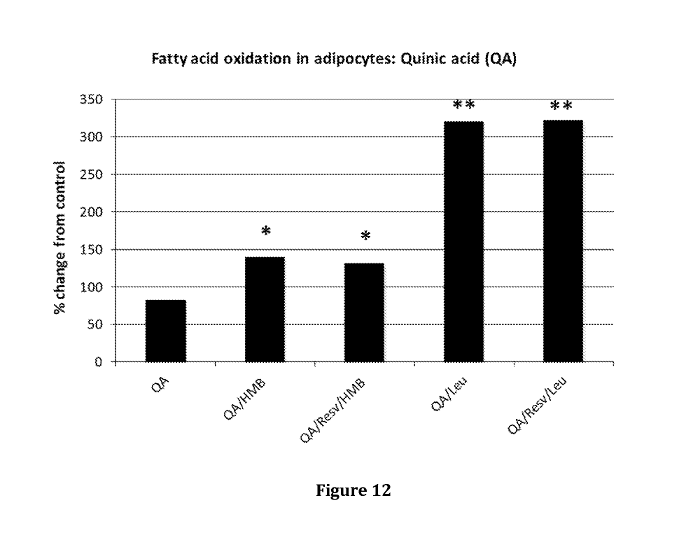

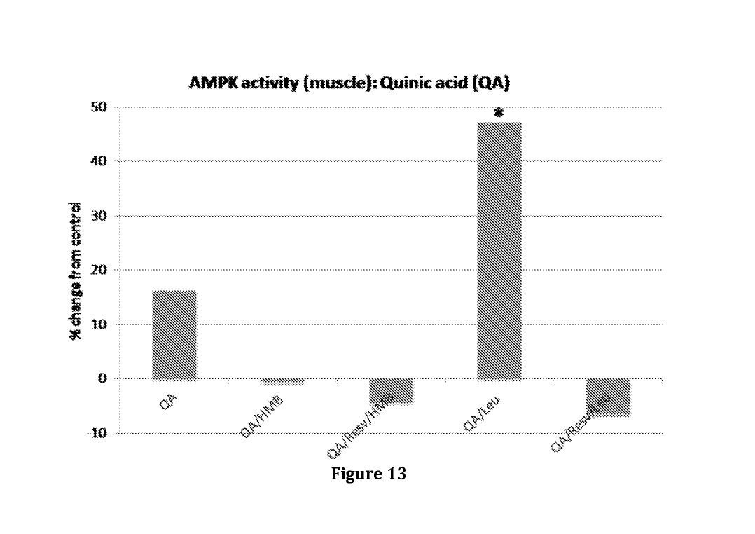

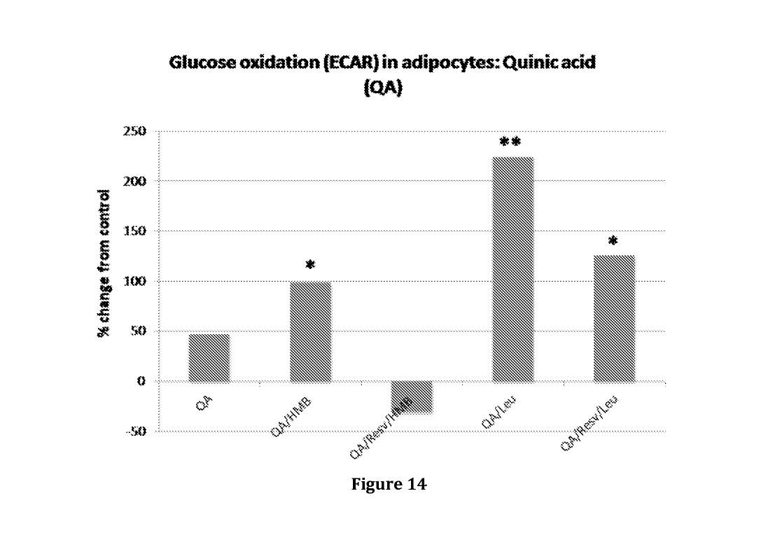

FIGS. 10 and 11 show the effects of the quinic acid combinations in adipocytes, and the quantitative data is summarized in FIG. 12. Quinic acid-HMB and quinic acid-leucine combinations produced robust increases in adipocyte fatty acid oxidation (141% for the quinic acid-HMB combination, p=0.05; 320% for the quinic acid-leucine combination, p=0.012; FIG. 12) and more modest increases in myotubes (˜30%, p=0.03). Unlike chlorogenic acid and caffeic acid, addition of resveratrol (200 nM) did not attenuate these effects. The quinic acid combinations appear not to exert their effects directly on Sirt1, as there was no short-term effect on Sirt1 activity, and instead acts upstream with a significant increase in AMPK activity (47%, p<0.0001; FIG. 13). Both the quinic acid-leucine and quinic acid-HMB combinations exerted significant effects on glucose utilization as measured by extracellular acidification responses to glucose addition in both adipocytes and myotubes (quinic acid-HMB, 99%, p=0.05; quinic acid-leucine, 224%, p=0.0003; FIG. 14).

Other Polyols:

As noted above, quinic acid was evaluated as a hydrolysis product of chlorogenic acid. To determine if the robust effects of quinic acid reflected the effects of a unique molecule (quinic acid) or polyols as a class of compounds, other polyols were evaluated, as follows. These data suggest that effects of quinic acid are not readily extrapolated to other polyols.

Sorbitol is a sugar alcohol analogue of glucose. Sorbitol dose-response curves indicate concentrations of 500 nM or below exert no effect; accordingly, this was the concentration used in synergy experiments. Addition of this level of sorbitol to either HMB or leucine resulted in stimulation of myotube fatty acid oxidation (44-70%, p=0.023). However, these effects are not significantly different from the independent effects of leucine and HMB in the absence of sorbitol, indicating no synergy.

Myo-inositol is a polyol metabolite of glucose. Myo-inositol dose-response curves indicate concentrations of 100 nM or below exert no effect; accordingly, this was the concentration used in synergy experiments. Combining 100 nM myo-inositol with leucine or HMB produced 60% increase in fat oxidation, comparable to the independent effects of leucine and HMB in the absence of myo-inositol, indicating no synergy.

Maltitol is a disaccharide made by hydrogenation of maltose. Maltitol dose-response curves indicate concentrations of 100 nM or below exert no effect; accordingly, this was the concentration used in synergy experiments. However, no synergy was noted.

Cinnamic Acid:

Cinnamic acid is a naturally occurring phenolic found in cinnamon oil. It bears strong structural homology to both caffeic acid and chlorogenic acid. Cinnamic acid dose-response curves indicate concentrations of 500 nM or below exert no effect; accordingly, this was the concentration used in synergy experiments.

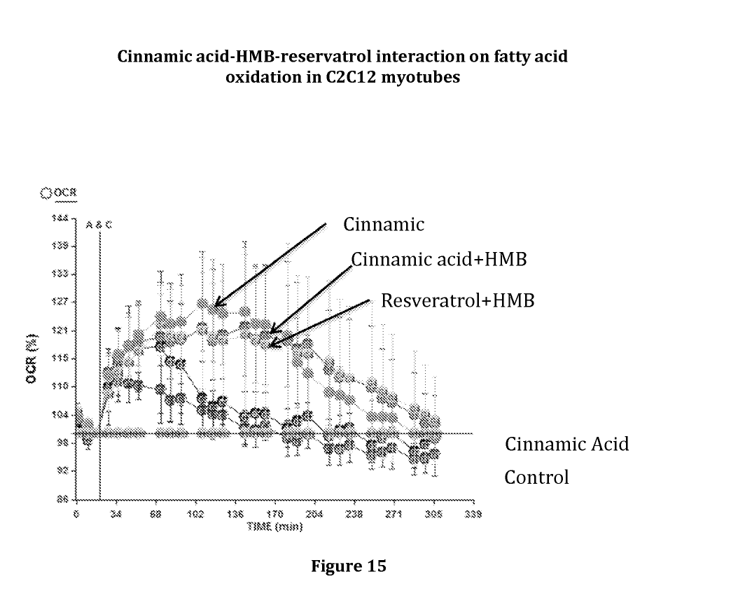

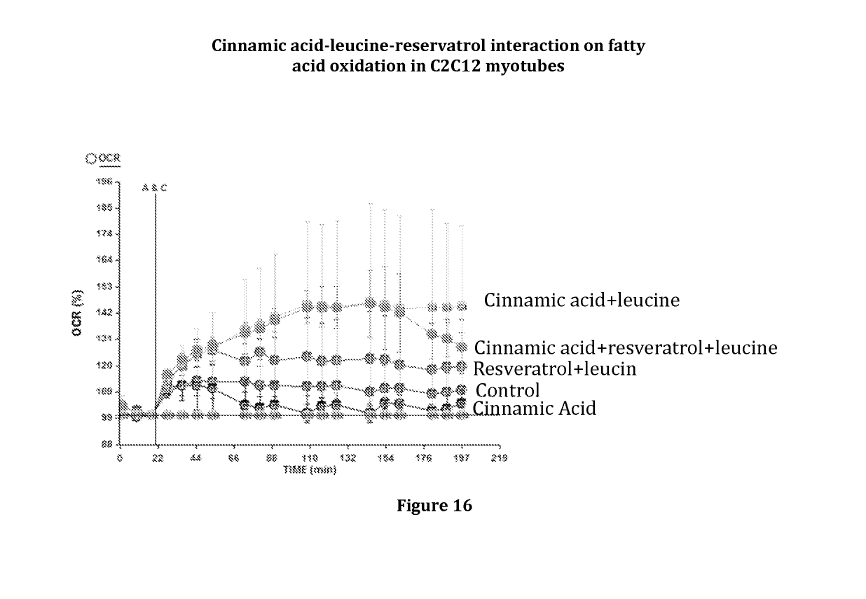

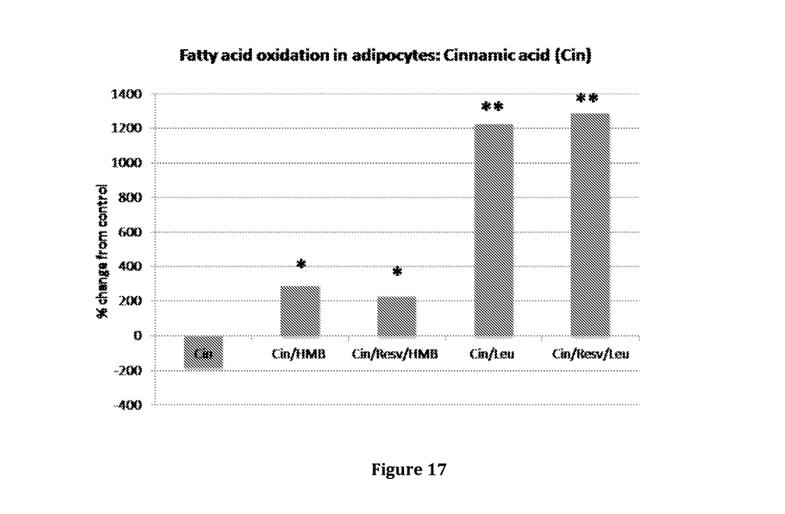

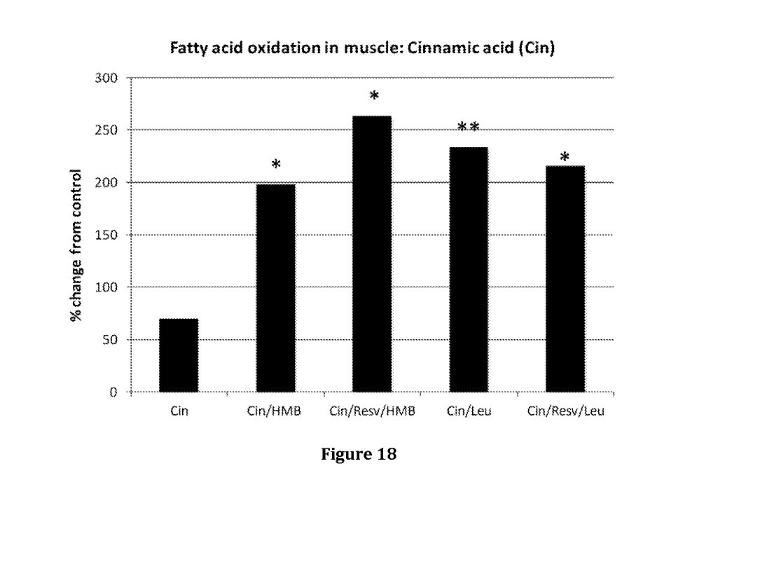

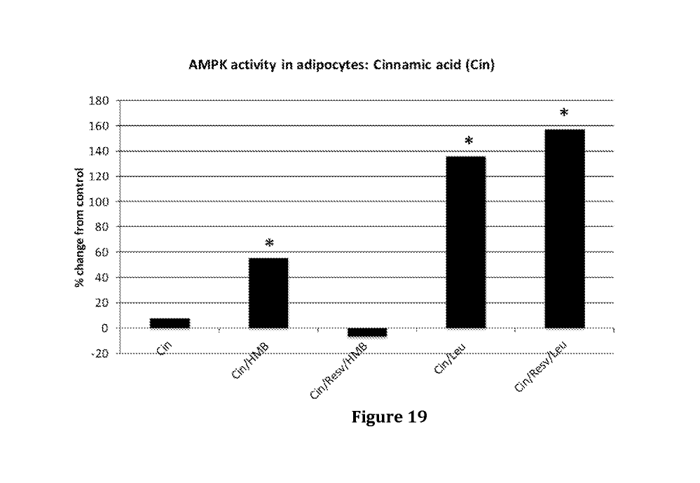

The cinnamic acid combinations exerted robust effects in both adipocytes and myotubes. FIGS. 15 and 16 show the effects of the cinnamic acid combinations in myotubes, and the quantitative data for adipocytes and myotubes is summarized in FIGS. 17 and 18, respectively. Cinnamic acid-HMB and cinnamic acid-leucine combinations increased adipocyte fatty acid oxidation by 290% (p=0.004) and 1227% (p=0.006), respectively (FIG. 17). In myotubes, the same combinations increased fatty acid oxidation by 199% (p=0.02) and 234% (p=0.05) (FIG. 18). Further, treatment of adipocytes with these cinnamic acid combinations to produce adipocyte conditioned media which was then applied to myotubes resulted in a 273% increase in myotube fatty acid oxidation (p=0.0002). As with quinic acid, these effects were not attenuated by the addition of 200 nM resveratrol and there was no short-term effect on Sirt1 activity. Instead, the primary effect of these combinations appears to be AMPK-mediated, with Sirt1 effects occurring downstream over a longer period of time, as the combinations resulted in 136-157% increases in AMPK activity (p=0.0001; FIG. 19).

Ferulic Acid:

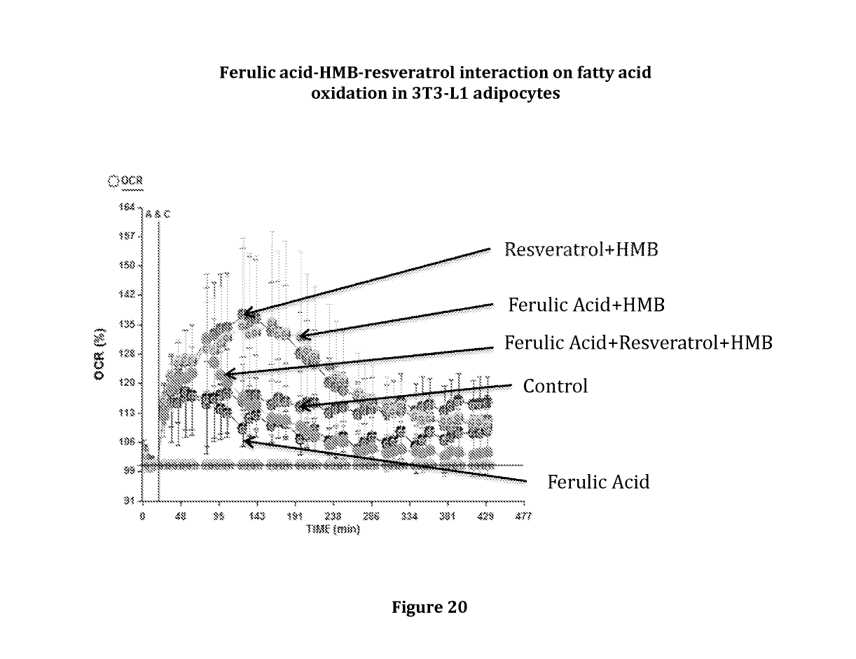

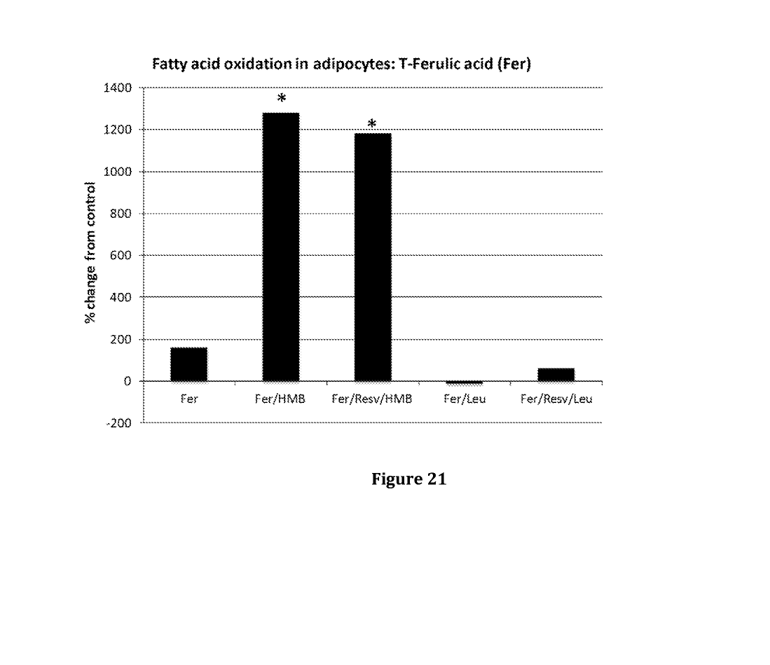

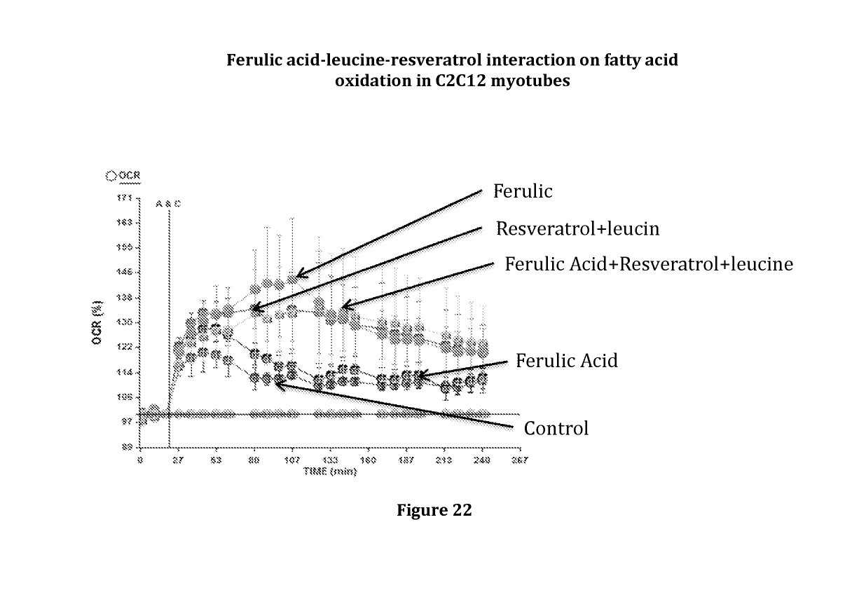

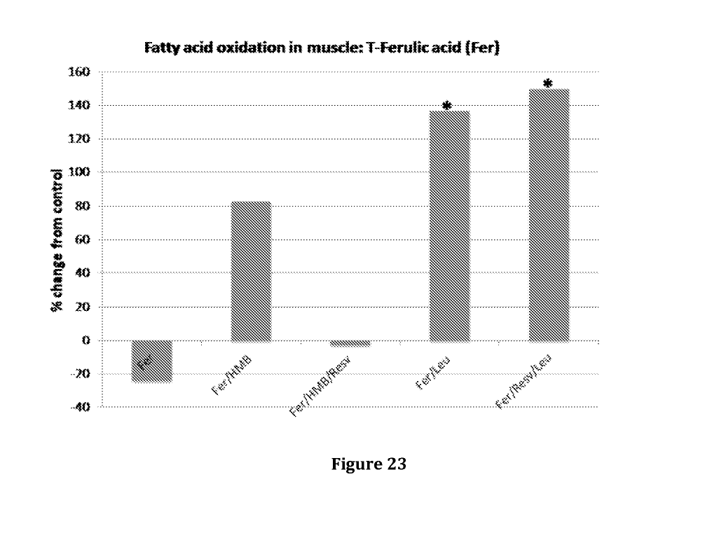

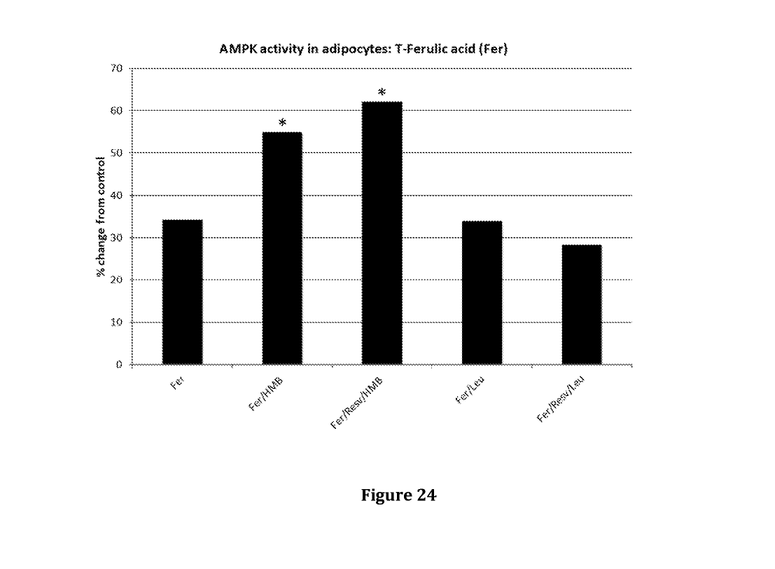

Ferulic acid is another hydroxycinnamic acid. Ferulic acid is naturally occurring in coffee and apples, as well as some other fruits, legumes and grains. Ferulic acid dose-response curves indicate concentrations of 500 nM or below exert no effect; accordingly, this was the concentration used in synergy experiments. Ferulic acid combinations exerted strong effects on fatty acid oxidation. The ferulic acid-HMB combination increased fatty acid oxidation by 1281% (p=0.018) in adipocytes (FIGS. 20 and 21) and by 82% in myotubes (p=0.05) (FIGS. 22 and 23). However, the ferulic acid-leucine combination exerted no significant effect in adipocytes (FIG. 21), but increased fatty acid oxidation by 137% in myotubes (p=0.034; FIG. 23). Similar to cinnamic acid, the effects of the ferulic acid-HMB combination in adipocytes and the ferulic acid-leucine combination in myocytes were not attenuated by the addition of resveratrol and there was no short-term direct effect on Sirt1 activity, but there was a significant stimulation of AMPK activity (55-62%, p=0.05; FIG. 24).

Piceatannol:

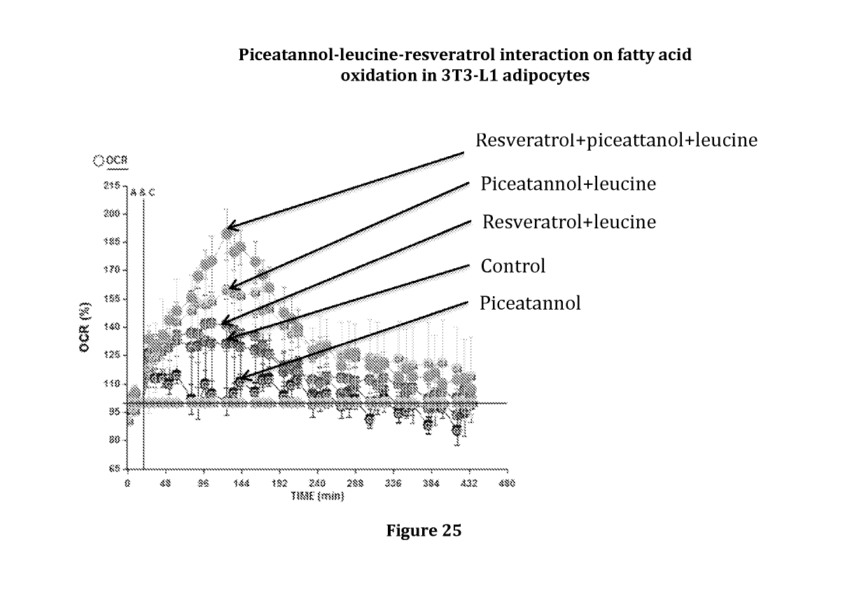

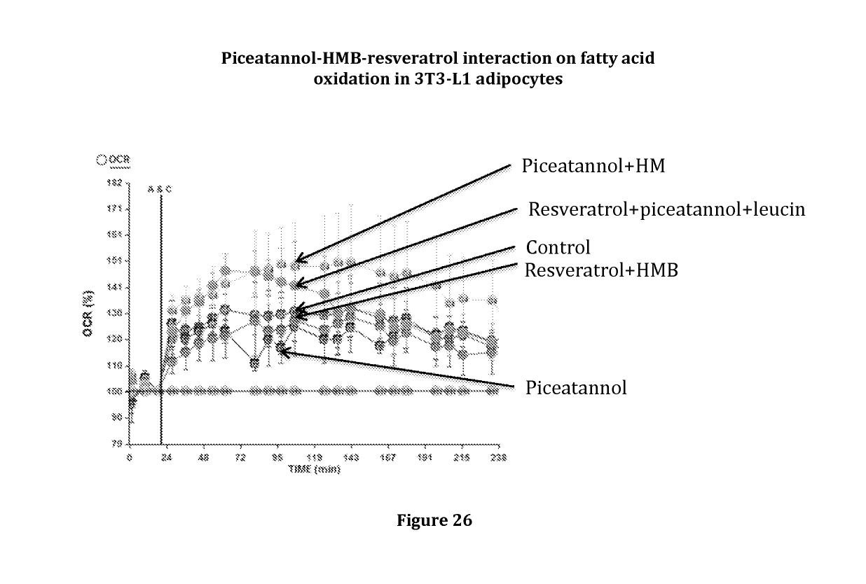

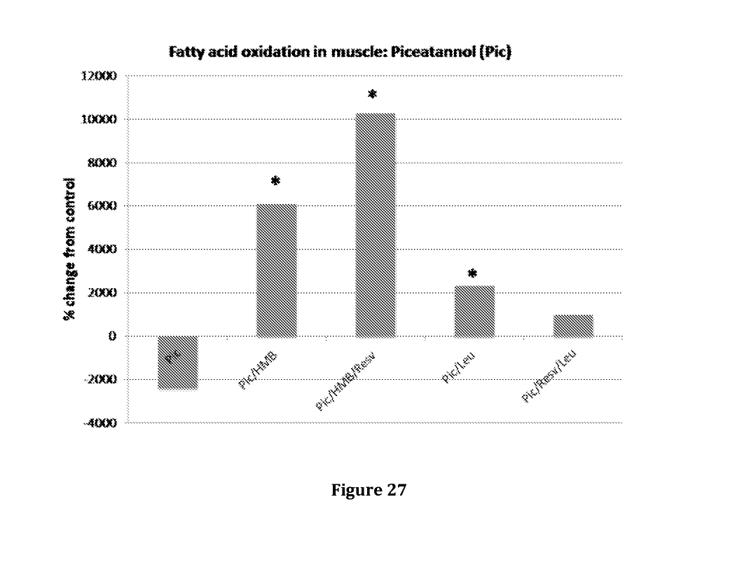

Piceatannol is a polyphenol classified as a stilbene. It is a metabolite of resveratrol and is naturally occurring in red wine. Piceatannol dose-response curves indicate concentrations of 1 nM or below exert no effect; accordingly, this was the concentration used in synergy experiments. To date, only fatty acid oxidation experiments have been conducted (FIGS. 25-27). Data from these experiments demonstrate significant effects of both combinations in both adipocytes and myotubes. The piceatannol-leucine combination elicited a 73% increase in fatty acid oxidation in adipocytes (p=0.05) and a 2301% increase in fatty acid oxidation in myotubes (p=0.039), and the piceatannol-HMB combination elicited a 60% increase in adipocytes (p=0.05) and a 6085% increase in myotubes (FIG. 27).

Ellagic Acid:

Ellagic acid is a large polyphenol naturally occurring in strawberries, raspberries and grapes, as well as a number of other plant products. This polyphenol failed to exert a significant effect in most of our assays, and dose-response curves of ellagic acid indicated little activity, even at high concentrations (50 μM).

Epigallocatechin Gallate (EGCG):

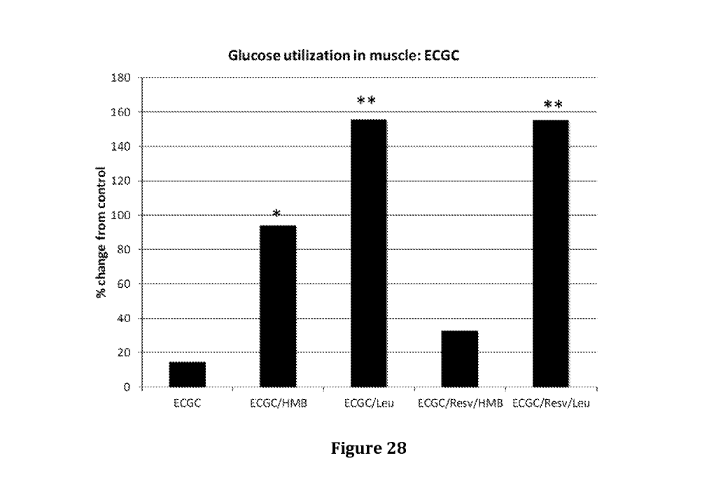

EGCG is a polyphenol ester of epigallocatechin and gallic acid. EGCG is the predominant catechin in green tea. Despite claims to the contrary, we find this compound to be minimally active in directly stimulating fatty acid oxidation and do not detect synergistic effects with either HMB or leucine in stimulating fatty acid oxidation. However, EGCG (1 μM) did exert significant effects on glucose utilization as measured by extracellular acidification. This level of EGCG exerted no independent effect on glucose utilization, but stimulated a 94% increase in glucose utilization when combined with HMB (p=0.015; FIG. 28) and a 156% increase in glucose utilization when combined with leucine (p=0.017; FIG. 28). Notably, adding resveratrol to this combination exerted no additional effect, but also did not attenuate the observed effects. The effects of these combinations on AMPK and Sirt1 activities have not yet been determined.

Fucoxanthin:

Fucoxanthin is a non-polyphenolic pigment found in brown seaweed (“Sea Mustard”; Undaria pinnatifida). Fucoxanthin dose-response curves indicate concentrations of 100 nM or below exert no effect; accordingly, this was the concentration used in synergy experiments.

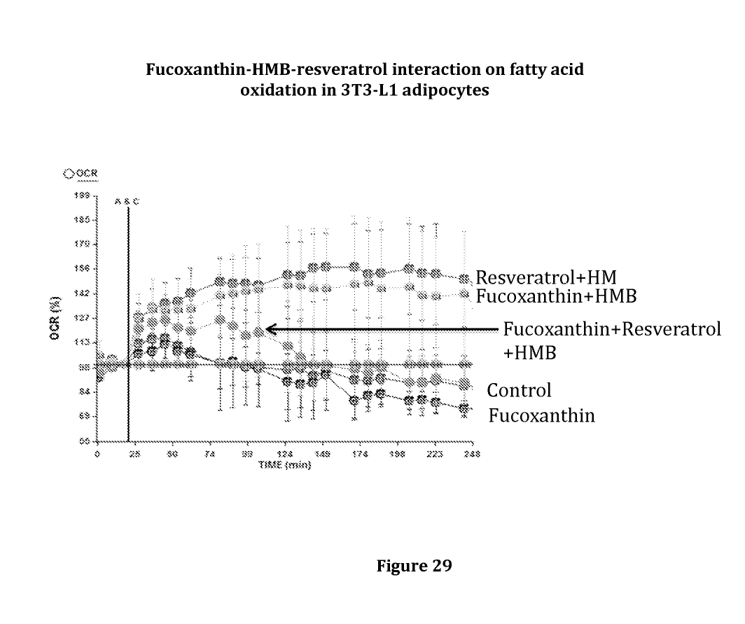

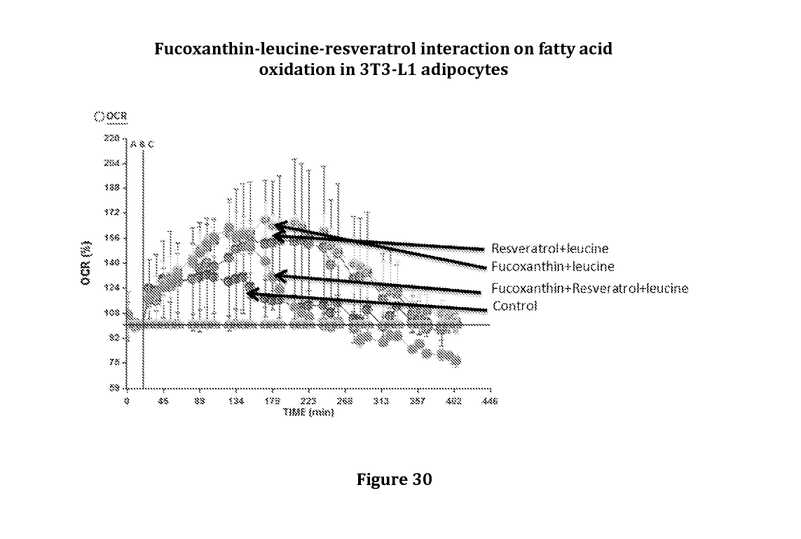

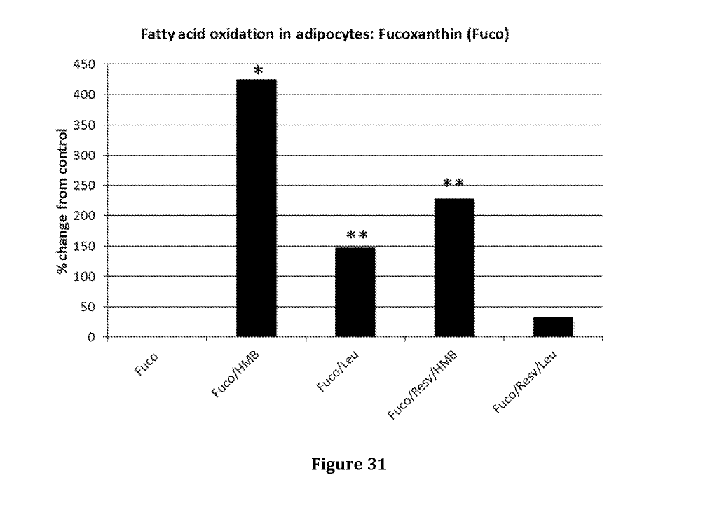

Fucoxanthin-HMB and fucoxanthin-leucine combinations both exerted potent effects on fatty acid oxidation in adipocytes (fucoxanthin-HMB, 425% increase, p=0.033; fucoxanthin-leucine, 148% increase, p=0.05; FIGS. 29-31) and myotubes (fucoxanthin-HMB, 236% increase, p=0.05; fucoxanthin-leucine, 82% increase, p=0.024). Addition of resveratrol neither attenuated nor augmented these effects.

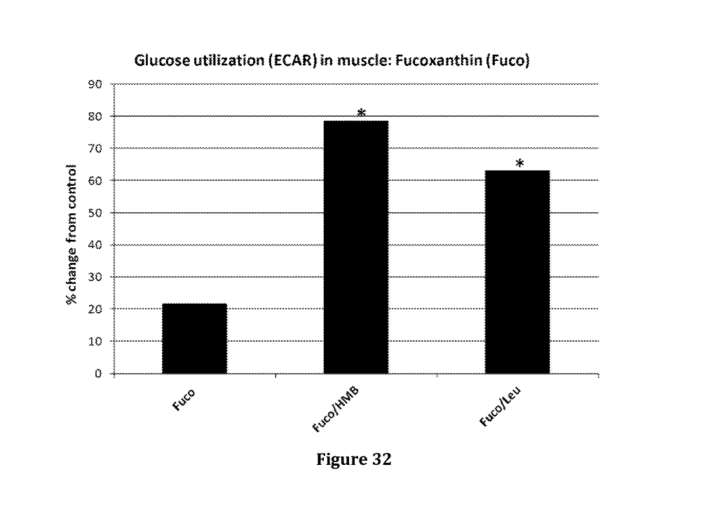

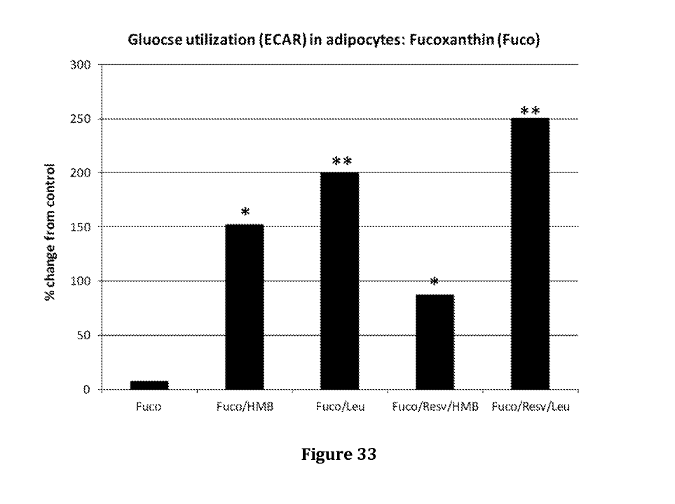

Fucoxanthin combination with both HMB and leucine significantly augmented glucose utilization in myotubes and adipocytes (FIGS. 32 and 33). The fucoxanthin-HMB combination resulted in a 59% increase (p=0.038) and the fucoxanthin-leucine combination resulted in a 63% increase (p=0.034) in myotubes (FIG. 32). In adipocytes, the fucoxanthin-HMB combination resulted in a 321% increase (p=0.02) and the fucoxanthin-leucine combination resulted in a 557% increase (p=0.003; FIG. 33).

The effects of the fucoxanthin combinations on AMPK and Sirt1 activity have not yet been determined.

Grape Seed Extract:

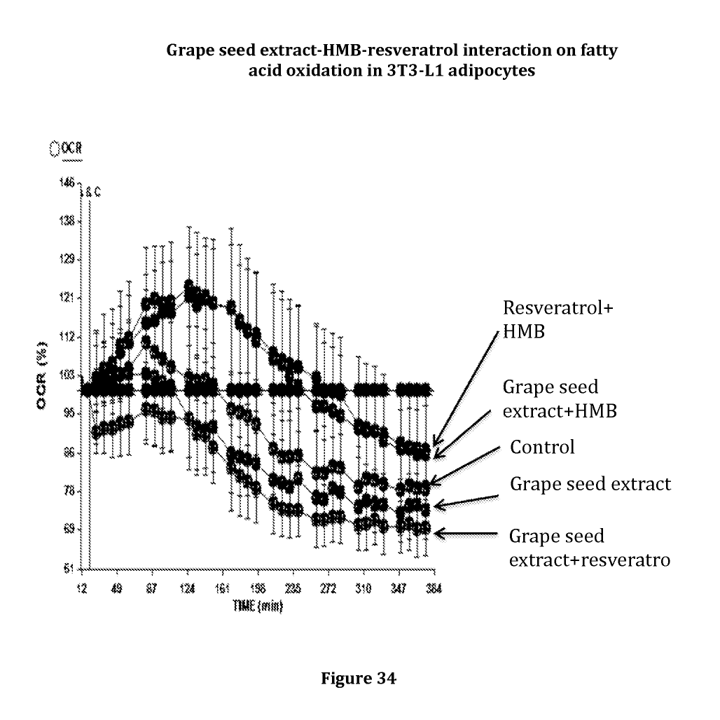

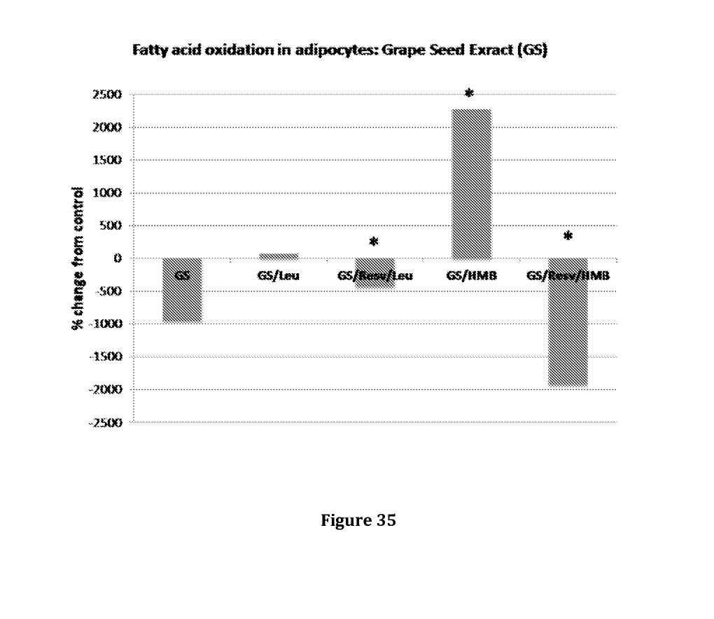

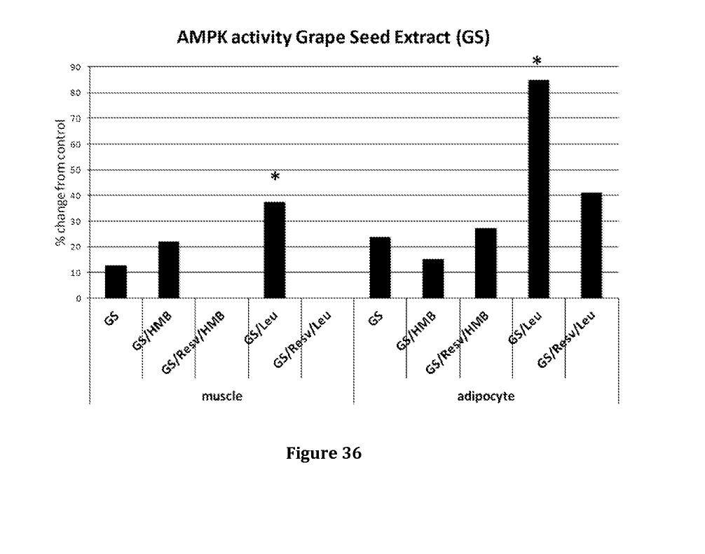

Grape seed extract (GSE) is an undifferentiated mixture of polyphenols, including resveratrol, and other naturally occurring compounds in grape. It was selected for study as a broad example of synergy with a naturally occurring group of polyphenols. Since it is a mixture, it is not possible to define concentrations in molar units, so mass units are used for this section. GSE dose-response curves indicate concentrations of 1 μg/mL or below exert no effect; accordingly, this was the concentration used in synergy experiments. GSE-leucine increased adipocyte fatty acid oxidation by 74%, but this did not reach statistical significance. The GSE-HMB combination increased fatty acid oxidation by 2262% (p=0.04; FIGS. 34 and 35). The effects of both combinations were attenuated by the addition of resveratrol to the combinations (FIG. 35). GSE-leucine and GSE-HMB combinations modestly increased both AMPK activity (40-80%, p<0.01; FIG. 36) and Sirt1 activity (15-20%, p<0.03).

Metformin:

Metformin, a biguanide, is a commonly prescribed oral hypoglycemic agent. Its known mechanism of action is via stimulation of AMPK, resulting in increased insulin sensitivity as well as increased fat oxidation. Thus, metformin, HMB, leucine, and several of the polyphenols discussed above converge on the same signaling pathways. Accordingly, we sought to determine whether combinations of metformin with these compounds exert a synergistic effect, thereby lowering the concentration of metformin necessary to achieve therapeutic effect.

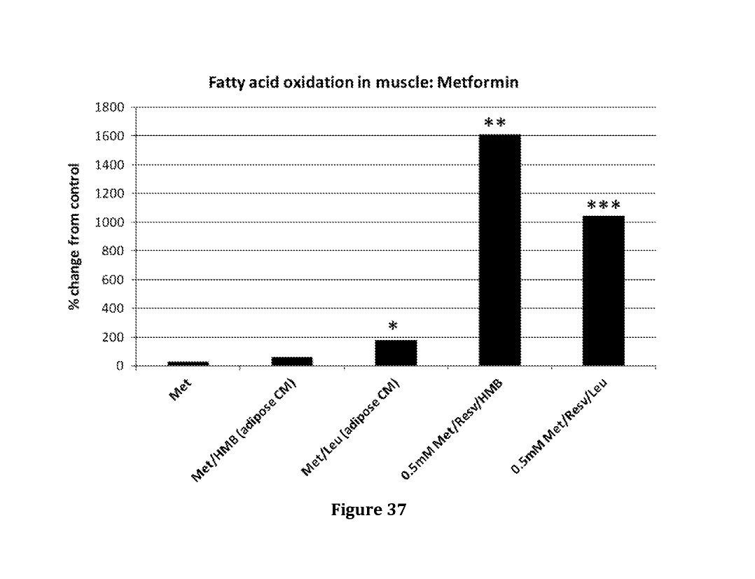

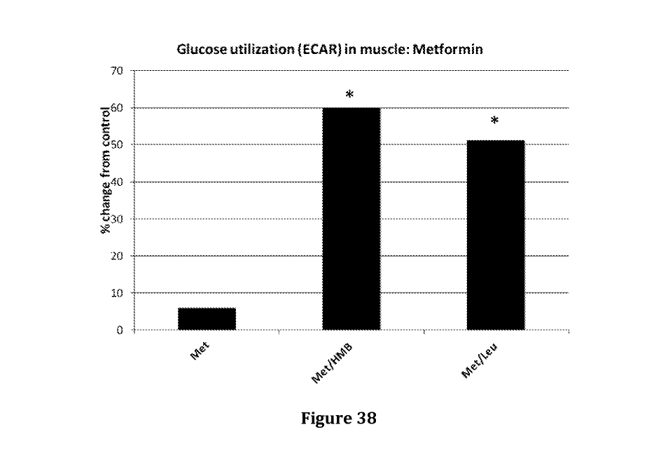

Metformin dose-response curves indicate concentrations of 0.1 mM or below exert no effect; accordingly, this was the concentration used in synergy experiments. This level is substantially lower than concentrations used to assess independent effects of metformin in cellular studies (2-10 mM). Combining metformin with resveratrol (200 nM) and HMB resulted in a 1607% increase in myotube fatty acid oxidation (p=0.0001; FIG. 37), while the metformin-leucine-resveratrol combination elicited a 1039% increase (p=0.001). Omitting resveratrol from the combinations resulted in statistically significant, but more modest, synergistic interactions with metformin (FIG. 37). Metformin-HMB elicited a 58% increase in myotube fatty acid oxidation (p=0.05) while metformin-leucine elicited a 176% increase (p=0.03). These combinations also significantly augmented glucose utilization in myotubes by 61 and 51%, respectively (p=0.028 for both). Both metformin-HMB and metformin-leucine stimulated myotube glucose utilization by 50-60% (p=0.03; FIG. 38).

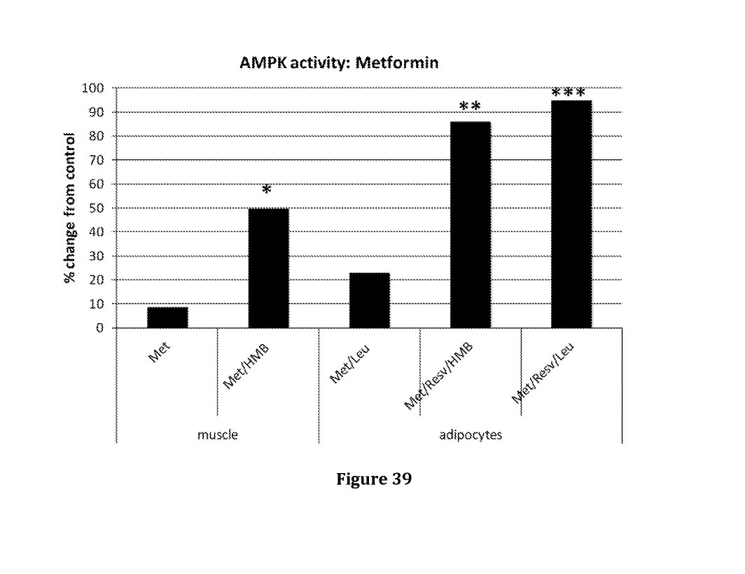

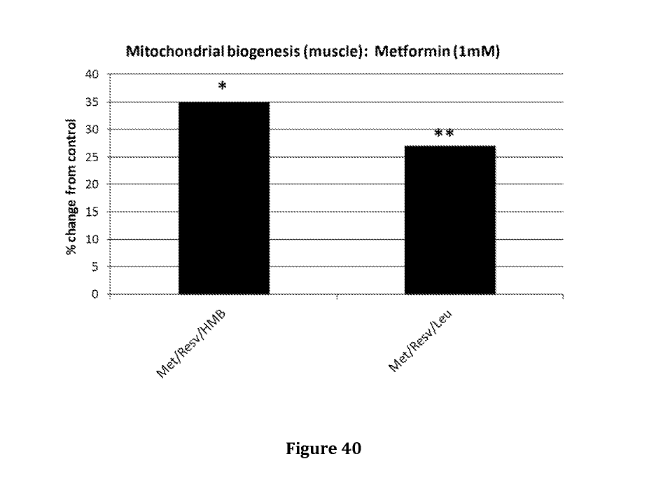

Consistent with these data, these combinations also significantly increased AMPK activity (FIG. 39). The metformin-HMB combination increased myotube AMPK activity by 50% (p=0.031) and the metformin-leucine combination by 22%. Inclusion of resveratrol (200 nM) significantly augmented these effects; metformin-HMB-resveratrol increased AMPK activity by 86% (p=0.026) and the metformin-leucine-resveratrol combination resulted in a 95% increase (p=0.017). These combinations exerted similar effects on Sirt1 activity. Metformin-HMB increased Sirt1 activity by 38% and 58% in adipocytes and myotubes, respectively (p=0.001 for both). Comparable effects were observed for mitochondrial biogenesis (metformin-HMB-resveratrol, 35%, p=0.001; metformin-leucine-resveratrol, 27%, p=0.013; FIG. 40).

Notably, combining metformin with either grape seed extract or chlorogenic acid resulted in similar stimulation of Sirt1 activity. Metformin-grape seed extract increased activity by 24% (p=0.001) and metformin-chlorogenic acid increased activity by 42% (p=0.004).

Rosiglitazone:

Rosiglitazone is an oral hypoglycemic agent in the thiazolidinedione (TZD) class. Its adverse event profile has raised significant concern, limiting its current use, although it is still approved. TZDs act by binding to peroxisome proliferator-activated receptor gamma (PPARγ). One of the targets of PPARγ is peroxisome proliferator-activated receptor gamma coactivator 1-alpha (PGC-1a), a regulator of mitochondrial biogenesis and fatty acid oxidation that is a downstream mediator of Sirt1. Accordingly, we sought to determine whether combinations of rosiglitazone with the compounds investigated here exert a synergistic effect, thereby lowering the concentration of metformin necessary to achieve therapeutic effect.

Rosiglitazone dose-response curves indicate concentrations below 1 nM exert no effect; accordingly, this was the concentration used in synergy experiments. This level is lower than that typically used in cell culture experiments (10 nM-10 μM) and is markedly lower than plasma levels typically achieved following IV or oral dosing (400 nM-1.7 μM).

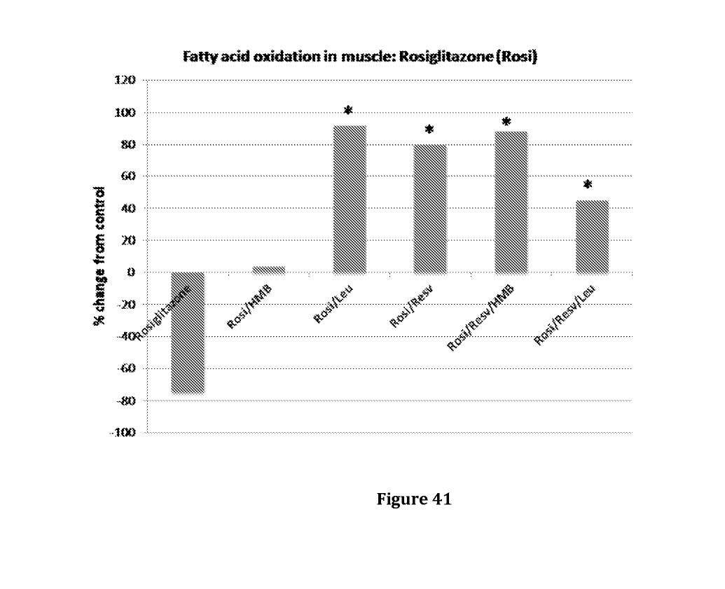

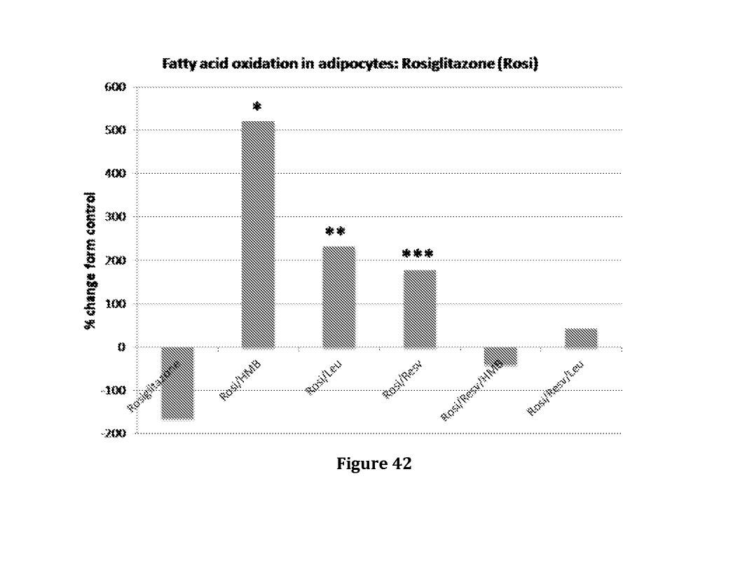

Combining rosiglitazone with either leucine or HMB resulted in significant stimulation of fatty acid oxidation in both myotubes (FIG. 41) and adipocytes (FIG. 42). The rosiglitazone-HMB combination stimulated fatty acid oxidation by 521% (p=0.004), and the rosiglitazone-leucine combination stimulated fatty acid oxidation by 231% (p=0.023) and myotube fatty acid oxidation by 92% (p=0.009). Combining rosiglitazone with resveratrol (200 nM) also resulted in stimulation of fatty acid oxidation (177%, p=0.003); however, adding resveratrol to the rosiglitazone-HMB or rosiglitazone-leucine combinations was not more effective than the combinations in the absence of resveratrol in myotubes and attenuated the effects of the combinations in adipocytes.

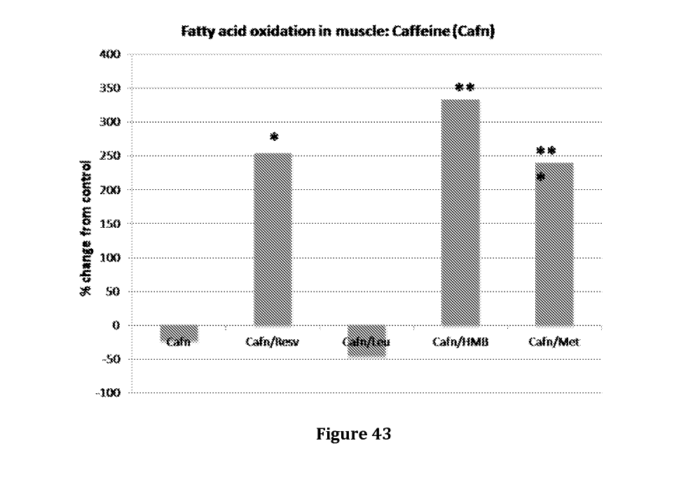

Combining rosiglitazone with either HMB or leucine resulted in marked increases in glucose utilization (FIG. 43). The rosiglitazone-HMB combination stimulated a 322% increase (p=0.05) and the rosiglitazone-leucine combination stimulated a 341% increase. A comparable increase was found when resveratrol (200 nM) was combined with rosiglitazone (415%, p=0.001), but adding resveratrol to either the rosiglitazone-HMB or rosiglitazone-leucine combinations did not further augment glucose utilization.

Phosphodiesterase (PDE) Inhibitors:

The effects of resveratrol on Sirt1 activation may be mediated, in part, via inhibiting cAMP Phosphodiesterase, resulting in up regulation of AMPK and subsequent activation of Sirt1 rather than a direct effect. However, other this effect may only be relevant at high (>50 μM) resveratrol concentrations. Accordingly we have evaluated the effects of various non-specific PDE inhibitors, as follows.

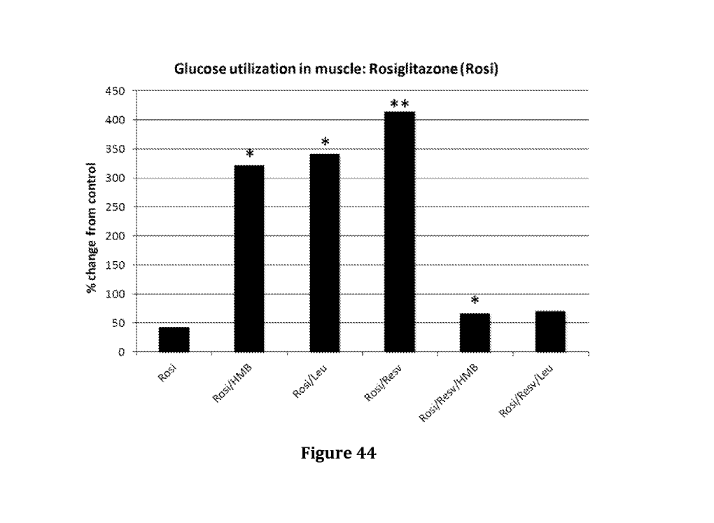

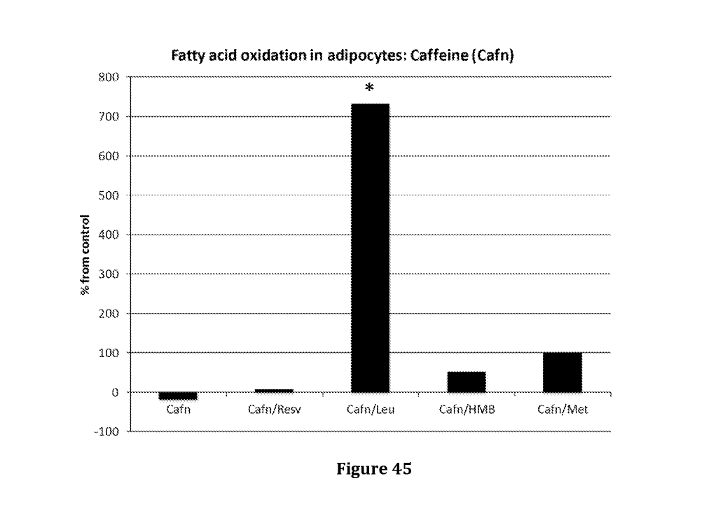

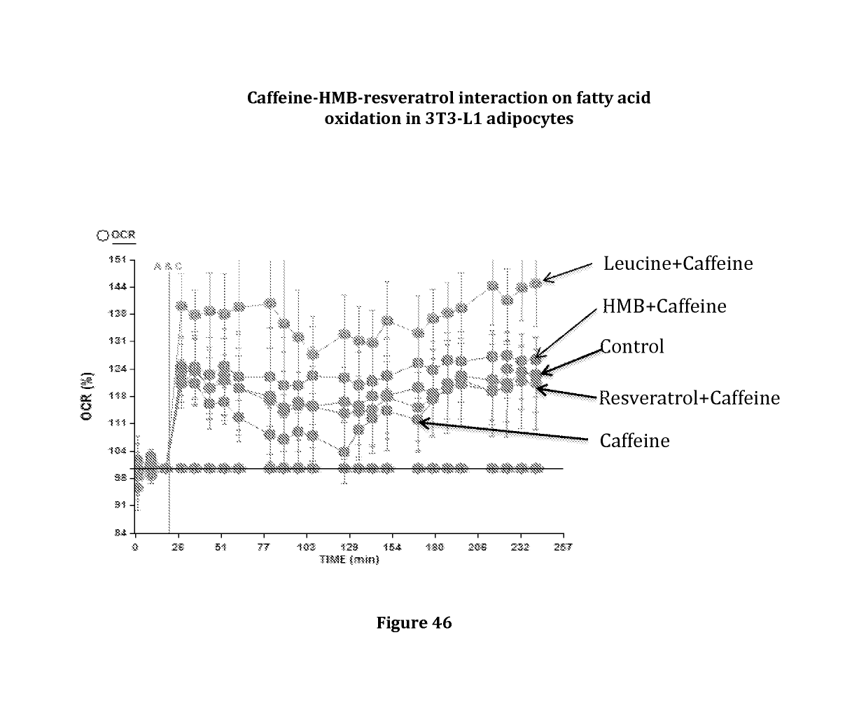

Caffeine is a naturally occurring methyl-xanthine found primarily in coffee, tea, guarana and verba mate. Caffeine is both an adenosine antagonist and a non-specific PDE inhibitor. Caffeine dose-response curves indicate concentrations below 10 nM exert no effect; accordingly, this was the concentration used in synergy experiments. This level is ˜0.1% of the plasma concentration observed following caffeine consumption (1-10 μM). Combining 10 nM of caffeine with resveratrol (200 nM) resulted in a 254% increase in fatty acid oxidation in myotubes (p=0.03; FIG. 44), while neither component exerted an independent effect. Combining caffeine with 0.5 mM leucine stimulated adipocyte fatty acid oxidation by 732% (p=0.008; FIGS. 45 and 46), and combining caffeine with 5 μM HMB resulted in a 334% increase in fat oxidation in myotubes (p=0.05; FIG. 44). The caffeine-leucine combination also markedly improved muscle cell glucose utilization as measured by extracellular acidification responses to glucose addition (574% improvement, p=0.003). Caffeine also exhibited significant synergy with metformin (0.1 mM), resulting in a 240% increase in myotube fatty acid oxidation (p=0.013; FIG. 44), although it did not exert a synergistic effect on glucose utilization.

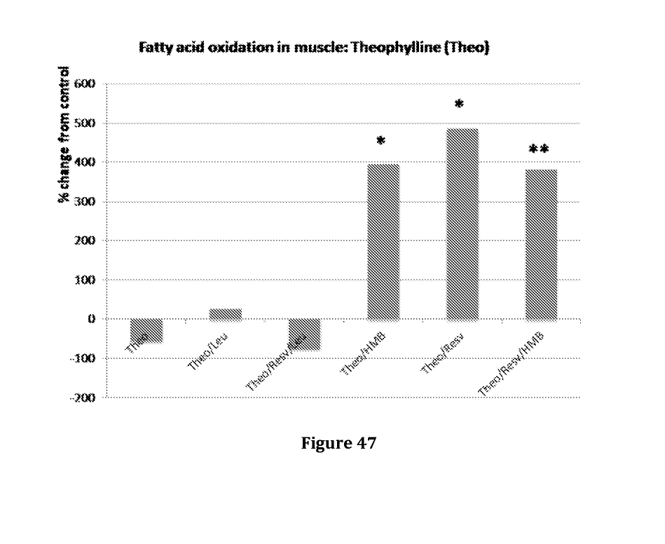

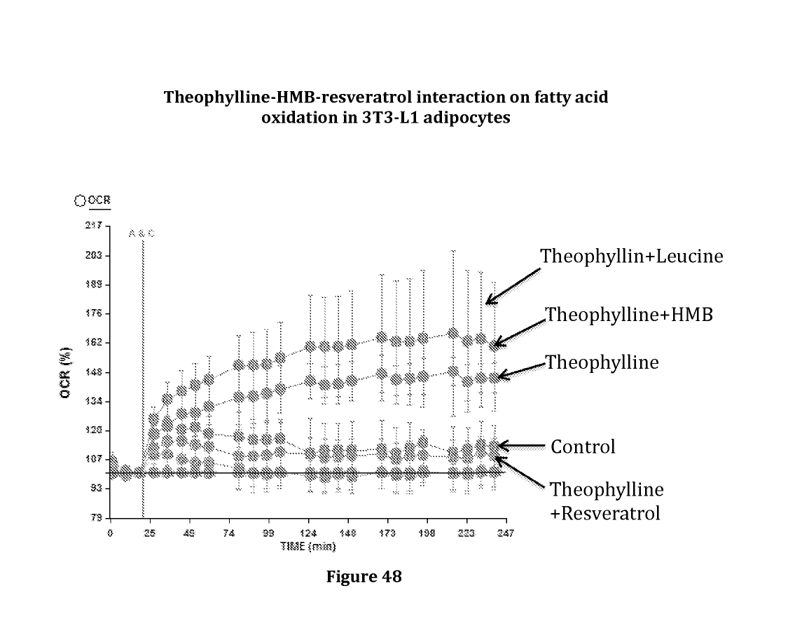

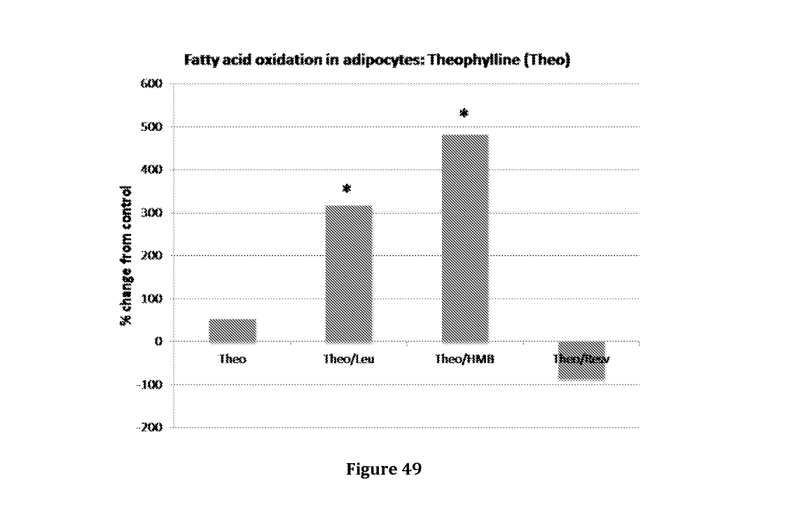

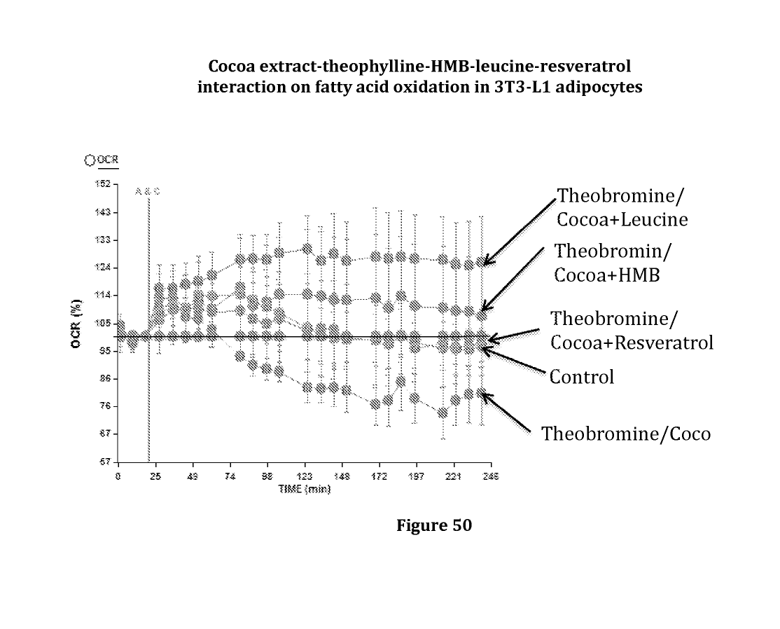

Theophylline is a metabolite of caffeine that is also naturally occurring in tea and cocoa. Theophylline dose-response curves indicate concentrations below 1 μM exert no effect; accordingly, this was the concentration used in synergy experiments. Combining theophylline with 5 μM HMB resulted in a 396% increase in myotube fatty acid oxidation (p=0.03; FIG. 47). Similar synergy occurred between theophylline and resveratrol (486%, p=0.03), while combining HMB, resveratrol and HMB did not further augment this effect (382%, p=0.05; FIG. 48). Theophylline exhibited a similar synergy with HMB and leucine in adipocytes (FIGS. 49 and 50), although no synergy was observed with resveratrol in adipocytes.

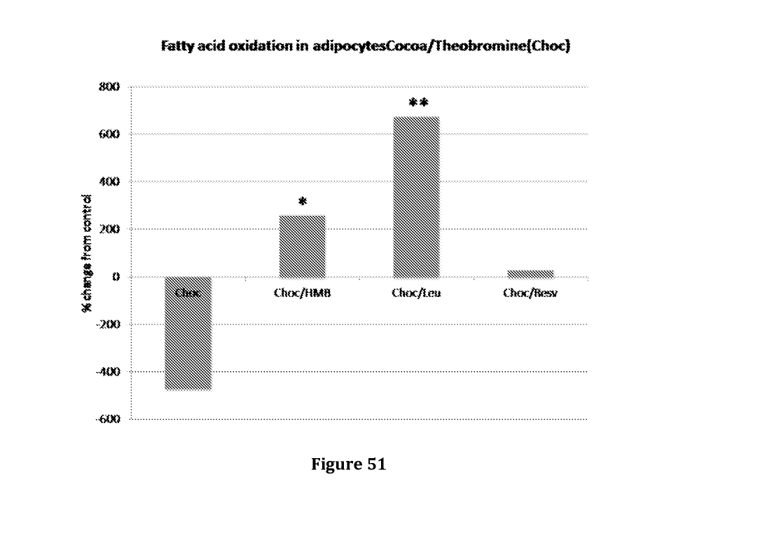

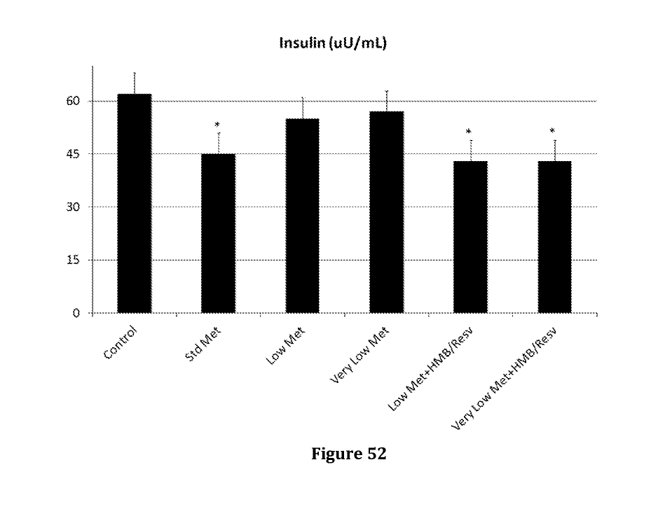

Theobromine is a naturally occurring methylxanthine found primarily in cocoa and dark chocolate, as well as verba mate and tea. Experiments were conducted with a cocoa extract standardized to 12% theobromine; dose-response curves indicated concentrations below 0.1 μg/mL exert no effect; accordingly, this was the concentration used in synergy experiments. Combining cocoa extract/theobromine with 5 μM HMB resulted in a 260% increase in fat oxidation (p=0.021), and the cocoa extract/theobromine combination with 0.5 mM leucine resulted in a 673% increase (p=0.00035) (FIGS. 51 and 52). Combining the cocoa extract/theobromine with resveratrol exerted no significant effect on fat oxidation (FIGS. 51 and 52).

Isobutylmethylxanthine (3-isobutyl-1-methylxanthine; IBMX) is a methyl xanthine similar to caffeine. It serves as both an adenosine antagonist and a non-specific PDE inhibitor. IBMX dose-response curves indicate concentrations below 50 nM exert no effect; accordingly, this was the concentration used in synergy experiments. IBMX exhibited weak but statistically significant synergy with HMB, but not leucine, in stimulating fat oxidation (73% increase, p=0.05) and glucose utilization (66%, p=0.05) in myotubes.

These data demonstrate significant synergistic effects of a several naturally occurring polyphenols on fat oxidation and glucose utilization when these polyphenols are combined with either HMB or leucine. These effects occur at levels which produce no independent effects and which are readily achievable via diet or supplementation. These effects, mediated via Sirt1 and AMPK signaling, are significantly more robust for several of the polyphenols than those we previously observed for a low dose of resveratrol combined with either HMB or leucine and more robust than effects observed by us and others for high dose resveratrol. Chlorogenic acid (a hydroxycinnamic acid) and its hydrolysis product, quinic acid, as well as compounds structurally related to chlorogenic acid (cinnamic acid, ferulic acid) exerted especially robust effects. Highly significant effects were also observed with the resveratrol metabolite piceatannol as well as with a non-polyphenolic compound from seaweed (fucoxanthin, a xanthophyll that exhibits a highly resonant structure commonly observed in polyphenols). These effects can also be recapitulated with naturally occurring non-specific PDE inhibitors. Thus, moderate levels of leucine and HMB can be utilized in synergistic combinations with a number of polyphenols and related compounds to stimulate AMPK and sirtuin signaling and achieve benefits comparable to or exceeding those found with high-dose resveratrol.

These data also demonstrate that leucine and HMB exhibit significant synergies with pharmaceuticals that converge on the same signaling pathways, thereby conferring efficacy to otherwise non-therapeutic doses of these drugs. This can be an effective strategy for decreasing the levels of these drugs required to achieve therapeutic efficacy, thereby attenuating side effects and adverse events otherwise associated with them.

### Example 3—Synergistic Effects of Metformin with Resveratrol-Hydroxymethylbutyrate Blend on Insulin Sensitivity in Diabetic Mice

Eight to ten week-old male diabetic db/db mice (C57BLKS/J-lepr^{db}/lepr^{db}) were randomized into six treatment groups (as described below) with 10 animals/group and kept on their diet for 2 weeks.
Group 1 (labeled “control group”): standard diet (AIN 93G) only
Group 2 (labeled “high Metformin” (here 300 mg/kg BW)): standard diet mixed with 1.5 g Metformin/kg diet (calculation: average food consumption=8 g/day, average BW=40 g, 300 mg×0.04 kg=12 mg Metformin/day/8 g food=1.5 mg Met/g diet)
Group 3 (labeled “low Metformin” (here 150 mg/kg BW): standard diet mixed with 0.75 g Metformin/kg diet
Group 4 (labeled “very low Metformin” (here 50 mg/kg BW): standard diet mixed with 0.25 g Metformin/kg diet
Group 5 (labeled “low Metformin plus Resv and CaHMB”): standard diet mixed with 0.75 g Metformin plus 12.5 mg Resveratrol and 2 g CaHMB/kg diet
Group 6 (labeled “very low Metformin plus Resv and CaHMB”): standard-diet mixed with 0.25 g Metformin plus 12.5 mg of Resveratrol and 2 g CaHMB/kg diet

Animals were housed in polypropylene cages at a room temperature of 22±2° C. and regime of 12 h light/dark cycle. The animals had free access to water and their experimental food throughout the experiment. At the of the treatment period (2 weeks) all animals were fasted overnight and humanely euthanized the next morning, and blood and tissues were collected for further experiments as described below.

Insulin Tolerance Test (ITT):

Insulin tolerance tests were performed at 2 μm on day 7. The mice were injected with insulin (0.75 U/kg) in ˜0.1 ml 0.9% NaCl intraperitoneally. A drop of blood (5 microliter) was taken from the cut tail vein before the injection of insulin and after 15, 30, 45, and 60 min for the determination of blood glucose. Change in blood glucose over the linear portion of the response curve was then calculated.

Insulin:

Blood Insulin in serum was measured via Insulin ELISA kit from Millipore (Cat. # EZRMI-13K).

Glucose:

Blood glucose was measured via Glucose Assay Kit from Cayman (Cat. # EZRMI-13K).

Statistical Analysis:

All data is expressed as mean±STD. Data was analyzed by one-way ANOVA, and significantly different group means (p<0.05) were separated by the least significant difference test using SPSS (SPSS Inc, Chicago, Ill.).

### Results

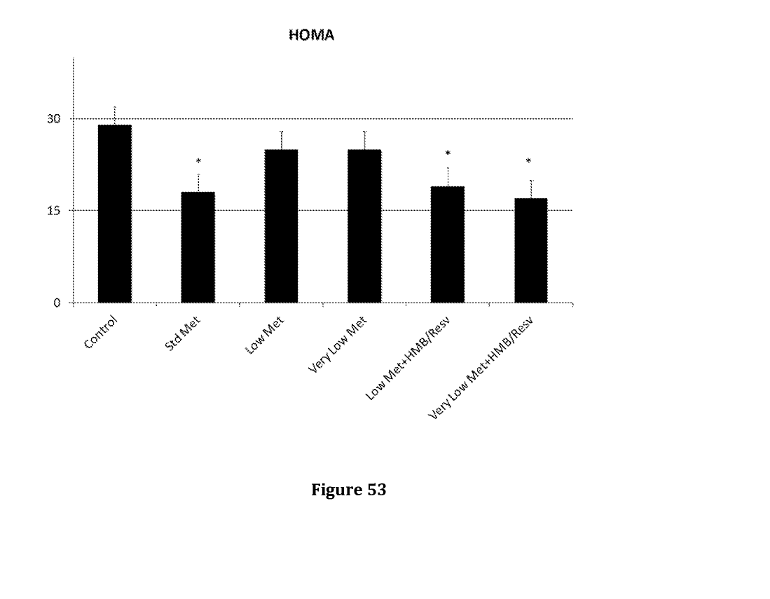

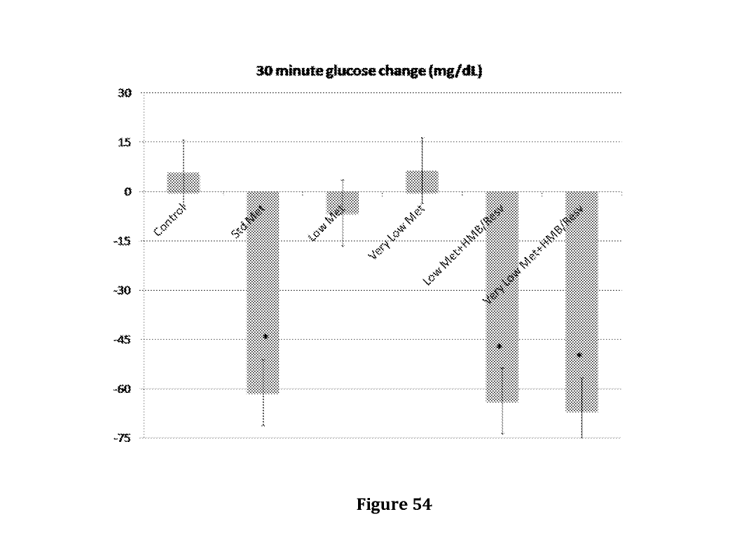

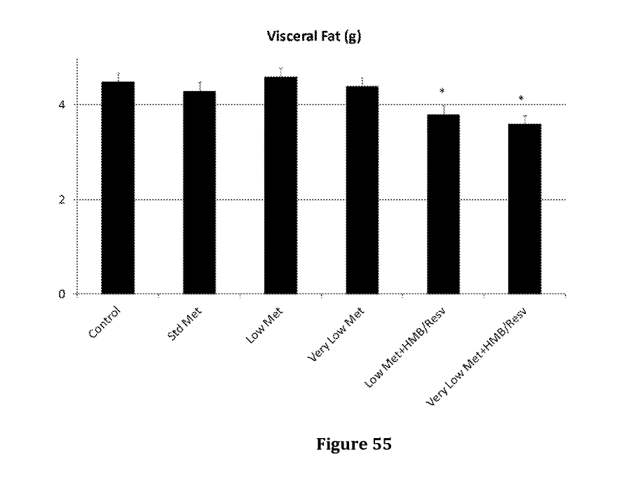

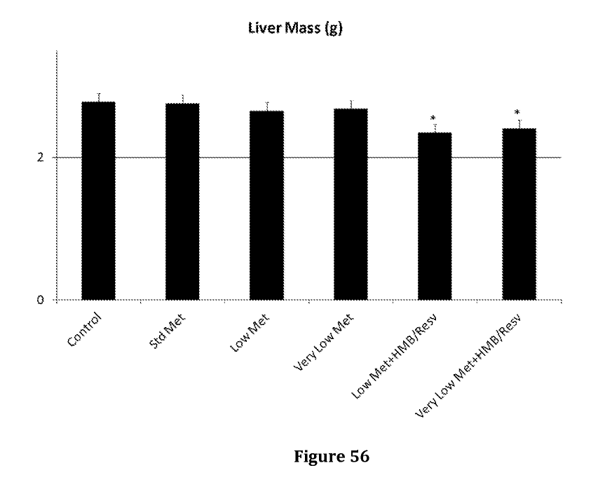

The high dose (300 mg/kg bw) reduced plasma insulin by 27% (from 62 to 45 uU/mL, p<0.02, FIG. 53) and in the HOMA_{IR} index by 35% (from 29 to 18 units, p<0.025, FIG. 54), but exerted no significant effect on plasma glucose in these highly insulin resistant animals. However, there were no significant effects on body composition. A low dose of metformin (here 150 mg/kg) and a very low dose (50 mg/kg) exerted no significant independent effects on any variable studied. In contrast, combining either the low or very low dose of metformin with HMB resulted in significant decreases in plasma insulin from 62 uU/mL to 43 uU/mL (p<0.02, FIG. 53) comparable to that seen with high dose metformin, and there was no significant difference between the low metformin-HMB blend versus the very low metformin-HMB blend. Consistent with this observation, the HOMA_{IR} index decreased from 29 units on the control diet to 19 on the low metformin-HMB blend and to 16 on the very low metformin-HMB blend (p<0.025, FIG. 54), reflecting an improvement in insulin sensitivity comparable to that found with high dose metformin. This is also reflected in the results of the insulin tolerance test; animals on the control, low-dose or very low-dose of metformin exhibited minimal changes in blood glucose in response to the insulin challenge (FIG. 55). In contrast, those on the standard metformin dose and those on either the low or very low dose of metformin combined with HMB exhibited ˜60 mg/dL decreases in blood glucose over the 30 minute linear portion of the response curve (p<0.02; FIG. 55). Moreover, the metformin-HMB blends reduced visceral adiposity (FIG. 56). Animals on the control diet had a mean visceral fat mass of 4.5 g, and this was not affected by metformin at any dosage in the absence of HMB. A low dose of metformin+HMB and a very low dose of metformin+HMB reduced visceral fat by ˜20%, to 3.8 and 3.6 g, respectively, (p<0.03; FIG. 56). These treatments also reduced liver mass, from 2.78 g (control) to 2.35 g and 2.41 g, respectively (p<0.05 for both, FIG. 57).

### Example 4: Effects of Nicotinic Acid/Leucine and Nicotinic Acid/Leucine/Resveratrol on Sirt1 Level in Muscle Cells In Vitro

The use of a composition comprising nicotinic acid and leucine as described herein was investigated, wherein the composition comprises free leucine and a sub-therapeutic amount of nicotinic acid. The composition activated Sirt1 in the muscle cells and can be used to ameliorate a hyperlipidemic condition. A composition further comprises resveratrol was investigated as well.

C2C12 mouse myoblasts (American Type Culture Collection) were plated at a density of 8000 cells/cm² (10 cm² dish) and grown in Dulbecco's modified eagle's medium (DMEM) containing 10% fetal bovine serum (FBS), and antibiotics (growth medium) at 37° C. in 5% CO2. For differentiation of C2C12 cells, cells were grown to 100% confluence, transferred to differentiation medium (DMEM with 2% horse serum and 1% penicillin-streptomycin), and fed with fresh differentiation medium every day until myotubes were fully formed (3 days).

A dose-response study was performed by administering the cells with different concentrations of nicotinic acid in order to find the sub-therapeutic amount of nicotinic acid that exerts no effect on the variable studied. Concentrations of nicotinic acid <100 nM alone were found to exert no effect, and experimental concentrations were therefore set below this level, at 10 nM. This sub-therapeutic level of nicotinic acid was then tested in combination with leucine and HMB. The leucine and HMB were at concentrations that have been previously shown to be attainable in diet or supplement while each having no therapeutic effect on these variables when administered alone (0.5 mM for leucine and 5 μM for HMB).

C2C12 cell myotubes were administered with 10 nM nicotinic acid (NA), 10 nM nicotinic acid with 0.5 mM leucine (NA/Leu), 10 nM nicotinic acid with 0.5 mM leucine and 200 nM resveratrol (NA/R/Leu), 200 nM resveratrol and 0.5 mM leucine (R/Leu), and 10 μM nicotinic acid for 24 hours.

Western blotting was performed with SIRT1 antibodies that were obtained from Cell Signaling (Danvers, Mass.). Protein was measured by BCA kit (Thermo Scientific). Total 35 μg of protein from the cell lysate was resolved on 10% Tris/HCL polyacrylamide gels (Criterion precast gel, Bio-Rad Laboratories, Hercules, Calif.), transferred to PVDF membranes, incubated in blocking buffer (3% BSA in TBS), incubated with primary antibody, washed and incubated with secondary horseradish peroxidase-conjugated antibody. Visualization and chemiluminescent detection were conducted using BioRad ChemiDoc instrumentation and software (Bio-Rad Laboratories, Hercules, Calif.). The band intensity was assessed using Image Lab 4.0 (Bio-Rad Laboratories, Hercules, Calif.), with correction for background and loading controls. Sirt1 was detected at 104-115 kDA. Data were analyzed via one-way analysis of variance and least significant difference test was used to separate significantly different group means.

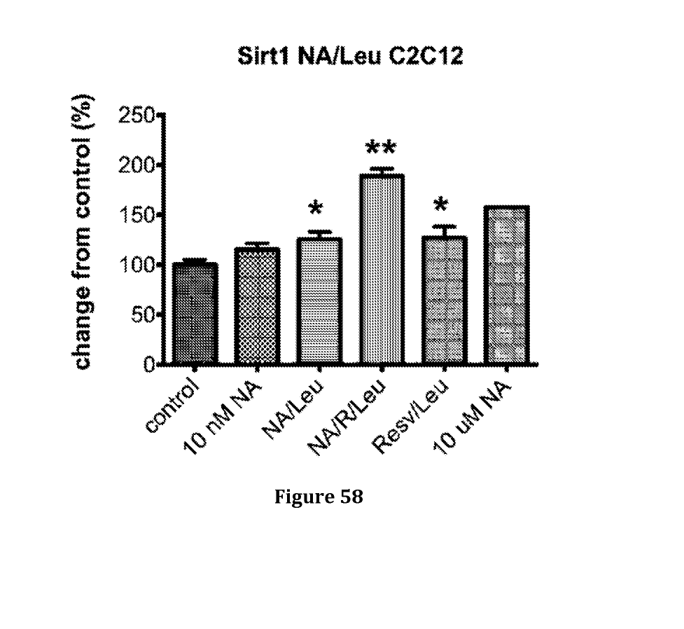

It was found that nicotinic acid-leucine synergistically stimulates Sirt1 in C2C12 myotubes, with an effect comparable to resveratrol-leucine (FIG. 58, p<0.05). Nicotinic acid alone did not have a significant effect on Sirt1 levels. The three-way combination of leucine (10 nM)/resveratrol (200 nM) and nicotinic acid (10 nM) exerted markedly greater effects, with a 200% increase in Sirt1 levels (p=0.0001).

### Example 5: Effects of Nicotinic Acid/Leucine and Nicotinic Acid/Leucine/Resveratrol on P-AMPK/AMPK Level in Fat Cells In Vitro

The use of a composition comprising nicotinic acid and leucine as described herein was investigated, wherein the composition comprises free leucine and a sub-therapeutic amount of nicotinic acid. The composition increased sirtuin pathway output including AMPK, a signaling molecule in the sirtuin pathway, and p-AMPK/AMPK level in the fat cells and can be used to ameliorate a hyperlipidemic condition. A composition further comprises resveratrol was investigated as well.

3T3-L1 preadipocytes (American Type Culture Collection) were plated at a density of 8000 cells/cm2 (10 cm2 dish) and grown in Dulbecco's modified eagle's medium (DMEM) containing 10% fetal bovine serum (FBS), and antibiotics (growth medium) at 37° C. in 5% CO2. Confluent 3T3-L1 preadipocytes were induced to differentiate into adipocytes with a standard differentiation medium consisting of DMEM medium supplemented with 10% FBS, 250 nM dexamethasone, 0.5 mM 3-Isobutyl-1-methylxanthine (IBMX) and 1% penicillin-streptomycin. Preadipocytes were maintained in this differentiation medium for 3 days and subsequently cultured in growth medium. Cultures were re-fed every 2-3 days to allow >90% cells to reach fully differentiation before conducting chemical treatment.

A dose-response study was performed by administering the cells with different concentrations of nicotinic acid in order to find the sub-therapeutic amount of nicotinic acid that exerts no effect on the variable studied. Concentrations of nicotinic acid <100 nM alone were found to exert no effect, and experimental concentrations were therefore set below this level, at 10 nM. This sub-therapeutic level of nicotinic acid was then tested in combination with leucine and HMB. The leucine and HMB were at concentrations that have been previously shown to be attainable in diet or supplement while each having no therapeutic effect on these variables when administered alone (0.5 mM for leucine and 5 μM for HMB).

Differentiated 3T3-L1 cells were administered with 10 nM nicotinic acid (NA), 10 nM nicotinic acid with 0.5 mM leucine (NA/Leu), 10 nM nicotinic acid with 0.5 mM leucine and 200 nM resveratrol (NA/R/Leu), 200 nM resveratrol and 0.5 mM leucine (R/Leu), and 10 μM nicotinic acid for 24 hours.

Western blotting was performed with antibodies against AMPK and Phospho-AMPKα (Thr172) obtained from Cell Signaling (Danvers, Mass.). Protein was measured by BCA kit (Thermo Scientific). Total 30 μg of protein from the cell lysate was resolved on 10% Tris/HCL polyacrylamide gels (Criterion precast gel, Bio-Rad Laboratories, Hercules, Calif.), transferred to PVDF membranes, incubated in blocking buffer (3% BSA in TBS), incubated with primary antibody (P-AMPK), washed and incubated with secondary horseradish peroxidase-conjugated antibody. Visualization and chemiluminescent detection were conducted using BioRad ChemiDoc instrumentation and software (Bio-Rad Laboratories, Hercules, Calif.). The band intensity was assessed using Image Lab 4.0 (Bio-Rad Laboratories, Hercules, Calif.), with correction for background and loading controls. AMPK was detected 62 kDA and P-AMPK was detected at 64-66 kDA. Data were analyzed via one-way analysis of variance and least significant difference test was used to separate significantly different group means.

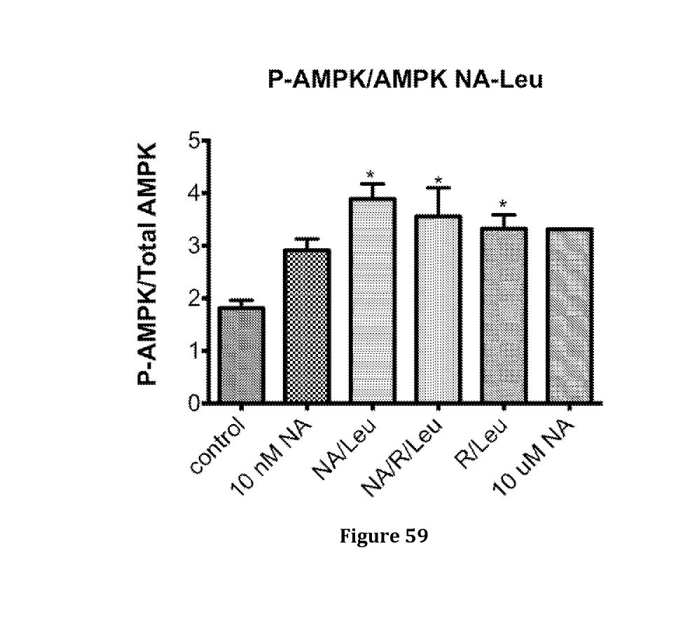

It was found that 10 nM nicotinic acid, when administered alone, had no significant effect on AMPK activation (FIG. 59). The combination of nicotinic acid-leucine significantly stimulated AMPK activation to comparable degree as leucine-resveratrol (p<0.01), as demonstrated by an increase in P-AMPK/AMPK, while the three-way combination of nicotinic acid-leucine-resveratrol was not significantly different from either of the two way leucine combinations (FIG. 59).

### Example 6: Effects of Nicotinic Acid/Leucine and Nicotinic Acid/Leucine/Resveratrol on Fat Content in C. elegans In Vivo

The use of a composition comprising (a) nicotinic acid and (b) leucine as described herein was investigated, wherein the composition comprises free leucine and a sub-therapeutic amount of nicotinic acid. The composition lowered lipid content in a subject after administration of the composition to the subject.

Caenorhabditis elegans (C elegans) worms (N2 Bristol wild-type) were obtained from the Caenorhabditis Genetics Center (CGC) at the University of Minnesota and grown on standard NGM plates with E. coli (OP50) as food source at 20 degree C. For treatments, eggs were hatched on a starved plate overnight. Then synchronized L1 larvae were transferred to E. coli fed NGM plates containing indicated treatments for about 35 hours to reach L4/young adult stage. All treatments were added to the agar. Treatments included 10 nM of nicotinic acid and 0.5 mM of leucine.

Fat content, protein content, fatty acid oxidation of the C elegans worm were measured using the methods described herein.

For Oil-Red Staining to quantify fat content, treated L4/young adult worms were washed off from plates three times with PBS and collected in a 15 ml conical tube, followed by centrifugation at 1000 g for 30 sec. The supernatant was discarded and the pellet was washed with 10 ml PBS. After centrifugation, the supernatant was discarded except 400 μl, which was transferred to a new 1.5 ml eppendorf tube. Then 500 μl of 2×MRWB (160 mM KCl, 40 mM NaCl, 14 mM Na2EGTA, 1 mM Spermidine HCl, 0.4 mM Spermine, 30 mM NaPIPES pH 7.4, 0.2% β-Mercaptoethanol) and 100 μl of 20% Paraformaldehyde were added and the samples were gently rocked for 60 min at room temperature. Then tubes were centrifuged at 1500 g for 30 sec, then aspirated and washed with PBS once, centrifuged again and aspirated to 300 μl. 700 μl of isopropanol was added, mixed by inverting the tube and incubated with gentle shaking for 15 min at room temperature. After centrifuging the tubes to remove the isopropanol, 1 ml of 60% filtered Oil-Red-O-dye solution (0.5 g Oil Red O in 100 ml anhydrous isopropanol, equilibrated for 2 days by stirring at RT, then 4 vol ddH2O was mixed with 6 vol dye solution and equilibrated for 15 min at RT, then filtered with 0.2 M pore size) was added to worms and rotated on shaker overnight. Worms were centrifuged at 1200 g for 30 sec, and followed by ddH2O washes for 4 times to remove any unbound stain. For quantification, the Oil Red O was eluted from the cells by addition of 100% isopropanol and the optical density of 200 μl aliquots (triplicates/sample) was determined at a wavelength of 540 nm using a Biotek Synergy HT Microplate Reader (BioTek, Winooski, Vt., USA). Data were normalized to protein content using the Pierce BCA protein assay kit.

To determine the protein content by western blot, treated L4/young adult worms were washed off from plates with M9 buffer and collected into microcentrifuge tubes. After centrifugation (500 g for 5 min), supernatant was removed to about 100 μl. Then 250 μl RIPA buffer plus Protease and Phosphatase inhibitor mix was added. Samples were homogenized, then centrifuged at 16,000 g for 10 min at 40° C. The clear supernatant was used for further experiments. Protein content was determined using the Pierce BCA protein assay kit.

Fatty acid oxidation was measured by measuring the palmitate-stimulated oxygen consumption rate with the XF 24 analyzer (Seahorse Bioscience, Billerica, Mass., USA) as previously described (Bruckbauer A, Zemel M B. Synergistic effects of metformin, resveratrol, and hydroxymethylbutyrate on insulin sensitivity. Diabetes, Met Synd Obesity 2013; 6:93-102) with slight modifications. Treated L4/young adult worms were washed off from plates with M9 buffer and collected into 15 ml conical tubes. After centrifugation (1000 g for 1 min), supernatant was removed and worm pellet was diluted to a concentration of 40 worms/μl. Worms were kept in ice water during plating to limit movement, and 5 μl of the worm solution was added to each well of a 24-well Seahorse islet plates (˜200 worms/well). Screens were inserted and 595 μl of M9 buffer with indicated treatments was added to each well. Each plate was cooled for 10 min before the start of the measurement. The temperature setting of the instrument was maintained at 29 degree C. during the experiment.

Data were analyzed via one-way analysis of variance and least significant difference test was used to separate significantly different group means.

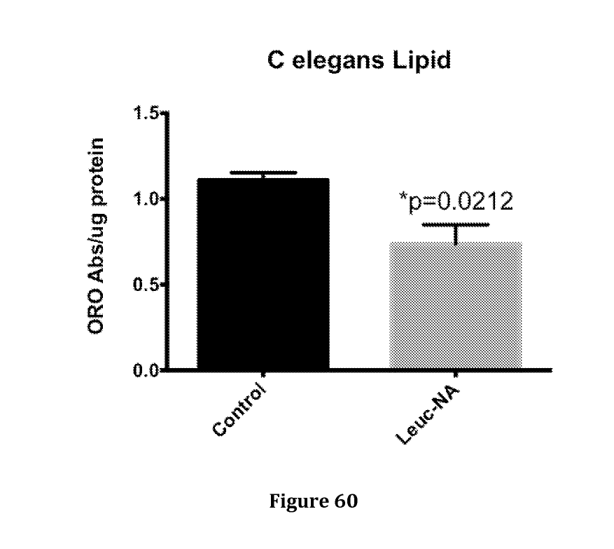

We measured the lipid content in C elegans as shown in FIG. 60. It was found that exposing C. elegans to a leucine (0.5 mM)-nicotinic acid (10 nM) combination for 24 hours resulted in a 33% decrease in total lipid content compared to the non-treated control group.

### Example 7: Effects of Nicotinic Acid/Leucine on Triglyceride, LDL, HDL Cholesterol Levels and Atherosclerotic Plaque Size In Vivo

To assess the efficacy of the subject compounds, mice were administered the subject compounds comprising (a) nicotinic acid and (b) leucine as described herein, wherein the composition comprises free leucine and a sub-therapeutic amount of nicotinic acid. The composition lowered the triglyceride, LDL and cholesterol levels in a mouse after administration of the composition to the mouse.

LDL receptor knockout (LDLRKO) mice were obtained from Jackson Laboratories (Bar Harbor, Me.), and housed in groups under room temperature in a humidity-controlled environment with a regular light and dark cycle. The mice were provided free access to an atherogenic western diet (WD) containing 0.21% cholesterol (by weight) and 40% calories from fat and water for 4 weeks prior to treatment. For treatment, the mice were given (a) the WD diet alone, (b) WD and 24 g of leucine/kg diet, (c) WD and 24 g of leucine/kg diet and 50 mg nicotinic acid/kg diet, (d) WD and 24 g of leucine/kg diet and 250 mg nicotinic acid/kg diet, or (e) WD and 1000 mg nicotinic acid/kg diet; this is approximately equivalent to a low therapeutic dose of nicotinic acid in hypercholesterolemic humans (˜1,500 mg/day). The treatments were administered for eight weeks continuously.

Blood samples of the mice in all groups were obtained from tails of the mice at following four and eight weeks of administration. The serum/plasma levels of triglyceride, total cholesterol and cholesterol esters were measured. Following treatment, food was removed for four hours and the animals were euthanized.

Blood was collected into EDTA-coated tubes to analyze plasma lipid and cholesterol profiles. Plasma total cholesterol (TC, Pointe Scientific, Canton, Mich.), free cholesterol (FC, Wako, Richmond, Va.) and triglyceride (TG, Wako, Richmond, Va.) concentrations were measured using enzymatic assays according to manufacturer's instructions. Cholesterol ester (CE) was calculated as the difference between TC and FC.

To assess atherosclerosis, the circulatory system was perfused following euthanasia with phosphate-buffered saline (PBS) before removing the heart and aorta. The upper one-third of the heart was dissected and embedded in Optimal Cutting Temperature Compound (Sakura Tissue-Tek, Torrance, Calif.), frozen, and stored at −80° C. Blocks were serially cut at 8 μm intervals and stained with hematoxylin and 0.5% Oil Red O (Sigma-Aldrich) to evaluate aortic sinus atherosclerotic intimal area. Atherosclerotic lesion area and Oil Red O positive area were quantified using Image-Pro Plus software (Media Cybernetics, Bethesda, Md.). Whole aorta (from sinotubular junction to iliac bifurcate) was dissected and fixed in 10% formalin, and the adventitia was cleaned. Aortas were opened along the longitudinal axis and pinned onto black silicon elastomer (Rubber-Cal, Santa Ana, Calif.) for the quantification of atherosclerotic lesion area. The percentage of total aortic surface covered with atherosclerotic lesions was quantified by Image-Pro Plus software (Media Cybernetics, Bethesda, Md.) and used to determine the total lesion area.

To assess macrophage infiltration, Sections of aortic sinus were immuno-stained with rat monoclonal antibody against macrophage-specific CD68 (Clone FA11, 1:75, AbD Serotec, Raleigh, N.C.) followed by staining with alkaline phosphatase-conjugated mouse anti-rat (for CD68, 1:50) secondary antibodies (Jackson ImmunoResearch laboratories, West Grove, Pa.). Control slides contain no primary antibody. The CD68-positive areas were analyzed using Image-Pro Plus software (Media Cybernetics, Bethesda, Md.).

### Results:

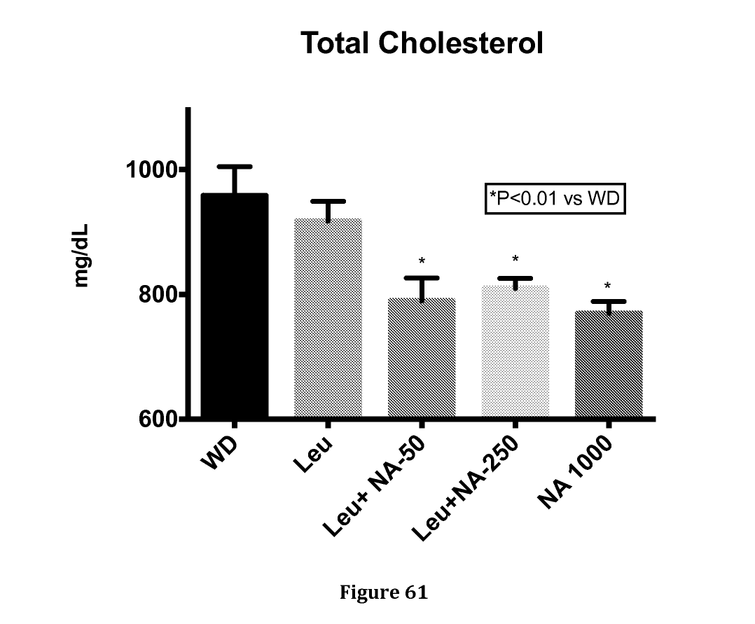

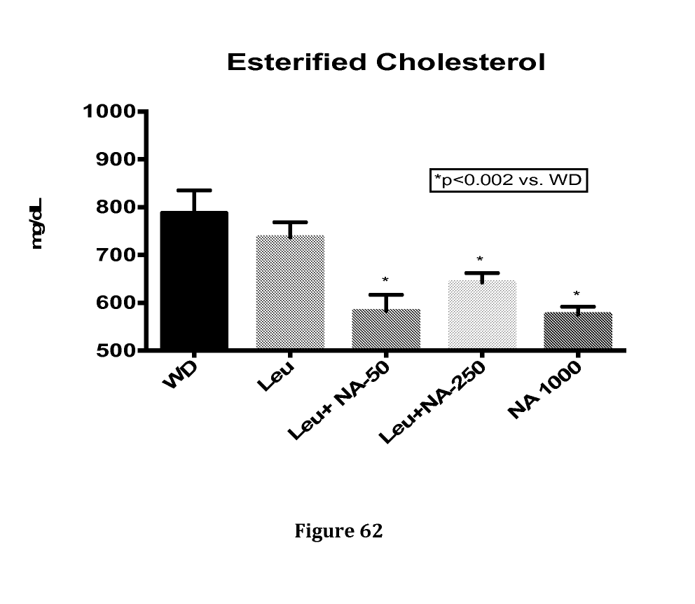

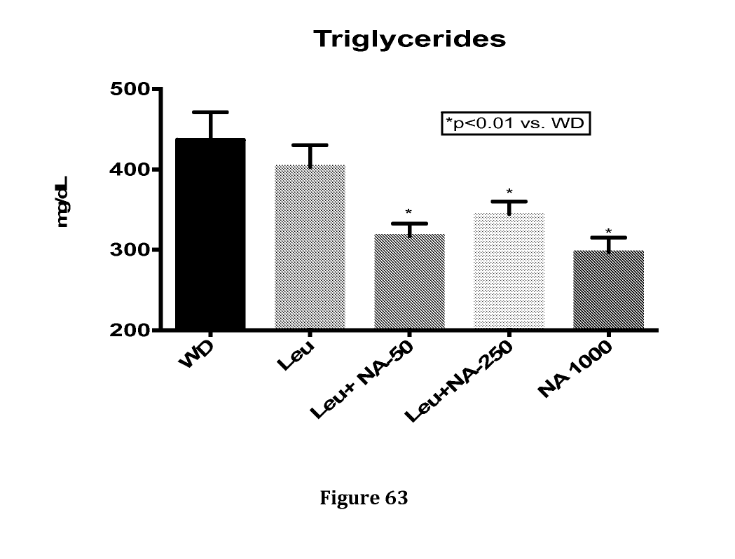

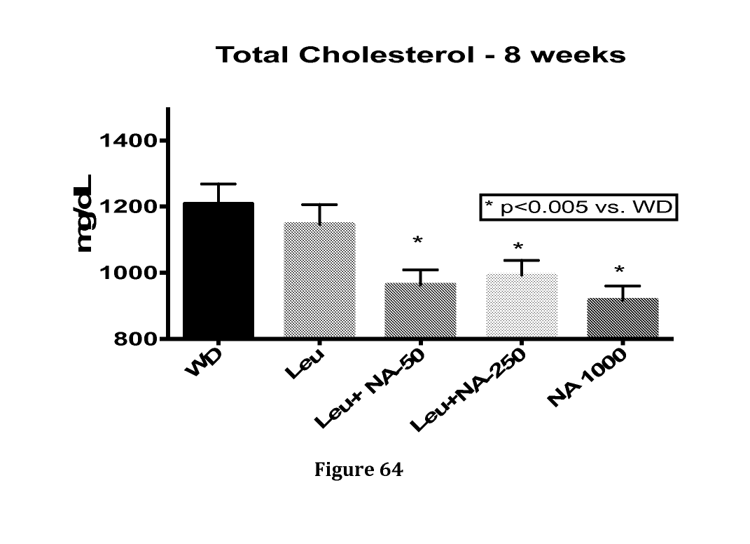

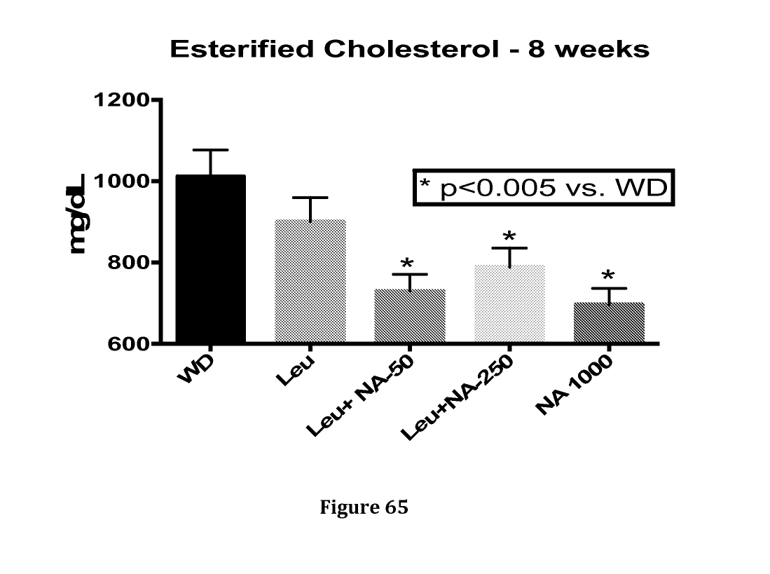

The atherogenic western diet resulted in profound elevations in plasma cholesterol (FIG. 61), cholesterol esters (FIG. 62), and trigylcyerides (FIG. 63) following four weeks of treatment. Addition of a therapeutic dose of nicotinic acid (1,000 mg/kg diet) resulted in a 20% decrease in total cholesterol (p<0.01, FIG. 61). Although leucine exerted no independent effect on total cholesterol, addition of leucine to sub-therapeutic doses of nicotinic acid (50 or 250 mg/kg diet) resulted in comparable decreases in total cholesterol to that found with the therapeutic dose (p<0.01, FIG. 61). Similarly, addition of a therapeutic dose of nicotinic acid (1,000 mg/kg diet) resulted in a 28% decrease in cholesterol esters (p<0.002, FIG. 62), and a statistically comparable decrease was found when leucine was added to sub-therapeutic doses of nicotinic acid (50 or 250 mg/kg diet) (p<0.002, FIG. 62). Leucine exerted no independent effect on cholesterol esters. Plasma triglycerides were similarly affected. Addition of a therapeutic dose of nicotinic acid (1,000 mg/kg diet) resulted in a 32% decrease in plasma triglycerides (p<0.01, FIG. 63), and a statistically comparable decrease in triglycerides was found when leucine was added to sub-therapeutic doses of nicotinic acid (50 or 250 mg/kg diet) (p<0.01, FIG. 63). These differences were sustained at the final (eight week) time point (FIGS. 64 and 65).

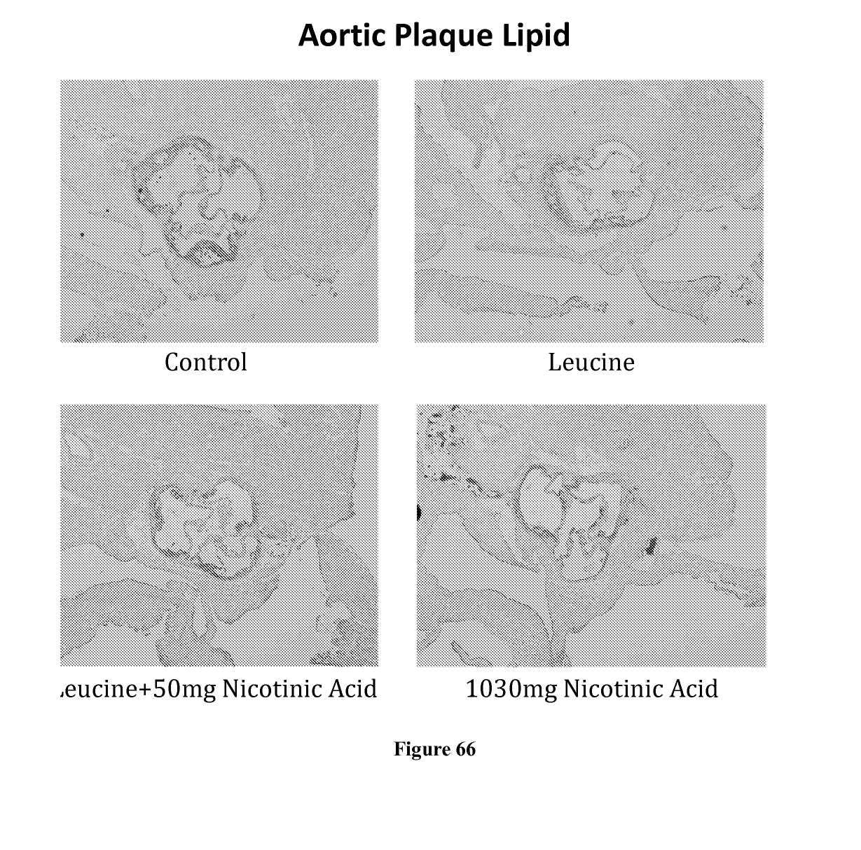

Addition of a therapeutic dose of nicotinic acid (1,000 mg/kg diet) to the western diet resulted in a ˜50% decrease in atherosclerotic lesion size (FIGS. 66-68) relative to the control fed the western diet alone.

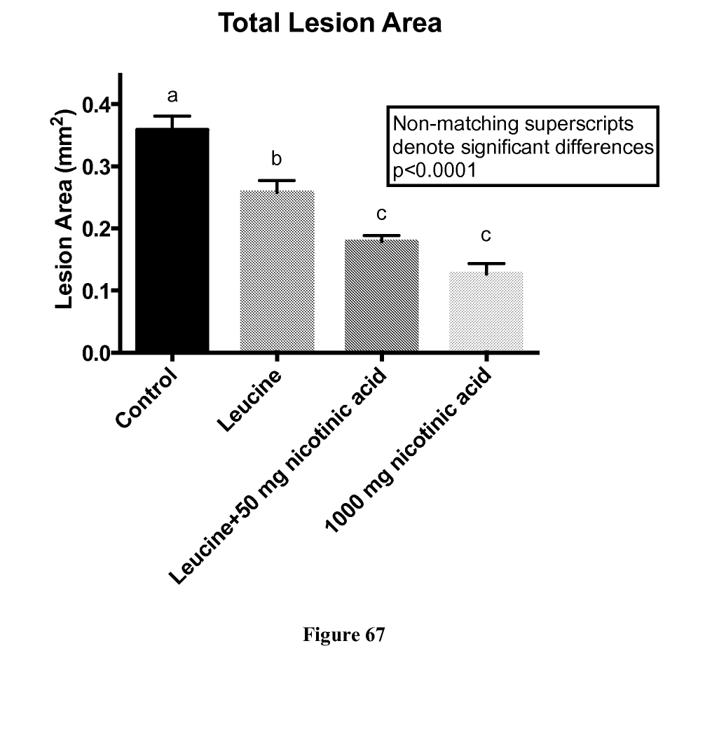

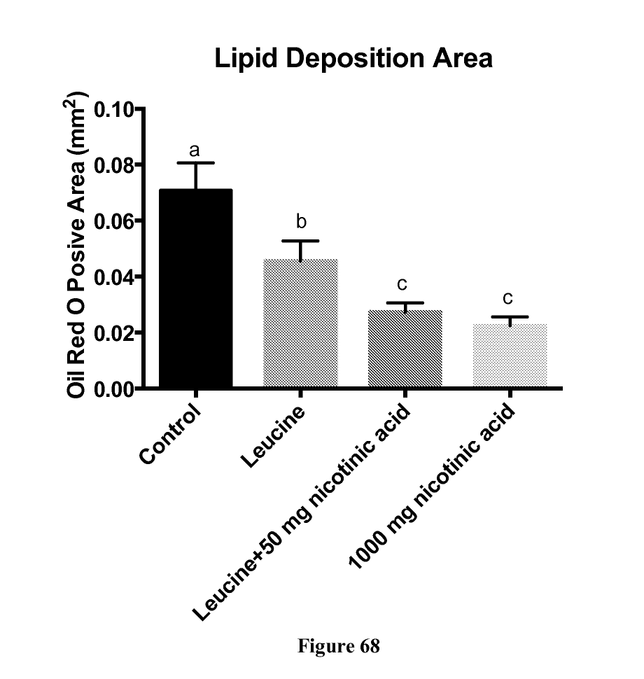

FIG. 66 shows Oil Red O stained aortic histology slides, which, in particular, the beneficial effects of administration of leucine along with 50 mg nicotinic acid in comparison to administration of leucine alone, 1000 mg nicotinic acid alone (positive control), and western diet alone (negative control). The histology slides shown in FIG. 66 were quantified as total lesion area in FIG. 67 and as lipid area (as observed by Oil Red O positive area) in FIG. 68. Addition of leucine to sub-therapeutic dose of nicotinic acid (50 mg/kg diet) resulted in a comparable decrease in lesion and lipid area to that found with the therapeutic dose (p<0.0001, FIGS. 67-68). Leucine exerted an independent effect on plaque area, but this effect was significantly less than that of sub-therapeutic nicotinic acid in combination with leucine (FIGS. 66-68).

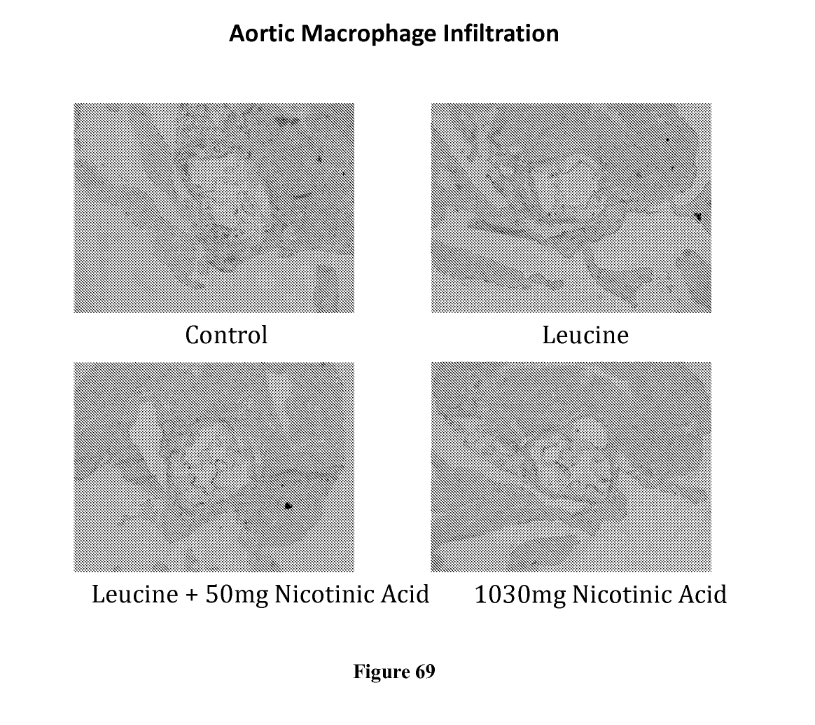

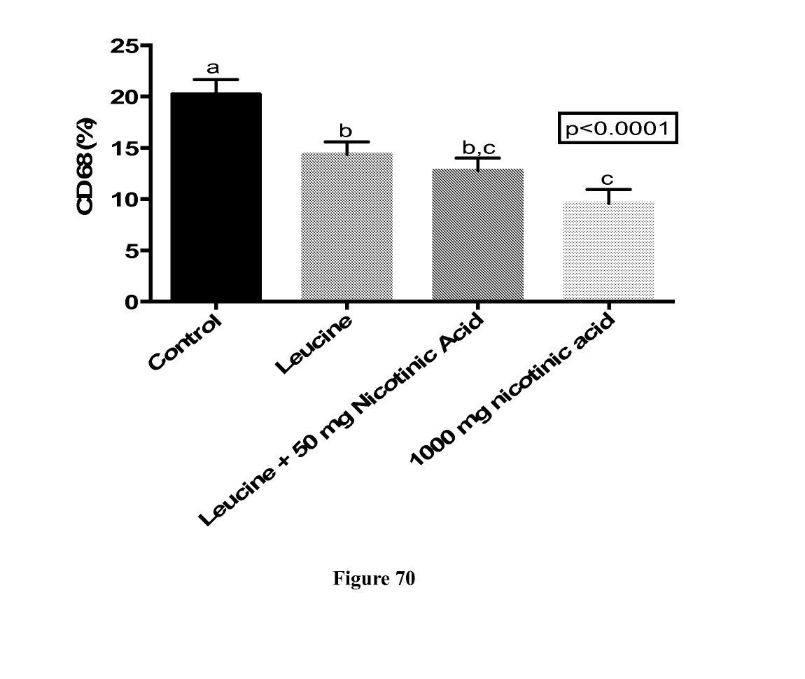

Addition of a therapeutic dose of nicotinic acid (1,000 mg/kg diet) to the Western diet also resulted in a ˜50% decrease in aortic macrophage infiltration (FIGS. 69-70). FIG. 69 shows CD68-positive area in representative histology slides, and this data is quantified as % CD68 positive area in the lesion in FIG. 70. Addition of leucine to sub-therapeutic dose of nicotinic acid (50 mg/kg diet) resulted in a comparable decrease in macrophage infiltration to that found with the therapeutic dose (p<0.0001, FIGS. 69-70). However, leucine also exerted a significant independent effect on reducing macrophage infiltration; this effect was not as great as the therapeutic dose of nicotinic acid, but was also not significantly different from the leucine-nicotinic acid combination (FIG. 70).

### Example 8: Effects of Nicotinic Acid/Leucine and Nicotinic Acid/Leucine/Resveratrol on Triglyceride, LDL, HDL and Cholesterol Levels in Human

To assess the efficacy of the subject compounds, humans are administered the subject compounds comprising (a) one or more agents selected from the group consisting of nicotinic acid, nicotinamide riboside, and nicotinic acid metabolite and (b) leucine and/or leucine metabolites as described herein, wherein the composition comprises free leucine and a sub-therapeutic amount of nicotinic acid. The composition can be used to lower triglyceride, LDL and cholesterol levels in a human after administering the composition to the human. A composition further comprising resveratrol is investigated as well.

Patients that are pre-diagnosed with hyperlipidemia are admitted for the randomized and double blind study. Each patient is administered orally (a) 50 mg nicotinic acid alone, (b) 50 mg of nicotinic acid and 550 mg of leucine, (c) 50 mg nicotinic acid, 550 mg leucine, and 50 mg resveratrol, (d) 550 mg leucine and 50 mg resveratrol, (e) 1500 mg nicotinic acid alone, and (f) placebo. The treatments are administered orally twice a day, for 60 days continuously.

Blood samples of the patients in all groups are obtained from at day 0, 30 and 60 after the administration. The serum/plasma levels of triglyceride, cholesterol, LDL and HDL are measured. Cutaneous vasodilation is measured by laser-Doppler flowmeter and the discomfort level described by the patients.

### Results:

For the treatment groups, group (a) and (f) may show similar, without statistically significant difference, levels of triglyceride, LDL, cholesterol and HDL as compared to day 0 values. Groups (b), (c) and (e) may exhibit significantly lower triglyceride, LDL and cholesterol levels, and significantly higher HDL level in the blood stream as compared to the respective day 0 values. Group (d) may exhibit minimal decrease in triglyceride, LDL and cholesterol levels.

It is also expected that only the patients receiving 1500 mg of nicotinic acid alone exhibit significant higher cutaneous vasodilation and more complaints from the patients as compared to all the other groups including placebo. The cutaneous vasodilation may be lower in the groups (a) to (d) as compared to group (e).

Overall, nicotinic acid with a dose that is 50 mg administered in conjunction with 550 mg leucine may exhibit similar effects on lowering the triglyceride, LDL, and cholesterol level as well as increasing the HDL level in patients as compared to 1.5 g nicotinic acid alone without increasing the cutaneous vasodilation significantly. 50 mg of nicotinic acid+550 mg leucine administered in conjunction with 50 mg resveratrol may exhibit similar effects.

### Example 9: Effects of Nicotinic Acid/Leucine and Nicotinic Acid/Leucine/Resveratrol on Atherosclerotic Plaque Size in Human

To assess the efficacy of the subject compounds, humans are administered the subject compounds as described herein, wherein the composition comprises free leucine and a sub-therapeutic amount of nicotinic acid. The composition can be used to reduce the size of atherosclerotic plaque in a human after administering the composition to the human. A composition further comprising resveratrol is investigated as well.

Patients that experience acute chest pain and are pre-diagnosed with hyperlipidemia are admitted for the randomized and double blind study. Each patient is administered orally (a) 50 mg nicotinic acid alone, (b) 50 mg of nicotinic acid and 550 mg of leucine, (c) 50 mg nicotinic acid, 550 mg leucine, and 50 mg resveratrol, (d) 550 mg leucine and 50 mg resveratrol, and (e) placebo. The treatments are administered orally twice a day, for 3 years continuously. The size of atherosclerotic plaque is measured at day 0, months 6, 12, 18, 24, 30 and 36 by quantitative coronary angiography.

### Results:

For the treatment groups, group (a) and (e) may show similar, without statistically significant difference, size of atherosclerotic lesion as compared to day 0 values. Groups (b) and (c) may exhibit significantly reduced atherosclerotic plaque size as compared to the respective day 0 values. Group (d) may exhibit minimal decrease in atherosclerotic plaque size.

### Example 10: Effects of Leucine-Nicotinic Acid on the Lifespan in C. elegans

The use of a composition comprising (a) nicotinic acid and (b) leucine as described herein was investigated, wherein the composition comprises free leucine and a sub-therapeutic amount of nicotinic acid. The composition synergistically extended the lifespan in a subject after administration of the composition to the subject.

Worms (N2 Bristol wild-type) were obtained from the Caenorhabditis Genetics Center (CGC) at the University of Minnesota and grown on standard NGM plates with E. coli (OP50) as food source at 20° C. Eggs were hatched on a starved plate overnight. Then synchronized L1 larvae were transferred to E. coli fed NGM plates containing indicated treatments for about 35 hours to reach L4/young adult stage. To study lifespan, 50 young adult worms were placed on NGM agar plates seeded with E. coli strain OP-50 (=day 1 of study). All treatments were added with the indicated concentrations to E. coli the agar plates. Treatments included 10 nM of nicotinic acid and 0.5 mM of leucine.

The worms were maintained at 20° C. throughout the duration of the study. Worms were transferred to new plates daily to eliminate progeny. Worms were scored as dead if they did not respond to repeated touch with a platinum pick. The study was continued until the last animal was dead. Data were analyzed via Kaplan-Meier survival curves using Prism 6 (GraphPad Software) and statistical significance was determined by the Log-rank (Mantel-Cox) test.

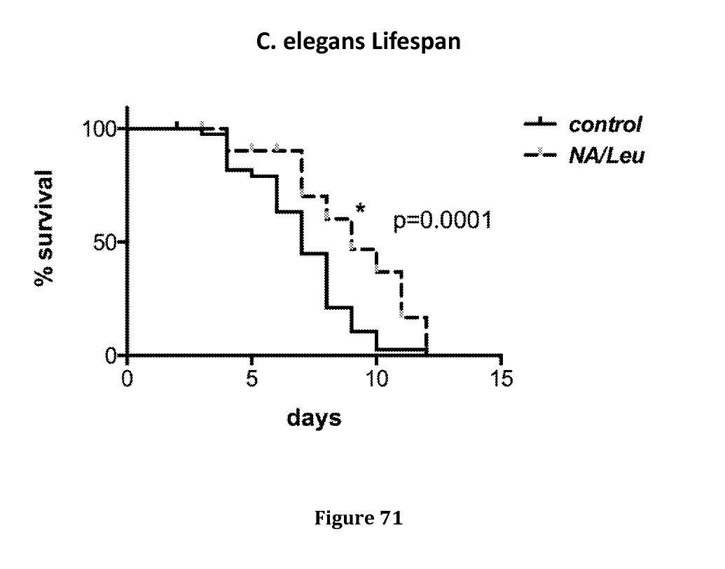

It was found that leucine (0.5 mM) and nicotinic acid (10 nM) each exerted no independent effect on lifespan, but when combined extended maximal lifespan under basal conditions, and extended median lifespan by 28% under conditions of oxidative stress induced by administration of paraquat (0.2 mM) (FIG. 71).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

### Example 11: Effect of Leucine/Metformin/Nicotinic Acid on Fatty Acid Oxidation, Lipid Content and Glucose Utilization

The use of a composition comprising nicotinic acid, metformin and leucine as described herein was investigated. The levels of nicotine acid (1 and 10 nM), metformin (10-100 μM) and leucine (0.3-0.5 mM) were selected such that these level (a) are attainable by sub-therapeutic dose oral administration and (b) had no independent effect on the variables studied.

Cell Culture:

C2C12 myoblasts (American Type Culture Collection) were plated at a density of 8000 cells/cm² (10 cm² dish) and grown in Dulbecco's modified eagle's medium (DMEM) containing 10% fetal bovine serum (FBS), and antibiotics at 37° C. in 5% CO₂. For differentiation of C2C12 cells, cells were grown to 100% confluence, transferred to differentiation medium (DMEM with 2% horse serum and 1% penicillin-streptomycin), and fed with fresh differentiation medium every day until myotubes were fully formed (3 days). Human HepG2 cells (American Type Culture Collection HB-8065) were grown and maintained in DMEM, containing 5.5 mM glucose, 10% FBS and antibiotics (1% penicillin-streptomycin) at 37° C. in 5% CO₂ in air. Medium was changed every 2 to 3 day and cells were sub-cultured at a ratio of 1:4 to 1:6 upon reaching 80% confluence.

Seahorse Fatty Acid Oxidation:

The palmitate-stimulated oxygen consumption rate was measured with the XF 24 analyzer (Seahorse Bioscience, Billerica, Mass., USA) as previously described (6-8) with slight modifications. Cells were seeded at 40,000 cells per well and differentiated as described above. Lipid accumulation was then induced by changing the media to DMEM containing either 25 mM glucose or 200 uM palmitate for 48 h. Media was then changed and the cells treated for 24 hours with the indicated treatments, washed twice with non-buffered carbonate-free pH 7.4 low glucose (2.5 mM) DMEM containing carnitine (0.5 mM), equilibrated with 550 μL of the same media in a non-CO₂ incubator for 45 minutes, and then inserted into the instrument for 15 minutes of further equilibration, followed by O₂ consumption measurement. Three successive baseline measures at five-minute intervals were taken prior to injection of palmitate (200 μM final concentration). Four successive 5-minute measurements of O₂ consumption were then conducted, followed by 10 minute re-equilibration and another 3-4 5-minute measurements. This measurement pattern was then repeated over a 4-6 hour period. Data for each sample were normalized to the pre-palmitate injection baseline for that sample and expressed as % change from that baseline. Pre-palmitate injection values were 371±14 pmol O₂/minute for myotubes and 193±11 pmol O₂/minute for adipocytes. The area under of the curve of O₂ consumption change from baseline for each sample was then calculated and used for subsequent analysis.

Lipid Content:

Lipid content was measured via Oil Red O staining. Following treatment, cells were washed twice with phosphate-buffered saline (PBS), incubated in 10% formalin for 10 minutes at room temperature, and the formalin removed and replaced with fresh 10% formalin for at least one additional hour. The formalin was then removed and the cells washed with 60% isopropanol for 5 minutes at room temperature and then dried at room temperature. Cells were then stained with Oil Red O. Oil Red O stock solution was prepared as 0.35% solution in isopropanol which was then stirred overnight, filtered (0.2μ filter); a working solution was prepared by mixing 6 mL Oil Red O stock solution with 4 mL distilled deionized water, allowing the solution to sit 20 minutes at room temperature followed by filtering (0.2μ). This Oil Red O working solution was added to the dried cells (100 μL in 96 well plates; 0.5 mL in 6-well plates) for 30 minutes, removed and immediately washed four times with distilled deionized water. Oil Red O was quantitated in cells by measuring optical density (OD) at 500 nm using a Biotek synergy HT Microplate Reader (BioTek, Winooski, Vt. USA). The Oil Red O was then eluted in 100% isopropanol (75 μL in 96 well plates; 0.5 mL in 6-well plates). OD of the eluent was then read at 500 nm as described above. Data were normalized to cell number or to protein content via the BCA assay.

Glucose Utilization:

In the absence of a fatty acid source and oxidative metabolism, glycolysis and subsequent lactate production results in extracellular acidification, which was also measured using a Seahorse Bioscience XF24 analyzer. Cells were prepared and equilibrated similar to the methods described above for fatty acid oxidation, with the exclusion of carnitine from the medium. Following instrument equilibration and three baseline measurements, glucose was injected to a final concentration of 10 mM in each well. Measurements were taken as described above utilizing the sensors for extracellular acidification rather than O₂ consumption. Insulin (final concentration of 5 nM) was added to some wells as a positive control and to some treatment and control wells to assess treatment effects on insulin response. Data for each sample were normalized to the pre-glucose injection baseline for that sample and expressed as % change from that baseline. The area under of the curve of extracellular acidification change from baseline for each sample was the calculated and used for subsequent analysis.

Statistics:

Data were analyzed via one-way analysis of variance and least significant difference test was used to separate significantly different group means.

### Results:

Glucose Utilization:

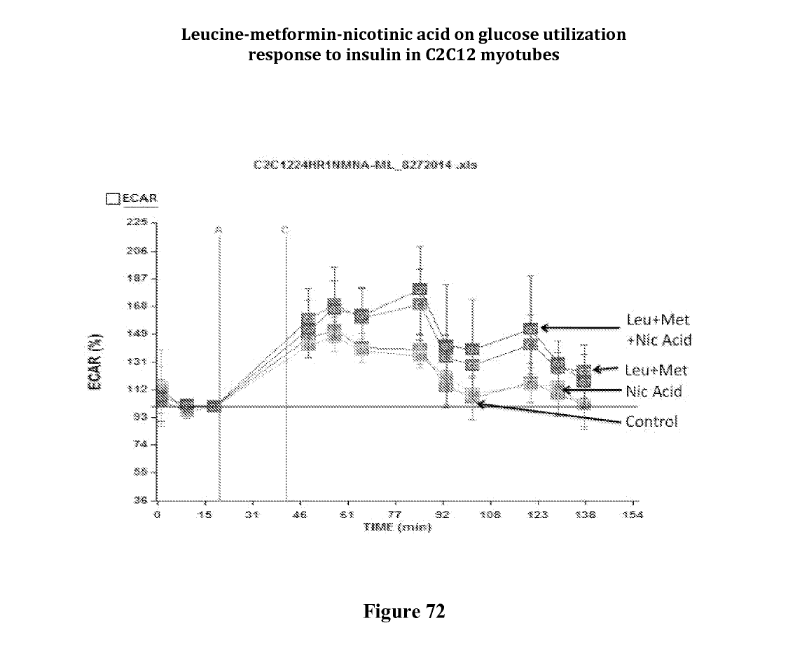

Leucine and metformin synergistically stimulated both basal and insulin-stimulated glucose utilization (FIGS. 72 and 73), consistent with our previous observations. Leucine and nicotinic acid exhibited no interactive or independent effects on glucose utilization at the concentrations utilized, and the combination of leucine, metformin and nicotinic acid exerted comparable effects to that of leucine-metformin in the absence of nicotinic acid (FIGS. 72 and 73). These effects were evident within 6 hours of incubation and sustained for the 24 hour experiments

Fat Oxidation:

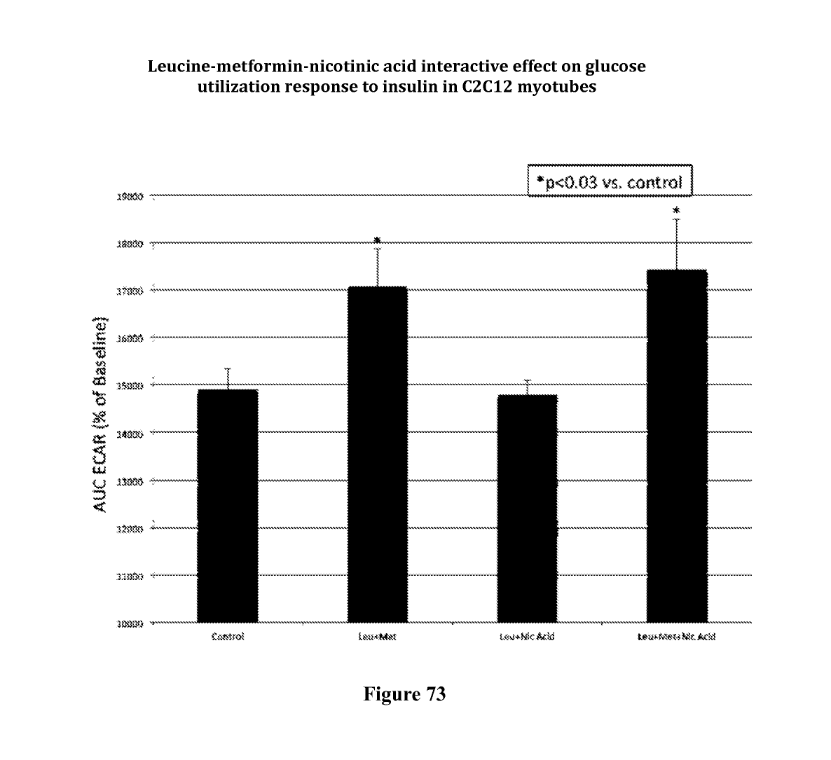

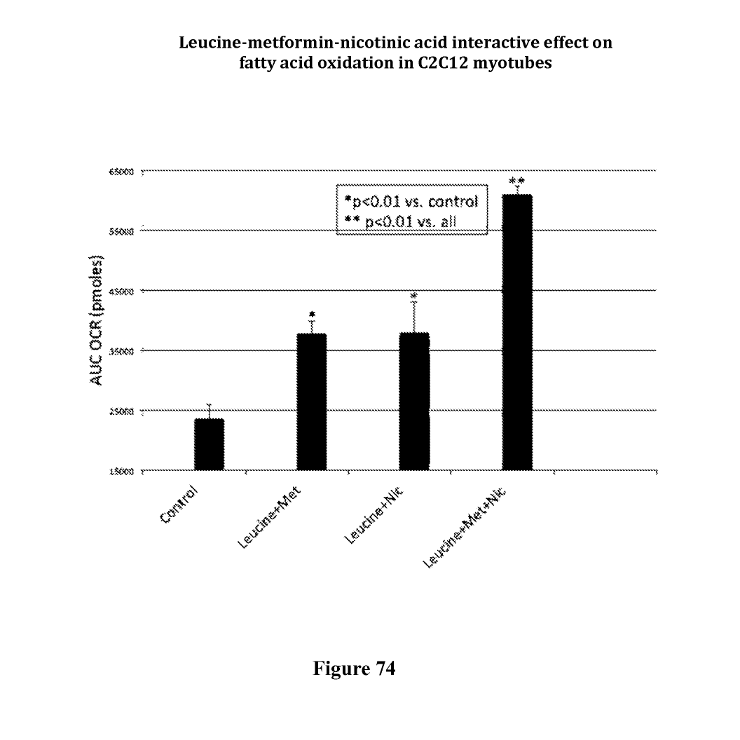

Leucine-metformin and leucine-nicotinic acid combinations exerted synergistic effects on hepatocyte fat oxidation, and the combination of leucine, metformin and nicotinic acid exerted a markedly greater effect on fat oxidation as compared to combinations of leucine and nicotinic acid or leucine and metformin (FIG. 74).

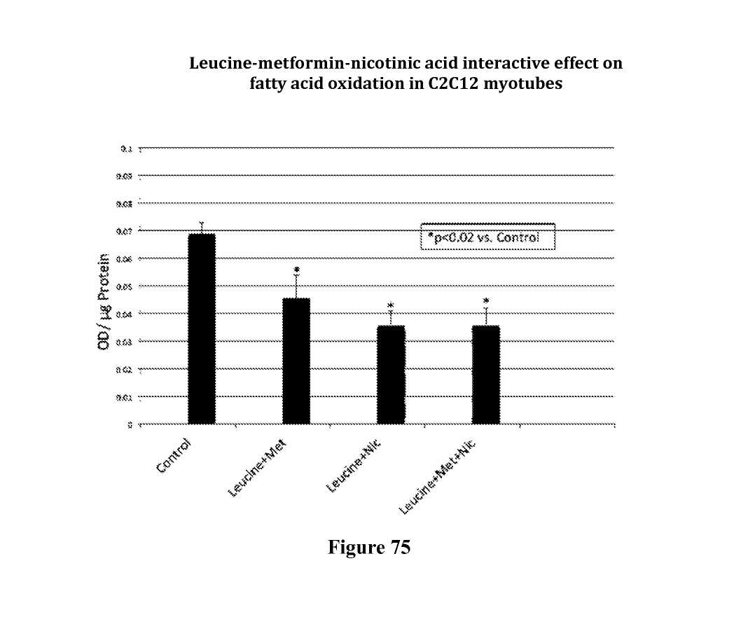

Leucine-metformin and leucine-nicotinic acid reduced hepatocyte lipids (FIG. 75), and the leucine-nicotinic acid combination exerted a greater effect than leucine-metformin. The three-way combination of leucine-metformin-nicotinic acid exerted similar effects to leucine-nicotinic acid in the absence of metformin (FIG. 75).

### Example 12: Effect of Leucine/Metformin/Resveratrol on Insulin Sensitivity, Weight Gain, Sirt1 Activity and Fatty Acid Oxidation in Diet-Induced Obesity Mice

The present study was designed to more comprehensively evaluate the long-term efficacy of leucine in augmenting the effects of metformin on insulin sensitivity in a mouse model of diet-induced obesity and insulin resistance and to determine whether resveratrol is a required component for this augmentation.

Leucine (Leu) has been found to activate Sirt1 and to potentiate other activators of the sirtuin/AMPK pathway, including resveratrol (Res), resulting in improvement of insulin sensitivity. Since metformin (Met) also converges on this pathway, we tested the effects on glycemic control of leu/met combinations with and without Res in a mouse model of high fat diet (HFD) induced insulin resistance.

Six to eight weeks old male C57/BL6 mice were purchased from Jackson Laboratories. Obesity and insulin resistance were induced via a high-fat diet (HFD) for 6 weeks. The animals were then randomized into one of the following groups (Table 3 and Table 4) with 10 animals/group and kept on their diet for 6 weeks.

TABLE 3
Treatment of diet-induced obesity mice with combinations of
leucine/metformin/resveratrol composition.

| Group No. | Leucine | Metformin | Resveratrol |
|---|---|---|---|
| 1a | Standard Diet | 0 | 0 |
| 2a | High Fat Diet | 0 | 0 |
| 3a | High Fat Diet + | 0 | 0 |
| | 24 g leucine/kg diet | | |
| 4a | High Fat Diet + | 0 | 12.5 mg/kg diet |
| | 24 g leucine/kg diet | | |
| 5a | High Fat Diet + | 0.25 g/kg diet | 12.5 mg/kg diet |
| | 24 g leucine/kg diet | | |
| 6a | High Fat Diet + | 0.15 g/kg diet | 12.5 mg/kg diet |
| | 24 g leucine/kg diet | | |
| 7a | High Fat Diet + | 0.05 g/kg diet | 12.5 mg/kg diet |
| | 24 g leucine/kg diet | | |
| 8a | High Fat Diet + | 1.5 g/kg diet | 12.5 mg/kg diet |
| | 24 g leucine/kg diet | | |

TABLE 4
Treatment of diet-induced obesity mice with combinations of
leucine/metformin composition.

| Group No. | Leucine | Metformin | Resveratrol |
|---|---|---|---|
| 1b | Standard Diet | 0 | 0 |
| 2b | High Fat Diet | 0 | 0 |
| 3b | High Fat Diet + | 0 | 0 |
| | 24 g leucine/kg diet | | |
| 4b | High Fat Diet + | 0 | 0 |
| | 24 g leucine/kg diet | | |
| 5b | High Fat Diet + | 0.25 g/kg diet | 0 |
| | 24 g leucine/kg diet | | |
| 6b | High Fat Diet + | 0.15 g/kg diet | 0 |
| | 24 g leucine/kg diet | | |
| 7b | High Fat Diet + | 0.5 g/kg diet | 0 |
| | 24 g leucine/kg diet | | |
| 8b | High Fat Diet + | 1.5 g/kg diet | 0 |
| | 24 g leucine/kg diet | | |

Animals were housed in polypropylene cages at a room temperature of 22° C. and regime of 12 h light/dark cycle. The animals had free access to water and their experimental food throughout the experiment. Body weight measurement and blood collection was performed every week. At the end of the treatment period (6 weeks) all animals were fasted overnight and humanely euthanized with CO₂ inhalation. Blood and tissues were collected for further experiments as described below.

This study and all animal procedures were performed under the auspices of an Institutional Animal Care and Use Committee-approved protocol of the Georgia State University and in accordance with PHS policy and recommendations of the Guide.

Insulin Tolerance Test (ITT):

Prior to each ITT, food was removed from the mice for 4 to 6 hours and basal blood glucose level was measured from tail snipping using an OneTouch Ultra Glucose meter (Lifespan, Milpitas, Calif.). Then the mice were injected with insulin (1.0 U/kg BW) in ˜0.1 ml 0.9% NaCl intraperitoneally. Blood glucose was then measured 15, 30, 60, 90 and 120 min after insulin injection. Change in blood glucose over the linear portion of the response curve was then calculated.

Glucose Tolerance Test (GTT):

Prior to each GTT, mice were fasted overnight (˜16 hours) and basal blood glucose level was measured from tail snipping using an OneTouch Ultra Glucose meter (Lifespan, Milpitas, Calif.). Then the mice were injected with glucose (1.2 g/kg BW) intraperitoneally. Blood glucose was then measured 15, 30, 60, 90 and 120 min after glucose injection. The area under the curve of the response curve was then calculated.

HOMA_{IR} Index:

The homeostasis model assessment of insulin resistance (HOMA_{IR}) was used as an index of changes in insulin sensitivity. HOMA_{IR} was calculated via standard formula from fasting plasma insulin and glucose as follows: HOMA_{IR}=[Insulin (μU/mL)×glucose (mM)]/22.5. The plasma glucose and insulin concentrations were measured using the Glucose Assay Kit from Cayman (Ann Arbor, Mich.) and the Insulin kit from Millipore (Billerica, Mass.), respectively.

Cell Culture:

Murine 3T3-L1 pre-adipocytes were grown in the absence of insulin in Dulbecco's modified Eagle's medium (DMEM, 25 mM glucose) containing 10% fetal bovine serum (FBS) and antibiotics (1% penicillin-streptomycin)(adipocyte medium) at 37° C. in 5% CO₂ in air. Confluent pre-adipocytes were induced to differentiate with a standard differentiation medium (DM2-L1, Zen-Bio Inc., NC). Pre-adipocytes were maintained in this differentiation medium for 3 days and subsequently cultured in adipocyte medium for further 8 to 10 days to allow at least 90% of cells to reach fully differentiation before treatment. Media was changed every 2-3 days; differentiation was determined microscopically via inclusion of fat droplets.

### Sirt1 Activity (Fleur-de-Lys):

Sirt1 activity was measured by using the Sirt1 Fluorimetric Drug Discovery Kit (BML-AK555, ENZO Life Sciences Inc., Farmingdale, N.Y., USA). The sensitivity and specificity of this assay kit was tested by Nin et al. (Nin et al., “Role of deleted in breast cancer 1 (DBC1) protein in SIRT1 deacetylase activation induced by protein kinase A and AMP-activated protein kinase.” J. Biol Chem 287, 23489-23501 Jul. 6, 2012). Sirt1 activity was assessed by the degree of deacetylation of a standardized substrate containing an acetylated lysine side chain. The substrate utilized was a peptide containing amino acids 379-382 of human p53 (Arg-His-Lys-Lys[Ac]), an established target of Sirt1 activity; Sirt1 activity was directly proportional to the degree of deacetylation of Lys-382. Samples were incubated with peptide substrate (25 μM), and NAD⁺ (500 μM) in a phosphate-buffered saline solution at 37° C. on a horizontal shaker for 45 minutes. The reaction was stopped with the addition of 2 mM nicotinamide and a developing solution that binds to the deacetylated lysine to form a fluorophore. Following 10 minutes incubation at 37° C., fluorescence was read in a plate-reading fluorimeter with excitation and emission wavelengths of 360 nm and 450 nm, respectively. Resveratrol (100 mM) served as a Sirt1 activator (positive control) and suramin sodium (25 mM) as a Sirt1 inhibitor (negative control). Sirt1 activity was measured in a modified assay using 5 μl of cell lysate. The lysates were prepared by homogenizing cells in ice-cold RIPA buffer plus protease inhibitor mix (Sigma Aldrich Corp., St. Louis, Mo., USA). After 10 min incubation on ice, the homogenate was centrifuged at 14,000 g and the supernatant was used for further experiments. Data for endogenous Sirt1 activation were normalized to cellular protein concentration measured via BCA-assay.

Western Blot:

The P-AMPK and AMPK antibody were obtained from Cell Signaling (Danvers, Mass.). Protein levels of cell extracts were measured by BCA kit (Thermo Scientific). For Western blot, 10 μg protein was resolved on 10% gradient polyacrylamide gels (Criterion precast gel, Bio-Rad Laboratories, Hercules, Calif.), transferred to PVDF membranes, incubated in blocking buffer (3% BSA in TBS) and then incubated with primary antibody (1:1000 dilution), washed and incubated with secondary horseradish peroxidase-conjugated antibody (1:10000 dilution). Visualization and chemiluminescent detection was conducted using BioRad ChemiDoc instrumentation and software (Bio-Rad Laboratories, Hercules, Calif.) and band intensity was assessed using Image Lab 4.0 (Bio-Rad Laboratories, Hercules, Calif.), with correction for background and loading controls.

Fatty Acid Oxidation:

Cellular oxygen consumption was measured using a Seahorse Bioscience XF24 analyzer (Seahorse Bioscience, Billerica, Mass.) in 24-well plates at 37° C., as described by Feige et al with slight modifications. Cells were seeded at 40,000 cells per well, differentiated as described above, treated for 24 hours with the indicated treatments, washed twice with non-buffered carbonate-free pH 7.4 low glucose (2.5 mM) DMEM containing carnitine (0.5 mM), equilibrated with 550 μL of the same media in a non-CO₂ incubator for 30 minutes, and then inserted into the instrument for 15 minutes of further equilibration. O₂ consumption was measured in three successive baseline measures at eight-minute intervals prior to injection of palmitate (200 μM final concentration). Post-palmitate-injection measurements were taken over a 3-hour period with cycles consisting of 10 min break and three successive measurements of O₂ consumption.

Statistical Analysis:

All data were expressed as mean±SEM, with the exception of Seahorse fatty acid oxidation, which is shown as mean±SD. Data were analyzed by one-way ANOVA, and significantly different group means (p<0.05) were separated by the least significant difference test using GraphPad Prism version 6 (GraphPad Software, La Jolla Calif. USA, www.graphpad.com).

### Result:

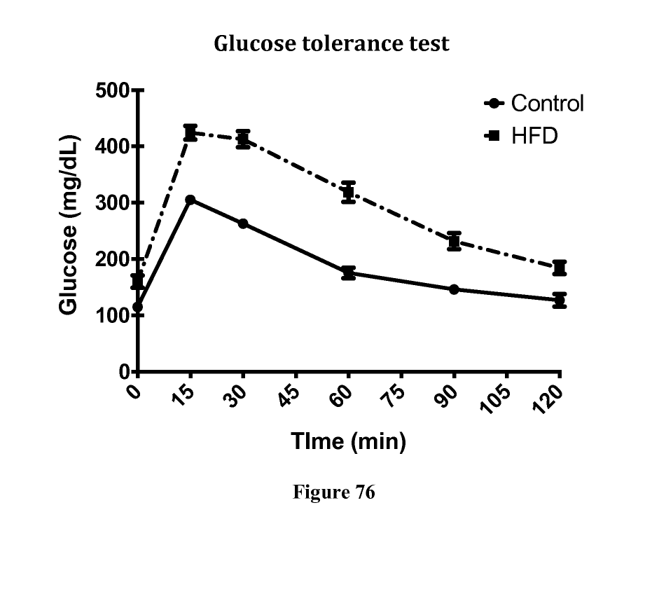

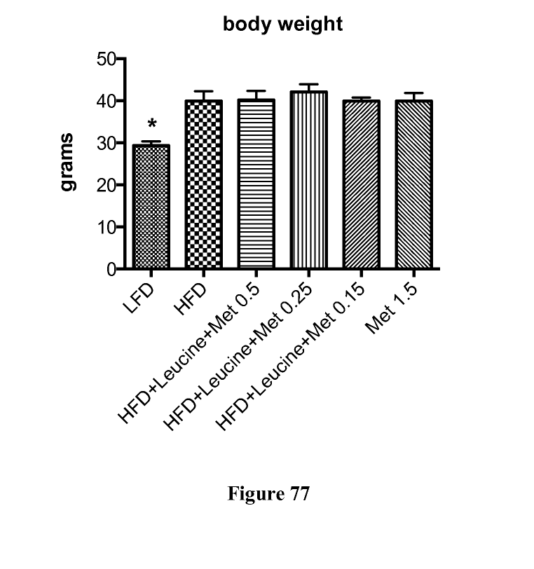

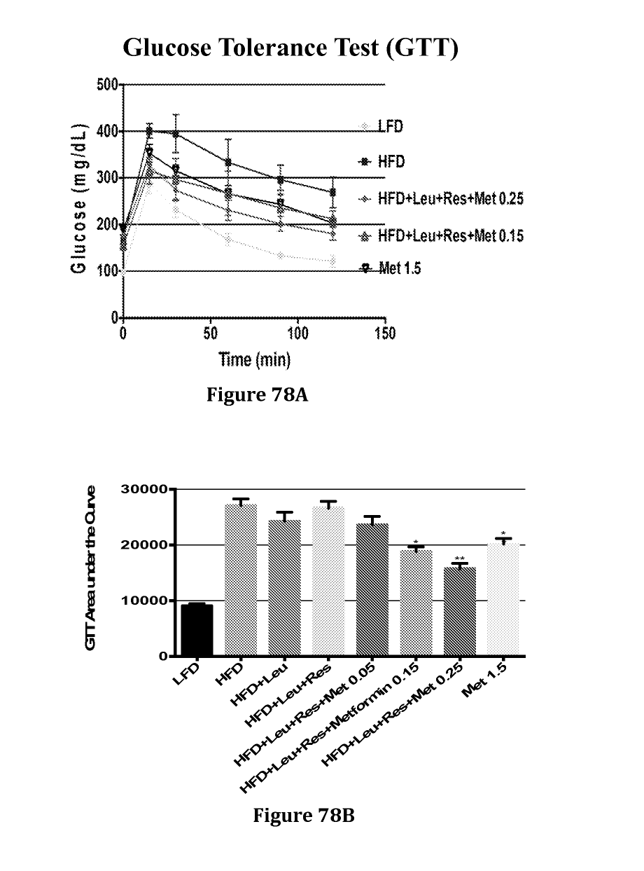

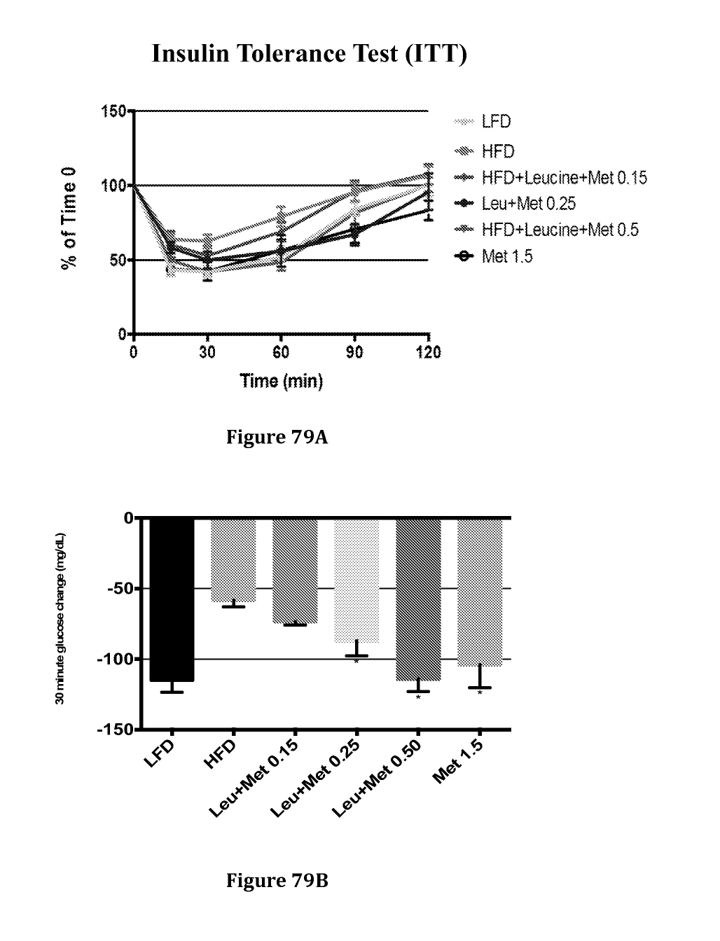

After 6 weeks of obesity induction with HFD and prior to treatments, glucose tolerance test (GTT) was performed in the control mice: Group 1a mice provided with standard diet and standard leucine, and Group 2a mice provided with high fat diet (HFD) with standard leucine. HFD caused significant fasting and postprandial hyperglycemia in the GTT indicating insulin resistance (FIG. 76) and a significant weight gain in Group 2a mice (FIG. 77). In the first study (Table 3), we evaluated the long-term efficacy of a combination of leucine/resveratrol with sub-therapeutic doses of metformin. FIG. 78A and FIG. 78B show the GTT and ITT after 5 weeks of treatment. The HFD induced fasting and post-prandial hyperglycemia was not significantly affected by the addition of either Leucine alone in Group 3a or the combination of Leucine with resveratrol in Group 4a (FIG. 78A and FIG. 78B). However, adding a sub-therapeutic level of 0.15 g metformin/kg diet to the leucine/resveratrol combination (Group 6a) reduced the HFD-induced hyperglycemia comparable to full-dose metformin, while adding 0.25 g metformin/kg diet (Group 5a) resulted in a significantly greater reduction in the area under the curve (FIG. 78B). Also the blood glucose response to insulin tolerance test (ITT) (FIG. 79A and FIG. 79B) was significantly improved in Group 5a mice treated with the 0.25 g metformin/resveratrol/leucine group, comparable to the effect of full-dose metformin in Group 8a (FIG. 79B).

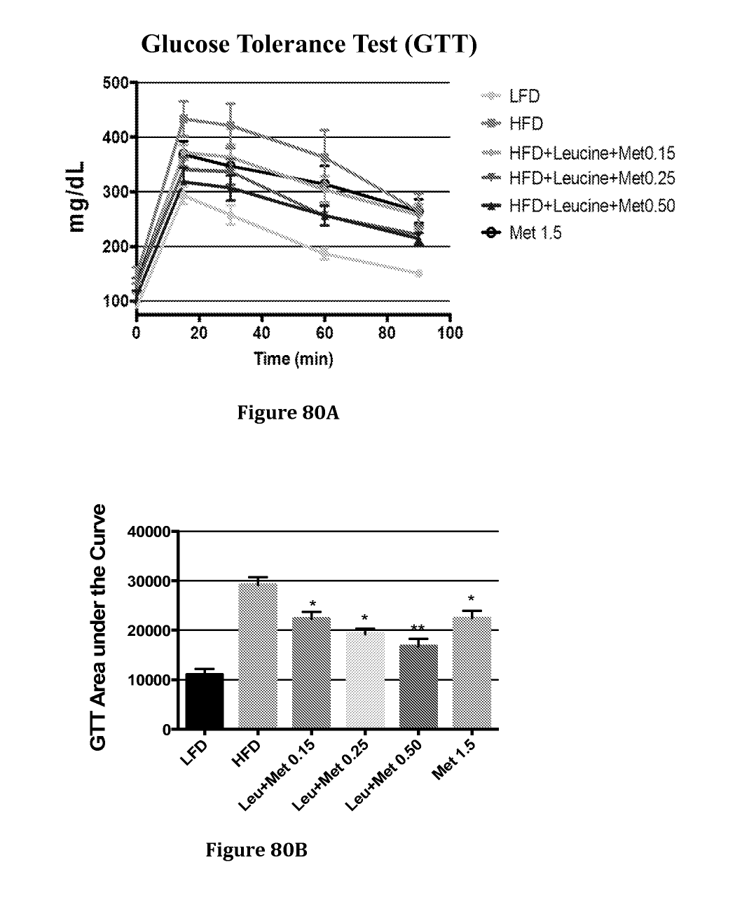

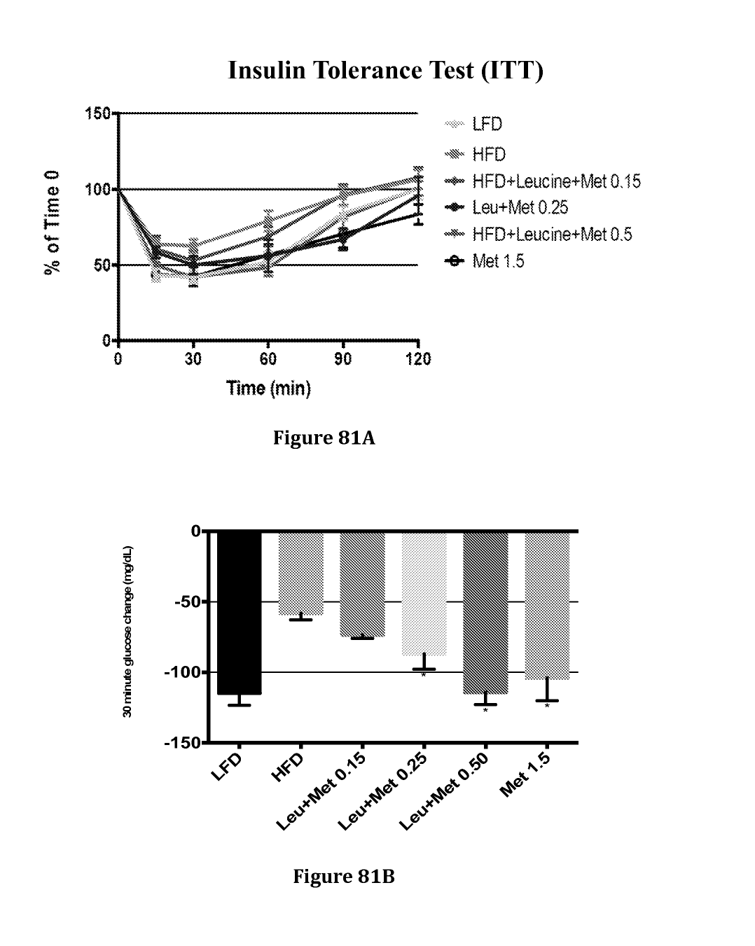

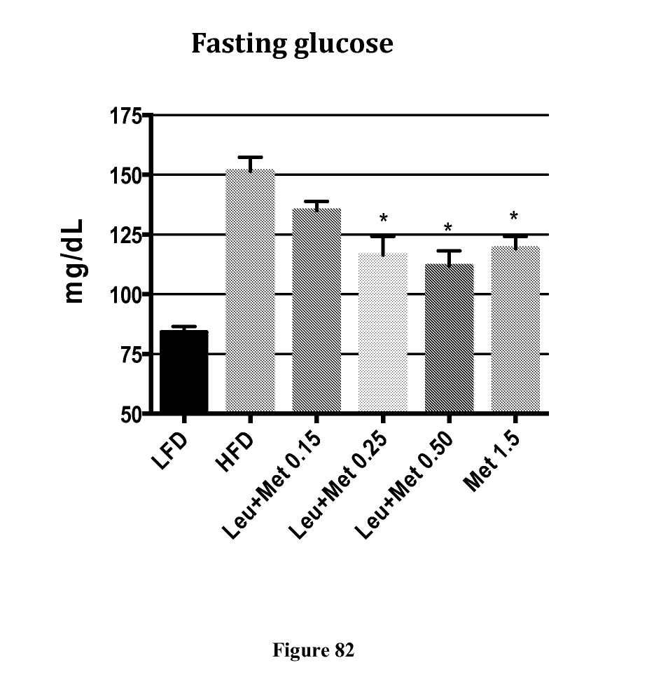

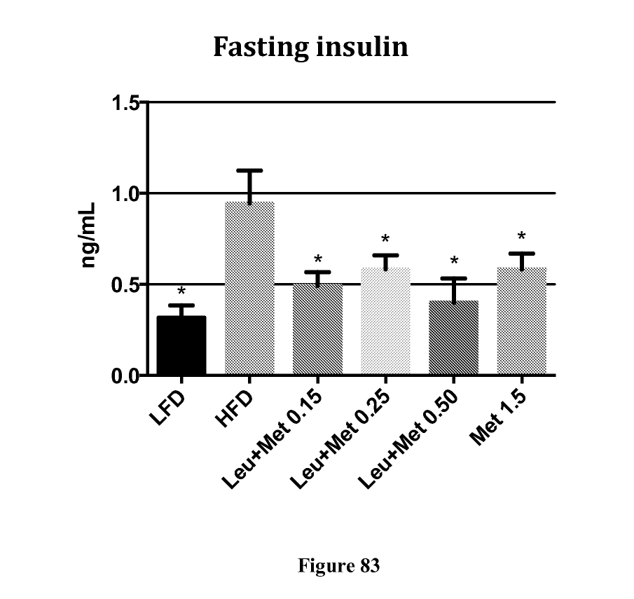

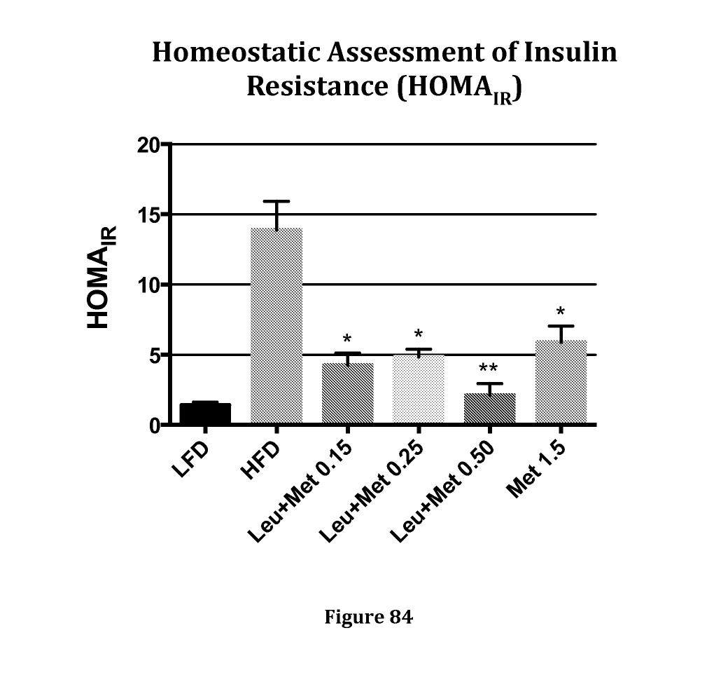

To test, whether the combination of leucine and metformin alone was capable to achieve the same therapeutic level on insulin sensitivity in the absence of resveratrol, we repeated the study without resveratrol in study 2 (Table 4). Since the lowest group of metformin (0.05 g/kg diet) did not show any effects in study 1 (Table 3), we did not continue that group in study 2 and instead included a group with a higher metformin concentration (0.5 g/kg diet). Similar to the result of study 1 (Table 3), the 0.15 metformin (Group 6b) and the 0.25 metformin (Group 5b) treatment groups exhibited reduced area under the glucose tolerance curve, similar to full dose metformin in Group 8b (FIG. 80), while the 0.5 metformin (Group 7b)/leucine group showed a significantly greater effect than mice receiving full-dose metformin (Group 8b) (FIGS. 80A-FIG. 80B and FIG. 81A and FIG. 81B). The ITT was improved by the 0.25 and 0.5 metformin/leucine group comparable to full dose metformin, but the 0.15 metformin/leucine group did not significantly affect this parameter (FIG. 81A and FIG. 81B), but the 0.15 metformin/leucine group did not significantly affect this parameter (FIG. 81A and FIG. 81B). The metformin/leucine groups significantly reduced fasting blood glucose (FIG. 82) and insulin levels (FIG. 83) comparably to full-dose metformin, and the 0.5 metformin/leucine group (Group 7b) exerted a significantly greater effect on HOMA_{IR}, resulting a HOMA_{IR} value not different from the LFD mice in Group 1b (FIG. 84). These results suggest that leucine and metformin can have synergistic effect on increasing insulin sensitivity in the absence of resveratrol.

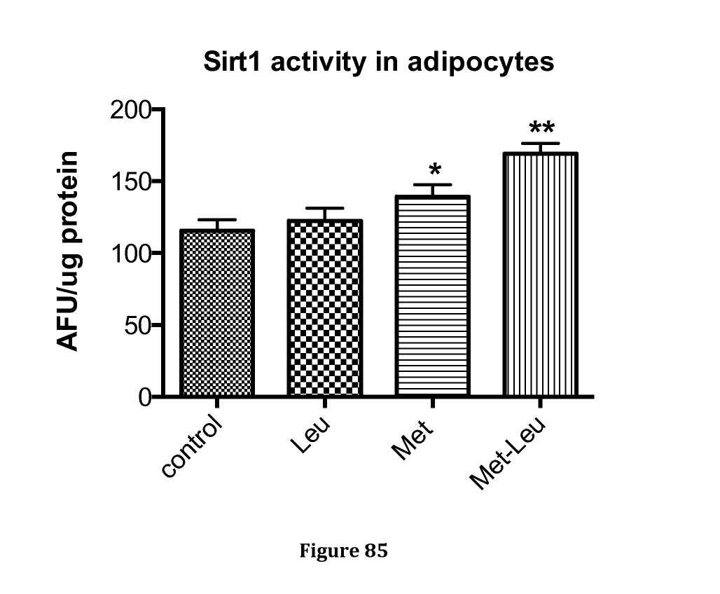

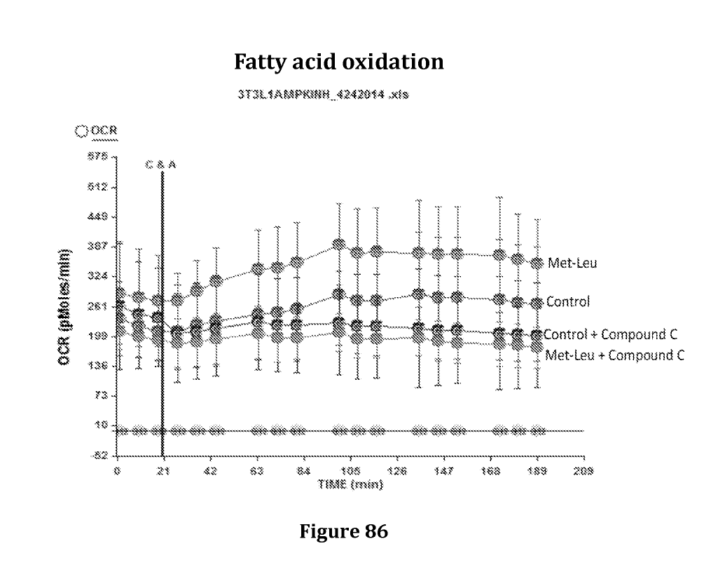

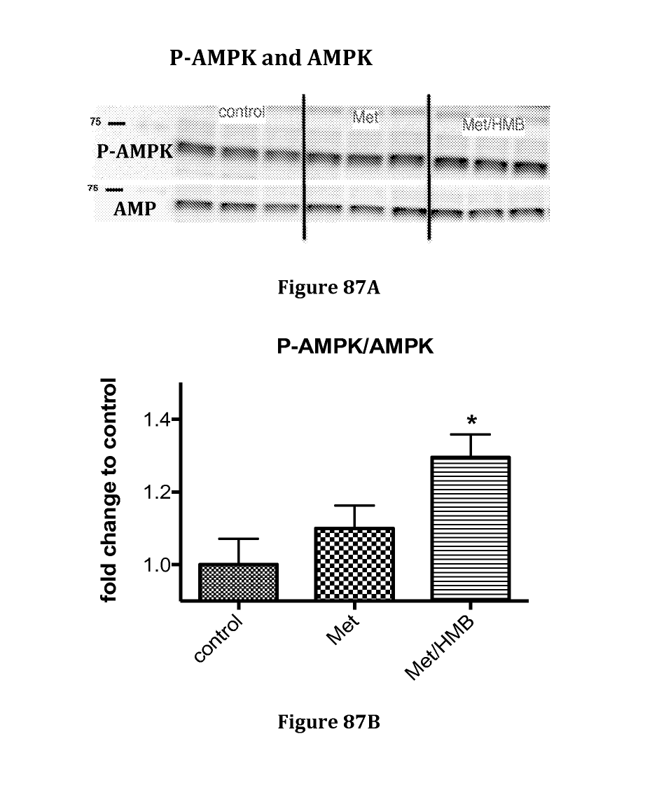

To test whether the synergistic effect of leucine or HMB, a metabolite of leucine, with metformin involve activation of the AMPK/Sirt1 pathway, Sirt1 activity was measured in the Met-Leu groups. The leucine/metformin combination induced a significant 46% increase in Sirt1 activity in adipocytes (FIG. 85). Similarly, the Met-HMB combination induced a significant 30% increase in the P-AMPK/AMPK ratio (FIG. 87A and FIG. 87B). To test whether the effects were dependent on AMPK activation, we measured fatty acid oxidation, an outcome measure of AMPK stimulation, in the presence and absence of an AMPK inhibitor. The palmitate-induced fatty acid oxidation was increased by 24-hour Met-Leu treatment compared to control (FIG. 86); however, the addition of the AMPK inhibitor Compound C completely blocked this increase (FIG. 86), indicating AMPK dependence.

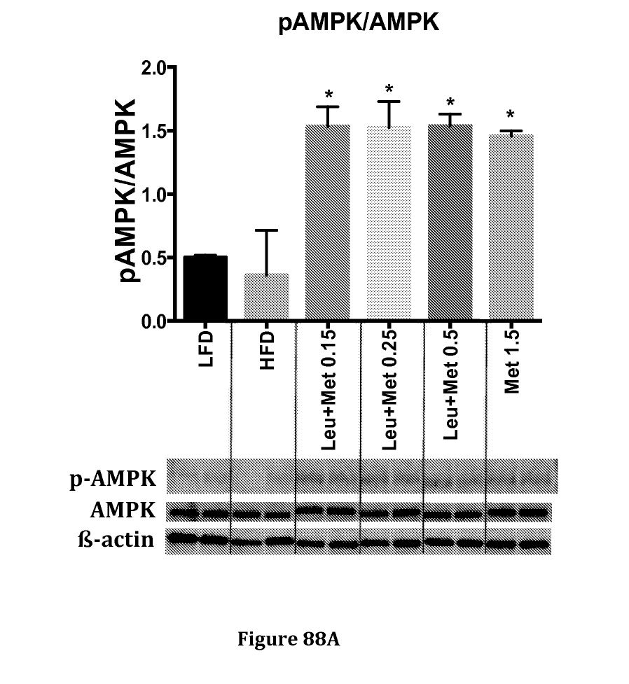

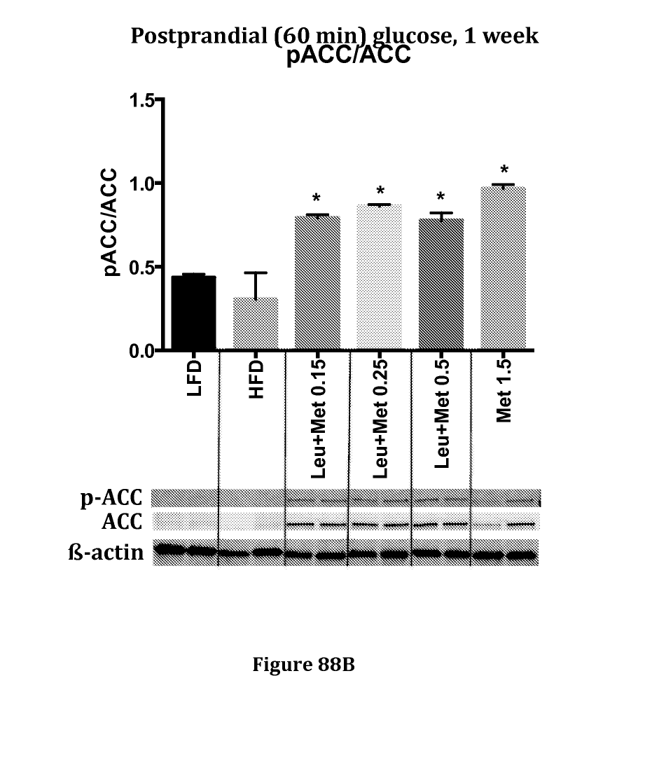

Consistent with the in vitro data, the P-AMPK/AMPK ratio as well as the P-ACC/ACC ratio, an AMPK downstream target, was up to three-fold up-regulated in muscle of the DIO− mice by all Leu-Met combinations comparable to full-dose metformin (FIG. 88A and FIG. 88B).

These data demonstrate that metformin synergizes with leucine to improve hyperglycemia and insulin resistance in a mouse model of obesity and insulin resistance. This synergy results in dose reduction of metformin up to 83% with no loss of efficacy in this model.

The metformin concentrations in this study were based on literature values of full therapeutic dose (300 mg/kg BW) and very low dose (50 mg/kg BW) metformin studies in mice. The very low dose was shown to have no independent effect on insulin, HOMA_{IR} and ITT. Similarly, the resveratrol concentration was chosen to be lower than other comparable low-dose mice studies (50 to 100 mg Res/kg diet) and did not exert independent effects on insulin sensitivity markers in our previous work. The leucine level used in the treatment groups was based on data demonstrating that this is sufficient to achieve an increase from normal fasting leucine (˜0.1 mM) to plasma levels of ˜0.5 mM. This dose was demonstrated in vitro to be necessary to activate Sirt1 signaling. However, as shown in FIGS. 78A-FIG. 78B and FIG. 79A-FIG. 79B, this concentration had no independent effect on GTT or ITT.

As described herein, leucine and HMB allosterically activate Sirt1 directly in a cell-free system, reducing the Km for NAD⁺ and thereby mimicking the effects of caloric restriction. This also allows other activators to stimulate Sirt1 at lower concentrations. For example, a low dose of Resveratrol (12.5 mg/kg diet) combined with either leucine or HMB produced significant improvement in adiposity, insulin sensitivity and inflammatory markers in diabetic mice, which was modulated by increases in Sirt1 and AMPK activity. These effects were superior to an almost 20 times higher dose of Resveratrol alone. However, this synergy is not exclusive to Resveratrol, and was found with other AMPK/Sirt1 activators (including metformin).

Most of metformin's glucose-lowering effects are mediated through the activation of the AMPK/Sirt1 axis, a key regulatory point of energy metabolism. A substantial body of evidence points to the mild inhibition of the mitochondrial chain complex 1 which results in increased AMP and reduced ATP, thereby activating AMPK. However, Ouyang et al (Ouyang et al., “Metformin activates AMP kinase through inhibition of AMP deaminase,” J Biol Chem 286, 1-11 Jan. 7, 2011) suggested that inhibition of complex 1 is inconsistent with metformin stimulation of fatty acid oxidation, and instead proposed metformin inhibition of AMP deaminase as the mechanism of increased AMPK activation. Evidence also supports an additional AMPK-independent mechanism, as glucose production was inhibited in mice lacking hepatic AMPK.

Since there is a bidirectional interaction between AMPK and Sirt1, metformin's effects also appear to be mediated by activation of Sirt1. Caton et al (Caton et al., “Metformin opposes impaired AMPK and SIRT1 function and deleterious changes in core clock protein expression in white adipose tissue of genetically-obese db/db mice,” Diabetes Obes Metab 13, 1097-1104 Dec. 13, 2011) demonstrated that metformin inhibits gluconeogenic gene expression by AMPK dependent and independent modulation of Sirt1 and GCN5 in the liver of diabetic mice and HepG2 cells. Sirt1 activity was increased as a consequence of an AMPK-mediated increase of nicotinamide phosphoribosyltransferase and an associated rise in NAD+/NADH ratio, as Compound C, an AMPK inhibitor blocked the metformin effects on P-AMPK, NAMPT, NAD+/NADH ratio and Sirt1 activity. In contrast, Compound C did not inhibit metformin-induced increases in Sirt1 protein levels, indicating an AMPK-independent stimulation. Although all these studies focused on metformin's action in liver, similar effects on AMPK/Sirt1 were also shown in peripheral tissues. For example, metformin reversed the impaired AMPK-Sirt signaling in white adipose tissue of db/db mice, enhanced the insulin-stimulated glucose uptake in muscle cells in a AMPK dependent manner, and inhibited the hyperglycemia-induced down regulation of the Sirt1/LKB1/AMPK pathway in retinal cells. The data presented in the current invention also indicate that AMPK and Sirt1 modulate the observed effects of the combination of metformin with leucine. Met-Leu effects on palmitate-induced fat oxidation were completely blocked by the addition of Compound C (FIG. 86) and both, P-AMPK/AMPK ratio and Sirt1 activity was up regulated in adipocytes (FIG. 85 and FIG. 87A-FIG. 87B). Moreover, P-AMPK/AMPK and the downstream target P-ACC/ACC were increased in muscle of the DIO-mice by the combinations as well as by full-dose Met (FIG. 88A and FIG. 88B).

In contrast with the salutary effects of leucine described here, others have proposed that elevated blood branched-chain amino acids, including leucine, may contribute to the development of insulin resistance and diabetes. However, this rise appears to be secondary to aberrant amino acid metabolism, specifically a down-regulation of the branched-chain α-ketoacid dehydrogenase (BCKD), the rate-limiting enzyme of BCAA catabolism, in liver and adipose tissue. Thus, it is likely that increased plasma BCAA is a consequence rather than a cause of insulin resistance. In support of this concept, data from a number of studies show that diets high in BCAA restore aberrant BCKD activity and improve glucose and insulin sensitivity.

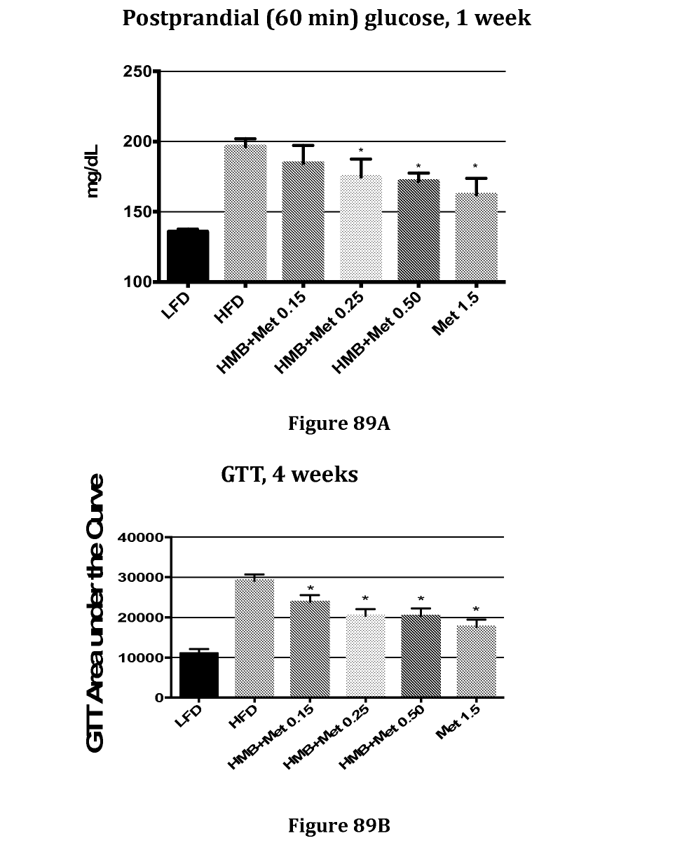

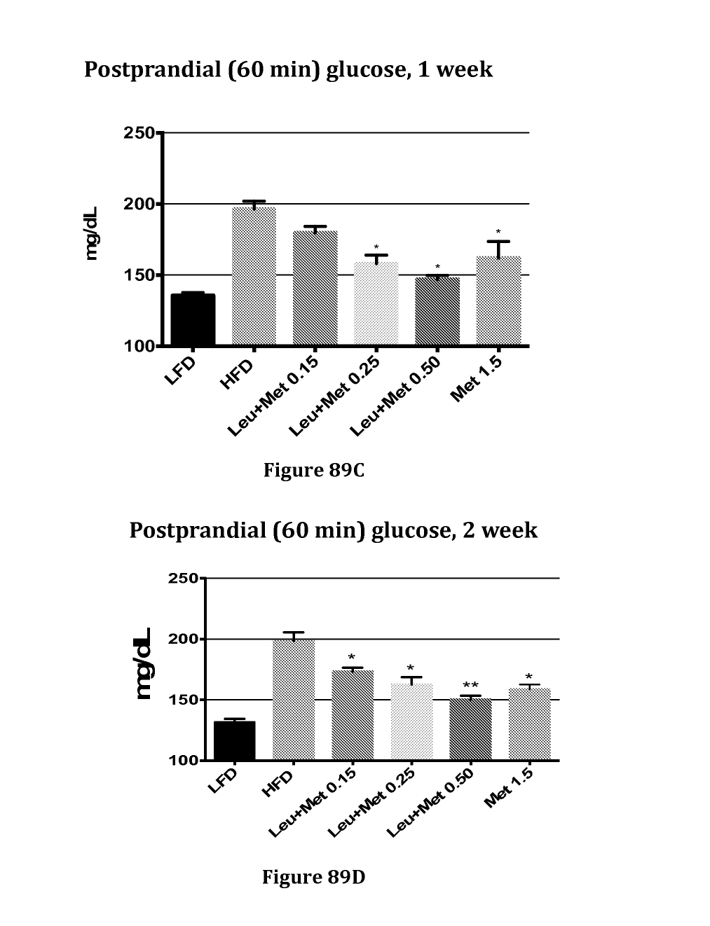

We previously demonstrated the short-term efficacy (2 weeks) of a Resv/HMB/metformin combination on insulin sensitivity in db/db mice. Therefore, the present study was designed to examine more comprehensively the long-term efficacy (6 weeks) of a leucine/metformin combination and to assess the necessity of resveratrol in this combination. Although leucine and its metabolite, HMB, exerted comparable effects in our previous in vitro studies, we also conducted a parallel animal study to compare HMB-metformin combinations with leucine-metformin combinations. FIG. 89A-FIG. 89D summarized the data for the HMB study. Although both, leucine and HMB exhibited qualitatively comparable outcomes for most parameters, the Leu-Met combinations exerted quantitatively superior effects on glycemic control in diet-induced obese mice (FIG. 89A-FIG. 89D). The HMB-Met 0.5 combination resulted in a reduction in postprandial glucose level comparable to full-dose metformin, the Leu-Met 0.5 effects were superior to full dose Met. Similarly, there was a significant greater reduction in the GTT in the Leu-Met 0.5 group compared to Met 1.5 than by HMB-Met 0.5 (FIG. 80B and FIG. 89A-FIG. 89D).

Our early observations of an interaction between leucine and resveratrol in activating Sirt1 suggested that resveratrol may be a necessary component in a leucine-metformin based combination for glycemic control. However, comparison of data from Study 1 and Study 2 demonstrate that resveratrol may be not a required component, as comparable effects were found in the presence (study 1, Table 3) and absence (study 2, Table 4) of resveratrol (FIGS. 78A-FIG. 78B, FIG. 79A-FIG. 79B, FIG. 80A-FIG. 80B, FIG. 81A-FIG. 81B).

The Met-Leu combination used in this study enabled a dose reduction of metformin up to 83% with no loss of efficacy (Leu-Met 0.25) and up to 66% (Leu-Met 0.5) with improved efficacy in some of the parameters; these are calculated to be equivalent to human doses of 250-500 mg/day. Most adverse effects of metformin are dose-dependent and are observed with reaching therapeutic doses (≥1500 mg/day). The most prominent symptoms are gastrointestinal such as nausea, vomiting, diarrhea and abdominal pain, which occur in up to 30% of patients and may lead to compliance issues and/or drug discontinuation. In addition, the presence of co-morbidities, particularly renal impairment, may limit or contraindicate the use of metformin at standard doses. Therefore, a combination that enables substantial metformin dose-reduction of metformin with no loss of glycemic control may be associated with better tolerability and may provide an alternative to people intolerant to full-dose metformin.

### Summary:

HFD for 6 weeks induced pronounced fasting and post-prandial hyperglycemia and hyperinsulinemia, which were not significantly affected by the addition of Leu (24 g/kg diet) with or without Res (12.5 mg/kg diet). However, adding sub-therapeutic levels of Met that exert no independent effects (0.05-0.25 g/kg diet) to Leu-Res resulted in dose-responsive reductions in fasting and post-prandial glucose (p<0.01) which were evident within 7 days of treatment and sustained for six weeks until sacrifice. Met (0.25 g/kg)-Res-Leu produced a comparable reduction in fasting glucose (30 mg/dL) to a standard therapeutic Met dose (1.5 g/kg diet; ˜300 mg/kg BW), as well as comparable glucose response to an insulin tolerance test and a significantly greater reduction in area under the curve (AUC) in glucose tolerance tests (GTT) (p<0.0001). This study was then repeated without Res, with comparable results. Leu-Met (0.25 g/kg) reduced blood glucose levels by 30 mg/dL (p<0.001), and the area under the GTT curve by 16% (p<0.001), similar to effects of therapeutic levels of Met (1.5 g/kg), while the Leu-Met (0.5 g/kg diet) resulted in greater improvements in glucose (43 mg/dL) GTT AUC (25%; p<0.001). These effects were accompanied with an increase in P-AMPK/AMPK ratio in muscle tissue, consistent with in vitro data in 3T3L1 adipocytes showing involvement of AMPK/Sirt1 pathway. Thus, adding Leu to Met enables a dose reduction of 66% with improved efficacy and of 83% with comparable efficacy to standard metformin, and Res is not a necessary component for this synergy.

Low dose metformin combined with leucine significantly improved glucose control, enabling substantial dose reduction of metformin (83% for comparable and 66% for greater efficacy than full-dose metformin). These effects are mediated, at least in part, by the activation of the AMPK/Sirt1 pathway. The addition of resveratrol does not improve these effects and is therefore unnecessary in the formulation for treatment of diabetes.

### Example 13: Effect of Leucine/Metformin/Nicotinic Acid on Insulin Sensitivity, Triglyceride, LDL, HDL, and Cholesterol Levels, and Atherosclerotic Plaque Size in Diabetic Mice

To assess the efficacy of the subject compounds, mice are administered the subject compounds comprising (a) nicotinic acid, (b) leucine, and (c) metformin as described herein, wherein the composition comprises free leucine and a sub-therapeutic amount of nicotinic acid and metformin. The composition provides a method of potentiating therapeutic effects for concomitant treatment of diabetes and hyperlipidemia, for example, increasing insulin sensitivity, lowering the triglyceride, LDL and cholesterol levels in a mouse after administration of the composition to the mouse

LDL receptor knockout (LDLRKO) mice are obtained from Jackson Laboratories (Bar Harbor, Me.), and housed in groups under room temperature in a humidity-controlled environment with a regular light and dark cycle. The mice are randomized into six treatment groups (as described below) with 10 animals/group. The mice are provided free access to an atherogenic western diet (WD) containing 0.21% cholesterol (by weight) and 40% calories from fat and water for 4 weeks prior to treatment (RD #12079B, Research Diets, Inc. New Brunswick, N.J.). For treatment, mice are provided combinations with leucine/metformin/nicotinic acid compositions as described in Table 5.

TABLE 5
Treatment of LDL receptor knockout mice (LDLRKO) with
combinations of leucine/metformin/nicotinic acid composition.

| Group No. | Leucine | Metformin | Nicotinic Acid |
|---|---|---|---|
| 1 | Standard Diet | 0 | 0 |
| 2 | Standard Diet | 1.5 g/kg diet | 0 |
| 3 | Standard Diet | 0 | 1000 mg/kg diet |
| 4 | Standard + 24 g | 0.25 g/kg diet | 0 |
| | Leucine/kg diet | | |
| 5 | Standard + 24 g | 0 | 50 mg/kg diet |
| | Leucine/kg diet | | |
| 6 | Standard + 24 g | 0.25 g/kg diet | 50 mg/kg diet |
| | Leucine/kg diet | | |

Blood samples of the mice in all groups are obtained from tails of the mice at following four and eight weeks of administration. The plasma levels of glucose, insulin, triglyceride, total cholesterol and cholesterol esters are measured. Following treatment, food is removed for four hours and the animals are euthanized.

Blood is collected into EDTA-coated tubes to analyze plasma glucose, insulin and lipid profiles. Plasma total cholesterol (TC, Pointe Scientific, Canton, Mich.), free cholesterol (FC, Wako, Richmond, Va.) and triglyceride (TG, Wako, Richmond, Va.) concentrations are measured using enzymatic assays according to manufacturer's instructions. Cholesterol ester (CE) is calculated as the difference between TC and FC.

Insulin Tolerance Test (ITT):

Insulin tolerance tests are performed at 2 pm on day 7, and following 4 and 8 weeks of treatment. The mice are injected with insulin (1.0 U/kg) in ˜0.1 ml 0.9% NaCl intraperitoneally. A drop of blood (5 microliter) is taken from the cut tail vein before the injection of insulin and after 15, 30, 45, and 60 min for the determination of blood glucose. Change in blood glucose over the linear portion of the response curve is then calculated.

Insulin:

Blood Insulin in serum is measured via Insulin ELISA kit from Millipore (Cat. # EZRMI-13K).

Glucose:

Blood glucose is measured via Glucose Assay Kit from Cayman (Cat. # EZRMI-13K).

To assess atherosclerosis, the circulatory system is perfused following euthanasia with phosphate-buffered saline (PBS) before removing the heart and aorta. The upper one-third of the heart is dissected and embedded in Optimal Cutting Temperature Compound (Sakura Tissue-Tek, Torrance, Calif.), frozen, and stored at −80° C. Blocks are serially cut at 8 μm intervals and stained with hematoxylin and 0.5% Oil Red O (Sigma-Aldrich) to evaluate aortic sinus atherosclerotic intimal area. Atherosclerotic lesion area and Oil Red O positive area are quantified using Image-Pro Plus software (Media Cybemetics, Bethesda, Md.). Whole aorta (from sinotubular junction to iliac bifurcate) is dissected and fixed in 10% formalin, and the adventitia is cleaned. Aortas are opened along the longitudinal axis and pinned onto black silicon elastomer (Rubber-Cal, Santa Ana, Calif.) for the quantification of atherosclerotic lesion area. The percentage of total aortic surface covered with atherosclerotic lesions is quantified by Image-Pro Plus software (Media Cybemetics, Bethesda, Md.) and will be used to determine the total lesion area.

To assess macrophage infiltration, Sections of aortic sinus are immuno-stained with rat monoclonal antibody against macrophage-specific CD68 (Clone FA11, 1:75, AbD Serotec, Raleigh, N.C.) followed by staining with alkaline phosphatase-conjugated mouse anti-rat (for CD68, 1:50) secondary antibodies (Jackson ImmunoResearch laboratories, West Grove, Pa.). Control slides contain no primary antibody. The CD68-positive areas are analyzed using Image-Pro Plus software (Media Cybemetics, Bethesda, Md.).

### Results:

### Treatment of Diabetes:

Mice in Group 1 (standard leucine diet, no metformin, no nicotinic acid) are expected to exhibit diabetes symptoms such as high plasma insulin and HOMA_{IR} index, which would indicate that the standard leucine diet alone has no effect on treatment of diabetes. Mice in Group 2 (standard leucine diet, high dose metformin, no nicotinic acid) are expected to have significantly lower plasma insulin and HOMA_{IR} index, suggesting that metformin can be effective in treating diabetes as described herein. Mice in Group 3 (standard leucine diet, high dose of nicotinic acid, no metformin) and mice in Group 5 (high leucine diet, low dose of nicotinic acid, no metformin) are expected to exhibit high plasma insulin and HOMA_{IR} index as with mice in Group 1, which would be consistent with the expectation that nicotinic acid alone or in combination with leucine does not treat diabetes. Mice in Group 4 (high leucine diet, low dose metformin, and no nicotinic acid) are expected to exhibit high plasma insulin HOMA_{IR} index, as mice in Group 2. However, providing mice in Group 6 (high dose leucine, low dose metformin, low dose nicotinic acid) are expected to exhibit significantly lowered plasma insulin and HOMA_{IR} index, as observed in mice in Group 2 and Group 4. It is expected that combining leucine and metformin can have synergistic effect on plasma insulin sensitivity and this synergistic effect can be augmented by high dose of leucine with low dose metformin, thereby reducing the dosage of metformin to sub-therapeutic level that is still capable of treating diabetes. The beneficial effects of combining metformin with leucine are not expected to be diminished by combination with nicotinic acid.

It is also expected that in the results of the insulin tolerance test; animals in Group 1, Group 3 and Group 5 exhibit no significant changes in blood glucose in response to the insulin challenge. In contrast, animals in Group 2, Group 4 and Group 6 are expected to exhibit significantly reduced blood glucose over the 30 minute linear portion of the response curve.

### Treatment of Hyperlipidemia:

To assess effects of on treating hyperlipidemia, mice are provided with the atherogenic diet with standard leucine for four weeks and dietary treatment for four weeks. At the final (eight week) time point, mice in Group 1 receiving only the atherogenic diet with standard leucine but no treatment can exhibit symptoms of hyperlipidemia such as profound elevations in plasma cholesterol, cholesterol esters and trigylcyerides.

Mice in Group 1 (standard leucine diet, no metformin, no nicotinic acid), 2 (standard leucine diet, high dose metformin, no nicotinic acid) and 4b (high leucine diet, low dose metformin, and no nicotinic acid) are expected to exhibit high plasma cholesterol, cholesterol esters and trigylcyerides, which is consistent with the expectation that leucine and/or metformin alone are insufficient to treat hyperlipidemia. However, mice in Group 3 (standard leucine diet, high dose nicotinic acid, no metformin) and Group 5 (high leucine diet, no metformin, low dose nicotinic acid) are expected to have significantly lowered plasma cholesterol, cholesterol esters and trigylcyerides, which would that nicotinic acid can be effective in treating hyperlipidemia. Mice in Group 6 (high leucine diet, low dose metformin and low dose nicotinic acid) are expected to exhibit reduced plasma cholesterol, cholesterol esters and trigylcyerides, as with mice in Group 3 and Group 5.

Mice in Groups 3, 5, and 6 are expected to exhibit a decrease atherosclerotic lesion size and decrease in aortic macrophage infiltration relative to the control fed with the western diet alone (Group 1). The effect of decreased atherosclerotic lesion size can be observed in Oil Red O stained aortic histology slides and quantified as total lesion area and lipid area. The effect of decreased aortic macrophage infiltration can be indicated by CD68-positive area in representative histology slides and can be quantified as % CD68 positive area in the lesion

This data may suggest that leucine and nicotinic acid have synergistic effect in treating hyperlipidemia as described herein, and a higher dose of leucine can significantly lower the required dose of nicotinic acid to sub-therapeutic level that is capable of treating hyperlipidemia effectively, as with the case of a high nicotinic acid treatment in Group 3.

### Example 14: Effect of Leucine/Guanidine Derivatives on Glucose Utilization and Fat Acid Oxidation

The use of a composition comprising leucine and guanidine derivatives as described herein was investigated. The levels of leucine (0.5 mM) and galegine (5.0 μM) were selected such that these levels (a) are attainable by sub-therapeutic dose oral administration and (b) had no independent effect on the variables studied.

Cell Culture:

C2C12 and 3T3-L1 preadipocytes (American Type Culture Collection) were plated at a density of 8000 cells/cm² (10 cm² dish) and grown in Dulbecco's modified eagle's medium (DMEM) containing 10% fetal bovine serum (FBS), and antibiotics (growth medium) at 37° C. in 5% CO₂. Confluent 3T3-L1 preadipocytes were induced to differentiate with a standard differentiation medium consisting of DMEM medium supplemented with 10% FBS, 250 nM dexamethasone, 0.5 mM 3-Isobutyl-1-methylxanthine (IBMX) and 1% penicillin-streptomycin. Preadipocytes were maintained in this differentiation medium for 3 days and subsequently cultured in growth medium. Cultures were re-fed every 2-3 days to allow >90% cells to reach fully differentiation before conducting chemical treatment. For differentiation of C2C12 cells, cells were grown to 100% confluence, transferred to differentiation medium (DMEM with 2% horse serum and 1% penicillin-streptomycin), and fed with fresh differentiation medium every day until myotubes were fully formed (3 days).

Seahorse Fatty Acid Oxidation:

The palmitate-stimulated oxygen consumption rate was measured with the XF 24 analyzer (Seahorse Bioscience, Billerica, Mass., USA) as previously described (6-8) with slight modifications. Cells were seeded at 40,000 cells per well and differentiated as described above. Media was then changed and the cells treated for 24 hours with the indicated treatments, washed twice with non-buffered carbonate-free pH 7.4 low glucose (2.5 mM) DMEM containing carnitine (0.5 mM), equilibrated with 550 μL of the same media in a non-CO₂ incubator for 45 minutes, and then inserted into the instrument for 15 minutes of further equilibration, followed by O₂ consumption measurement. Three successive baseline measures at five-minute intervals were taken prior to injection of palmitate (200 μM final concentration). Four successive 5-minute measurements of O₂ consumption were then conducted, followed by 10 minute re-equilibration and another 3-4 5-minute measurements. This measurement pattern was then repeated over a 4-6 hour period. Data for each sample were normalized to the pre-palmitate injection baseline for that sample and expressed as % change from that baseline. Pre-palmitate injection values were 371±14 pmol O₂/minute for myotubes and 193±11 pmol O₂/minute for adipocytes. The area under of the curve of O₂ consumption change from baseline for each sample was then calculated and used for subsequent analysis.

Glucose Utilization:

In the absence of a fatty acid source and oxidative metabolism, glycolysis and subsequent lactate production results in extracellular acidification, which was also measured using a Seahorse Bioscience XF24 analyzer. Cells were prepared and equilibrated similar to the methods described above for fatty acid oxidation, with the exclusion of carnitine from the medium. Following instrument equilibration and three baseline measurements, glucose was injected to a final concentration of 10 mM in each well. Measurements were taken as described above utilizing the sensors for extracellular acidification rather than O₂ consumption. Insulin (final concentration of 5 nM) was added to some wells as a positive control and to some treatment and control wells to assess treatment effects on insulin response. Data for each sample were normalized to the pre-glucose injection baseline for that sample and expressed as % change from that baseline. The area under of the curve of extracellular acidification change from baseline for each sample was the calculated and used for subsequent analysis.

Statistics:

Data were analyzed via one-way analysis of variance and least significant difference test was used to separate significantly different group means.

### Results:

### Glucose Utilization:

Galegine:

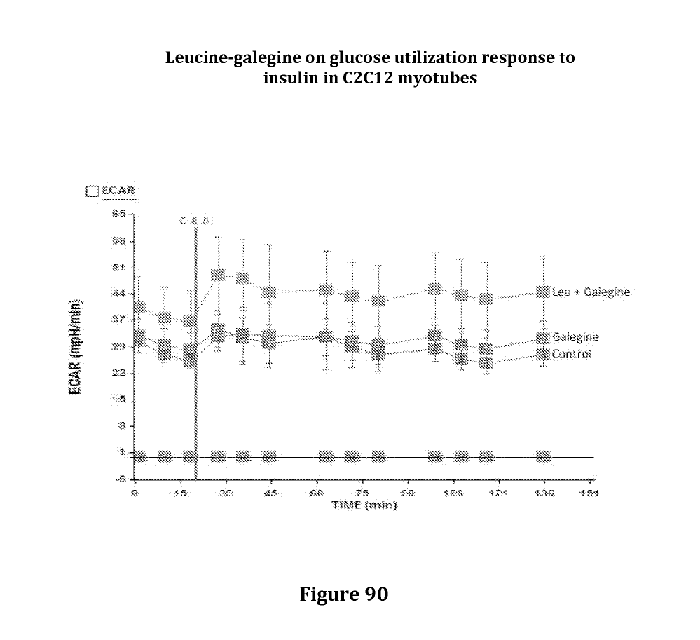

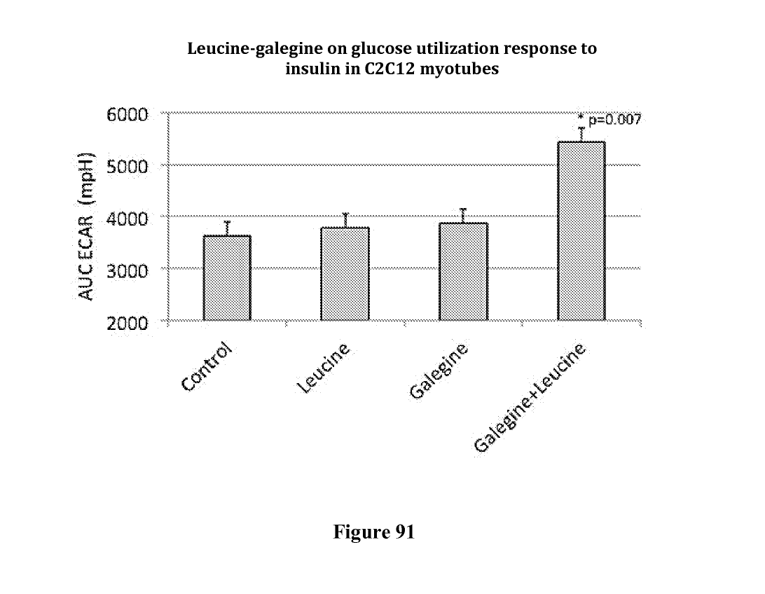

Dose response curves demonstrated no independent effect of galegine on basal or insulin-stimulated glucose utilization at concentrations up to 500 μM, while published literature shows no effect below ˜100 μM (Mooney et al., “Mechanisms underlying the metabolic actions of galegine that contribute to weight loss in mice,” Br. J Pharmacol 153, 1669-1677 Feb. 25, 2008). Accordingly, the synergistic combination of leucine 0.5 and 5.0 μM galegine was studied. Administration of 5 μM galegine or 0.5 mM leucine alone exerted no effect on glucose utilization; however, administering the combination of galegine and leucine synergistically enhanced glucose utilization, resulting in a 50% increase in insulin-stimulated glucose utilization (p=0.007; FIG. 90 and FIG. 91). Reducing the galegine concentration to 0.5 μM still resulted in a modest (13%) stimulation of glucose utilization when combined with leucine (p<0.05).

Guanidine:

Guanidine dose-responsively stimulated glucose utilization, with a no-effect threshold of 0.1-1 μM. Accordingly, leucine synergy was evaluated at a guanidine concentration of 10 nM. The combination of leucine and guanidine at this concentration did not stimulate basal or insulin-stimulated glucose utilization, indicating no synergy.

Dimethylguanidine:

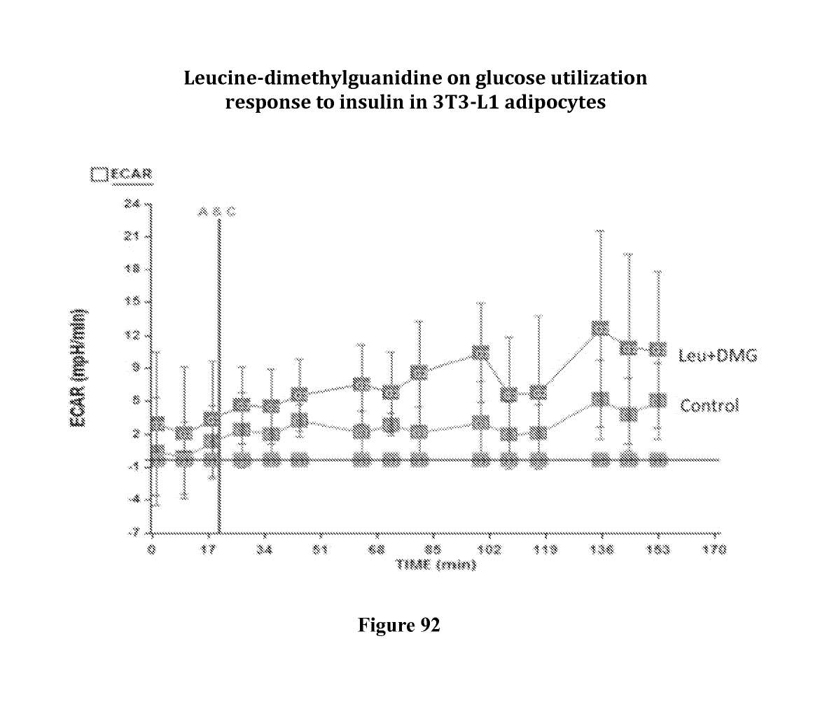

Dimethylguanidine stimulation of glucose utilization exhibited a no-effect threshold of 100 μM-1 mM. Accordingly, leucine synergy was evaluated at 10 μM. The leucine and dimethylguanidine combination did not significantly affect basal glucose utilization. However, the combination elicited a marked augmentation of insulin-stimulated glucose utilization (144%; p=0.05; FIG. 92).

### Fat Oxidation:

Galegine:

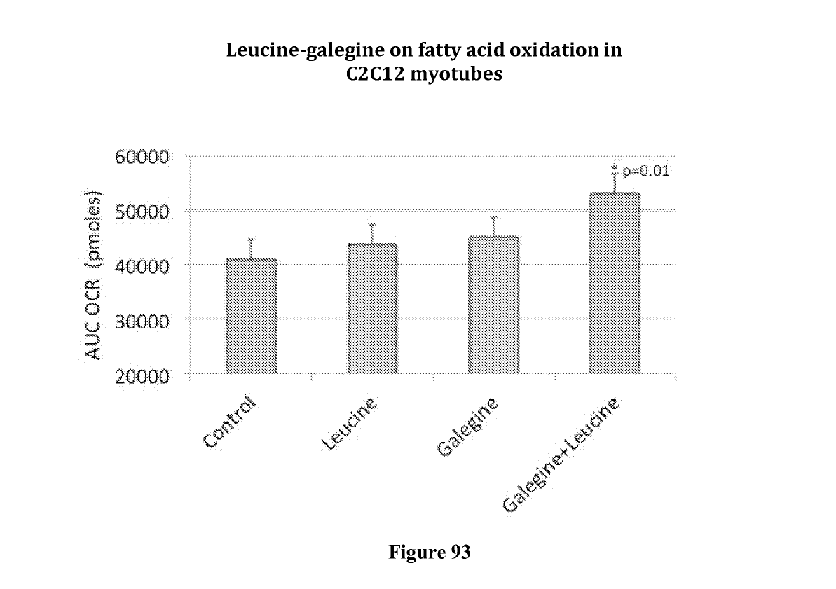

Administering the combination of galegine and leucine synergistically enhanced stimulation of fat oxidation by 40% (FIG. 93).

These data demonstrate that leucine can augment the effects of guanidine compounds, including galegine and dimethylguanidine, but not guanidine, on insulin-mediated glucose disposal.

### Example 15 Effect of the Combination of Leucine/Metformin/Nicotinic Acid on Insulin Sensitivity, Triglyceride, LDL, HDL, and Cholesterol Levels, and Atherosclerotic Plaque Size in Diabetic Mice

To assess the efficacy of the subject compounds, a total of 30 male mice were administered the subject compounds comprising (a) nicotinic acid, (b) leucine, and (c) metformin as described herein, wherein the composition comprises free leucine and a sub-therapeutic amount of nicotinic acid and metformin (e.g. see the amount of respective agents in Table 6). The data demonstrate potentiation of therapeutic effects for concomitant treatment of diabetes and hyperlipidemia. In particular, increasing insulin sensitivity, lowering the triglyceride, LDL and/or cholesterol levels in a mouse after administration of the composition to the test mice were observed.

LDL receptor knockout (Ldlr−/−; Mus musculus) male mice were obtained from Jackson Laboratories (Bar Harbor, Me.), and housed in groups under room temperature in a humidity-controlled environment with a regular light and dark cycle. The mice were approximately 20-25 g weighed to the nearest 0.1 g, and approximately 6 weeks old. The mice were healthy and had never been used in other experimental procedures. The Ldlr−/− mice were selected since these mice have been used as a model to mimic human atherosclerosis. The dietary/oral route of exposure was selected since it is generally known as the route for possible treatment of atherosclerosis. The mice were randomized into three treatment groups (as described below) with 10 animals/group. The mice were provided free access to an atherogenic western diet (WD) containing 0.21% cholesterol (by weight) and 40% calories from fat and water for 4 weeks prior to treatment (RD #12079B, Research Diets, Inc. New Brunswick, N.J.). For treatment, mice were provided combinations with leucine/metformin/nicotinic acid compositions as described in Table 6, for a period of 8 weeks.

TABLE 6
Treatment of LDL receptor knockout mice (Ldlr−/−; Mus musculus)
with combinations of leucine/metformin/nicotinic acid composition.

| Group No. | Leucine | Metformin | Nicotinic Acid |
|---|---|---|---|
| 1 | Atherogenic | 0 | 0 |
| | Diet | | |
| 2 | Atherogenic | 0 | 1000 mg/kg diet |
| | Diet | | |
| 3 | Atherogenic | 0.5 g/kg diet | 50 mg/kg diet |
| | Diet + | | |
| | 24 g Leucine/ | | |
| | kg diet | | |

In general, blood was collected into EDTA-coated tubes to analyze plasma glucose, insulin and lipid profiles. Plasma total cholesterol (TC, Pointe Scientific, Canton, Mich.), free cholesterol (FC, Wako, Richmond, Va.) and triglyceride (TG, Wako, Richmond, Va.) concentrations were measured using enzymatic assays according to manufacturer's instructions.

In general, mice were humanely euthanized and necropsied for observation at the end of their in-life portion (e.g. Day 56) or at the day of moribund sacrifice.

Blood samples of the mice in all groups were obtained for lipid profile at day 1 and Day 56. The serum fraction of the blood was prepared from blood collected in tubes with no anticoagulant. Approximately 70 μl of serum was prepared from blood collected in tubes with no anticoagulant for clinical analysis. The plasma levels of triglyceride, cholesterol, and LDL cholesterol were measured administration.

Approximately 100 μl of serum on Day 28 and approximately 250 μl of serum on Day 56 were collected and frozen at −20±4° C. and analyzed for insulin as one (1) batch at the end of the study by ELISA kit (#10-1247-10; Mercodia). Following treatment, food was removed for four hours and the animals are euthanized.

Insulin:

Blood Insulin in serum was measured via Insulin ELISA kit (#10-1248-10; Mercodia).

Glucose:

Blood glucose was measured via a One Touch Ultra® Blood Glucose Monitoring System.

In general, to assess atherosclerosis, the circulatory system was perfused following euthanasia with phosphate-buffered saline (PBS) before removing the heart and aorta. The upper one-third of the heart was dissected and embedded in Optimal Cutting Temperature Compound (Sakura Tissue-Tek, Torrance, Calif.), frozen, and stored at −80° C. Blocks were serially cut at 8 μm intervals and stained with hematoxylin and 0.5% Oil Red O (Sigma-Aldrich) to evaluate aortic sinus atherosclerotic intimal area. Atherosclerotic lesion area and Oil Red O positive area were quantified using Image-Pro Plus software (Media Cybemetics, Bethesda, Md.). Whole aorta (from sinotubular junction to iliac bifurcate) was dissected and fixed in 10% formalin, and the adventitia was cleaned. Aortas were opened along the longitudinal axis and pinned onto black silicon elastomer (Rubber-Cal, Santa Ana, Calif.) for the quantification of atherosclerotic lesion area. The percentage of total aortic surface covered with atherosclerotic lesions was quantified by Image-Pro Plus software (Media Cybemetics, Bethesda, Md.) and was used to determine the total lesion area.

In general, to assess macrophage infiltration, sections of aortic sinus were immuno-stained with rat monoclonal antibody against macrophage-specific CD68 (Clone FA11, 1:75, AbD Serotec, Raleigh, N.C.) followed by staining with alkaline phosphatase-conjugated mouse anti-rat (for CD68, 1:50) secondary antibodies (Jackson ImmunoResearch laboratories, West Grove, Pa.). Control slides contain no primary antibody. The CD68-positive areas were analyzed using Image-Pro Plus software (Media Cybemetics, Bethesda, Md.).

### Results:

### Treatment of Diabetes:

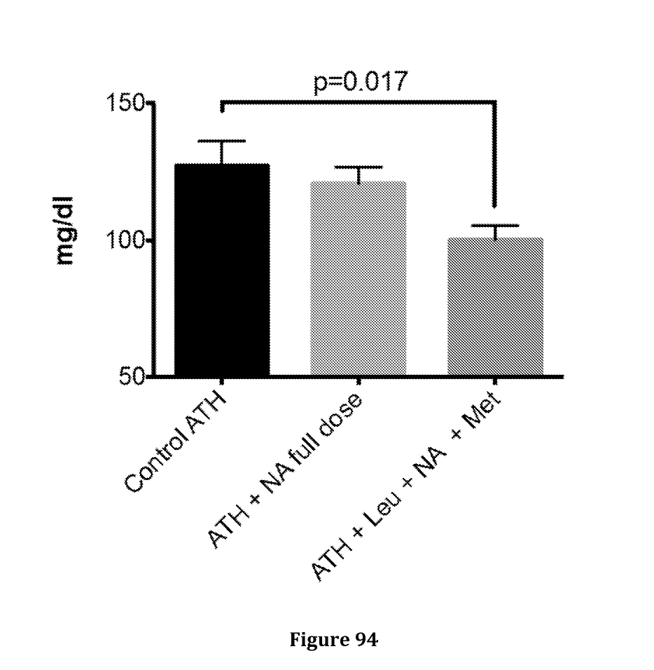

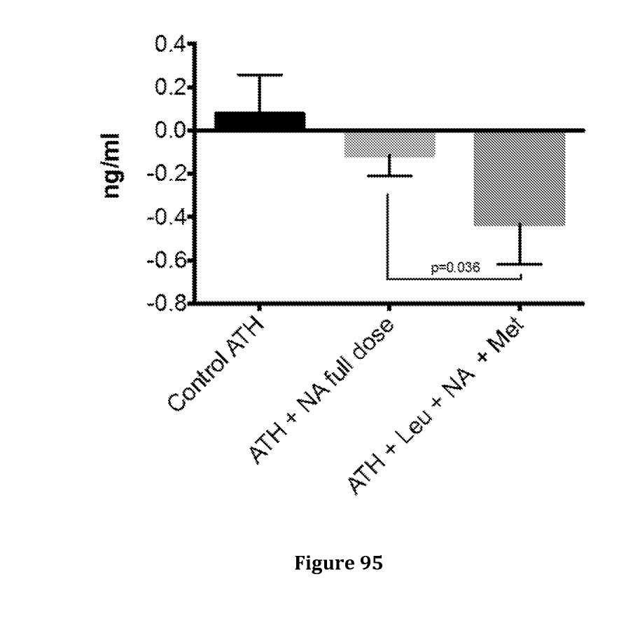

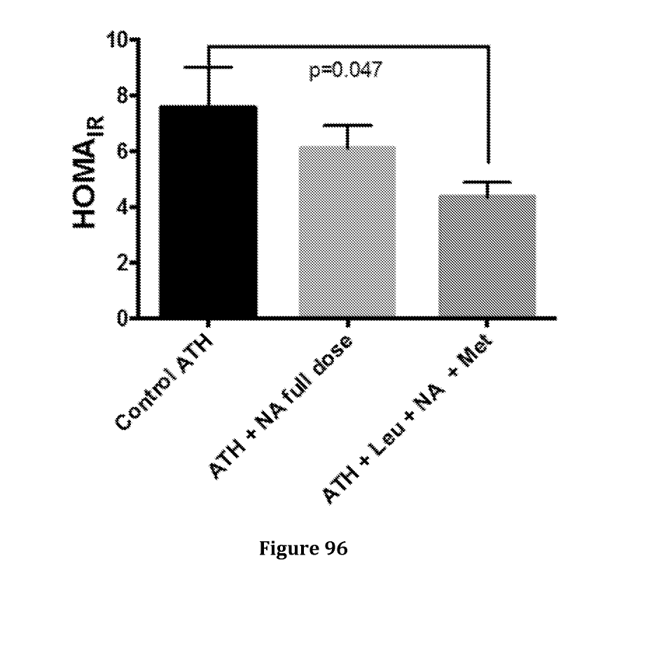

Although the Ldlr−/− mouse is not a model of diabetes per se, providing mice with supplemental leucine, low dose metformin, and low dose nicotinic acid still exert modest effects on glycemic control in these mice. Following 8 weeks of treatment, mice in Group 2 treated with high dose nicotinic acid (1000 mg/kg diet) exhibited no change in blood glucose (FIG. 94) or plasma insulin (FIG. 95) compared to mice in Group 1 on the Atherogenic Diet (no metformin, no nicotinic acid). In contrast, providing mice with supplemental leucine (24 g/kg diet), low dose metformin (0.5 g/kg diet), and low dose nicotinic acid (50 mg/kg diet) in Group 3 resulted in a significant decrease in fasting blood glucose concentration (FIG. 94). This was accompanied by a significant reduction in fasting insulin concentration (FIG. 95), suggesting improvement in insulin sensitivity. This increase in insulin sensitivity was confirmed via calculation of Homeostatic Assessment of Insulin Resistance (HOMAir), which was significantly reduced with the addition of leucine, low-dose metformin and low-dose nicotinic acid (FIG. 96).

### Treatment of Hyperlipidemia:

To assess effects of on treating hyperlipidemia, mice were provided with the atherogenic diet with standard leucine for four weeks and dietary treatment for eight weeks. At the final (Day 56) time point, mice in Group 1 receiving only the Atherogenic diet with standard leucine but no treatment exhibited symptoms of hyperlipidemia such as profound elevations in plasma LDL cholesterol, cholesterol and triglycerides.

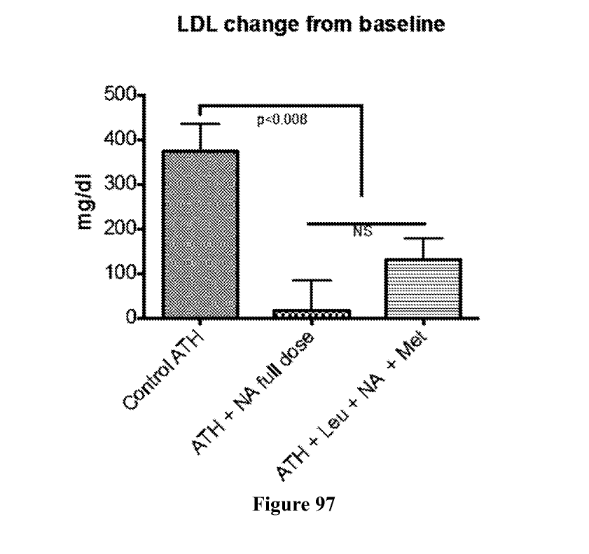

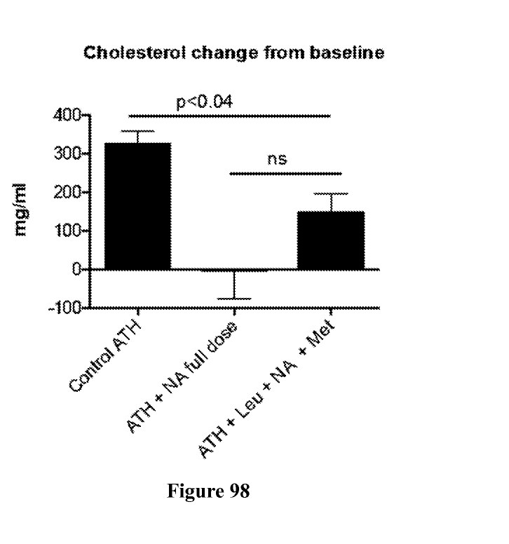

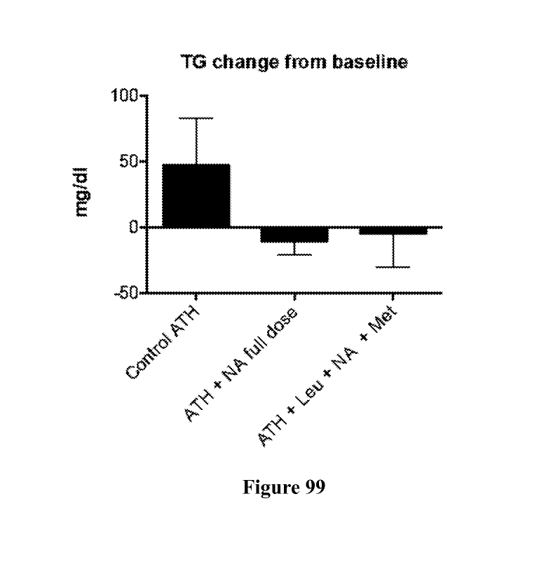

FIG. 97, FIG. 98 and FIG. 99 show the effect of the disclosed composition on plasma level LDL cholesterol, cholesterol and triglycerides at Day 56 compared to baseline level. Mice in Group 1 (Atherogenic Diet, no metformin, no nicotinic acid) exhibited high plasma LDL cholesterol (FIG. 97), cholesterol (FIG. 98) and triglycerides (FIG. 99) which indicated that the level of leucine found in the standard diet is not sufficient to treat hyperlipidemia. Mice in Group 2 (Atherogenic Diet, no metformin, high dose nicotinic acid (1000 mg/kg diet)) exhibited significantly lower plasma LDL cholesterol (FIG. 97), cholesterol (FIG. 98) and triglycerides (FIG. 99), which was consistent with the expectation that nicotinic acid can treat hyperlipidemia. Mice in Group 3 (Atherogenic Diet, leucine (24 g/kg diet), low dose metformin (0.5 g/kg diet), and low dose nicotinic acid (50 mg/kg diet)) exhibited significantly low plasma LDL cholesterol (FIG. 97), cholesterol (FIG. 98) and triglycerides (FIG. 99), which was consistent with the expectation that leucine and/or metformin in combination with nicotinic acid can effectively treat hyperlipidemia.

This data suggested that leucine and nicotinic acid have synergistic effect in treating hyperlipidemia as described herein, and a higher dose of leucine can significantly lower the required dose of nicotinic acid to sub-therapeutic level that was capable of treating hyperlipidemia effectively, as with the case of a high nicotinic acid treatment in Group 3.

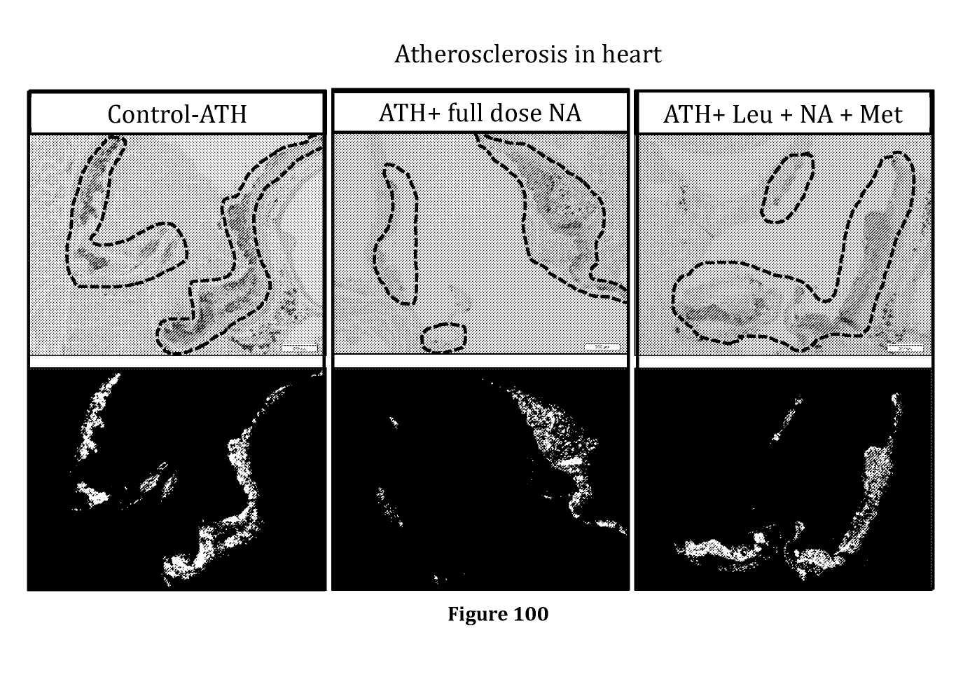

Atherosclerosis was assessed, histological images (FIG. 100) shows that mice in Group 1 (Atherogenic Diet only) exhibited atherosclerosis; such symptom was reduced by adding nicotinic acid to the diet, as shown in Group 2 mice (Atherogenic Diet+full dose nicotinic acid). Adding leucine and low dose metformin, along with low dose nicotinic acid to the diet also reduced atherosclerosis significantly, as shown in Group 3 mice (Atherogenic Diet+dose nicotinic acid+leucine+low dose metformin). The histological data of hearts is consistent with the expectation that leucine and metformin have synergistic effect in treating hyperlipidemia as described herein, and adding the combination of leucine and metformin to the diet can lower the dose of nicotinic acids required to exert effectiveness in treating hyperlipidemia.

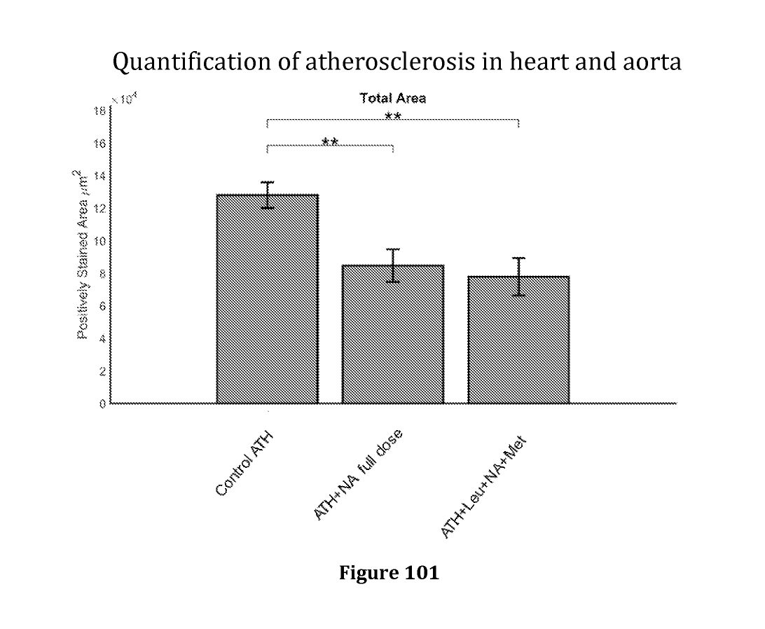

Atherosclerosis was quantified by measuring the positively stained area of heart and aorta collected from mice in Group 1, Group 2 and Group 3. In consistent with the histological images, Group 1 mice (Atherogenic Diet only) exhibited larger area of Oil Red O staining, which visualized atherosclerosis lesions (FIG. 101). Mice in Group 2 and Group 3 had significantly less Oil Red O staining area in the heart and aorta, suggesting that nicotinic acid is effective in treating hyperlipidemia, and the effect is enhanced by adding low dose metformin and leucine such that a lower dose or subtherapeutic dose of nicotinic acid is required to achieve similar effect.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A composition comprising:
(a) at least 250 mg of leucine and/or at least 25 mg of one or more leucine metabolites, wherein the one or more leucine metabolites are selected from the group consisting of keto-isocaproic acid (KIC), alpha-hydroxy-isocaproic acid, and HMB; and
(b) at least 1 mg of one or more agents selected from the group consisting of nicotinic acid, nicotinamide riboside, and nicotinic acid metabolite; and
(c) at least 0.1 mg of one or more anti-diabetic agents.

2. The composition of claim 1, wherein the weight percentage of component (a) is between about 80%-98% of the total composition, wherein the weight percentage of component (b) is between about 1%-5% of the total composition, and wherein the weight percentage of component (c) is between about 1%-15% of the total composition.

3.-6. (canceled)

7. The composition of claim 1, wherein the amount of leucine and/or one or more leucine metabolites is less than 1 g.

8. (canceled)

9. The composition of claim 1, wherein the amount of the one or more agents selected from the group consisting of nicotinic acid, nicotinamide riboside, and nicotinic acid metabolite is less than 250 mg.

10. The composition of claim 1, wherein the amount of the one or more agents selected from the group consisting of nicotinic acid, nicotinamide riboside, and nicotinic acid metabolite is less than 1 g.

11. (canceled)

12. The composition of claim 1, wherein the amount of the one or more anti-diabetic agents is between 0.1-2550 mg.

13. The composition of claim 1, wherein the amount of the one or more anti-diabetic agents is between 0.1-500 mg.

14. The composition of claim 1, wherein the amount of the one or more anti-diabetic agents is between 1-200 mg.

15.-18. (canceled)

19. The composition of claim 1, wherein the one or more anti-diabetic agent is selected from the group consisting of biguanide, metformin, phenformin, buformin, galegine, dimethylguanidine, guanide, thiazolidinedione, rosiglitazone, meglitinides, alpha glucosidase inhibitors, sulfonylureas, incretins, ergot alkaloids, DPP inhibitors, and any combination thereof.

20. (canceled)

21. The composition of claim 1, wherein the component (a) in the composition is leucine, wherein the component (b) in the composition is nicotinic acid, and wherein the component (c) in the composition is metformin.

22. (canceled)

23. (canceled)

24. The composition of claim 1, wherein the component (c) in the composition is an analog of metformin, or a precursor of metformin.

25. The composition of claim 1, wherein the molar ratio of component (a) to component (b) in said composition is greater than about 20.

26. The composition of claim 1, wherein the molar ratio of component (a) to component (c) in said composition is greater than about 20.

27. The composition of claim 1, wherein the composition is substantially free of nicotinamide.

28. (canceled)

29. The composition of claim 1, wherein the composition is substantially free of nicotinic acid metabolites.

30. The composition of claim 1, wherein the composition is substantially free of each of nicotinyl CoA, nicotinuric acid, nicotinate mononucleotide, nicotinate adenine dinucleotide, and nicotinamide adenine dinucleotide.

31. The composition of claim 1, wherein the composition is substantially free of each of alanine, glycine, glutamic acid, and proline.

32. The composition of claim 1, wherein the composition is substantially free of each amino acid selected from the group consisting of alanine, arginine, asparagine, aspartic acid, cysteine, glutamic acid, glutamine, glycine, histidine, lysine, methionine, phenylalanine, proline, serine, threonine, tryptophan, valine, isoleucine and tyrosine.

33.-48. (canceled)

49. The composition of claim 1, wherein of the leucine and/or one or more leucine metabolites is in a free form or salt form.

50. The composition of claim 1, wherein the composition is formulated for oral administration.

51. The composition of claim 1, wherein the composition is a tablet, a capsule, a pill, a granule, an emulsion, a gel, a plurality of beads encapsulated in a capsule, a powder, a suspension, a liquid, a semi-liquid, a semi-solid, a syrup, a slurry or a chewable form.

52. (canceled)

53. The composition of claim 1, wherein component (a) and component (b) and component (c) are separately packaged or mixed.

54. (canceled)

55. The composition of claim 1, further comprising one or more therapeutic agents that is capable of lowering lipid accumulation, and/or increasing fat oxidation, and/or increasing insulin sensitivity, and/or increasing glucose utilization.

56. The composition of claim 55, wherein the one or more therapeutic agents is selected from the group consisting of HMG-CoA inhibitor, fibrate, bile acid sequestrant, ezetimibe, lomitapide, phytosterols, CETP antagonist, orlistat, and any combination thereof.

57. A method of reducing atherosclerotic plaque size or lipid accumulation in a subject in need thereof, comprising administering to said subject a dose of a composition of claim 1.

58. (canceled)

59. A method of increasing insulin sensitivity, fat oxidation and/or glucose utilization in a subject in need thereof, comprising administering to said subject the composition of claim 1 to effect an increasing the insulin sensitivity in the subject.

60. (canceled)

61. (canceled)

62. A method of treating diabetes and/or hyperlipidemia comprising administering to the subject a composition of claim 1.

63. (canceled)

64. A kit comprising a multi-day supply of unit dosages of the composition of claim 1 and instructions directing the administration of said multi-day supply over a period of multiple days.

65.-89. (canceled)
